(19) United States
(12) Patent Application Publication
(10) Pub. No.: US 2024/0417475 A1
(43) Pub. Date: Dec. 19, 2024

(54) BISPECIFIC CD16A BINDERS

(71) Applicant: Affimed GmbH, Mannheim (DE)

(72) Inventors: Joachim KOCH, Mannheim (DE); Jens PAHL, Mannheim (DE); Thorsten ROSS, Mannheim (DE); Jana-Julia SIEGLER, Mannheim (DE); Holger DULAT, Mannheim (DE)

(21) Appl. No.: 18/707,020

(22) PCT Filed: Nov. 3, 2022
(86) PCT No.: PCT/IB2022/060614

§ 371 Date: May 2, 2024

Related U.S. Application Data
(60) Provisional application No. 63/369,602, filed on Jul. 27, 2022.

(30) Foreign Application Priority Data
Nov. 3, 2021 (EP) 21206329.1
Dec. 10, 2021 (EP) 21213774.9
Jul. 27, 2022 (EP) 22187329.2

(51) Int. Cl. C07K 16/28; A61K 39/00; A61P 35/00

(52) U.S. Cl. CPC C07K 16/283; A61P 35/00; C07K 16/28; A61K 2039/505; C07K 2317/31; C07K 2317/622; C07K 2317/732; C07K 2317/92

(57) ABSTRACT

The present invention relates to a bispecific antibody construct comprising (a) a first binding domain (A), which is capable of specifically binding to a first target (A) that is CD16A on the surface of an immune effector cell, wherein the first binding domain comprises: (i) a VL region comprising CDR-L1 as depicted in SEQ ID NO: 4, a CDR-L2 as depicted in SEQ ID NO: 5, and a CDR-L3 as depicted in SEQ ID NO: 6; and (ii) a VH region as depicted in SEQ ID NO: 7; and (b) a second binding domain (B), which is capable of specifically binding to a second target (B′) that is an antigen on the surface of a target cell, wherein the second target (B′) is FOLR1. The present invention also relates to related nucleic acid molecules, vectors, host cells, methods of producing the antibody constructs, pharmaceutical compositions, medical uses, and kits.

### CROSS-REFERENCE TO RELATED APPLICATIONS

The present application claims the priority benefit of U.S. Provisional Patent Application No. 63/369,602, filed Jul. 27, 2022; European Patent Application Nos. EP 22187329.2, filed Jul. 27, 2022; EP 21213774.9, filed Dec. 10, 2021, and EP 21206329.1, filed Nov. 3, 2021, each of which is incorporated herein by reference in its entirety.

### REFERENCE TO SEQUENCE LISTING SUBMITTED ELECTRONICALLY VIA EFS-WEB

The content of the electronically submitted sequence listing (Name: 5146_002PC01_Seglisting_ST26.xml; Size: 197,996 bytes; and Date of Creation: Nov. 3, 2022) is herein incorporated by reference in its entirety.

### FIELD OF THE DISCLOSURE

The present invention relates to a bispecific antibody construct comprising a first binding domain (A), which is capable of specifically binding to a first target (A′) that is CD16A on the surface of an immune effector cell; and a second binding domain (B), which is capable of specifically binding to a second target (B′) that is an antigen on the surface of a target cell, wherein said second target (B′) is FOLR1. The present invention also relates to related nucleic acid molecules, vectors, host cells, methods of producing the antibody constructs, pharmaceutical compositions, medical uses, and kits.

### BACKGROUND

Natural killer cells are cytotoxic, IFN-γ and TNF-α producing innate lymphoid cells that are considered the first line of defense against virus-infected cells and cancer cells (Cerwenka and Lanier 2001). The cytotoxic potential of NK cells can be utilized in cancer immunotherapy by redirecting NK cell lysis to tumor cells and stimulating the activating receptor CD16A, also known as FcγRIIIA, expressed on the surface of NK cells. NK cells are equipped with multiple activating and inhibitory receptors on their surface jointly regulating NK cell activation and triggering of effector functions. Several of these receptors play a pivotal role for NK cell mediated recognition, killing of cancer cells and cytokine secretion. CD16A activation promotes NK cell proliferation and memory-like cytotoxicity against cancer cells (Pahl et al 2018 Cancer Immunol Res; 6(5), 517-27; DOI: 10.1158/2326-6066.CIR-17-0550). Upon ligation, CD16A induces a potent series of signals resulting in cytokine production and cytotoxic effector activity via antibody dependent cellular cytotoxicity (ADCC). In this respect, tumor-specific monoclonal antibodies (mABs), such as rituximab, that recognize tumor-selective antigens, such as CD20, on the surface of tumor cells are described to induce NK cell-mediated anti-tumor activity via ADCC (Wang et al., Front. Immunol., 2015, 6:368, doi: 10.3389/fimmu.2015.00368).

However, also directing NK cells for tumor cell lysis using bi- or multispecific antibodies is considered a potent immunotherapeutic approach and offers opportunities for increasing specificity, potency, and utilizing novel mechanisms of action. Bispecific antibodies consisting of one arm which binds CD16A and another which binds a tumor-associated antigen (e.g., CD19) have been developed (Kellner et al 2011 Cancer Lett. 303(2): 128-139). WO 2006/125668 and Reusch et al, MABS, 2014, 6:3:728-739 describe an antigen-binding protein—a bispecific tandem diabody—for engagement of CD16A and its use for natural killer (NK) cell therapy. The cytotoxic activity of NK cells can be enhanced by increasing the avidity through multivalent binding to CD16A, e.g., using constructs with bivalent binding to CD16A (WO2019/198051 Affimed GmbH).

Activation-induced down-regulation/shedding of CD16 on activated NK cells is caused by proteolytic cleavage of its extracellular portion by A disintegrin and metalloproteinase (ADAM17) (Romee at al., Blood, 2013, 121 (18):3599-3608), or membrane type 6 matrix metalloproteinase (MMP25) (Peruzi et al., J. Immunol., 2013, 191:955-957). However, CD16 shedding does not immediately recover, suggesting that once NK cells are activated and CD16 is down-regulated, their capacity for ADCC is impaired for several days (Goodier et al., Front. Immunol., 2016, 7:384). Moreover, ADAM17 mediated CD16 shedding is also described to limit the efficacy of rituximab or trastuzumab antibody therapies that involve ADCC (Romee at al., Blood, 2013, 121 (18):3599-3608). Hence, down-regulation of CD16 expression on activated immune effector cells may limit or regulate their activity and ADCC-mediated cytotoxicity. On the other side, the usage of CD16 inhibitors and NK cells transfected to express a non-cleavable form of CD16 revealed that CD16 shedding upon NK cell activation may be considered important for the detachment of NK cells from opsonized target cells, thereby sustaining NK cell survival and increasing serial engagement of target cells (Srpan et al., J. Cell. Biol., 2018, 217(9):3267-3283).

In particular, in solid tumors with limited presence of NK cells, CD16 shedding would be a great disadvantage for the ability of an effector cell to kill multiple tumor cell targets. Overexpression of Folate Receptor 1 (FOLR1) by a large number of solid tumors including ovarian, breast (particularly TNBC), renal, lung, colorectal (CRC), kidney (particularly ccRCC), pancreatic (particularly PDAC), endometrial and brain has been described in the art (Hartmann et al., Int J Cancer 2007, 121(5): 938-42; Yang et al., Human Cancer Biology 2007, 13(9): 2557-2567; Scaranti et al., Nat Rev Clin Oncol. 2020, 17(6): 349-359). Hence, due to its high expression in solid tumors, FOLR1 may be an attractive therapeutic target for the development of anti-cancer agents. A range of FOLR1-targeting approaches, including folic acid derivatives, folate drug-conjugates and small molecules, vaccines, terpolymer-based nanocapsules, T-cell therapies and monoclonal antibodies, have been developed for clinical application for both imaging and therapeutic purposes (Cheung et al., Oncotarget 2016, 7(32): 52553-52574, Bellotti et al., biomedicines 2021, 9, 1275).

However, there is still a need in the art for the provision of highly efficient therapeutic approaches for treating FOLR1 expressing cancer disease, such as anti-CD16A bispecific antibody constructs for use in immuno-oncology therapies to induce immune effector cell activation by binding to CD16A, thereby allowing for high cytokine production and long-lasting target cell killing by the activated NK cells. The present invention addresses this need as indicated herein.

### SUMMARY

The present invention is based at least partly on the surprising finding that a bispecific antibody construct comprising a high-affinity anti-CD16A first binding domain and a second binding domain for an antigen on the surface of a target cell, wherein said target antigen is FOLR1, can efficiently activate and redirect immune effector cells for ADCC, thereby avoiding CD16A shedding and immediate inactivation of the engaged effector cells. Specifically, the present inventors surprisingly observed that the high-affinity anti-CD16A binding domain comprised by the antibody construct of the present invention strongly stabilizes CD16A expression on NK effector cells after activation when compared to low-affinity anti-CD16A binding domains, despite the presence of target cells. In this respect, immune effector cells activated by the bispecific antibody construct of the present invention show high cytotoxic activity and induce target cell lysis without activation-induced CD16A shedding. This is particularly beneficial for the treatment of solid tumors with limited presence of NK cells, since CD16 shedding would be a great disadvantage for the ability of an effector cell to kill multiple tumor cell targets. As shown in Examples 1, 2 and 12, the bispecific antibodies of the present invention comprising a specific CD16A binding domain (also named CD16a1 anti-CD16A effector domain or CD16a1 domain herein) show a higher affinity to human CD16A when compared to other CD16A binding domains (see FIGS. 1, 2 and 16A-16C and Tables 3 and 15). Moreover, as shown in Examples 5, the bispecific antibodies of the present invention comprising a specific CD16A binding domain show a significant CD16 shedding inhibition effect on stimulated NK cells when compared to shedding inhibition induced by other CD16A binding domains (see FIGS. 5A-5C and 6A-6C). Nonetheless, as demonstrated in Example 4, the bispecific antibodies of the present invention comprising a specific CD16A binding domain also show high lysis potential with EC₅₀ values in the low picomolar concentration range against target cells (see FIGS. 4 and 17) and show a low target cell-independent activation (see FIGS. 7 and 18A-18B). In sum, antibody constructs comprising the high-affinity anti-CD16A binding domain of the invention have surprisingly a high cytotoxic activity although these constructs prevent CD16A shedding upon NK cell activation.

The antibody constructs of the present invention can thus be useful for tumor therapy, in particular solid tumors, because they are not only capable of activating NK cells via high-affinity binding of CD16A receptor, but also achieve long-lasting activation of NK cells without loss of CD16A. Thus, the bispecific antibody constructs of the present invention lead to a high affinity binding of effector NK cells via CD16A and effective killing of target cells by cell mediated cytotoxicity, thereby allowing for sustained effector cell activation. The antibody constructs of the present invention can thus be useful for efficiently targeting various cancer diseases, in particular solid tumors overexpressing FOLR1 on the cell surface, and must be considered superior to antibody constructs comprising low-affinity CD16A binding domains (e.g. the CD16a2 or CD16a4 anti-CD16A effector domain disclosed herein). The antibody constructs of the present invention can thus be useful for treating solid tumors such as ovarian, breast (particularly TNBC), renal, lung, colorectal (CRC), kidney (particularly ccRCC), pancreatic (particularly PDAC), endometrial and brain tumors.

In particular, the present invention relates to a bispecific antibody construct comprising (a) a first binding domain (A), which is capable of specifically binding to a first target (A′) that is CD16A on the surface of an immune effector cell, wherein the first binding domain comprises: (i) a VL region comprising CDR-L1 as depicted in SEQ ID NO: 4, a CDR-L2 as depicted in SEQ ID NO: 5, and a CDR-L3 as depicted in SEQ ID NO: 6; and (ii) a VH region as depicted in SEQ ID NO: 7 or SEQ ID NO: 94; and (b) a second binding domain (B), which is capable of specifically binding to a second target (B′) that is an antigen on the surface of a target cell, wherein the second target (B′) is FOLR1.

The present invention also relates to a nucleic acid molecule comprising a sequence encoding an antibody construct of the invention.

The present invention also relates to a vector comprising a nucleic acid molecule of the invention.

The present invention also relates to a host cell comprising a nucleic acid molecule of the invention or a vector of the invention.

The present invention also relates to a method of producing an antibody construct of the invention, said method comprising culturing a host cell of the invention under conditions allowing the expression of the antibody construct of the invention and optionally recovering the produced antibody construct from the culture.

The present invention also relates to a pharmaceutical composition comprising an antibody construct of the invention, or produced by the method of the invention.

The present invention also relates to an antibody construct of the invention for use in therapy.

The present invention also relates to a method of treatment or amelioration of a proliferative disease, a tumorous disease, a viral disease or an immunological disorder, comprising the step of administering to a subject in need thereof the antibody construct of the invention, or produced by the method of the invention.

The present invention also relates to a kit comprising an antibody construct of the invention, or produced by the method of the invention, a nucleic acid molecule of the invention, a vector of the invention, and/or a host cell of the invention.

### BRIEF DESCRIPTION OF THE DRAWINGS

FIG. 1: Detection of CD16A interaction with CD16A binding domains. CD123×CD16A ICE binding to human CD16A^{158V}, CD16A^{158F} and cynomolgus CD16 was measured by SPR using a multivalent multi-cycle kinetic set up at 37° C. (n=3; ²) n=1) with biotin captured recombinant CD16A^{158V}, CD16A^{158F} and cynomolgus CD16 (ligand) and scFv-IgAb_268 (CD16a1×CD123-1), scFv-IgAb_148 (CD16a2×CD123-1), scFv-IgAb_264 (CD16a1×CD123-2) (analyte). Affinity and kinetic parameters were evaluated for interaction with human CD16A and cynomolgus CD16 using a 1:1 Binding model. All molecules show high interaction to human CD16A as well as to cynomolgus CD16 with apparent affinities in the range of K_{D} 0.195 nM-2.48 nM.

FIG. 2: Binding of CD123×CD16A constructs to cell lines expressing human CD16A. Binding of antibody constructs to huCD16A-transfected CHO cells measured by flow cytometry, depicting the median fluorescent intensity (MFI) of titrated scFv-IgAb_268 (CD123-1×CD16a1, black dot), scFv-IgAb_148 (CD123-1×CD16a2, black triangle) and a negative control molecule (scFv-IgAb_139, CD123×RSV, grey dot) relative to overall CD16 expression as detected by the anti-human CD16 antibody clone 3G8. Exp. No. RHU 066

FIG. 3: Cell surface retention of anti-CD123 antibodies on NK cells. Enriched primary human NK cells were preloaded with 100 μg/mL scFv-IgAb_268 (CD123-1×CD16a1), Fc-enhanced anti-CD123 IgG1 (IgAb_338), or scFv-IgAb_148 (CD123-1×CD16a2) on ice, washed, and then incubated at 37° C. for the indicated time periods in an excess volume of complete RPMI 1640 medium to allow dissociation and to prevent re-association. Residual antibodies at each time point were determined by flow cytometry, and median fluorescence intensity (MFI) values at time-point 0 were taken to be 100%, and the percentages of remaining antibody were analysed and plotted by non-linear regression using GraphPad Prism.

FIG. 4: ADCC against CD123+ EOL-1 cells by anti-CD123 antibodies. Concentration-dependent induction of tumor cell lysis by bispecific antibody constructs scFv-IgAb_268 (CD123-1×CD16a1), scFv-IgAb_267 (CD123-2×CD16a1), scFv-IgAb_265 (CD123-1×CD16a2) and scFv-IgAb_264 (CD123-2×CD16a2) using NK cells as effector cells in 4 h calcein-release cytotoxicity assays. Calcein-labeled EOL-1 target cells were incubated with human NK cells as effector cells at an E:T ratio of 5:1 in the presence of serial dilutions of the respective antibodies in duplicates. Target and effector cells without (w/o) antibodies were used as a negative control (ctrl), and killing of targets by effectors in the absence of antibodies were determined in quadruplicate on each plate. The experiments were carried out in biological duplicates, and one representative resulting diagram is shown. All four CD123×CD16A scFv-IgAb constructs induced NK cell-dependent lysis against EOL-1 cells at similar maximal efficacy in the low picomolar concentration range.

FIGS. 5A-5C: Shedding inhibition of CD16A on activated NK cells. Enriched primary human NK cells were preloaded with 100, 10, 1 μg/mL CD123-1×CD16a1 scFv-IgAb_268 (FIG. 5A), CD123-1×CD16a2 scFv-IgAb_148 (FIG. 5B) or Fc-enhanced anti-CD123 IgG1 (IgAb_338) (FIG. 5C) on ice, washed, and then stimulated with PMA/Ionomycin (PMA Iono) at 37° C. for 4 h. CD16 expression was determined by flow cytometry and analysed using FlowJo Software. Exp. No.: NSC 026.

FIGS. 6A-6C: Shedding inhibition of CD16A on activated NK cells. Enriched primary human NK cells were preloaded with 100, 10, 1 μg/mL CD123-1×CD16a1 scFv-IgAb_268 (FIG. 6A), CD123-1×CD16a2 scFv-IgAb_148 (FIG. 6B) or Fc-enhanced anti-CD123 IgG1 (IgAb_338) (FIG. 6C) on ice, washed, and then stimulated with PMA/Ionomycin (PMA Iono) at 37° C. for 4 h. Median fluorescence intensity (MFI) values of CD16 expression were determined by flow cytometry and analysed using FlowJo Software. After subtracting the fluorescence intensity values of the cells stained with the secondary reagents alone, the MFI values were plotted using the GraphPad Prism software. Statistical significance was assessed using paired Students t-test. ns: p>0.05; * p<0.05.

FIG. 7: Target cell-independent activation of NK cells by anti-CD123 antibodies. Enriched primary human NK cells were preloaded with 100, 10, 1 μg/mL CD123-1×CD16a1 scFv-IgAb_268 (A), CD123-1×CD16a2 scFv-IgAb_148 (B) or Fc-enhanced anti-CD123 IgG1 (IgAb_338) (C) on ice, washed, and then stimulated with PMA Ionomycin at 37° C. for 4 h. CD16 expression was determined by flow cytometry and analysed using FlowJo Software. Exp. No.: NSC 026

FIG. 8: Target cell-dependent activation of NK cells by anti-CD123 antibodies. CMFDA-labeled EOL-1 cells were co-cultured with buffy coat-, derived allogeneic NK cells (5×10⁴) at 1:1 cell ratio for 24 h in the presence titrated antibodies (CD123a1×CD16a1 scFv-IgAb_268, CD123-2×CD16a1 scFv-IgAb_267, CD123-1×CD16a2 scFv-IgAb_265, CD123-1×CD16a2 scFv-IgAb_264) or control molecules (scFv-IgAb_239, scFv-IgAb_238) starting at a concentration of 50 μg/mL followed by six 10-fold serial dilutions. Up-regulation of the NK cell activation marker CD137 on NK cells was analysed by flow cytometry. All four CD123×CD16A scFv-IgAb constructs specifically induced the up-regulation of the activation marker CD137, wherein antibody constructs constituting the anti-CD16A CD16a1 domain reached a peak in the percentages of CD137+NK cells at 0.05 μg/mL, followed by decreasing percentages of CD137+NK cells at higher concentrations. Non-CD123-targeting RSV×CD16A control antibody constructs failed to induce NK cell activation in response to EOL-1 cells.

FIGS. 9A-9B: Binding of CD123×CD16A constructs to CD123+ and CD123− tumor cell lines. Binding of four CD123×CD16A antibody constructs CD123a1×CD16a1 scFv-IgAb_268, CD123-2×CD16a1 scFv-IgAb_267, CD123-1×CD16a2 scFv-IgAb_265, CD123-1×CD16a2 scFv-IgAb 264) to CD123+ EOL-1 cells, CD123− A-431 cells and CD123− Karpas-299 cells were analysed by flow cytometry. All four CD123×CD16A scFv-IgAb constructs showed comparable binding to CD123+ EOL-1 cells (FIG. 9A). In contrast, to CD123− A431 cells, scFv-IgAb_268 comprising the CD123-1 and the CD16a1 binding domains showed lowest potential for unspecific binding (FIG. 9B). Overall, scFv-IgAb_268 showed least unspecific binding to CD123-A-431 cells, followed by scFv-IgAb_265, followed by scFv-IgAb_267, followed by scFv-IgAb_264 across different antibody construct batches tested.

FIG. 10: Structure information and description of a preferred bispecific antibody construct.

FIG. 11: Structure information and description of a preferred bispecific antibody construct.

FIGS. 12A-12B: Depletion of CD123+ primary leukemic blasts from peripheral blood and bone marrow of AML patients by anti-CD123 antibodies. The percentage of depletion primary leukemic blasts of PB and BM of AML patients after 24-hours co-culture with buffy coat-derived allogeneic NK cells at an 1:1 effector to target (E:T) cell ratio in the presence of titrated AFM28 (CD123×CD16A scFv-IgAb_268, black squares), Fc-enhanced anti-CD123 IgG talacotuzumab (IgAb_338, gray triangles), a negative control molecule (RSV×CD16A scFv-IgAb_239, black circles) without antibody addition (black crosses). (FIG. 12A) Representative dose-response data from the AML 2 sample. (FIG. 12B) Data of four AML PB and BM samples at 0.002 μg/mL (10 pM) antibody constructs (single measurements).

FIGS. 13A-13B: ADCC against CD123+ BMMCs from patients diagnosed with AML and HR-MDS cells by anti-CD123 antibodies. Concentration-dependent induction of tumor cell lysis by bispecific antibody construct scFv-IgAb_268 (CD123-1×CD16a1), using allogeneic healthy donor NK cells as effector cells in 24 h cytotoxicity assays. Bone marrow samples from patients diagnosed with (FIG. 13A) AML or (FIG. 13B) high-risk MDS containing CD123⁺ target cells were incubated with human NK cells as effector cells at an E:T ratio of 1:1 in the presence of serial dilutions of the antibody in triplicates. Killing of targets by effectors in the absence of antibodies (0 pM) were determined in triplicate in each sample. The experiments were carried out in biological triplicates (AML) and biological duplicates (MDS), and one representative resulting diagram is shown. The scFv-IgAb_268 construct induced NK cell-dependent lysis against CD34⁺/CD123⁺ and CD34^{neg}/CD123⁺ cells (comprising leukemic blasts, leukemic stem cells and BM-MDSC) in the low picomolar concentration range. The CD34⁺/CD123^{neg} hematopoetic stem cell (HSC) compartment remained largely unaffected.

FIG. 14: IL-6 release in cynomolgus monkeys upon AFM28 (scFv-IgAb_268) infusion start. scFv-IgAb_268-induced TL-6 release in cynomolgus monkeys during repeated weekly i.v. dosing at three dose levels. Serum collection points are indicated in hours after start of infusion on the respective dosing day.

FIG. 15: Depletion of CD123⁺ basophils in the peripheral blood of cynomolgus upon AFM28 (scFv-IgAb_268) dosing. Animals received either vehicle or 4, 20 and 100 mg/kg by a two-hour chair infusion. Blood was collected on two pre-dose occasions, 24 h after the first dose, pre-dose on days 5, 15 22 and 29 as well as on day 43 for the recovery animals. Absolute basophil counts (CD3−/CD14−/CD20−/CD159a−/HiLA−DR−/FceRla+) were determined in whole blood by flow cytometry.

FIGS. 16A-16C: Binding of FOLR1×CD16A antibody constructs to cell lines expressing human CD16A and cynomolgus CD16.

FIG. 17: ADCC against FOLR1+ A2780 cells by anti-FOLR1 antibody constructs.

FIGS. 18A-18B: Target cell-independent activation of NK cells by anti-FOLR1 antibodies. Enriched human NK cells were cultured for 24 h with titrated concentrations of scFv-IgAb_273, scFv-IgAb_274, scFv-IgAb_275, or without (w/o) antibodies as a control (ctrl). The mean fluorescence intensity (MFI) of CD69 (FIG. 18A) and CD137 (FIG. 18B) were assessed by flow cytometry and plotted by non-linear regression using GraphPad Prism. Mean and SD values of three independent experiments are shown.

FIGS. 19A-19B: Binding of FOLR1×CD16A antibody constructs to cell lines expressing human FOLR1 and cynomolgus FOLR1. Binding of antibody constructs to huFOLR1-transfected CHO cells (FIG. 19A) and cyFOLR1-transfected CHO cells (FIG. 19B) measured by flow cytometry, depicting the median fluorescent intensity (MFI) of titrated scFv-IgAb_381 and scFv-IgAb_387.

FIG. 20: Structure information of a FOLR1×CD16A bispecific antibody construct.

FIG. 21: scFv-IgAb_387-induced lysis of target cells in the presence of soluble FOLR1. 4 h calcein-release cytotoxicity assay with A2780 and A-549 target cells and enriched human NK cells as effector cells at an E:T ratio of 2.5:1 in the presence of scFv-IgAb_387 and 2×, 25×, and 50× molar excess of soluble FOLR1 (sFOLR1) or soluble mesothelin (sMSLN). Mean values and SD of triplicates from one representative experiment are presented.

FIG. 22: scFv-IgAb_387-induced ADCP of FOLR1⁺ target cells. In vitro differentiated human macrophages were co-cultured with CMFDA-labeled HeLa and HCC-78 target cells at an E:T ratio of 5:1 in the presence of serial dilutions of FOLR1/CD16A scFv-IgAb_387, anti-FOLR1 IgG1 IgAb_355, RSV/CD16A scFv-IgAb_444, and FOLR1/RSV scFv-IgAb_162 or without antibody (w/o) for 4 h. The percentage of phagocytosed target cells was determined by flow cytometry and plotted. One representative out of three independent experiments is presented.

FIG. 23: Mice were treated with vehicle or scFv-IgAb_387 either in the therapeutic (2101-THE) or prophylactic (2101-PRO) setting. Tumor size was measured twice per week for 35 days. Mean tumor volume with SEM for each group is shown.

FIGS. 24A-24D: Mice were treated with the indicated treatments and euthanized at the termination of the experiment (35 days after tumor cell inoculation). Blood (FIG. 24A), spleen (FIG. 24B) and tumor (FIG. 24C and FIG. 24D) were harvested for flow cytometry analyses. Absolute number (right graph) and percentage (left graph) of the indicated cell populations are shown as mean with SEM. Each dot represents an analyzed tumor.

FIG. 25: scFv-IgAb_387-induced MCP-1 and IP-10 in 24 h PBMC cultures in the presence and absence of target cells. PBMC alone (PBMC only) or co-cultures of PBMC with FOLR1⁺ A-549, FOLR1⁺ OVCAR-3, or FOLR1⁻ Raji as target cells at an effector-to-target (E:T) 20:1 in the presence or absence (w/o) of increasing concentrations of FOLR1×CD16A scFv-IgAb_387 were incubated for 24 h, and MCP-1 and IP-10 were quantified in the supernatant by multiplexing. CD3/CD28 activator beads were added as a positive control. Mean values and standard deviation (SD) of three independent experiments are plotted.

FIG. 26: FOLR1×CD16A scFv-IgAb_induced cytokine release in 24 h cultures of PBMC in the absence and presence of target cells. PBMC alone or PBMC together with FOLR1+A-549, FOLR1⁺ OVCAR-3, or FOLR1⁻ Raji cells at an effector-to-target (E:T) ratio of 20:1 were cultured in the presence of 100 μg/mL scFv-IgAb_387 for 24 h. Mean cytokine concentrations in pg/mL from three independent experiments are plotted.

FIGS. 27A-27G: Mediation of ADCC by scFv-IgAb_387 and control antibodies on various tumor cell lines in 4 h calcein-release assays. Calcein-labeled target cell lines such as NCI-H2110 (FIG. 27A), OVCAR-3 (FIG. 27B), IGROV1 (FIG. 27C), A2780 (FIG. 27D), KARPAS-299 (FIG. 27E), MAD-MB-231 (FIG. 27F), SKBR-3 (FIG. 27G) were co-cultured with enriched primary human NK cells as effector cells at an E:T ratio of 5:1 in the presence of serial dilutions of FOLR1×CD16A scFv-IgAb_387, anti-FOLR1 IgG1 IgAb_355, RSV×CD16A scFv-IgAb_444, and FOLR1×RSV scFv-IgAb_162, or without antibody (w/o) for 4 h. After incubation, fluorescent calcein-released from lysed target cells was used to calculated specific target cell lysis. Mean lysis values and SD from duplicates are plotted. For each tumor cell line, the results from one representative experiment are presented.

FIG. 28: Binding of scFv-IgAb_387 and control antibody construct to NK cells in presence and absence of polyclonal human IgG. NK cells were incubated with increasing concentrations of biotinylated scFv-IgAb_387, biotinylated scFv-IgAb_444 (anti-RSV×CD16A), biotinylated scFv-IgAb_162 (anti-FOLR1×RSV), or biotinylated anti-FOLR1 IgG1 antibody comprising wild-type Fc (IgAb_335) and biotinylated 3G8 (murine anti human CD16) at 37° C. in presence or absence of 10 mg/mL polyclonal human IgG. Cell surface-bound antibodies were detected with streptavidin-FITC followed by flow cytometric analysis. Data of one representative experiment is shown out of four experiments. MFI, median fluorescence intensity.

FIGS. 29A-29B: Binding of scFv-IgAb_387 and control molecules to FOLR1 expressing tumor cells. scFv-IgAb_387 (FOLR1×CD16A) and control antibodies scFv-IgAb_444 (RSV×CD16A), scFv-IgAb_162 (FOLR1×RSV), and IgAb_335 (anti-FOLR1 IgG1) were titrated on IGROV-1 cells expressing high levels of FOLR1 (FIG. 29A) and OVCAR-3 cells expressing medium levels of FOLR1 (FIG. 29B) and incubated at 37° C. As positive control for all cell lines anti-FOLR1 mAb (clone LK26) has been used. Cell surface bound antibodies were analyzed by flow cytometry, and median fluorescence intensities (MFI) were plotted.

FIGS. 30A-30B: Binding of scFv-IgAb_387 on to recombinant human CD16A antigens. One 96-well ELISA plate, each, was coated with (FIG. 30A) human CD16A 158V, (FIG. 30B) human CD16A 158F. Antibodies were applied in 3-fold serial dilutions starting at 50 nM. Data shown is one of three (FIG. 30A) or four (FIG. 30B) replicate experiments.

FIG. 31: Binding of scFv-IgAb_387 to recombinant FOLR1 antigen in ELISA. One 96-well ELISA plate was coated with human FOLR1. Antibodies were applied in 3-fold serial dilutions starting at 5 nM. Data shown is one of three replicate experiments.

### DEFINITIONS

The term “binding domain” characterizes in connection with the present invention a domain which is capable of specifically binding to/interacting with/recognizing a given target epitope or a given target site on the target molecules (antigens), i.e. CD16A on the surface of an immune effector cell, and a target cell surface antigen, respectively. The structure and/or function of the first binding domain (recognizing CD16A), and also the structure and/or function of the second binding domain (recognizing the target cell surface antigen, in particular FOLR1), is/are preferably based on the structure and/or function of an antibody, e.g. of a full-length or whole immunoglobulin molecule and/or is/are drawn from the variable heavy chain (VH) and/or variable light chain (VL) domains of an antibody or fragment thereof.

The term “specifically binding”, as used herein means that the binding domain preferentially binds or recognizes the target even when the binding partner is present in a mixture of other molecules or other structures. The binding may be mediated by covalent or non-covalent interactions or a combination of both. In preferred embodiments, “simultaneous binding to a target cell and an immune effector cell” comprises the physical interaction between the binding domains and their targets on the cells, but preferably also includes the induction of an action mediated by the simultaneous binding of the two cells. Such an action may be an immune effector function of the immune effector cell, such as a cytotoxic effect.

The term “antibody construct” refers to a molecule in which the structure and/or function is/are based on the structure and/or function of an antibody, e.g., of a full-length or whole immunoglobulin molecule and/or is/are drawn from the variable heavy chain (VH) and/or variable light chain (VL) domains of an antibody or fragment thereof Δn antibody construct is hence capable of binding to its specific target or antigen. Furthermore, the binding region of an antibody construct defined in the context of the invention comprises the minimum structural requirements of an antibody which allow for the target binding. For the first binding domain (A) this minimum requirement are defined by the presence of a VL region comprising the three light chain CDRs (i.e. CDR1, CDR2 and CDR3 of the VL region) and the presence of a VH region comprising the three heavy chain CDRs (i.e. CDR1, CDR2 and CDR3 of the VH region). For the second binding domain (B) this minimum requirement may e.g. be defined by the presence of at least the three light chain CDRs (i.e. CDR1, CDR2 and CDR3 of the VL region) and/or the three heavy chain CDRs (i.e. CDR1, CDR2 and CDR3 of the VH region), preferably of all six CDRs. An alternative approach to define the minimal structure requirements of an antibody is the definition of the epitope of the antibody within the structure of the specific target, respectively, the protein domain of the target protein composing the epitope region (epitope cluster) or by reference to a specific antibody competing with the epitope of the defined antibody. The antibodies on which the constructs defined in the context of the invention are based include for example monoclonal, recombinant, chimeric, deimmunized, humanized and human antibodies.

The first binding domain of an antibody construct defined in the context of the invention comprises the above referred groups of CDRs. Those CDRs are comprised in the framework of an antibody light chain variable region (VL) and an antibody heavy chain variable region (VH). The second binding domain of an antibody construct defined in the context of the invention may e.g. comprise the above referred groups of CDRs. Preferably, those CDRs are comprised in the framework of an antibody light chain variable region (VL) and an antibody heavy chain variable region (VH); however, it does not have to comprise both. Fd fragments, for example, have two VH regions and often retain some antigen-binding function of the intact antigen-binding region.

Examples for the format of antibody fragments, antibody variants or binding domains include (1) a Fab fragment, a monovalent fragment having the VL, VH, CL and CHi domains; (2) a F(ab′)₂ fragment, a bivalent fragment having two Fab fragments linked by a disulfide bridge at the hinge domain; (3) an Fd fragment having the two VH and CHi domains; (4) an Fv fragment having the VL and VH domains of a single arm of an antibody, (5) a dAb fragment (Ward et al., (1989) Nature 341:544-546), which has a VH domain; (6) an isolated complementarity determining region (CDR), and (7) a single chain Fv (scFv), the latter being preferred (for example, derived from an scFv-library). Examples for embodiments of antibody constructs according to the invention are e.g. described in WO 00/006605, WO 2005/040220, WO 2008/119567, WO 2010/037838, WO 2013/026837, WO 2013/026833, US 2014/0308285, US 2014/0302037, WO2014/144722, WO 2014/151910, and WO 2015/048272.

An antibody construct as defined in the context of the invention may comprise a fragment of a full-length antibody, such as VH, VHH, VL, (s)dAb, Fv, Fd, Fab, Fab′, F(ab′)₂ or “r IgG” (“half antibody”). Antibody constructs as defined in the context of the invention may also comprise modified fragments of antibodies, also called antibody variants, such as scFv, di-scFv or bi(s)-scFv, scFv-Fc, scFv-zipper, scFab, Fab₂, Fab₃, diabodies, single chain diabodies, tandem diabodies (Tandab's), tandem di-scFv, tandem tri-scFv, “multibodies” such as triabodies or tetrabodies, and single domain antibodies such as nanobodies or single variable domain antibodies comprising merely one variable domain, which might be VHH, VH or VL, that specifically bind an antigen or epitope independently of other V regions or domains.

As used herein, the terms “single-chain Fv,” “single-chain antibodies” or “scFv” refer to single polypeptide chain antibody fragments that comprise the variable regions from both the heavy and light chains, but lack the constant regions. Generally, a single-chain antibody further comprises a polypeptide linker between the VH and VL domains which enables it to form the desired structure which would allow for antigen binding. A preferred linker for this purpose is a glycine serine linker, which preferably comprises from about 15 to about 30 amino acids. Preferred glycine serine linkers may have one or more repeats of GGS, GGGS (SEQ ID NO: 41), or GGGGS (SEQ ID NO: 46). Such linker preferably comprises 5, 6, 7, 8, 9 and/or 10 repeats of GGS, preferably (GGS)₆ (SEQ ID NO 44) (which are preferably used for scFvs having the arrangement VH-VL), or preferably (GGS)₇ (SEQ ID NO: 45) (which are preferably used for scFvs having the arrangement VL-VH). Single chain antibodies are discussed in detail by Plueckthun in The Pharmacology of Monoclonal Antibodies, vol. 1 13, Rosenburg and Moore eds. Springer-Verlag, New York, pp. 269-315 (1994). Various methods of generating single chain antibodies are known, including those described in U.S. Pat. Nos. 4,694,778 and 5,260,203; International Patent Application Publication No. WO 88/01649; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041. In specific embodiments, single-chain antibodies can also be human, and/or humanized and/or synthetic. The term “bi-scFv” or “ta-scFv” (tandem scFv) as used herein refers to two scFv that are fused together. Such a bi-scFv or ta-scFv may comprise a linker between the two scFv moieties. Generally, the arrangement of the VH and VL domains on the polypeptide chain within each of the scFv may be in any order. This means that the “bi-scFv” of “ta-scFv” can be arranged in the order VH(1)-VL(1)-VH(2)-VL(2), VL(1)-VH(1)-VH(2)-VL(2), VH(1)-VL(1)-VL(2)-VH(2), or VL(1)-VH(1)-VL(2)-VH(2), where (1) and (2) stand for the first and second scFv, respectively.

The term “double Fab” as used herein refers to two Fab fragments that are fused together, which are preferably staggered. Here, a first chain of a first Fab is N-terminally fused to a first chain of a second Fab, or a second chain of a first Fab is N-terminally fused to a second chain of a second Fab, or both, the first chain of a first Fab and the second chain of a first Fab are fused to first and second chains of a second Fab, respectively. A linker may be present between the fused chains of the first and second Fab. The first and second chains of the first and second Fab can be individually selected from a light chain-derived chain of a Fab (VL-CL), a heavy chain derived chain of a Fab (VH-CHi), as long as each Fab contains a VH, a VL, a CHi, and a CL. As an illustrative example, the light chain-derived chain of the first Fab can be fused to the light chain derived-chain of the second Fab. As another illustrative example, the heavy chain-derived chain of the first Fab can be fused to the heavy chain derived-chain of the second Fab. As a further illustrative example, the heavy chain-derived chain of the first Fab can be fused to the light chain derived-chain of the second Fab. In some double Fabs, both chains of the two Fabs are fused together. For example, the light chain-derived chain of the first Fab can be fused to the light chain derived-chain of the second Fab while the heavy chain-derived chain of the first Fab can be fused to the heavy chain derived-chain of the second Fab. Alternatively, the light chain-derived chain of the first Fab can be fused to the heavy chain derived-chain of the second Fab while the heavy chain-derived chain of the first Fab can be fused to the light chain derived-chain of the second Fab. A fusion of two Fab chains may optionally comprise a linker. Suitable and preferred linkers comprise the upper hinge sequence (SEQ ID NO: 54) or glycine serine linkers with about up to 20 amino acids, preferably up to 10 amino acids, or most preferably 10 amino acids, e.g. two repeats of GGGGS (SEQ ID NO: 46). Glycine serine linkers comprised in a double Fab may have one or more repeats of GGS, GGGS (SEQ ID NO: 41), or GGGGS (SEQ ID NO: 46), such as one, two, three, or four repeats.

As used herein, a “diabody” or “Db” refers to an antibody construct comprising two binding domains, which may be constructed using heavy and light chains disclosed herein, as well as by using individual CDR regions disclosed herein. Typically, a diabody comprise a heavy chain variable domain (VH) connected to a light chain variable domain (VL) by a linker which is too short to allow pairing between the two domains on the same chain. Preferred linkers for this purpose include glycine serine linkers with about up to 12 amino acids, preferably up to about 10 amino acids. Preferred glycine serine linkers may have one or more repeats of GGS, GGGS (SEQ ID NO: 41), or GGGGS (SEQ ID NO: 46). A preferred linker is (GGS)₂ SEQ ID NO: (42). Another preferred linker is (GGS)₃ SEQ ID NO: (43). Accordingly, the VH and VL domains of one fragment are forced to pair with the complementary VH and VL domains of another fragment, thereby forming two antigen-binding sites. A diabody can be formed by two separate polypeptide chains, each comprising a VH and a VL. Alternatively, all four variable domains can be comprised in one single polypeptide chain comprising two VH and two VL domains. In such a case, the diabody can also be termed “single chain diabody” or “scDb”. Typically, a scDb comprises the two chains of a non-single chain diabody that are fused together, preferably via a linker. A preferred linker for this purpose is a glycine serine linker, which preferably comprises from about 15 to about 30 amino acids. Preferred glycine serine linkers may have one or more repeats of GGS, GGGS (SEQ ID NO: 41), or GGGGS (SEQ ID NO: 46). Such linker preferably comprises 5, 6, 7, 8, 9, and/or 10 repeats of GGS, preferably (GGS)₆, (SEQ ID NO 44) or preferably (GGS)₇ (SEQ ID NO: 45). On the polypeptide chain, the variable domains of a scDb can be arranged (from N to C terminus) in a VL-VH-VL-VH or VH-VL-VH-VL order. Similarly, the spatial arrangement of the four domains in the tertiary/quaternary structure can be in a VL-VH-VL-VH or VH-VL-VH-VL order. The term diabody does not exclude the fusion of further binding domains to the diabody.

In the context of the present invention, the definition of the term “antibody construct” includes monovalent, bivalent and polyvalent/multivalent constructs, i.e. monovalent, bivalent, trivalent, or even higher valency for first and second target bound by the first and second binding domain, wherein the antibody construct is necessarily bispecific as described elsewhere herein, i.e. comprises specificities for two different antigens or targets. The term “valent” denotes the presence of a determined number of antigen-binding domains in the antigen-binding protein. A natural IgG has two antigen-binding domains and is bivalent. For examples, the bispecific antibody constructs of the present invention may comprise one, two or more first binding domains (A) against CD16A and one, two or more second binding domains (B) against a second target on the surface of a target cell, preferably a hematological target cell, as defined elsewhere herein. Moreover, the definition of the term “antibody construct” includes molecules consisting of only one polypeptide chain as well as molecules consisting of more than one polypeptide chain, which chains can be either identical (homodimers, homotrimers or homo oligomers) or different (heterodimer, heterotrimer or heterooligomer). Examples for the above identified antibodies and variants or derivatives thereof are described inter alia in Harlow and Lane, Antibodies a laboratory manual, CSHL Press (1988) and Using Antibodies: a laboratory manual, CSHL Press (1999), Kontermann and Dubel, Antibody Engineering, Springer, 2nd ed. 2010 and Little, Recombinant Antibodies for Immunotherapy, Cambridge University Press 2009.

The term “bispecific” as used herein refers to an antibody construct which is “essentially bispecific”, i.e., comprise specificities for two different antigens or targets, but no further specificity against a third or further antigen or target. Specifically, the bispecific antibody construct of the present invention comprises a (first) binding domain that binds to one antigen or target (here: CD16A) and a (second) binding domain that binds to another antigen or target (here: the target cell surface antigen) which is not CD16A. Accordingly, antibody constructs as defined in the context of the invention comprise specificities for two different antigens or targets. For example, the first binding domain does preferably bind to an extracellular epitope of an NK cell receptor of one or more of the species selected from human, Macaca spec. and rodent species, and the second binding domain does preferably bind to an extracellular epitope of a target cell surface antigen.

“CD16A” or “CD16a” refers to the activating receptor CD16A, also known as FcγRIIIA, expressed on the cell surface of NK cells. CD16A is an activating receptor triggering the cytotoxic activity of NK cells. The amino acid sequence of human CD16A is given in UniProt entry P08637 (version 212 of 12 Aug. 2020) as well as in SEQ ID NO: 50. The affinity of antibodies for CD16A directly correlates with their ability to trigger NK cell activation, thus higher affinity towards CD16A reduces the antibody dose required for activation. The antigen-binding site of the antigen-binding protein binds to CD16A, but preferably not to CD16B. For example, an antigen-binding site comprising heavy (VH) and light (VL) chain variable domains binding to CD16A, but not binding to CD16B, may be provided by an antigen-binding site which specifically binds to an epitope of CD16A which comprises amino acid residues of the C-terminal sequence SFFPPGYQ (positions 201-208 of SEQ ID NO: 50) and/or residues G147 and/or Y158 of CD16A which are not present in CD16B.

“CD16B” refers to receptor CD16B, also known as FcγRIIIB, expressed on neutrophils and eosinophils. The receptor is glycosylphosphatidyl inositol (GPI) anchored and is understood to not trigger any kind of cytotoxic activity of CD16B positives immune cells. The amino acid sequence of human CD16B is given in UniProt entry 075015 (version 212 of 12 Aug. 2020) as well as in SEQ ID NO: 52.

The term “target cell” describes a cell or a group of cells, which is/are the target of the mode of action applied by the antibody construct of the invention. This cell/group of cells comprise e.g. pathological cells, which are eliminated or inhibited by engaging these cells with the effector cell via the antibody construct of the invention. A preferred target cell is a cancer cell.

The term “CD16A shedding” or “shedding of CD16A” refers to the down-modulation/down-regulation/degradation of FcγRIIIA expressed on the cell surface of immune effector cells such as NK cells after binding and activation of immune effector cells by a CD16A binding domain, e.g. an antibody. “CD16A” shedding is typically mediated by A disintegrin and metalloproteinase (ADAM17), or membrane type 6 matrix metalloproteinase (MMP25) and describes a proteolytic process that regulates the cell surface density of said surface molecules on immune effector cells. “CD16A shedding” is known as activation-induced down-regulation as described e.g. in Romee at a1., Blood, 2013, 121 (18):3599-3608), Peruzi et al., J. Immunol., 2013, 191:955-957, Goodier et al., Front. Immunol., 2016, 7:384, and Srpan et al., J. Cell. Biol., 2018, 217(9):3267-3283, and the capacity of immune effector cells after CD16A shedding may then be impaired for several days.

The term “target cell surface antigen” refers to an antigenic structure expressed by a cell and which is present at the cell surface such that it is accessible for an antibody construct as described herein. It may be a protein, preferably the extracellular portion of a protein, a peptide that is presented on the cell surface in an MHC context (including HLA-A2, HLA-A11, HLA-A24, HLA-B44, HLA-C4) or a carbohydrate structure, preferably a carbohydrate structure of a protein, such as a glycoprotein. It is preferably a tumor associated or tumor restricted antigen. The target cell surface antigens particularly envisaged in the context of the present invention is FOLR1 as defined elsewhere herein. It is envisaged that CD16A is not a target cell surface antigen of the present invention.

The term “antibody construct” of the invention is essentially bispecific, i.e. may not encompass further specificities resulting in antibody constructs such as tri- or tetraspecific antibody constructs, the latter ones including four or more binding domains, or constructs having more than four (e.g. five, six . . . ) specificities.

Given that the antibody constructs as defined in the context of the invention are bispecific, they do not occur naturally and they are markedly different from naturally occurring products. A “bispecific” antibody construct is hence an artificial hybrid antibody having two distinct binding sides with different specificities. Bispecific antibody constructs can be produced by a variety of methods including fusion of hybridomas or linking of Fab′ fragments. See, e.g., Songsivilai & Lachmann, Clin. Exp. Immunol. 79:315-321 (1990).

The binding domains and the variable domains (VH/VL) of the antibody construct of the present invention may or may not comprise peptide linkers (spacer peptides). The term “peptide linker” comprises in accordance with the present invention an amino acid sequence by which the amino acid sequences of one (variable and/or binding) domain and another (variable and/or binding) domain of the antibody construct defined herein are linked with each other. The peptide linkers can also be used to fuse one domain to another domain of the antibody construct defined herein. In such cases, the peptide linker may also be referred to as a “connector”. Such a connector is preferably a short linker, which preferably has a length of about 10 nm or less, preferably about 9 nm or less, preferably about 8 nm or less, preferably about 7 nm or less, preferably about 6 nm or less, preferably about 5 nm or less, preferably about 4 nm or less, or even less. The length of the linker is preferably determined as described by Rossmalen et a1 Biochemistry 2017, 56, 6565-6574, which also describes suitable linkers that are well known to the skilled person. An example for a connector is a glycine serine linker or a serine linker, which preferably comprise no more than about 75 amino acids, preferably not more than about 50 amino acids. In illustrative examples, a suitable linker comprises one or more (e.g. 1, 2, 3, 4, 5, 6, 7, or 8) GGGGS sequences (SEQ ID NO: 46), such as (GGGGS)₂ (SEQ ID NO: 47), (GGGGS)₄ (SEQ ID NO: 48), or preferably (GGGGS)₆ (SEQ ID NO: 49). Other illustrative examples for linkers are shown in SEQ ID NOs: 42-45. A preferred technical feature of such peptide linker is that it does not comprise any polymerization activity.

The antibody constructs as defined in the context of the invention are preferably “in vitro generated antibody constructs”. This term refers to an antibody construct according to the above definition where all or part of the variable region (e.g., at least one CDR) is generated in a non-immune cell selection, e.g., an in vitro phage display, protein chip or any other method in which candidate sequences can be tested for their ability to bind to an antigen. This term thus preferably excludes sequences generated solely by genomic rearrangement in an immune cell in an animal. A “recombinant antibody” is an antibody made through the use of recombinant DNA technology or genetic engineering.

The term “monoclonal antibody” (mAb) or monoclonal antibody construct as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic side or determinant on the antigen, in contrast to conventional (polyclonal) antibody preparations which typically include different antibodies directed against different determinants (or epitopes). In addition to their specificity, the monoclonal antibodies are advantageous in that they are synthesized by the hybridoma culture, hence uncontaminated by other immunoglobulins. The modifier “monoclonal” indicates the character of the antibody as being obtained from a substantially homogeneous population of antibodies, and is not to be construed as requiring production of the antibody by any particular method.

For the preparation of monoclonal antibodies, any technique providing antibodies produced by continuous cell line cultures can be used. For example, monoclonal antibodies to be used may be made by the hybridoma method first described by Koehler et al., Nature, 256: 495 (1975), or may be made by recombinant DNA methods (see, e.g., U.S. Pat. No. 4,816,567). Examples for further techniques to produce human monoclonal antibodies include the trioma technique, the human B-cell hybridoma technique (Kozbor, Immunology Today 4 (1983), 72) and the EBV-hybridoma technique (Cole et al., Monoclonal Antibodies and Cancer Therapy, Alan R. Liss, Inc. (1985), 77-96).

Hybridomas can then be screened using standard methods, such as enzyme-linked immunosorbent assay (ELISA) and surface plasmon resonance (BIACORE™) analysis, to identify one or more hybridomas that produce an antibody that specifically binds with a specified antigen. Any form of the relevant antigen may be used as the immunogen, e.g., recombinant antigen, naturally occurring forms, any variants or fragments thereof, as well as an antigenic peptide thereof. Surface plasmon resonance as employed in the BIAcore system can be used to increase the efficiency of phage antibodies which bind to an epitope of a target cell surface antigen, (Schier, Human Antibodies Hybridomas 7 (1996), 97-105; Malmborg, J. Immunol. Methods 183 (1995), 7-13). Another exemplary method of making monoclonal antibodies includes screening protein expression libraries, e.g., phage display or ribosome display libraries. Phage display is described, for example, in Ladner et al., U.S. Pat. No. 5,223,409; Smith (1985) Science 228:1315-1317, Clackson et ai, Nature, 352: 624-628 (1991) and Marks et al., J. Mol. Biol., 222: 581-597 (1991).

In addition to the use of display libraries, the relevant antigen can be used to immunize a non-human animal, e.g., a rodent (such as a mouse, hamster, rabbit or rat). In one embodiment, the non-human animal includes at least a part of a human immunoglobulin gene. For example, it is possible to engineer mouse strains deficient in mouse antibody production with large fragments of the human Ig (immunoglobulin) loci. Using the hybridoma technology, antigen-specific monoclonal antibodies derived from the genes with the desired specificity may be produced and selected. See, e.g., XENOMOUSE™, Green et al. (1994) Nature Genetics 7:13-21, US 2003-0070185, WO 96/34096, and WO 96/33735.

A monoclonal antibody can also be obtained from a non-human animal, and then modified, e.g., humanized, deimmunized, rendered chimeric etc., using recombinant DNA techniques known in the art. Examples of modified antibody constructs include humanized variants of non-human antibodies, “affinity matured” antibodies (see, e.g. Hawkins et al. J. Mol. Biol. 254, 889-896 (1992) and Lowman et al., Biochemistry 30, 10832-10837 (1991)) and antibody mutants with altered effector function(s) (see, e.g., U.S. Pat. No. 5,648,260, Kontermann and Dubel (2010), loc. cit. and Little (2009), loc. cit).

In immunology, affinity maturation is the process by which B cells produce antibodies with increased affinity for antigen during the course of an immune response. With repeated exposures to the same antigen, a host will produce antibodies of successively greater affinities. Like the natural prototype, the in vitro affinity maturation is based on the principles of mutation and selection. The in vitro affinity maturation has successfully been used to optimize antibodies, antibody constructs, and antibody fragments. Random mutations inside the CDRs are introduced using radiation, chemical mutagens or error-prone PCR. In addition, the genetic diversity can be increased by chain shuffling. Two or three rounds of mutation and selection using display methods like phage display usually results in antibody fragments with affinities in the low nanomolar range.

A preferred type of an amino acid substitutional variation of the antibody constructs involves substituting one or more hypervariable region residues of a parent antibody (e. g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sides (e. g. 6-7 sides) are mutated to generate all possible amino acid substitutions at each side. The antibody variants thus generated are displayed in a monovalent fashion from filamentous phage particles as fusions to the gene III product of M13 packaged within each particle. The phage-displayed variants are then screened for their biological activity (e. g. binding affinity) as herein disclosed. In order to identify candidate hypervariable region sides for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the binding domain and, e.g., human target cell surface antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

The monoclonal antibodies and antibody constructs of the present disclosure specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is/are identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81: 6851-6855 (1984)). Chimeric antibodies of interest herein include “primitized” antibodies comprising variable domain antigen-binding sequences derived from a non-human primate (e.g., Old World Monkey, Ape etc.) and human constant region sequences. A variety of approaches for making chimeric antibodies have been described. See e.g., Morrison et a1., Proc. Natl. Acad. Sci U.S.A. 81:6851, 1985; Takeda et al., Nature 314:452, 1985, Cabilly et a1., U.S. Pat. No. 4,816,567; Boss et al., U.S. Pat. No. 4,816,397; Tanaguchi et al., EP 0171496; EP 0173494; and GB 2177096.

An antibody, antibody construct, antibody fragment or antibody variant may also be modified by specific deletion of human T cell epitopes (a method called “deimmunization”) by the methods disclosed for example in WO 98/52976 or WO 00/34317. Briefly, the heavy and light chain variable domains of an antibody can be analyzed for peptides that bind to MHC class II; these peptides represent potential T cell epitopes (as defined in WO 98/52976 and WO 00/34317). For detection of potential T cell epitopes, a computer modeling approach termed “peptide threading” can be applied, and in addition a database of human MHC class II binding peptides can be searched for motifs present in the VH and VL sequences, as described in WO 98/52976 and WO 00/34317. These motifs bind to any of the 18 major MHC class II DR allotypes, and thus constitute potential T cell epitopes. Potential T cell epitopes detected can be eliminated by substituting small numbers of amino acid residues in the variable domains, or preferably, by single amino acid substitutions. Typically, conservative substitutions are made. Often, but not exclusively, an amino acid common to a position in human germline antibody sequences may be used. Human germline sequences are disclosed e.g. in Tomlinson, et al. (1992) J. Mol. Biol. 227:776-798; Cook, G. P. et al. (1995) Immunol. Today Vol. 16 (5): 237-242; and Tomlinson et a1. (1995) EMBO J. 14: 14:4628-4638. The V BASE directory provides a comprehensive directory of human immunoglobulin variable region sequences (compiled by Tomlinson, L A. et al. MRC Centre for Protein Engineering, Cambridge, UK). These sequences can be used as a source of human sequence, e.g., for framework regions and CDRs. Consensus human framework regions can also be used, for example as described in U.S. Pat. No. 6,300,064.

“Humanized” antibodies, antibody constructs, variants or fragments thereof (such as Fv, Fab, Fab′, F(ab′)2 or other antigen-binding subsequences of antibodies) are antibodies or immunoglobulins of mostly human sequences, which contain (a) minimal sequence(s) derived from non-human immunoglobulin. For the most part, humanized antibodies are human immunoglobulins (recipient antibody) in which residues from a hypervariable region (also CDR) of the recipient are replaced by residues from a hypervariable region of a non-human (e.g., rodent) species (donor antibody) such as mouse, rat, hamster or rabbit having the desired specificity, affinity, and capacity. In some instances, Fv framework region (FR) residues of the human immunoglobulin are replaced by corresponding non-human residues. Furthermore, “humanized antibodies” as used herein may also comprise residues which are found neither in the recipient antibody nor the donor antibody. These modifications are made to further refine and optimize antibody performance. The humanized antibody may also comprise at least a portion of an immunoglobulin constant region (Fc), typically that of a human immunoglobulin. For further details, see Jones et al., Nature, 321: 522-525 (1986); Reichmann et al., Nature, 332: 323-329 (1988); and Presta, Curr. Op. Struct. Biol., 2: 593-596 (1992).

Humanized antibodies or fragments thereof can be generated by replacing sequences of the Fv variable domain that are not directly involved in antigen binding with equivalent sequences from human Fv variable domains. Exemplary methods for generating humanized antibodies or fragments thereof are provided by Morrison (1985) Science 229:1202-1207; by Oi et al. (1986) BioTechniques 4:214; and by U.S. Pat. Nos. 5,585,089; 5,693,761; 5,693,762; 5,859,205; and 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

Humanized antibodies may also be produced using transgenic animals such as mice that express human heavy and light chain genes, but are incapable of expressing the endogenous mouse immunoglobulin heavy and light chain genes. Winter describes an exemplary CDR grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

A humanized antibody can be optimized by the introduction of conservative substitutions, consensus sequence substitutions, germline substitutions and/or back mutations. Such altered immunoglobulin molecules can be made by any of several techniques known in the art, (e.g., Teng et al., Proc. Natl. Acad. Sci. U.S.A., 80: 7308-7312, 1983; Kozbor ei al., Immunology Today, 4: 7279, 1983; Olsson et al., Meth. Enzymol., 92: 3-16, 1982, and EP 239 400).

The term “human antibody”, “human antibody construct” and “human binding domain” includes antibodies, antibody constructs and binding domains having antibody regions such as variable and constant regions or domains which correspond substantially to human germline immunoglobulin sequences known in the art, including, for example, those described by Kabat et a1. (1991) (loc. cit.). The human antibodies, antibody constructs or binding domains as defined in the context of the invention may include amino acid residues not encoded by human germline immunoglobulin sequences (e.g., mutations introduced by random or side-specific mutagenesis in vitro or by somatic mutation in vivo), for example in the CDRs, and in particular, in CDR3. The human antibodies, antibody constructs or binding domains can have at least one, two, three, four, five, or more positions replaced with an amino acid residue that is not encoded by the human germline immunoglobulin sequence. The definition of human antibodies, antibody constructs and binding domains as used herein, however, also contemplates “fully human antibodies”, which include only non-artificially and/or genetically altered human sequences of antibodies as those can be derived by using technologies or systems such as the Xenomouse. Preferably, a “fully human antibody” does not include amino acid residues not encoded by human germline immunoglobulin sequences.

In some embodiments, the antibody constructs defined herein are “isolated” or “substantially pure” antibody constructs. “Isolated” or “substantially pure”, when used to describe the antibody constructs disclosed herein, means an antibody construct that has been identified, separated and/or recovered from a component of its production environment. Preferably, the antibody construct is free or substantially free of association with all other components from its production environment. Contaminant components of its production environment, such as that resulting from recombinant transfected cells, are materials that would typically interfere with diagnostic or therapeutic uses for the polypeptide, and may include enzymes, hormones, and other proteinaceous or non-proteinaceous solutes. The antibody constructs may e.g. constitute at least about 5%, or at least about 50% by weight of the total protein in a given sample. It is understood that the isolated protein may constitute from 5% to 99.9% by weight of the total protein content, depending on the circumstances. The polypeptide may be made at a significantly higher concentration through the use of an inducible promoter or high expression promoter, such that it is made at increased concentration levels. The definition includes the production of an antibody construct in a wide variety of organisms and/or host cells that are known in the art. In preferred embodiments, the antibody construct will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Ordinarily, however, an isolated antibody construct will be prepared by at least one purification step.

According to the present invention, binding domains are in the form of one or more polypeptides. Such polypeptides may include proteinaceous parts and non-proteinaceous parts (e.g. chemical linkers or chemical cross-linking agents such as glutaraldehyde). Proteins (including fragments thereof, preferably biologically active fragments, and peptides, usually having less than 30 amino acids) comprise two or more amino acids coupled to each other via a covalent peptide bond (resulting in a chain of amino acids).

The term “polypeptide” or “polypeptide chain” as used herein describes a group of molecules, which usually consist of more than 30 amino acids. The terms “peptide”, “polypeptide” and “protein” also refer to naturally modified peptides/polypeptides/proteins wherein the modification is affected e.g. by post-translational modifications like glycosylation, acetylation, phosphorylation and the like. A “peptide”, “polypeptide” or “protein” when referred to herein may also be chemically modified such as pegylated. Such modifications are well known in the art and described herein below. The above modifications (glycosylation, pegylation etc.) also apply to the antibody constructs of the invention.

Preferably the binding domain which binds to CD16A, and/or the binding domain which binds to the target cell surface antigen is/are human binding domains. Antibodies and antibody constructs comprising at least one human binding domain avoid some of the problems associated with antibodies or antibody constructs that possess non-human such as rodent (e.g. murine, rat, hamster or rabbit) variable and/or constant regions. The presence of such rodent derived proteins can lead to the rapid clearance of the antibodies or antibody constructs or can lead to the generation of an immune response against the antibody or antibody construct by a patient. In order to avoid the use of rodent derived antibodies or antibody constructs, human or fully human antibodies/antibody constructs can be generated through the introduction of human antibody function into a rodent so that the rodent produces fully human antibodies.

The ability to clone and reconstruct megabase-sized human loci in YACs and to introduce them into the mouse germline provides a powerful approach to elucidating the functional components of very large or crudely mapped loci as well as generating useful models of human disease. Furthermore, the use of such technology for substitution of mouse loci with their human equivalents could provide unique insights into the expression and regulation of human gene products during development, their communication with other systems, and their involvement in disease induction and progression.

An important practical application of such a strategy is the “humanization” of the mouse humoral immune system. Introduction of human immunoglobulin (Ig) loci into mice in which the endogenous Ig genes have been inactivated offers the opportunity to study the mechanisms underlying programmed expression and assembly of antibodies as well as their role in B-cell development. Furthermore, such a strategy could provide an ideal source for production of fully human monoclonal antibodies (mAbs)—an important milestone towards fulfilling the promise of antibody therapy in human disease. Fully human antibodies or antibody constructs are expected to minimize the immunogenic and allergic responses intrinsic to mouse or mouse-derivatized mAbs and thus to increase the efficacy and safety of the administered antibodies/antibody constructs.

The use of fully human antibodies or antibody constructs can be expected to provide a substantial advantage in the treatment of chronic and recurring human diseases, such as inflammation, autoimmunity, and cancer, which require repeated compound administrations.

One approach towards this goal was to engineer mouse strains deficient in mouse antibody production with large fragments of the human Ig loci in anticipation that such mice would produce a large repertoire of human antibodies in the absence of mouse antibodies. Large human Ig fragments would preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains should yield high affinity antibodies against any antigen of interest, including human antigens. Using the hybridoma technology, antigen-specific human mAbs with the desired specificity could be readily produced and selected. This general strategy was demonstrated in connection with the generation of the first XenoMouse mouse strains (see Green et al. Nature Genetics 7:13-21 (1994)). The XenoMouse strains were engineered with yeast artificial chromosomes (YACs) containing 245 kb and 190 kb-sized germline configuration fragments of the human heavy chain locus and kappa light chain locus, respectively, which contained core variable and constant region sequences. The human Ig containing YACs proved to be compatible with the mouse system for both rearrangement and expression of antibodies and were capable of substituting for the inactivated mouse Ig genes. This was demonstrated by their ability to induce B cell development, to produce an adult-like human repertoire of fully human antibodies, and to generate antigen-specific human mAbs. These results also suggested that introduction of larger portions of the human Ig loci containing greater numbers of V genes, additional regulatory elements, and human Ig constant regions might recapitulate substantially the full repertoire that is characteristic of the human humoral response to infection and immunization. The work of Green et al. was recently extended to the introduction of greater than approximately 80% of the human antibody repertoire through introduction of megabase sized, germline configuration YAC fragments of the human heavy chain loci and kappa light chain loci, respectively. See Mendez et al. Nature Genetics 15:146-156 (1997) and U.S. patent application Ser. No. 08/759,620.

The production of the XenoMouse mice is further discussed and delineated in U.S. patent application Ser. No. 07/466,008, Ser. No. 07/610,515, Ser. No. 07/919,297, Ser. No. 07/922,649, Ser. No. 08/031,801, Ser. No. 08/112,848, Ser. No. 08/234,145, Ser. No. 08/376,279, Ser. No. 08/430,938, Ser. No. 08/464,584, Ser. No. 08/464,582, Ser. No. 08/463,191, Ser. No. 08/462,837, Ser. No. 08/486,853, Ser. No. 08/486,857, Ser. No. 08/486,859, Ser. No. 08/462,513, Ser. No. 08/724,752, and Ser. No. 08/759,620; and U.S. Pat. Nos. 6,162,963; 6,150,584; 6,114,598; 6,075,181, and 5,939,598 and Japanese Patent Nos. 3 068 180 B2, 3 068 506 B2, and 3 068 507 B2. See also Mendez et al. Nature Genetics 15:146-156 (1997) and Green and Jakobovits J. Exp. Med. 188:483-495 (1998), EP 0 463 151 B1, WO 94/02602, WO 96/34096, WO 98/24893, WO 00/76310, and WO 03/47336.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more VH genes, one or more DH genes, one or more JH genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806; 5,625,825; 5,625,126; 5,633,425; 5,661,016; 5,770,429; 5,789,650; 5,814,318; 5,877,397; 5,874,299; and 6,255,458 each to Lonberg and Kay, U.S. Pat. Nos. 5,591,669 and 6,023.010 to Krimpenfort and Berns, U.S. Pat. Nos. 5,612,205; 5,721,367; and U.S. Pat. No. 5,789,215 to Berns et al., and U.S. Pat. No. 5,643,763 to Choi and Dunn, and GenPharm International U.S. patent application Ser. No. 07/574,748, Ser. No. 07/575,962, Ser. No. 07/810,279, Ser. No. 07/853,408, Ser. No. 07/904,068, Ser. No. 07/990,860, Ser. No. 08/053,131, Ser. No. 08/096,762, Ser. No. 08/155,301, Ser. No. 08/161,739, Ser. No. 08/165,699, Ser. No. 08/209,741. See also EP 0 546 073 B1, WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175. See further Taylor et a1. (1992), Chen et al. (1993), Tuaillon et al. (1993), Choi et al. (1993), Lonberg et al. (1994), Taylor et al. (1994), and Tuaillon et al. (1995), Fishwild et al. (1996).

Kirin has also demonstrated the generation of human antibodies from mice in which, through microcell fusion, large pieces of chromosomes, or entire chromosomes, have been introduced. See European Patent Application Nos. 773 288 and 843 961. Xenerex Biosciences is developing a technology for the potential generation of human antibodies. In this technology, SCID mice are reconstituted with human lymphatic cells, e.g., B and/or T cells. Mice are then immunized with an antigen and can generate an immune response against the antigen. See U.S. Pat. Nos. 5,476,996; 5,698,767; and 5,958,765.

Human anti-mouse antibody (HAMA) responses have led the industry to prepare chimeric or otherwise humanized antibodies. It is however expected that certain human anti-chimeric antibody (HACA) responses will be observed, particularly in chronic or multi-dose utilizations of the antibody. Thus, it would be desirable to provide antibody constructs comprising a human binding domain against the target cell surface antigen and a human binding domain against CD16 in order to vitiate concerns and/or effects of HAMA or HACA response.

The term “epitope” refers to a side on an antigen to which a binding domain, such as an antibody or immunoglobulin, or a derivative, fragment or variant of an antibody or an immunoglobulin, specifically binds. An “epitope” is antigenic and thus the term epitope is sometimes also referred to herein as “antigenic structure” or “antigenic determinant”. Thus, the binding domain is an “antigen interaction site”. Said binding/interaction is also understood to define a “specific recognition”.

“Epitopes” can be formed both by contiguous amino acids or non-contiguous amino acids juxtaposed by tertiary folding of a protein. A “linear epitope” is an epitope where an amino acid primary sequence comprises the recognized epitope. A linear epitope typically includes at least 3 or at least 4, and more usually, at least 5 or at least 6 or at least 7, for example, about 8 to about 10 amino acids in a unique sequence.

A “conformational epitope”, in contrast to a linear epitope, is an epitope wherein the primary sequence of the amino acids comprising the epitope is not the sole defining component of the epitope recognized (e.g., an epitope wherein the primary sequence of amino acids is not necessarily recognized by the binding domain). Typically, a conformational epitope comprises an increased number of amino acids relative to a linear epitope. With regard to recognition of conformational epitopes, the binding domain recognizes a three-dimensional structure of the antigen, preferably a peptide or protein or fragment thereof (in the context of the present invention, the antigenic structure for one of the binding domains is comprised within the target cell surface antigen protein). For example, when a protein molecule folds to form a three-dimensional structure, certain amino acids and/or the polypeptide backbone forming the conformational epitope become juxtaposed enabling the antibody to recognize the epitope. Methods of determining the conformation of epitopes include, but are not limited to, x-ray crystallography, two-dimensional nuclear magnetic resonance (2D-NMR) spectroscopy and site-directed spin labelling and electron paramagnetic resonance (EPR) spectroscopy.

The interaction between the binding domain and the epitope or the region comprising the epitope implies that a binding domain exhibits appreciable affinity for the epitope/the region comprising the epitope on a particular protein or antigen (here: e.g. CD16A and/or the target cell surface antigen, respectively) and, generally, does not exhibit significant reactivity with proteins or antigens other than e.g. CD16A, the other antigen on the surface of an immune effector cell, and/or the target cell surface antigen. “Appreciable affinity” includes binding with an affinity of about 10⁻⁶ M (KD) or stronger. Preferably, binding is considered specific when the binding affinity is about 10⁻¹² to 10⁻¹¹ M, 10⁻¹² to 10⁻⁹ M, 10⁻¹² to 10⁻¹⁰ M, 10⁻¹¹ to 10⁻⁸ g M, preferably of about 10⁻¹¹ to 10⁻⁹ M. Whether a binding domain specifically reacts with or binds to a target can be tested readily by, inter alia, comparing the reaction of said binding domain with a target protein or antigen with the reaction of said binding domain with proteins or antigens other than e.g. the CD16A, and/or the target cell surface antigen.

The term “does not essentially/substantially bind” or “is not capable of binding” means that a binding domain of the present invention does not bind a protein or antigen other e.g. the CD16A, and/or the target cell surface antigen, i.e., does not show reactivity of more than 30%, preferably not more than 20%, more preferably not more than 10%, particularly preferably not more than 9%, 8%, 7%, 6% or 5% with proteins or antigens other than e.g. the CD16A, and/or the target cell surface antigen, whereby binding to e.g. the CD16A, and/or the target cell surface antigen, respectively, is set to be 100%.

Specific binding is believed to be affected by specific motifs in the amino acid sequence of the binding domain and the antigen. Thus, binding is achieved as a result of their primary, secondary and/or tertiary structure as well as the result of secondary modifications of said structures. The specific interaction of the antigen-interaction-side with its specific antigen may result in a simple binding of said side to the antigen. Moreover, the specific interaction of the antigen-interaction-side with its specific antigen may alternatively or additionally result in the initiation of a signal, e.g. due to the induction of a change of the conformation of the antigen, an oligomerization of the antigen, etc.

The term “variable” refers to the portions of the antibody or immunoglobulin domains that exhibit variability in their sequence and that are involved in determining the specificity and binding affinity of a particular antibody (i.e., the “variable domain(s)”). The pairing of a variable heavy chain (VH) and a variable light chain (VL) together forms a single antigen-binding side.

Variability is not evenly distributed throughout the variable domains of antibodies; it is concentrated in sub-domains of each of the heavy and light chain variable regions. These sub-domains are called “hypervariable regions” or “complementarity determining regions” (CDRs). The more conserved (i.e., non-hypervariable) portions of the variable domains are called the “framework” regions (FRM or FR) and provide a scaffold for the six CDRs in three dimensional space to form an antigen-binding surface. The variable domains of naturally occurring heavy and light chains each comprise four FRM regions (FR1, FR2, FR3, and FR4), largely adopting a β-sheet configuration, connected by three hypervariable regions, which form loops connecting, and in some cases forming part of, the β-sheet structure. The hypervariable regions in each chain are held together in close proximity by the FRM and, with the hypervariable regions from the other chain, contribute to the formation of the antigen-binding side (see Kabat et al., loc. cit.).

The terms “CDR”, and its plural “CDRs”, refer to the complementarity determining region of which three make up the binding character of a light chain variable region (CDR-L1, CDR-L2 and CDR-L3) and three make up the binding character of a heavy chain variable region (CDR-H1, CDR-H2 and CDR-H3). CDRs contain most of the residues responsible for specific interactions of the antibody with the antigen and hence contribute to the functional activity of an antibody molecule: they are the main determinants of antigen specificity.

The exact definitional CDR boundaries and lengths are subject to different classification and numbering systems. CDRs may therefore be referred to by Kabat, Chothia, contact or any other boundary definitions, including the numbering system described herein. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat (an approach based on cross-species sequence variability), Chothia (an approach based on crystallographic studies of antigen-antibody complexes), and/or MacCallum (Kabat et al., loc. cit; Chothia et al., J. Mol. Biol, 1987, 196: 901-917; and MacCallum et al., J. Mol. Biol, 1996, 262: 732). Still another standard for characterizing the antigen binding side is the AbM definition used by Oxford Molecular's AbM antibody modeling software. See, e.g., Protein Sequence and Structure Analysis of Antibody Variable Domains. In: Antibody Engineering Lab Manual (Ed.: Duebel, S. and Kontermann, R., Springer-Verlag, Heidelberg). To the extent that two residue identification techniques define regions of overlapping, but not identical regions, they can be combined to define a hybrid CDR. However, the numbering in accordance with the so-called Kabat system is preferred.

Typically, CDRs form a loop structure that can be classified as a canonical structure. The term “canonical structure” refers to the main chain conformation that is adopted by the antigen binding (CDR) loops. From comparative structural studies, it has been found that five of the six antigen binding loops have only a limited repertoire of available conformations. Each canonical structure can be characterized by the torsion angles of the polypeptide backbone. Correspondent loops between antibodies may, therefore, have very similar three dimensional structures, despite high amino acid sequence variability in most parts of the loops (Chothia and Lesk, J. Mol. Biol., 1987, 196: 901; Chothia et al., Nature, 1989, 342: 877; Martin and Thornton, J. Mol. Biol, 1996, 263: 800). Furthermore, there is a relationship between the adopted loop structure and the amino acid sequences surrounding it. The conformation of a particular canonical class is determined by the length of the loop and the amino acid residues residing at key positions within the loop, as well as within the conserved framework (i.e., outside of the loop). Assignment to a particular canonical class can therefore be made based on the presence of these key amino acid residues.

The term “canonical structure” may also include considerations as to the linear sequence of the antibody, for example, as catalogued by Kabat (Kabat et al., loc. cit.). The Kabat numbering scheme (system) is a widely adopted standard for numbering the amino acid residues of an antibody variable domain in a consistent manner and is the preferred scheme applied in the present invention as also mentioned elsewhere herein. Additional structural considerations can also be used to determine the canonical structure of an antibody. For example, those differences not fully reflected by Kabat numbering can be described by the numbering system of Chothia et al. and/or revealed by other techniques, for example, crystallography and two- or three-dimensional computational modeling. Accordingly, a given antibody sequence may be placed into a canonical class which allows for, among other things, identifying appropriate chassis sequences (e.g., based on a desire to include a variety of canonical structures in a library). Kabat numbering of antibody amino acid sequences and structural considerations as described by Chothia et al., loc. cit. and their implications for construing canonical aspects of antibody structure, are described in the literature. The subunit structures and three-dimensional configurations of different classes of immunoglobulins are well known in the art. For a review of the antibody structure, see Antibodies: A Laboratory Manual, Cold Spring Harbor Laboratory, eds. Harlow et al., 1988. A global reference in immunoinformatics is the three-dimensional (3D) structure database of IMGT (international ImMunoGenetics information system) (Ehrenmann et al., 2010, Nucleic Acids Res., 38, D301-307). The IMGT/3D structure-DB structural data are extracted from the Protein Data Bank (PDB) and annotated according to the IMGT concepts of classification, using internal tools. Thus, IMGT/3Dstructure-DB provides the closest genes and alleles that are expressed in the amino acid sequences of the 3D structures, by aligning these sequences with the IMGT domain reference directory. This directory contains, for the antigen receptors, amino acid sequences of the domains encoded by the constant genes and the translation of the germline variable and joining genes. The CDR regions of our amino acid sequences were preferably determined by using the IMGT/3Dstructure database.

The CDR3 of the light chain and, particularly, the CDR3 of the heavy chain may constitute the most important determinants in antigen binding within the light and heavy chain variable regions. In some antibody constructs, the heavy chain CDR3 appears to constitute the major area of contact between the antigen and the antibody. In vitro selection schemes in which CDR3 alone is varied can be used to vary the binding properties of an antibody or determine which residues contribute to the binding of an antigen. Hence, CDR3 is typically the greatest source of molecular diversity within the antibody-binding side. H3, for example, can be as short as two amino acid residues or greater than 26 amino acids.

In a classical full-length antibody or immunoglobulin, each light (L) chain is linked to a heavy (H) chain by one covalent disulfide bond, while the two H chains are linked to each other by one or more disulfide bonds depending on the H chain isotype. The CH domain most proximal to VH is usually designated as CHi. The constant (“C”) domains are not directly involved in antigen binding, but exhibit various effector functions, such as antibody-dependent, cell-mediated cytotoxicity and complement activation. The Fc region of an antibody is comprised within the heavy chain constant domains and is for example able to interact with cell surface located Fc receptors.

The sequence of antibody genes after assembly and somatic mutation is highly varied, and these varied genes are estimated to encode 10¹⁰ different antibody molecules (Immunoglobulin Genes, 2nd ed., eds. Jonio et al., Academic Press, San Diego, CA, 1995). Accordingly, the immune system provides a repertoire of immunoglobulins. The term “repertoire” refers to at least one nucleotide sequence derived wholly or partially from at least one sequence encoding at least one immunoglobulin. The sequence(s) may be generated by rearrangement in vivo of the V, D, and J segments of heavy chains, and the V and J segments of light chains. Alternatively, the sequence(s) can be generated from a cell in response to which rearrangement occurs, e.g., in vitro stimulation. Alternatively, part or all of the sequence(s) may be obtained by DNA splicing, nucleotide synthesis, mutagenesis, and other methods, see, e.g., U.S. Pat. No. 5,565,332. A repertoire may include only one sequence or may include a plurality of sequences, including ones in a genetically diverse collection.

The antibody construct defined in the context of the invention may also comprise additional domains, which are e.g. helpful in the isolation of the molecule or relate to an adapted pharmacokinetic profile of the molecule. Domains helpful for the isolation of an antibody construct may be selected from peptide motives or secondarily introduced moieties, which can be captured in an isolation method, e.g. an isolation column. Non-limiting embodiments of such additional domains comprise peptide motives known as Myc-tag, HAT-tag, HA-tag, TAP-tag, GST-tag, chitin binding domain (CBD-tag), maltose binding protein (MBP-tag), Flag-tag, Strep-tag and variants thereof (e.g. Strepll-tag) and His-tag. All herein disclosed antibody constructs characterized by the identified CDRs may comprise a His-tag domain, which is generally known as a repeat of consecutive His residues in the amino acid sequence of a molecule, preferably of five, and more preferably of six His residues (hexa-histidine). The His-tag may be located e.g. at the N- or C-terminus of the antibody construct, preferably it is located at the C-terminus. Most preferably, a hexa-histidine tag is linked via peptide bond to the C-terminus of the antibody construct according to the invention. Additionally, a conjugate system of PLGA-PEG-PLGA may be combined with a poly-histidine tag for sustained release application and improved pharmacokinetic profile.

Amino acid sequence modifications of the antibody constructs described herein are also contemplated, as long as the minimal structural limitations of the first binding domain of the antibody construct of the present invention are maintained. For example, it may be desirable to improve the binding affinity and/or other biological properties of the antibody construct. Amino acid sequence variants of the antibody constructs are prepared by introducing appropriate nucleotide changes into the antibody constructs nucleic acid, or by peptide synthesis. All of the below described amino acid sequence modifications should result in an antibody construct which still retains the desired biological activity (i.e. binding to CD16A, and/or the target cell surface antigen) of the unmodified parental molecule.

The term “amino acid” or “amino acid residue” typically refers to an amino acid having its art recognized definition such as an amino acid selected from the group consisting of: alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (Ile or I): leucine (Leu or L); lysine (Lys or K); methionine (Met or M); phenylalanine (Phe or F); proline (Pro or P); serine (Ser or S); threonine (Thr or T); tryptophan (Trp or W); tyrosine (Tyr or Y); and valine (Val or V), although modified, synthetic, or rare amino acids may be used as desired. Generally, amino acids can be grouped as having a nonpolar side chain (e.g., Ala, Cys, Ile, Leu, Met, Phe, Pro, Val); a negatively charged side chain (e.g., Asp, Glu); a positively charged sidechain (e.g., Arg, His, Lys); or an uncharged polar side chain (e.g., Asn, Cys, Gln, Gly, His, Met, Phe, Ser, Thr, Trp, and Tyr).

Amino acid modifications include, for example, deletions from, and/or insertions into, and/or substitutions of, residues within the amino acid sequences of the antibody constructs. Any combination of deletion, insertion, and substitution is made to arrive at the final construct, provided that the final construct possesses the desired characteristics. The amino acid changes also may alter post-translational processes of the antibody constructs, such as changing the number or position of glycosylation sites.

For example, in particular in context of the second binding domain of the antibody construct, 1, 2, 3, 4, 5, or 6 amino acids may be inserted, substituted or deleted in each of the CDRs (of course, dependent on their length), while 1, 2, 3, 4, 5, 6, 7, 8, 9, 10, 11, 12, 13, 14, 15, 16, 17, 18, 19, 20, or 25 amino acids may be inserted, substituted or deleted in each of the FRs. Preferably, amino acid sequence insertions into the antibody construct include amino- and/or carboxyl-terminal fusions ranging in length from 1, 2, 3, 4, 5, 6, 7, 8, 9 or 10 residues to polypeptides containing a hundred or more residues, as well as intra-sequence insertions of single or multiple amino acid residues. An insertional variant of the antibody construct defined in the context of the invention includes the fusion to the N-terminus or to the C-terminus of the antibody construct of an enzyme or the fusion to a polypeptide.

The sites of greatest interest for substitutional mutagenesis include (but are not limited to) the CDRs of the heavy and/or light chain of the second binding domain, in particular the hypervariable regions, but FR alterations in the heavy and/or light chain are also contemplated.

The substitutions are preferably conservative substitutions as described herein. Preferably, 1, 2, 3, 4, 5, 6, 7, 8, 9, or 10 amino acids may be substituted in a CDR, while 1, 2, 3, 4, 5, 6, 7, 8, 9, 10, 11, 12, 13, 14, 15, 16, 17, 18, 19, 20, or 25 amino acids may be substituted in the framework regions (FRs), depending on the length of the CDR or FR. For example, if a CDR sequence encompasses 6 amino acids, it is envisaged that one, two or three of these amino acids are substituted. Similarly, if a CDR sequence encompasses 15 amino acids it is envisaged that one, two, three, four, five or six of these amino acids are substituted.

A useful method for identification of certain residues or regions of the antibody constructs that are preferred locations for mutagenesis is called “alanine scanning mutagenesis” as described by Cunningham and Wells in Science, 244: 1081-1085 (1989). Here, a residue or group of target residues within the antibody construct is/are identified (e.g. charged residues such as arg, asp, his, lys, and glu) and replaced by a neutral or negatively charged amino acid (most preferably alanine or polyalanine) to affect the interaction of the amino acids with the epitope.

Those amino acid locations demonstrating functional sensitivity to the substitutions are then refined by introducing further or other variants at, or for, the sites of substitution. Thus, while the site or region for introducing an amino acid sequence variation is predetermined, the nature of the mutation per se needs not to be predetermined. For example, to analyze or optimize the performance of a mutation at a given site, alanine scanning or random mutagenesis may be conducted at a target codon or region, and the expressed antibody construct variants are screened for the optimal combination of desired activity. Techniques for making substitution mutations at predetermined sites in the DNA having a known sequence are well known, for example, M13 primer mutagenesis and PCR mutagenesis. Screening of the mutants is done using assays of antigen binding activities, such as for the binding to e.g. CD16a, and/or the target cell surface antigen binding.

Generally, if amino acids are substituted in one or more or all of the CDRs of the heavy and/or light chain, it is preferred that the then-obtained “substituted” sequence is at least 60% or at least 65%, more preferably at least 70% or at least 75%, even more preferably at least 80% or at least 85%, and particularly preferably at least 90% or at least 95% identical to the “original” CDR sequence. This means that it is dependent of the length of the CDR to which degree it is identical to the “substituted” sequence. For example, a CDR having 5 amino acids is preferably at least 80% identical to its substituted sequence in order to have at least one amino acid substituted. Accordingly, the CDRs of the antibody construct may have different degrees of identity to their substituted sequences, e.g., CDRL1 may have at least 80%, while CDRL3 may have at least 90%.

Preferred substitutions (or replacements) are conservative substitutions. However, any substitution (including non-conservative substitution) is envisaged as long as the antibody construct retains its capability to bind to CD16A via the first binding domain, and/or to the target cell surface antigen via the second binding domain and/or the CDRs of the second binding domain have an identity to the then substituted sequence (at least 60% or at least 65%, more preferably at least 70% or at least 75%, even more preferably at least 80% or at least 85%, and particularly preferably at least 90% or at least 95% identical to the “original” CDR sequence).

Conservative substitutions are shown in Table 1 under the heading of “preferred substitutions”. If such substitutions result in a change in biological activity, then more substantial changes, denominated “exemplary substitutions” in Table 1, or as further described below in reference to amino acid classes, may be introduced and the products screened for a desired characteristic.

TABLE 1
Amino acid substitutions

| Original | Exemplary Substitutions | Preferred Substitutions |
|---|---|---|
| Ala (A) | val, leu, ile | val |
| Arg (R) | lys, gln, asn | lys |
| Asn (N) | gln, his, asp, lys, arg | gln |
| Asp (D) | glu, asn | glu |
| Cys (C) | ser, ala | ser |
| Gln (Q) | asn, glu | asn |
| Glu (E) | asp, gln | asp |
| Gly (G) | ala | ala |
| His (H) | asn, gln, lys, arg | arg |
| Ile(I) | leu, val, met, ala, phe | leu |
| Leu (L) | norleucine, ile, val, met, ala | lie |
| Lys (K) | arg, gln, asn | arg |
| Met (M) | leu, phe, ile | leu |
| Phe (F) | leu, val, ile, ala, tyr | tyr |
| Pro (P) | ala | ala |
| Ser (S) | thr | thr |
| Thr (T) | ser | ser |
| Trp (W) | tyr, phe | tyr |
| Tyr (Y) | trp, phe, thr, ser | phe |
| Val (V) | ile, leu, met, phe, ala | leu |

Substantial modifications in the biological properties of the antibody construct of the present invention are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties: (1) hydrophobic: norleucine, met, ala, val, leu, ile; (2) neutral hydrophilic: cys, ser, thr, asn, gln; (3) acidic: asp, glu; (4) basic: his, lys, arg; (5) residues that influence chain orientation: gly, pro; and (6) aromatic: trp, tyr, phe.

Non-conservative substitutions will entail exchanging a member of one of these classes for another class. Any cysteine residue not involved in maintaining the proper conformation of the antibody construct may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

For amino acid sequences, sequence identity and/or similarity is determined by using standard techniques known in the art, including, but not limited to, the local sequence identity algorithm of Smith and Waterman, 1981, Adv. Appl. Math. 2:482, the sequence identity alignment algorithm of Needleman and Wunsch, 1970, J. Mol. Biol. 48:443, the search for similarity method of Pearson and Lipman, 1988, Proc. Nat. Acad. Sci. U.S.A. 85:2444, computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., 1984, Nucl. Acid Res. 12:387-395, preferably using the default settings, or by inspection. Preferably, percent identity is calculated by FastDB based upon the following parameters: mismatch penalty of 1; gap penalty of 1; gap size penalty of 0.33; and joining penalty of 30, “Current Methods in Sequence Comparison and Analysis,” Macromolecule Sequencing and Synthesis, Selected Methods and Applications, pp 127-149 (1988), Alan R. Liss, Inc.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, 1987, J. Mol. Evol. 35:351-360; the method is similar to that described by Higgins and Sharp, 1989, CABIOS 5:151-153. Useful PILEUP parameters including a default gap weight of 3.00, a default gap length weight of 0.10, and weighted end gaps.

Another example of a useful algorithm is the BLAST algorithm, described in: Altschul et a1., 1990, J. Mol. Biol. 215:403-410; Altschul et al., 1997, Nucleic Acids Res. 25:3389-3402; and Karin et al., 1993, Proc. Natl. Acad. Sci. U.S.A. 90:5873-5787. A particularly useful BLAST program is the WU-BLAST-2 program which was obtained from Altschul et al., 1996, Methods in Enzymology 266:460-480. WU-BLAST-2 uses several search parameters, most of which are set to the default values. The adjustable parameters are set with the following values: overlap span=1, overlap fraction=0.125, word threshold (T)=ll. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., 1993, Nucl. Acids Res. 25:3389-3402. Gapped BLAST uses BLOSUM-62 substitution scores; threshold T parameter set to 9; the two-hit method to trigger ungapped extensions, charges gap lengths of k a cost of 10+k; Xu set to 16, and Xg set to 40 for database search stage and to 67 for the output stage of the algorithms. Gapped alignments are triggered by a score corresponding to about 22 bits.

Generally, the amino acid homology, similarity, or identity between individual variant CDRs or VH/VL sequences are at least 60% to the sequences depicted herein, and more typically with preferably increasing homologies or identities of at least 65% or 70%, more preferably at least 75% or 80%, even more preferably at least 85%, 90%, 91%, 92%, 93%, 94%, 95%, 96%, 97%, 98%, 99%, and almost 100%. In a similar manner, “percent (%) nucleic acid sequence identity” with respect to the nucleic acid sequence of the binding proteins identified herein is defined as the percentage of nucleotide residues in a candidate sequence that are identical with the nucleotide residues in the coding sequence of the antibody construct. A specific method utilizes the BLASTN module of WU-BLAST-2 set to the default parameters, with overlap span and overlap fraction set to 1 and 0.125, respectively.

Generally, the nucleic acid sequence homology, similarity, or identity between the nucleotide sequences encoding individual variant CDRs or VH/VL sequences and the nucleotide sequences depicted herein are at least 60%, and more typically with preferably increasing homologies or identities of at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%, and almost 100%. Thus, a “variant CDR” or a “variant VH/VL region” is one with the specified homology, similarity, or identity to the parent CDR/VH/VL defined in the context of the invention, and shares biological function, including, but not limited to, at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR or VH/VL.

In one embodiment, the percentage of identity to human germline of the antibody constructs according to the invention is ≥70% or ≥75%, more preferably ≥80% or ≥85%, even more preferably ≥90%, and most preferably ≥91%, ≥92%, ≥93%, ≥94%, ≥95% or even ≥96%. Identity to human antibody germline gene products is thought to be an important feature to reduce the risk of therapeutic proteins to elicit an immune response against the drug in the patient during treatment. Hwang & Foote (“Immunogenicity of engineered antibodies”; Methods 36 (2005) 3-10) demonstrate that the reduction of non-human portions of drug antibody constructs leads to a decrease of risk to induce anti-drug antibodies in the patients during treatment. By comparing an exhaustive number of clinically evaluated antibody drugs and the respective immunogenicity data, the trend is shown that humanization of the V-regions of antibodies makes the protein less immunogenic (average 5.1% of patients) than antibodies carrying unaltered non-human V regions (average 23.59% of patients). A higher degree of identity to human sequences is hence desirable for V-region based protein therapeutics in the form of antibody constructs. For this purpose of determining the germline identity, the V-regions of VL can be aligned with the amino acid sequences of human germline V segments and J segments (vbase.mrc-cpe.cam.ac.uk/) using Vector NTI software and the amino acid sequence calculated by dividing the identical amino acid residues by the total number of amino acid residues of the VL in percent. The same can be for the VH segments (vbase.mrc-cpe.cam.ac.uk/) with the exception that the VH CDR3 may be excluded due to its high diversity and a lack of existing human germline VH CDR3 alignment partners. Recombinant techniques can then be used to increase sequence identity to human antibody germline genes.

As used herein, the term “CD123” refers to the Cluster of Differentiation 123 protein, also known as “interleukin-3 receptor”, is an antigenic determinant found on pluripotent progenitor cells, basophils and plasmacytoid dendritic cells (pDCs) as well as some conventional dendritic cells (cDCs) among peripheral blood mononuclear cells. The human and murin amino acid and nucleic acid sequences can be found in a public database, such as GenBank, UniProt and Swiss-Prot. For example, the amino acid sequence of human CD123 can be found as UniProt/Swiss-Prot Accession No. P26951 and the nucleotide sequence encoding of the human CD123 can be found at Accession No. NM_002183. As used herein, “CD123” includes proteins comprising mutations, e.g., point mutations, fragments, insertions, deletions and splice variants of full length wild-type CD123. CD123 is a biomarker in hematolymphoid malignancies (El Achi et al., Cancers (Basel)., 2020, 12(11): 3087) and particularly expressed across acute myeloid leukemia (AML) subtypes, including leukemic stem cells (Seattle Genetics Initiates Phase 1 Trial of SGN-CD123A for Patients with Relapsed or Refractory Acute Myeloid Leukemia September 2016).

As used herein, the term “FOLR1” also known as “Folate Receptor 1”, “Folate Receptor Alpha”, “Folate Binding Protein” (FBP” or “Ovarian Cancer-Associated Antigen” is a receptor with high affinity for folic acid and for several reduced folic acid derivatives and mediates delivery of 5-methyltetrahydrofolate to the interior of cells (Henderson, Annu Rev Nutr. 1990; 10:319-35). The human and murin amino acid and nucleic acid sequences can be found in a public database, such as GenBank, UniProt and Swiss-Prot. For example, the amino acid sequence of human FOLR1 can be found as UniProt/Swiss-Prot Accession No. P15328 and the nucleotide sequence encoding of the human FOLR1 can be found at Accession No. BT007158.1. FOLR1 can be overexpressed by a number of epithelial-derived tumors including ovarian, breast (particularly TNBC), renal, lung, colorectal (CRC), kidney (particularly ccRCC), pancreatic (particularly PDAC), endometrial and brain (Scaranti et al., Nat Rev Clin Oncol. 2020, 17(6): 349-359). Due to its high expression in some tumors, FOLR1 is an attractive therapeutic target for the development of anti-cancer agents in order to limit toxic side-effects on off-target tissues. As used herein, “FOLR1” includes proteins comprising mutations, e.g., point mutations, fragments, insertions, deletions and splice variants of full length wild-type FOLR1.

“Overexpressing” or “overexpression” as used herein in respect to FOLR1 refers to an increase of the amount of FOLR1 on the surface of a cell in a pathological context, such as a proliferative disease, a tumorous disease, a viral disease or an immunological disorder, when compared to the expression of FOLR1 on the surface of a cell of the same tissue in a non-pathological context. For example, “overexpressing” or “overexpression” as used herein in respect to FOLR1 refers to an increase of the amount of FOLR1 on the surface of a tumorous cell when compared to the expression of FOLR1 on the surface of a non-tumorous cell of the same tissue.

The term “immune effector cell” as used herein may refer to any leukocyte or precursor involved e.g. in defending the body against cancer, diseases induced by infectious agents, foreign materials or autoimmune reactions. For example, the immune effector cells comprise B lymphocytes (B cells), T lymphocytes (T cells, including CD4+ and CD8+ T cells), NK cells, NKT cells, monocytes, macrophages, dendritic cells, mast cells, granulocytes such as neutrophils, basophils and eosinophils, innate lymphoid cells (ILCs, which comprise ILC-1, ILC-2 and ILC-3) or any combinations thereof. Preferably, the term immune effector cell refers to an NK cell, an ILC-1 cell, a NKT cell, a macrophage, a monocyte, and/or a T cell, such as a CD8+ T cell or a T6 T cell.

Natural killer (NK) cells are CD56+CD3− large granular lymphocytes that can kill virally infected and transformed cells, and constitute a critical cellular subset of the innate immune system (Godfrey J, et al. Leuk Lymphoma 2012 53:1666-1676). Unlike cytotoxic CD8+T lymphocytes, NK cells launch cytotoxicity against tumor cells without the requirement for prior sensitization and can also eradicate MHIC-I-negative cells (Narni-Mancinelli E, et al. Int Immunol 2011 23:427-431). NK cells are safer effector cells, as they may avoid the potentially lethal complications of cytokine storms (Morgan R A, et al. Mol Ther 2010 18:843-851), tumor lysis syndrome (Porter D L, et al. N Engl J Med 2011 365:725-733), and on-target, off-tumor effects.

Monocytes are produced by the bone marrow from haematopoietic stem cell precursors called monoblasts. Monocytes circulate in the bloodstream for about one to three days and then typically move into tissues throughout the body. They constitute between three to eight percent of the leukocytes in the blood. In the tissue monocytes mature into different types of macrophages at different anatomical locations. Monocytes have two main functions in the immune system: (1) replenish resident macrophages and dendritic cells under normal states, and (2) in response to inflammation signals, monocytes can move quickly (approx. 8-12 hours) to sites of infection in the tissues and divide/differentiate into macrophages and dendritic cells to elicit an immune response. Monocytes are usually identified in stained smears by their large bilobate nucleus.

Macrophages are potent effectors of the innate immune system and are capable of at least three distinct anti-tumor functions: phagocytosis, cellular cytotoxicity, and antigen presentation to orchestrate an adaptive immune response. While T cells require antigen-dependent activation via the T cell receptor or the chimeric immunoreceptor, macrophages can be activated in a variety of ways. Direct macrophage activation is antigen-independent, relying on mechanisms such as pathogen associated molecular pattern recognition by Toll-like receptors (TLRs). Immune-complex mediated activation is antigen dependent but requires the presence of antigen-specific antibodies and absence of the inhibitory CD47-SIRPa interaction.

T cells or T lymphocytes can be distinguished from other lymphocytes, such as B cells and natural killer cells (NK cells), by the presence of a T-cell receptor (TCR) on the cell surface. They are called T cells because they mature in the thymus (although some also mature in the tonsils). There are several subsets of T cells, each with a distinct function.

T helper cells (TH cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including TH1, TH2, TH3, TH17, TH9, or TFH, which secrete different cytokines to facilitate a different type of immune response.

Cytotoxic T cells (TC cells, or CTLs) destroy virally infected cells and tumor cells, and are also implicated in transplant rejection. These cells are also known as CD8+ T cells since they express the CD8 glycoprotein at their surface. These cells recognize their targets by binding to antigen associated with MHC class I molecules, which are present on the surface of all nucleated cells. Through IL-10, adenosine and other molecules secreted by regulatory T cells, the CD8+ cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4+ Treg cells have been described-naturally occurring Treg cells and adaptive Treg cells.

Natural killer T (NKT) cells (not to be confused with natural killer (NK) cells) bridge the adaptive immune system with the innate immune system. Unlike conventional T cells that recognize peptide antigens presented by major histocompatibility complex (MIC) molecules, NKT cells recognize glycolipid antigen presented by a molecule called CD1d.

As used herein, the term “half-life extensions domain” relates to a moiety that prolongs serum half-life of the antibody construct. The half-life extension domain may comprise a portion of an antibody, such as an Fc part of an immunoglobulin, a hinge domain, a CH2 domain, a CH3 domain, and/or a CH4 domain. Although less preferred, a half-life extension domain can also comprise elements that are not comprised in an antibody, such as an albumin binding peptide, an albumin binding protein, or transferrin to name only a few. A half-life extension domain preferably does not have an immune-modulatory function. If a half-life extension domain comprises a hinge, CH2 and/or CH3 domain, the half-life extension domain preferably does not essentially bind to an Fc receptor. This can e.g. be achieved through “silencing” of the Fcγ receptor binding domain.

As used herein, “silencing” of the Fc or Fcγ receptor binding domain refers to any modification that reduces binding of a CH2 domain to an Fc receptor, in particular an Fcγ receptor. Such modification can be done by replacement and/or deletion of one or more amino acids that are involved in Fc(γ) receptor-binding. Such mutations are well known in the art and have e.g. been described by Saunders (2019, Front. Immunol. 10:1296). For example, a mutation can be located at any one of the positions 233, 234, 235, 236, 237, 239, 263, 265, 267, 273, 297, 329, and 331. Examples for such mutations are: deletion of Glu 233->Pro, Glu 233, Leu 234->Phe, Leu 234->Ala, Leu 234->Gly, Leu 234->Glu, Leu 234->Val, deletion of Leu 234, Leu 235->Glu, Leu 235->Ala, Leu 235->Arg, Leu 235->Phe, deletion of Leu 235, deletion of Gly 236, Gly 237->Ala, Ser 239->Lys, Val 263->Leu, Asp 265->Ala, Ser 267->Lys, Val 273->Glu, Asn 297->Gly, Asn 297->Ala, Lys 332->Ala, Pro 329->Gly, Pro 331->Ser and combinations thereof. Preferably, such a modification comprises one or both of Leu 234->Ala and Leu 235->Ala (also known as “LALA” mutation). Preferably, such a modification further comprises a Pro 329->Gly mutation, also known as “LALA-PG” mutation (Leu 234->Ala, Leu 235->Ala, and Pro 329->Gly). Preferably, such a modification comprises 1, 2, or 3 of the mutations Leu 234->Phe, Leu 235->Glu, and Asp 265->Ala, more preferably all three of these mutations. The combination Leu 234->Phe, Leu 235->Glu, and Asp 265->Ala, which is a preferred modification in the context of the present invention, is also known as “FEA” mutation. Preferably, such a modification further comprises Asn 297->Gly. Such a preferred modification comprises the mutations Leu 234->Phe, Leu 235->Glu, Asp 265->Ala, and Asn 297->Gly.

The term “treatment” refers to both therapeutic treatment and prophylactic or preventative measures. Treatment includes the application or administration of the formulation to the body, an isolated tissue, or cell from a patient who has a disease/disorder, a symptom of a disease/disorder, or a predisposition toward a disease/disorder, with the purpose to cure, heal, alleviate, relieve, alter, remedy, ameliorate, improve, or affect the disease, the symptom of the disease, or the predisposition toward the disease.

The term “amelioration” as used herein refers to any improvement of the disease state of a patient having a tumor or cancer or a metastatic cancer as defined elsewhere herein, by the administration of an antibody construct according to the invention to a subject in need thereof.

Such an improvement may also be seen as a slowing or stopping of the progression of the tumor or cancer or metastatic cancer of the patient.

The term “disease” refers to any condition that would benefit from treatment with the antibody construct or the pharmaceutic composition described herein. This includes chronic and acute disorders or diseases including those pathological conditions that predispose the mammal to the disease in question.

The term “tumorous diseases” or “tumor disease” refers to a disease characterized by the presence or development of a tumor. A “tumor” is an abnormal growth of cells that serves no purpose. Tumors are divided into benign tumors, i.e. non-malignant tumors, and malignant tumors, i.e. cancerous tumors/cancer. While benign tumors grow slowly, have distinct borders and do not invade nearby tissue/do not spread to other parts of the body, malignant tumor can grow quickly, have irregular borders, often invade surrounding tissue and spread to other parts of the body called metastasis (Patel, JAMA Oncol, 2020, 6(9):1488).

“Tumors of the hematopoietic and lymphoid tissues” are tumors that affect the blood, bone marrow, lymph, and lymphatic system (Vardiman et al.; Blood, 2009, 114(5): 937-51).

“Solid tumors” refer to new growths of tissue, i.e. an abnormal mass of tissues, that usually does not contain cysts or liquid areas. These can occur anywhere in the body. Solid tumors may be benign (not cancer), or malignant (cancer). One speaks of benign tumors when tumors do not grow through (infiltrate) the surrounding tissue and do not form secondary tumors (metastases).

Malignant solid tumors, on the other hand, destroy surrounding tissue and can spread to other parts of the body. Malignant neoplasms are also known as cancer. It is particularly envisaged that “solid tumors” in the context of the present invention address malignant solid tumors, selected from the group consisting of brain cancer, head and neck cancer, lung cancer, esophageal cancer, gastric cancer, hepatocellular carcinoma, small intestine cancer, colorectal cancer, pancreatic cancer, breast cancer, ovarian cancer, cervical cancer, endometrial cancer, prostate cancer, renal cancer, bladder cancer, thyroid cancer, skin cancer, melanoma, and sarcoma, preferably ovarian, breast, renal, lung, colorectal, and brain cancer. A “neoplasm” is an abnormal growth of tissue, usually but not always forming a mass. When also forming a mass, it is commonly referred to as a “tumor”. Neoplasms or tumors can be benign, potentially malignant (pre-cancerous), or malignant. Malignant neoplasms are commonly called cancer. They usually invade and destroy the surrounding tissue and may form metastases, i.e., they spread to other parts, tissues or organs of the body. Hence, the term “metastatic cancer” encompasses metastases to other tissues or organs than the one of the original tumor. For the purposes of the present invention, they are also encompassed by the terms “tumor” or “cancer”.

“Proliferating diseases” are characterized by an excessive proliferation of cells and turnover of cellular matrix as described e.g. in Sporn and Harris, The American Journal of Medicine, 1981, 70(6): 1231-1236.

“Viral diseases” are diseases caused by intrusion of pathogenic viruses, and infectious virus particles (virions) attach to, that enter susceptible cells (Taylor et al., PNAS, 2021, 106(42): 17046-17051). Viruses can have various structural characteristics and can comprises inter alia double-stranded DNA families (such as Adenoviridae, Papillomaviridae and Polyomaviridae), partly double-stranded DNA viruses (such as Hepadnaviridae), single-stranded DNA viruses (such as Parvoviridae), positive single-stranded RNA families (three non-enveloped such as Astroviridae, Caliciviridae and Picornaviridae, four enveloped such as Coronaviridae, Flaviviridae, Retroviridae and Togaviridae), negative single-stranded RNA families (such as Arenaviridae, Bunyaviridae, Filoviridae, Orthomyxoviridae, Paramyxoviridae and Rhabdoviridae), and viruses with double-stranded RNA genome.

“Immunological disorders” are diseases or conditions caused by a dysfunction of the immune system and include allergy, asthma, autoimmune diseases, autoinflammatory syndromes and immunological deficiency syndromes.

The terms “subject in need” or those “in need of treatment” includes those already with the disorder or disease, as well as those in which the disorder or disease is to be prevented. The subject in need or “patient” includes human and other mammalian subjects that receive either prophylactic or therapeutic treatment.

The term “pharmaceutical composition” relates to a composition which is suitable for administration to a patient, preferably a human patient. The particularly preferred pharmaceutical composition of this invention comprises one or a plurality of the antibody construct(s) of the invention, preferably in a therapeutically effective dose. Preferably, the pharmaceutical composition further comprises suitable formulations of one or more (pharmaceutically effective) carriers, stabilizers, excipients, diluents, solubilizers, surfactants, emulsifiers, preservatives and/or adjuvants. Acceptable constituents of the composition are preferably nontoxic to recipients at the dosages and concentrations employed. Pharmaceutical compositions of the invention include, but are not limited to, liquid, frozen, and lyophilized compositions.

“Pharmaceutically acceptable carrier” means any and all aqueous and non-aqueous solutions, sterile solutions, solvents, buffers, e.g. phosphate buffered saline (PBS) solutions, water, suspensions, emulsions, such as oil/water emulsions, various types of wetting agents, liposomes, dispersion media and coatings, which are compatible with pharmaceutical administration, in particular with parenteral administration. The use of such media and agents in pharmaceutical compositions is well known in the art, and the compositions comprising such carriers can be formulated by well-known conventional methods.

The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts or doses effective for this use will depend on the condition to be treated (the indication), the delivered antibody construct, the therapeutic context and objectives, the severity of the disease, prior therapy, the patient's clinical history and response to the therapeutic agent, the route of administration, the size (body weight, body surface or organ size) and/or condition (the age and general health) of the patient, and the general state of the patient's own immune system. The proper dose can be adjusted according to the judgment of the attending physician such that it can be administered to the patient once or over a series of administrations, and in order to obtain the optimal therapeutic effect.

The term “kit” as used herein means two or more components—one of which corresponding to the antibody construct, the pharmaceutical composition, the vector or the host cell of the invention—packaged together in a container, recipient or otherwise. A kit can hence be described as a set of products and/or utensils that are sufficient to achieve a certain goal, which can be marketed as a single unit.

### DETAILED DESCRIPTION

Innate immune effector cells (e.g. natural killer (NK) cells, macrophages) are activated by a complex mechanism of several different signaling pathways. NK cells and macrophages can be harnessed in cancer immunotherapy by redirecting NK cell lysis or macrophage-induced phagocytosis to tumor cells through stimulation of the activating antigen CD16A (FcγRIIIA) expressed on their cell surface. CD16A is associated with the signaling adaptor CD3ζ chain containing an immunoreceptor tyrosine-based activation motif (ITAM), initiating signaling cascades that ultimately mediate ADCC and antibody dependent cellular phagocytosis (ADCP) in NK cells and macrophages, respectively. Signaling via CD16A has been reported sufficient to activate the cytotoxic activity of NK cells.

However, in circumstances of e.g. an immunosuppressive tumor, microenvironment stimulation via CD16A may be suboptimal or insufficient for maximal anti-tumor activity. Therefore, targeting of an additional surface antigen on NK cells, macrophages, or other immune cell types such as, but not limited to, CD8+ αβ T cells or γδ T cells may improve or maximize anti-tumor activity.

However, even though activation-induced down-regulation/shedding of CD16, in particular CD16A, on activated NK cells is known to impair their activity, thereby decreasing NK cell responses at individual cell-cell contacts, CD16 shedding has lately been described as beneficial for the detachment of NK cells from opsonized target cells, which may sustain NK cell survival and reduce activation-induced death (Srpan et al., J. Cell. Biol., 2018, 217(9):3267-3283). Contrary to this teaching, the present invention aims at providing an antibody construct that is capable of activating immune effector cells such as NK cells via binding to CD16A on the surface of said effector cells without having the risk of activation-induced down-regulation/shedding of CD16A. This may be achieved by a specific high-affinity anti-CD16A binding domain (named CD16a1 anti-CD16A effector domain or CD16a1 domain herein) comprised by the antibody constructs of the present invention. This might be achieved a) by inhibiting shedding below a threshold that provides a compromise to sufficiently inhibit shedding leading to increased activation of NK cells while avoiding impairment of NK cell activity and/or b) by avoiding apoptotic death of NK cells due to excessive inhibition of CD16A shedding. Thus, the antibody construct of the present invention is capable of specifically activating immune effector cells for ADCC induced phagocytosis towards target cell antigens, specifically FOLR1, thereby leading to an efficient lysis of said target cells without loss of activity and efficiency due to activation-induced CD16A degradation.

The present invention thus envisions an antibody construct comprising a specific first binding domain (A), which is capable of specifically binding to a first target (A′) that is CD16A on the surface of an immune effector cell, and a second binding domain (B), which is capable of specifically binding to a second target (B′) that is an antigen on the surface of a target cell, wherein said second target (B′) is FOLR1, thereby reducing the rate of activation-induced CD16A shedding on the surface of said effector cells.

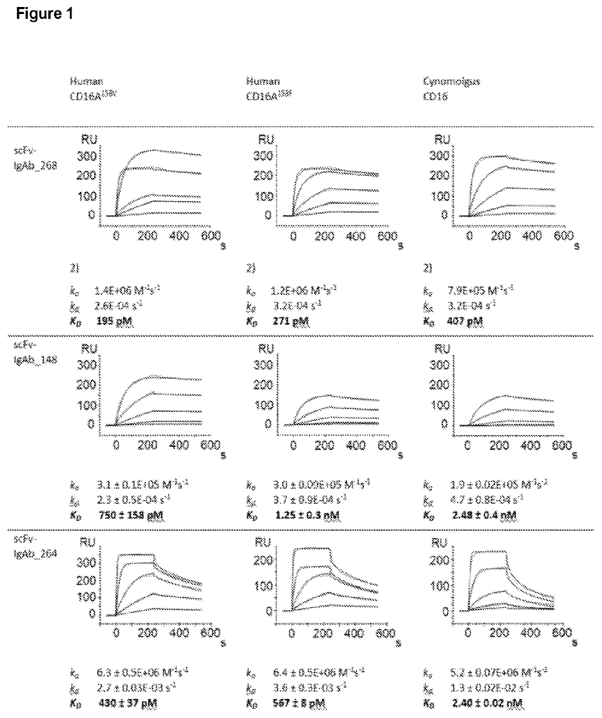

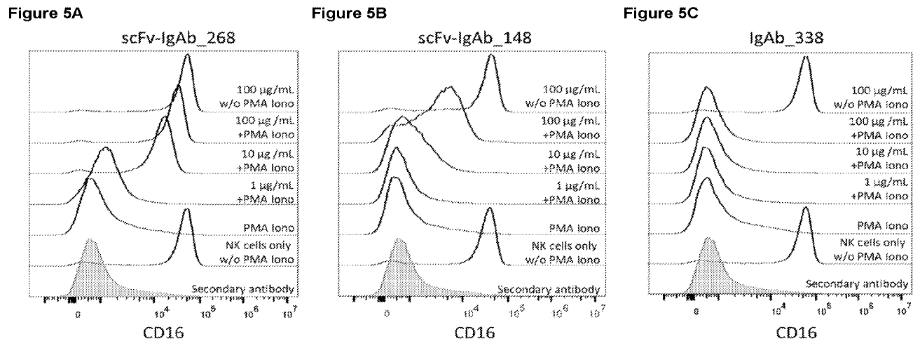

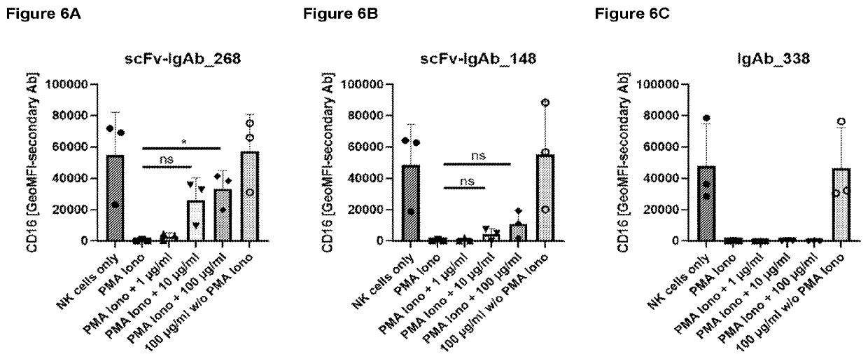

The inventors of the present application believe that the specific CD16A binding domain comprised by the antibody construct of the present invention is particularly beneficial when compared to known low-affinity CD16A binding domains (such as the CD16a2 or CD16a4 effector domain, also named CD16a2 or CD16a4 domain, described herein). This is so because the CD16A binding domain of the present invention provides for a high-affinity binding to CD16A on the surface of immune effector cells (see FIGS. 1, 2 and 16A-16C and Tables 3 and 15), but does not result in more potent induction of CD16A loss in presence of target cells (such as CD123 positive (+) cells). As demonstrated in FIGS. 5A-5C and 6A-6C of the present application, said CD16a1 binding domain leads to a stabilization of CD16A receptor levels at various antibody concentrations, despite the presence of target cells. While the presence of circulating target cells (such as CD123+ cells in peripheral blood) results in rapid activation of NK cells and loss of CD16A from the cell surface upon infusion of a bispecific CD123×CD16A-targeting antibody comprising a low-affinity anti-CD16A binding domain variants (such as CD16a2), the bispecific CD123×CD16A antibody constructs of the present invention that have a high-affinity anti-CD16A binding domain led to a stabilization of CD16A receptor levels, despite the presence of (CD123+) target cells.

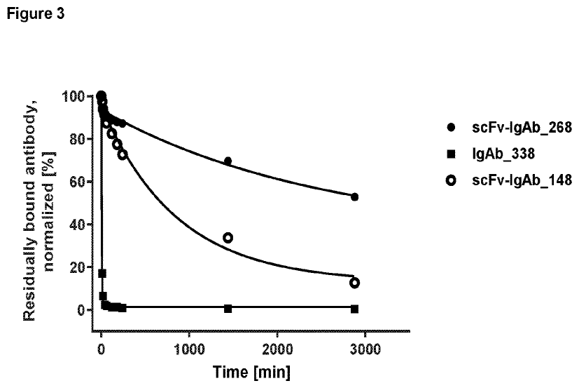

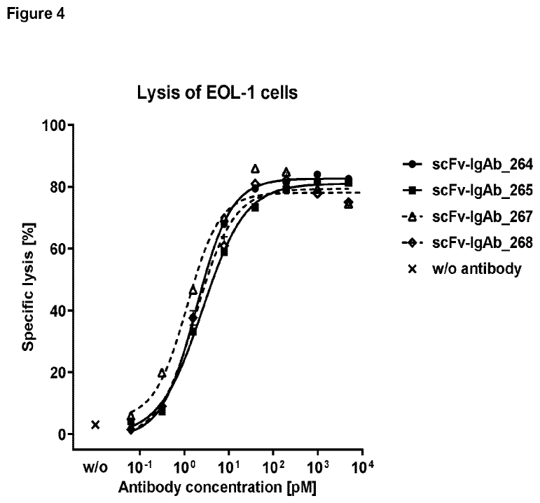

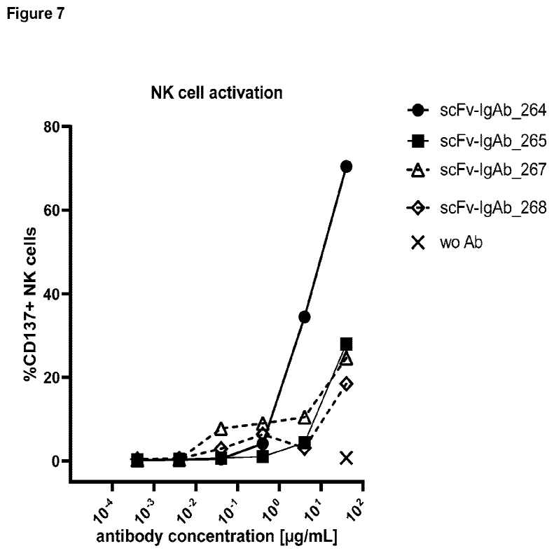

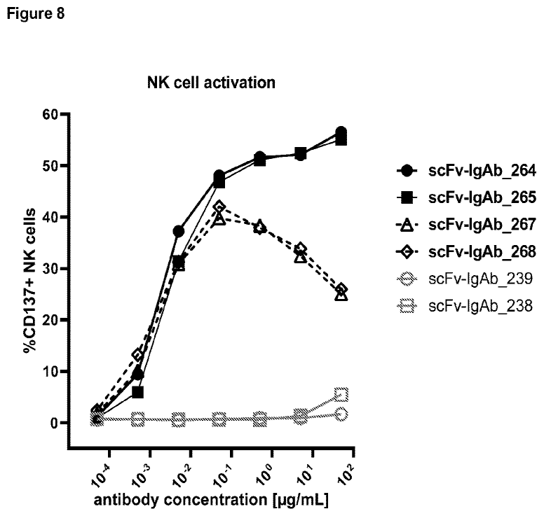

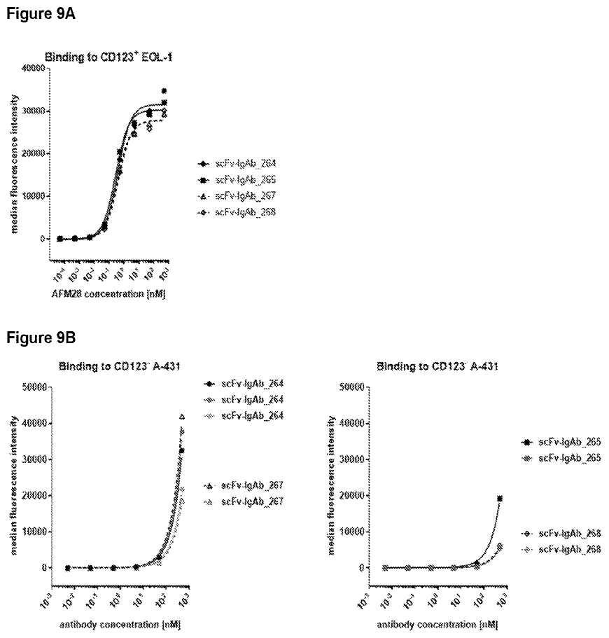

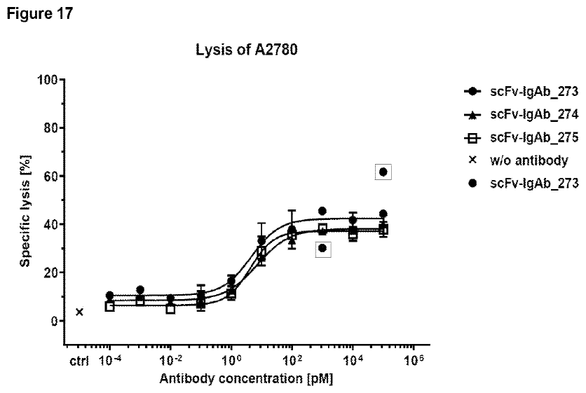

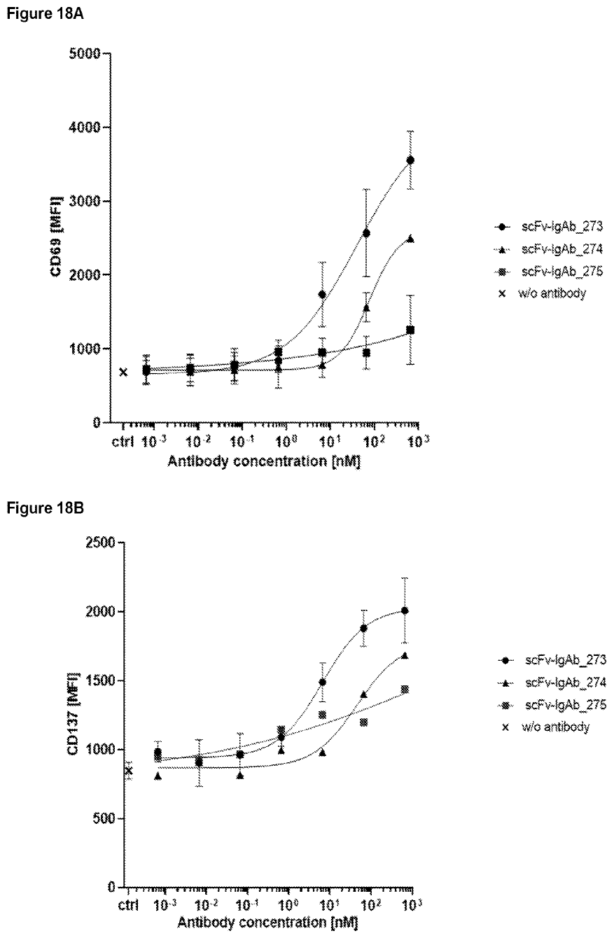

Moreover, the antibody constructs of the present invention comprising the CD16a1 anti-CD16A effector domain described herein show a substantial longer retention on NK cells when compared to the described CD16a2 anti-CD16A effector domain (see FIG. 3 and Table 5). Further, the antibody constructs of the present invention comprising the CD16a1 or CD16a3 anti-CD16A effector domain described herein induce NK cell-dependent lysis against target cells at similar maximal efficacy when compared to other antibody constructs comprising low-affinity anti-CD16A binding domains (see FIGS. 4 and 17). In addition, the inventors of the present application observed that the antibody constructs of the present invention comprising the CD16a1 or CD16a3 anti-CD16A effector domain described herein show the lowest unspecific activity, i.e. up-regulation of the activation marker CD137 on NK cells in the absence of target cells (see FIGS. 7 and 18A-18B). Moreover, the antibody constructs of the present invention comprising the CD16a1 anti-CD16A effector domain described herein is capable of inducing specific CD137 up-regulation on NK cells in response to target cells (FIG. 8), thereby showing the lowest potential for unspecific binding (FIG. 9A-9B).

However, the prior art has lately reported on clear advantages of CD16 shedding on activated NK cells which may sustain NK cell survival (Srpan et al., J. Cell. Biol., 2018, 217(9):3267-3283). Contrary thereto, the inventors of the present application found that the anti-CD16A binding domain comprised by the bispecific antibody construct of the present invention does not lead to activation-induced death of CD16A+ immune effector cells despite the low degree of CD16A shedding. Instead, CD16A+ NK cells that were activated by a CD123×CD16A bispecific antibody construct comprising the specific anti-CD16A binding domain of the present invention stably express CD16A, but did not show activation-induced death. Instead, CD16A+ NK cells activated by the bispecific antibody construct of the present invention were available for effective target cell killing.

Hence, the antibody constructs according to the present invention comprising the high-affinity anti-CD16A binding domain described herein show several advantages when compared to anti-CD16A binding domains that show lower affinity towards CD16A. Nonetheless, the use of a high-affinity anti-CD16A binding domain (comprising the CD16a1 or CD16a3 binding domain described herein) to stabilize CD16A must be considered counter-intuitive and would not have been obvious for a person skilled in the art, since high-affinity engagement typically results in greater activation of NK cells, and loss of CD16A. Thus, one would typically try to reduce potency to prevent CD16A loss by using a lower-affinity domain in an attempt to drive selectivity of ADCC towards cells expressing higher levels of FOLR1 and away from cells with lower levels of expression. Contrary to this expectation, a lower-affinity anti-CD16A binding domain variant (e.g. CD16a2 described herein) did not stabilize CD16A and resulted in increased CD16 shedding when compared to anti-CD16A binding domain CD16a1. However, this is expected to have a detrimental effect on therapeutic activity of a said low affinity bispecific antibody construct because CD16A will be shed quickly on circulating NK cells before these cells may be able to reach their intended tumor targets. This is relevant in a disease context, because when treating in particular solid tumors characterized by a limited presence of NK cells with a bispecific FOLR1×CD16A antibody, one would want to avoid immediate inactivation of NK cells due to CD16 shedding.

Therefore, the antibody constructs of the present invention provide a novel approach to enable tumor cell targeting with NK cell-mediated ADCC without inducing CD16A loss and sustained NK cell survival. In sum, the present invention is based at least partly on the surprising finding that a bispecific antibody construct comprising a high-affinity anti-CD16A first binding domain and a second binding domain for an antigen on the surface of a target cell, specifically FOLR1, can efficiently kill said target cells, thereby avoiding CD16A shedding and immediate inactivation of engaged NK cells. The antibody constructs of the present invention can thus be useful for tumor therapy, in particular solid tumors overexpressing FOLR1, because they are not only capable of activating NK cells via high-affinity binding of CD16A receptor, but also achieve long-lasting activation of NK cells without CD16A degradation.

The antibody constructs of the invention are characterized to induce a low degree of CD16A shedding or no CD16A shedding on the surface of the immune effector cell, preferably NK cells, bound by said antibody in the presence of target cells. It is understood that an NK cell from peripheral blood has around 10⁶ CD16A receptors on the surface (Peipp et al., Oncotarget, 2015, vol 6, no 31: 32075-32088). Without wishing to be bound by theory, the inventors of the present application believe that a maximum of about 50% CD16 shedding appears to be meaningful for sustaining effector cell activity, in particular NK cell activity, after binding to an anti-CD16A binding domain. The degree of CD16A shedding can be measured by flow cytometry, as essentially described in Example 5. Such an assay is preferably conducted as follows. PBMCs are isolated from buffy coats by density gradient centrifugation. The buffy coat samples are diluted with a two-to-threefold volume of PBS, layered on a cushion of Lymphoprep and centrifuged at 800×g for 25 min at room temperature w/o brake. PBMC located in the interface are collected and washed 3 times with PBS before they are cultured in complete RPMI 1640 medium overnight without stimulation. For the enrichment of NK cells PBMC are harvested from overnight cultures and used for one round of negative selection using the EasySep™ Human NK Cell Enrichment Kit for the immunomagnetic isolation of untouched human NK cells and the Big Easy EasySep™ Magnet according to the manufacturer's instructions. NK cells are then suspended in a volume of 1 mL at a density of 10-15×10⁶ cells/mL in pre-chilled complete RPMI 1640 medium. Antibody constructs are added to a concentration of 100, 10 and 1 μg/mL and incubated for 45 min on ice. Afterwards cells are washed with in complete RPMI 1640 medium and transferred to 96-well round-bottom plate. NK cells are incubated with or without 50 ng/mL phorbol-12-myristat-13-acetat (PMA) and 0.5 μM ionomycin for 4 h at 37° C. After the stimulation cells are washed with FACS buffer (PBS containing 2% heat-inactivated FCS, and 0.1% sodium azide). To detect the CD16 level, cells are restained with 100 μg/mL of the respective anti-CD16A antibody followed by incubation with 15 μg/mL FITC-conjugated goat anti-mouse IgG and stained with fixable viability stain eFluor™ 780 to exclude dead cells. After the last washing step cells are resuspended in 0.2 mL of FACS buffer and the fluorescence of cells are measured using a flow cytometer, and median fluorescence intensities of the cell samples are calculated. After subtracting the fluorescence intensity values of the cells stained with the secondary reagents alone, the MFI values are plotted using the GraphPad Prism software. Figures are generated using FlowJo Software.

In some embodiments, a “low degree of CD16A shedding” on the surface of the immune effector cell, preferably NK cells, means that the degree of CD16A shedding when using a test molecule, such as a bispecific antibody construct comprising the antiCD16A binding domain of the present invention, is not more than about 50%. The degree of CD16A shedding on effector cells caused by an antibody construct of the invention is preferably about 45% or lower, more preferably about 40% or lower, more preferably about 35% or lower, more preferably about 30% or lower, more preferably about 25% or lower, more preferably about 20% or lower, more preferably about 18% or lower, more preferably about 16% or lower, more preferably about 14% or lower, more preferably about 12% or lower, more preferably about 11% or lower, more preferably about 10% or lower, preferably determined at a concentration of 100 μg/mL. In some even more preferred embodiments, the degree of CD16A shedding when using an antibody construct of the invention is even lower, such as preferably about 9% or lower, more preferably about 8% or lower, more preferably about 7% or lower, more preferably about 6% or lower, more preferably about 5% or lower, more preferably about 4% or lower, more preferably about 3% or lower, more preferably about 2% or lower, or more preferably about 1% or lower, or most preferably non-detectable with an assay essentially described herein, preferably as defined supra, preferably determined at a concentration of 100 μg/mL.

In some embodiments, an antibody construct of the invention induces a degree of CD16A shedding that is lower as compared to the control antibody construct such as scFv-IgAb_148 (SEQ ID NOs: 92-93), scFv-IgAb 264 (SEQ ID NOs: 82-83) and scFv-IgAb 265 (SEQ ID NOs: 84-85) comprising a low-affinity anti-CD16A binding domain, preferably determined at a concentration of 100 μg/mL of the test antibody and the control antibody.

In some embodiments, an antibody construct of the invention induces a degree of CD16A shedding that is lower as compared to the control antibody construct such as scFv-IgAb_381 (SEQ ID NOs: 140-141), scFv-IgAb_273 (SEQ ID NOs: 134-135) and scFv-IgAb_274 (SEQ ID NOs: 136-137) comprising a low-affinity anti-CD16A binding domain, preferably determined at a concentration of 100 μg/mL of the test antibody and the control antibody.

The antibody constructs of the invention may further be characterized to induce a low degree of apoptotic death of immune effector cells, preferably NK cells, or no apoptotic death of immune effector cells, preferably NK cells, when bound to said effector cells in the presence of target cells. Hence, the antibody constructs of the invention are characterized to avoid apoptosis induction of immune effector cells, preferably NK cells, due to excessive inhibition of CD16A shedding.

In some embodiments, a “low degree of apoptotic death” means that the degree of immune effector cell apoptosis when using a test molecule, such as a bispecific antibody construct comprising the anti-CD16A binding domain of the present invention, is not more than about 50%. The degree of immune effector cell apoptosis caused by an antibody construct of the invention is preferably about 45% or lower, more preferably about 40% or lower, more preferably about 35% or lower, more preferably about 30% or lower, more preferably about 25% or lower, more preferably about 20% or lower, more preferably about 18% or lower, more preferably about 16% or lower, more preferably about 14% or lower, more preferably about 12% or lower, more preferably about 11% or lower, more preferably about 10% or lower, preferably determined at a concentration of 100 μg/mL. In some even more preferred embodiments, the degree of immune effector cell apoptosis of an antibody of the invention is even lower, such as preferably about 9% or lower, more preferably about 8% or lower, more preferably about 7% or lower, more preferably about 6% or lower, more preferably about 5% or lower, more preferably about 4% or lower, more preferably about 3% or lower, more preferably about 2% or lower, or more preferably about 1% or lower, or most preferably non-detectable, preferably determined at a concentration of 100 μg/mL.

In some embodiments, an antibody construct of the invention induces a degree of immune effector cell apoptosis that is lower as compared to the control antibody construct such as scFv-IgAb_148 (SEQ ID NOs: 92-93), scFv-IgAb_264 (SEQ ID NOs: 82-83) and scFv-IgAb_265 (SEQ ID NOs: 84-85) comprising a low-affinity anti-CD16A binding domain, preferably determined at a concentration of 100 μg/mL of the test antibody and the control antibody.

In some embodiments, an antibody construct of the invention induces a degree of immune effector cell apoptosis that is lower as compared to the control antibody construct such as scFv-IgAb_381 (SEQ ID NOs: 140-141), scFv-IgAb_273 (SEQ ID NOs: 134-135) and scFv-IgAb_274 (SEQ ID NOs: 136-137) comprising a low-affinity anti-CD16A binding domain, preferably determined at a concentration of 100 μg/mL of the test antibody and the control antibody.

As set forth herein above, the present invention relates to a bispecific antibody construct, comprising (a) a first binding domain (A), which is capable of specifically binding to a first target (A′) that is CD16A on the surface of an immune effector cell, wherein the first binding domain comprises: (i) a VL region comprising CDR-L1 as depicted in SEQ ID NO: 4, a CDR-L2 as depicted in SEQ ID NO: 5, and a CDR-L3 as depicted in SEQ ID NO: 6; and (ii) a VH region as depicted in SEQ ID NO: 7 or SEQ ID NO: 94; and (b) a second binding domain (B), which is capable of specifically binding to a second target (B′) that is an antigen on the surface of a target cell, wherein the second target (B′) is FOLR1.

The first binding domain (A) is capable of specifically binding CD16A, which preferably includes the capacity to discriminate between CD16A and CD16B. With other words, the first binding domain (A) preferably binds CD16A with higher affinity than CD16B, which may be at least about 10-fold higher, at least about 100-fold higher, or at least about 1000-fold higher. More preferably, the first binding domain does not essentially bind CD16B. It is thus understood that the first binding domain is preferably not a non-silenced CH2 domain, i.e. a CH2 domain that is capable of binding both CD16A and CD16B.

Accordingly the first binding domain preferably binds to an epitope of CD16A which comprises amino acid residues of the C-terminal sequence SFFPPGYQ (positions 201-209 of SEQ ID NO: 50), and/or residue G147 and/or residue Y158 of CD16A, which are not present in CD16B. It is preferred in the context of the present invention that the first binding domain, which binds CD16A on the surface of an effector cell binds to an epitope on CD16A, which is membrane proximal relative to the physiological Fcγ receptor binding domain of CD16A. A binding domain that specifically binds to an epitope comprising Y158 is preferred, because this epitope is proximal to the cell membrane and thus further contributes to reducing the likelihood of simultaneously binding a second immune effector cell.

In some preferred embodiments, the first binding domain (A) comprises a pair of VH- and VL-chains having a sequence as depicted in the pairs of sequences selected form the group consisting of SEQ ID NOs: 7 and 8 and SEQ ID NOs: 94 and 95.

In some preferred embodiments, the first binding domain (A) comprises a VL region as depicted in SEQ ID NO: 8 or SEQ ID NO: 95 and a VH region as depicted in SEQ ID NO: 7 or SEQ ID NO: 94.

The first binding domain (A) is preferably derived from an antibody. The first binding domain (A) preferably comprises a VH and a VL domain of an antibody. Preferred structures for the first binding domain (A) include a Fv, a scFv, a Fab, or a VL and VH pair which may be comprised in a diabody (Db), scDb or a double Fab. Preferably, the first binding domain (A) is a scFv. Equally preferred the first binding domain (A) is a Fv. Equally preferred, the first binding domain (A) is a Fab. Equally preferred, the first binding domain (A) is a Db. Equally preferred, the first binding domain (A) is a scDb. Equally preferred, the first binding domain (A) is double Fab. Most preferred, the first binding domain (A) is a scFv.

In some preferred embodiments, the first binding domain (A) of the antibody construct of the present invention is a scFv having the amino acid sequence as depicted in SEQ ID NO: 9. In some preferred embodiments, the first binding domain (A) of the antibody construct of the present invention is a scDb having the amino acid sequence as depicted in SEQ ID NO: 10.

In some preferred embodiments, the first binding domain (A) of the antibody construct of the present invention is a scFv having the amino acid sequence as depicted in SEQ ID NO: 96. In some preferred embodiments, the first binding domain (A) of the antibody construct of the present invention is a scDb having the amino acid sequence as depicted in SEQ ID NO: 97.

A control antibody construct comprising a low-affinity anti-CD16A binding domain as described herein may comprises a first binding domain (A), which is capable of specifically binding to CD16A on the surface of an immune effector cell, wherein the first binding domain comprises: (i) a VL region comprising CDR-L1 as depicted in SEQ ID NO: 11, a CDR-L2 as depicted in SEQ ID NO: 12, and a CDR-L3 as depicted in SEQ ID NO: 13, and (ii) a VH region as depicted in SEQ ID NO: 17 or 104; and (b) a second binding domain (B), which is capable of specifically binding to a second target (B′) that is an antigen on the surface of a target cell. The first binding domain (A) of said control antibody construct may comprises a pair of VH- and VL-chains having a sequence as depicted in the pairs of sequences selected form the group consisting of SEQ ID NOs: 17 or 104 and 18 or 105. Said control antibody construct may have a scFv first binding domain (A) as depicted in SEQ ID NO: 19 or 106. Said control antibody construct may also have a scDb first binding domain (A) as depicted in SEQ ID NO: 20 or 107.

In the context of the present invention, the second binding domain (B) of the antibody construct of the present invention that is specific for a second target (B′) that is an antigen on the surface of a target cells, specifically a tumor associated antigen, is specific for FOLR1, i.e. FOLR1 is target (B′).

The cell surface antigen FOLR1 on the surface of target cells is connected with specific disease entities as also described elsewhere herein. Specifically, FOLR1 is a cell surface antigen characteristic for solid cancer, e.g. ovarian (e.g., high grade serous ovarian cancer or epithelial ovarian cancer), breast (particularly TNBC), renal, lung (e.g., non small cell lung cancer (NSCLC) or mesotheliomas), colorectal (CRC), kidney (particularly ccRCC), pancreatic (particularly PDAC), endometrial (e.g., non-malignant endometrial cancer) and brain cancer.

Antibodies against FOLR1 are well known in the art and are e.g. described in U.S. Pat. No. 9,522,196B2 and WO2014104270. Specific examples for anti-human FOLR1 antibodies are farletuzumab (INN number 9067—sequences have also been disclosed under drugbank.ca/drugs/DB05595/polypeptide_sequences.fasta and the IMTG database (imtg.org under the INN number 9067)) and mirvetuximab (INN number 10187—sequence has been disclosed in the IMTG database (imtg.org under the INN number 10187)).

In the context of the present invention, the second binding domain (B) which is specific for FOLR1 preferably comprises a VH domain comprising the following three heavy chain CDRs and a VH domain comprising the following three light chain CDRs: a CDR-H1 as depicted in SEQ ID NO: 114 or 124, a CDR-H2 as depicted in SEQ ID NO: 115 or 125, a CDR-H3 as depicted in SEQ ID NO: 116 or 126, a CDR-L1 as depicted in SEQ ID NO: 117 or 127, a CDR-L2 as depicted in SEQ ID NO: 118 or 128, a CDR-L3 as depicted in SEQ ID NO: 119 or 129.

Hence, it is particularly envisage that the second binding domain (B) that is specific for FOLR1 comprises a VH domain comprising the following three heavy chain CDRs and a VH domain comprising the following three light chain CDRs: a CDR-H1 as depicted in SEQ ID NO: 114, a CDR-H2 as depicted in SEQ ID NO: 115, a CDR-H3 as depicted in SEQ ID NO: 116, a CDR-L1 as depicted in SEQ ID NO: 117, a CDR-L2 as depicted in SEQ ID NO: 118, a CDR-L3 as depicted in SEQ ID NO: 119.

It is also particularly envisage that the second binding domain (B) that is specific for FOLR1 comprises a VH domain comprising the following three heavy chain CDRs and a VH domain comprising the following three light chain CDRs: a CDR-H1 as depicted in SEQ ID NO: 124, a CDR-H2 as depicted in SEQ ID NO: 125, a CDR-H3 as depicted in SEQ ID NO: 126, a CDR-L1 as depicted in SEQ ID NO: 127, a CDR-L2 as depicted in SEQ ID NO: 128, a CDR-L3 as depicted in SEQ ID NO: 129.

In some preferred embodiments, the second binding domain (B) specific for FOLR1 comprises a pair of VH- and VL-chains having a sequence as depicted in the pair of sequences having at least 85%, 90%, 95%, 96%, 97%, 98%, 99%, or 100% identity each to SEQ ID NOs: 120 and 121, respectively, or having at least 85%, 90%, 95%, 96%, 97%, 98%, 99%, or 100% identity each to SEQ ID NOs: 130 and 131, respectively.

In some preferred embodiments, the second binding domain (B) specific for FOLR1 comprises a pair of VH- and VL-chains having a sequence as depicted in the pair of sequences of SEQ ID NOs: 120 and 121 or SEQ ID NOs: 130 and 131.

In some preferred embodiments, the second binding domain (B) specific for FOLR1 comprises a pair of VH- and VL-chains having a sequence as depicted in the pair of sequences having at least 85%, 90%, 95%, 96%, 97%, 98%, 99%, or 100% identity each to SEQ ID NOs: 120 and 121, respectively.

In some preferred embodiments, the second binding domain (B) specific for FOLR1 comprises a pair of VH- and VL-chains having a sequence as depicted in SEQ ID NOs: 120 and 121.

In some preferred embodiments, the second binding domain (B) specific for FOLR1 comprises a pair of VH- and VL-chains having a sequence as depicted in the pair of sequences having at least 85%, 90%, 95%, 96%, 97%, 98%, 99%, or 100% identity each to SEQ ID NOs: 130 and 131, respectively.

In some preferred embodiments, the second binding domain (B) specific for FOLR1 comprises a pair of VH- and VL-chains having a sequence as depicted in SEQ ID NOs: 130 and 131.

In some preferred embodiments, the second binding domain (B) specific for FOLR1 is a scFv having the amino acid sequence having at least 85%, 90%, 95%, 96%, 97%, 98%, 99%, or 100% identity each to SEQ ID NO: 122 or 132. In some preferred embodiments, the second binding domain (B) specific for FOLR1 is a scFv having the amino acid sequence as depicted in SEQ ID NO: 122 or 132. In some preferred embodiments, the second binding domain (B) specific for FOLR1 is a scFv having the amino acid sequence having at least 85%, 90%, 95%, 96%, 97%, 98%, 99%, or 100% identity each to SEQ ID NO: 123 or 133. In some embodiments, the second binding domain (B) specific for FOLR1 is a scDb having the amino acid sequence as depicted in SEQ ID NO: 123 or 133.

A second binding domain (B) specific for CD123 as disclosed herein preferably comprises a VH domain comprising the following three heavy chain CDRs and a VH domain comprising the following three light chain CDRs: a CDR-H1 as depicted in SEQ ID NO: 21, a CDR-H2 as depicted in SEQ ID NO: 22, a CDR-H3 as depicted in SEQ ID NO: 23, a CDR-L1 as depicted in SEQ ID NO: 24, a CDR-L2 as depicted in SEQ ID NO: 25, a CDR-L3 as depicted in SEQ ID NO: 26.

The second binding domain (B) specific for CD123 may comprise a pair of VH- and VL-chains having a sequence as depicted in the pair of sequences of SEQ ID NOs: 27 and 28.

The second binding domain (B) specific for CD123 may be a scFv having the amino acid sequence as depicted in SEQ ID NO: 29. The second binding domain (B) specific for CD123 may be a scDb having the amino acid sequence as depicted in SEQ ID NO: 30.

The second binding domain (B) of the antibody construct that is specific for CD123 may comprise a VH domain comprising the following three heavy chain CDRs and a VH domain comprising the following three light chain CDRs: a CDR-H1 as depicted in SEQ ID NO: 31, a CDR-H2 as depicted in SEQ ID NO: 32, a CDR-H3 as depicted in SEQ ID NO: 33, a CDR-L1 as depicted in SEQ ID NO: 34, a CDR-L2 as depicted in SEQ ID NO: 35, a CDR-L3 as depicted in SEQ ID NO: 36.

The second binding domain (B) specific for CD123 may comprise a pair of VH- and VL-chains having a sequence as depicted in the pair of sequences of SEQ ID NOs: 37 and 38.

The second binding domain (B) specific for CD123 may be a scFv having the amino acid sequence as depicted in SEQ ID NO: 39. The second binding domain (B) specific for CD123 may be a scDb having the amino acid sequence as depicted in SEQ ID NO: 40.

The second binding domain (B) is also preferably derived from an antibody. The second binding domain (B) preferably comprises a VH and a VL domain of an antibody. Preferred structures for the second binding domain (B) include a Fv, a scFv, a Fab, or a VL and VH pair which may be comprised in a diabody (Db), scDb or a double Fab. Preferably, the second binding domain (B) is a scFv. Equally preferred the second binding domain (B) is a Fv. Equally preferred, the second binding domain (B) is a Fab. Equally preferred, the second binding domain (B) is a Db. Equally preferred, the second binding domain (B) is a scDb. Equally preferred, the second binding domain (B) is double Fab. Most preferred, the second binding domain (B) is a scFv.

In the context of the present invention, it is particularly envisaged that the antibody construct binds to target cell and an immune effector cell simultaneously.

The antibody construct of the present invention may comprise a third domain (C), which comprises a half-life extension domain as described herein. The half-life extension domain may comprise a CH2 domain, in which the Fcγ receptor binding domain of the CH2 domain is silenced. The half-life extension domain may comprise two such CH2 domains. Whenever a half-life extension domain comprises a CH2 domain, the Fc receptor binding domain of the CH2 domain is silenced. The half-life extension domain may comprise a CH3 domain. The half-life extension domain may comprise two CH3 domains. The half-life extension domain may comprise a hinge domain. The half-life extension domain may comprise two hinge domains. The half-life extension domain may comprise a CH2 domain and a CH3 domain. In such a case, the CH2 domain and CH3 domain are preferably fused to each other, preferably in the (amino to carboxyl) order CH2 domain-CH3 domain. Non-limiting examples for such fusions are shown in SEQ ID NOs: 66-81. The half-life extension domain may comprise a hinge domain and a CH2 domain. In such a case, the hinge domain and the CH2 domain are preferably fused to each other, preferably in the (amino to carboxyl) order hinge domain-CH2 domain. The half-life extension domain may comprise a hinge domain, a CH2 domain, and a CH3 domain. In such a case, the hinge domain, the CH2 domain, and CH3 domain are preferably fused to each other, preferably in the (amino to carboxyl) order hinge domain-CH2 domain-CH3 domain. The half-life extending domain may comprise two hinge domain-CH2 domain elements, two CH2 domain-CH3 domain elements, or two hinge domain-CH2 domain-CH3 domain elements. In such a case the two fusions may be located on two different polypeptide strands. Alternatively, the fusions can be located on the same polypeptide strand. An illustrative example for two hinge domain-CH2 domain-CH3 domain elements that are located on the same polypeptide strand is the “single chain Fc” or “scFc” format. Here, both hinge-CH2-CH3 subunits are fused together via a linker that allows assembly of a Fc domain. A preferred linker for this purpose is a glycine serine linker, which preferably comprises from about 20 to about 40 amino acids. Preferred glycine serine linkers may have one or more repeats of GGS, GGGS (SEQ ID NO: 41), or GGGGS (SEQ ID NO: 46). Such linker preferably comprises 4-8 repeats (e.g. 4, 5, 6, 7, or 8 repeats) of GGGGS. Such a linker is preferably (GGGGS)₆, (SEQ ID NO 49). Further scFc constant domains are known in the art and inter alia described in WO 2017/134140.

Generally, the antibody constructs of the present invention can be monovalent, bivalent, trivalent, or have an even higher valency for any one of the first target (A′) and the second target (B′). The antibody constructs of the disclosure may thus comprise one, two, three, or even more of any one of the first binding domain (A) and the second binding domain. It is preferred for the antibody construct of the invention that it is at least monovalent for the first target (A′) and at least monovalent for the second target (B′). It is also preferred for the antibody construct of the invention that it is at least monovalent for the first target (A′) and bivalent for the second target (B′). It is further preferred for the antibody construct of the invention that it is at least bivalent for the first target (A′) and at least bivalent for the second target (B′). It also preferred for the antibody construct of the invention that it is at least bivalent for the first target (A′) and at least trivalent for the second target (B′). It also preferred for the antibody construct of the invention that it is at least bivalent for the first target (A′) and at least monovalent for the second target (B′). Most preferred, the antibody construct of the invention is bivalent for the first target (A′) and bivalent for the second target (B′).

Hence, it is preferred for the antibody construct of the invention that it comprises at least one first binding domains (A) and at least one second binding domains (B). It is further preferred for the antibody construct of the invention that it comprises at least one first binding domains (A) and at least two second binding domains (B). It is further preferred for the antibody construct of the invention that it comprises at least two first binding domains (A) and at least two second binding domains (B). It is further preferred for the antibody construct of the invention that it comprises at least two first binding domains (A) and at least three second binding domains (B). It is further preferred for the antibody construct of the invention that it comprises at least two first binding domains (A) and at least one second binding domain (B). Most preferred, the antibody construct of the invention comprises two first binding domains (A) and two second binding domains (B).

Disclosed herein are antibody construct comprising one first binding domain (A) specifically binding to CD16A and one second binding domain (B) specifically binding against CD123. These antibody constructs may comprise one first binding domain (A) specifically binding to CD16A and two second binding domains (B) specifically binding against CD123. These antibody constructs may also comprise two first binding domains (A) specifically binding to CD16A and one second binding domain (B) specifically binding against CD123. These antibody constructs may also comprise two first binding domains (A) specifically binding to CD16A and two second binding domains (B) specifically binding against CD123.

It is particularly preferred for the antibody construct of the present invention that it comprises one first binding domain (A) specifically binding to CD16A and one second binding domain (B) specifically binding against FOLR1. It is also preferred for the antibody construct of the present invention that it comprises one first binding domain (A) specifically binding to CD16A and two second binding domains (B) specifically binding against FOLR1. It is also preferred for the antibody construct of the present invention that it comprises two first binding domains (A) specifically binding to CD16A and one second binding domain (B) specifically binding against FOLR1. It is also preferred for the antibody construct of the present invention that it comprises two first binding domains (A) specifically binding to CD16A and two second binding domains (B) specifically binding against FOLR1.

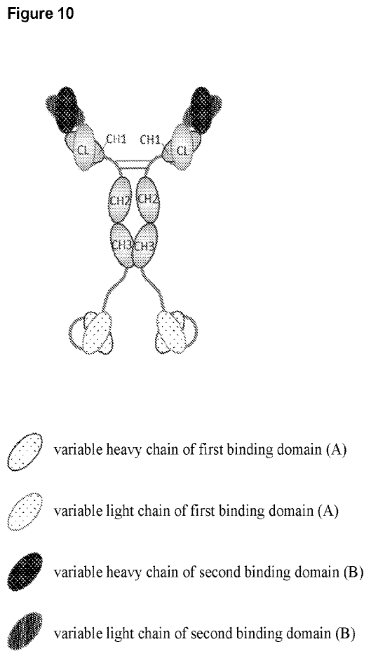

In a preferred embodiment, the first binding domain (A) is fused to a C terminus of a Fc region. Such a fusion format is illustratively shown in FIG. 10. The first binding domain (A) may be fused to a constant domain of an antibody via a linker. Such a linker is preferably a short linker, which preferably has a length of about 10 nm or less, preferably about 9 nm or less, preferably about 8 nm or less, preferably about 7 nm or less, preferably about 6 nm or less, preferably about 5 nm or less, preferably about 4 nm or less, or even less. The length of the linker is preferably determined as described by Rossmalen et a1 Biochemistry 2017, 56, 6565-6574, which also describes suitable linkers that are well known to the skilled person. An example for a suitable linker is a glycine serine linker or a serine linker, which preferably comprise no more than about 75 amino acids, preferably not more than about 50 amino acids. In illustrative example, a suitable linker comprises one or more (e.g. 1, 2, 3, 4, 5, 6, 7, or 8) GGGGS sequences (SEQ ID NO: 46), such as (GGGGS)₂ (SEQ ID NO: 47), (GGGGS)₄ (SEQ ID NO: 48), or preferably (GGGGS)₆ (SEQ ID NO: 49). Other illustrative examples for linkers are shown in SEQ ID NOs: 42-45. The first binding domain (A) is preferably scFv fragments that is fused to a C terminus of a Fc domain, preferably via the VL domain of the scFv. Accordingly, the arrangement of the polypeptide chain (from N to C) is preferably . . . -CH2-CH3-VL-VH, optionally with a linker between the Fc and the scFv. The second binding domain can be located at any suitable position of the antibody construct. Where the antibody construct comprises a Fc region, the second binding domain (B) can be located N terminal of the Fc region, either directly or linked via at least a part of a hinge domain. Other linkers disclosed herein can also be used to link the third binding domain to the Fc domain. A hinge domain is however preferred for this purpose. The second binding domain (B) can be any suitable structure disclosed herein, while a scFv structure is preferred.

An antibody construct of the invention is preferably in a format as essentially shown in FIG. 10 and which is also referred to as “scFv-IgAb” herein. Such an antibody construct comprises an immunoglobulin that has one scFv fragments fused to the C terminus of each of the two heavy chains, optionally via a linker, which is preferably a connector, disclosed herein. Said scFvs form the first binding domain (A). Two second binding domains (B) are formed by the binding sites of the immunoglobulin. The scFv-IgAb format may comprise four polypeptide chains, two light chains in the arrangement VL(B)-CL, and two heavy chains each fused to a scFv in the arrangement VH(B)-CH1-hinge-CH2-CH3-VL(A)-VH(A) (or less preferred VH(B)-CH1-hinge-CH2-CH3-VH(A)-VL(A)). The letters in parenthesis stand for first binding domain (A) and the second binding domain (B), respectively. For example, VL(A) stands for a VL domain of a first binding domain (A), while VH(B) stands for a VH domain of a second binding domain (B). Illustrative examples for such antibody constructs are shown in SEQ ID NOs: 86-87, and 88-98.

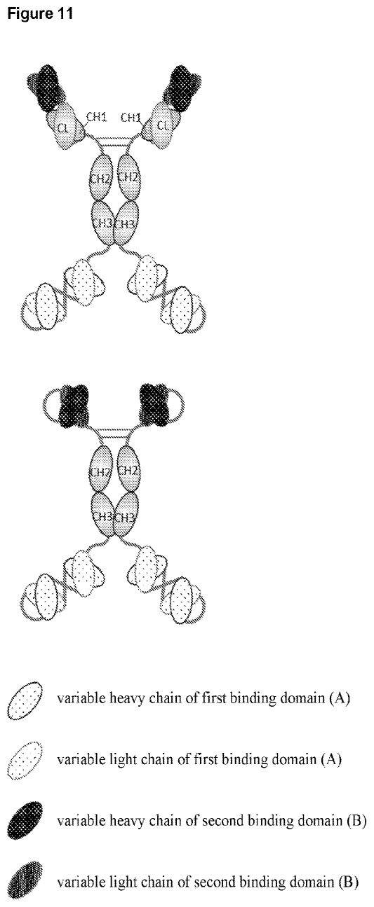

In another preferred embodiment, two first binding domains (A) are fused to two C termini of a Fc region, wherein the two first binding domains (A) are preferably fused together in form of a diabody or single chain diabody, preferably via a VL domain of a first binding domain (A). Such a fusion formats are illustratively shown in FIG. 11. The first binding domains (A) may be fused to a constant domain of an antibody via a linker. Such a linker is preferably a short linker, which preferably has a length of about 10 nm or less, preferably about 9 nm or less, preferably about 8 nm or less, preferably about 7 nm or less, preferably about 6 nm or less, preferably about 5 nm or less, preferably about 4 nm or less, or preferably even less. The length of the linker is preferably determined as described by Rossmalen et a1 Biochemistry 2017, 56, 6565-6574, which also describes suitable linkers that are well known to the skilled person. An example for a suitable linker is a glycine serine linker or a serine linker, which preferably comprises not more than about 75 amino acids, preferably not more than about 50 amino acids. In illustrative examples, a suitable linker comprises one or more GGGGS sequences (SEQ ID NO: 46), such as (GGGGS)₂ (SEQ ID NO: 47), (GGGGS)₄ (SEQ ID NO: 48), or preferably (GGGGS)₆ (SEQ ID NO: 49). Other illustrative examples for linkers are shown in SEQ ID NOs: 42-45. The first binding domains (A) are preferably scDb fragments that are fused to two C termini of a Fc domain, preferably via a VL domain of the scDb. Accordingly, the arrangement of on the polypeptide chain (from N to C) is preferably . . . -CH2-CH3-VL-VH-VL-VH, optionally with a linker between the Fc and the scDb.

The second binding domain can be located at any suitable position of the antibody construct. Where the antibody construct comprises a Fc region, the second binding domain (B) can be located N terminal of the Fc region, either directly or linked via at least a part of a hinge domain. Other linkers disclosed herein can also be used to link the third binding domain to the Fc domain. A hinge domain is however preferred for this purpose. The second binding domain (B) can be any suitable structure disclosed herein, while a Fab structure is preferred.

An antibody construct of the invention is also preferably in a format as essentially shown in FIG. 11 and which is also referred to as “scDb-IgAb”. Such an antibody construct comprises an immunoglobulin that has one scDb fragments fused to the C terminus of each of the two heavy chains, optionally via a linker, which is preferably a connector, disclosed herein. Said scFvs forms the first binding domain (A). Two second binding domains (B) are formed by the binding sites of the immunoglobulin. The scDb-IgAb format may comprise four polypeptide chains, two light chains in the arrangement VL(B)-CL, and two heavy chains each fused to a scDb in the arrangement VH(B)-CH1-hinge-CH2-CH3-VL(A)-VH(A)-VL(A)-VH(A) (or less preferred VH(B)-CH1-hinge-CH2-CH3-VH(A)-VL(A)-VH(A)-VL(H)). Also envisaged is the arrangement VH(B)-hinge-CH2-CH3-VL(A)-VH(A)-VL(A)-VH(A) (or less preferred VH(B)-hinge-CH2-CH3-VH(A)-VL(A)-VH(A)-VL(H)). The letters in parenthesis stand for first binding domain (A) and the second binding domain (B), respectively. For example, VL(A) stands for a VL domain of a first binding domain (A), while VH(B) stands for a VH domain of a second binding domain (B).

Generally, a hinge domain comprised in an antibody construct of the disclosure may comprise a full length hinge domain, such as a hinge domain shown in SEQ ID NO: 53. The hinge domain may also comprise a shortened and/or modified hinge domain. A shortened hinge domain may comprise the upper hinge domain as e.g. shown in SEQ ID NO: 54 or the middle hinge domain as e.g. shown in SEQ ID NO: 55, but not the entire hinge domain, with the latter being preferred. Preferred hinge domains in the context of the invention show modulated flexibility relative to an antibody construct having the wild type hinge domain as described in Dall'Acqua et a1 (J Immunol. 2006 Jul. 15; 177(2):1129-38) or in WO 2009/006520. Moreover, preferred hinge domains are characterized to consist of less than 25 aa residues. More preferably, the length of the hinge is 10 to 20 aa residues. A hinge domain comprised in an antibody construct of the disclosure may also comprise or consists of the IgG2 subtype hinge sequence ERKCCVECPPCP (SEQ ID NO: 56), the IgG3 subtype hinge sequence ELKTPLDTTHTCPRCP (SEQ ID NO: 57) or ELKTPLGDTTHTCPRCP (SEQ ID NO: 58), and/or the IgG4 subtype hinge sequence ESKYGPPCPSCP (SEQ ID NO: 59). Further hinge domains that can be used in the context of the present invention are known to the skilled person and are e.g. described in WO 2017/134140.

An antibody construct of the present invention is preferably a bispecific antibody construct comprising (a) a first binding domain (A), which is capable of specifically binding to a first target (A′) that is CD16A on the surface of an immune effector cell comprising: (i) a VL region comprising CDR-L1 as depicted in SEQ ID NO: 4, a CDR-L2 as depicted in SEQ ID NO: 5, and a CDR-L3 as depicted in SEQ ID NO: 6, and a VH region as depicted in SEQ ID NO: 7 or SEQ ID NO: 94, wherein said first binding domain is a scFv; (b) a second binding domain which is capable of specifically binding to a second target (B′) that is FOLR1 on the surface of a target cell, comprising a VL region comprising a CDR-H1 as depicted in SEQ ID NO: 114, a CDR-H2 as depicted in SEQ ID NO: 115, a CDR-H3 as depicted in SEQ ID NO: 116, a CDR-L1 as depicted in SEQ ID NO: 117, a CDR-L2 as depicted in SEQ ID NO: 118, a CDR-L3 as depicted in SEQ ID NO: 119, wherein said second binding domain is a Fab; and (c) a third binding domain comprising two of the hinge domain-CH2 domain-CH3 domain elements, preferably as depicted in SEQ ID NOs: 53 and 67; wherein the first binding domain (A) is fused to the C terminus of a CH3 domain of the third domain and the second binding domain (B) is fused to the N terminus of a hinge region of the third domain.

Alternatively, an antibody construct of the present invention is preferably a bispecific antibody construct comprising (a) a first binding domain (A), which is capable of specifically binding to a first target (A′) that is CD16A on the surface of an immune effector cell comprising: (i) a VL region comprising CDR-L1 as depicted in SEQ ID NO: 4, a CDR-L2 as depicted in SEQ ID NO: 5, and a CDR-L3 as depicted in SEQ ID NO: 6, and a VH region as depicted in SEQ ID NO: 7 or SEQ ID NO: 94, wherein said first binding domain is a scFv; (b) a second binding domain which is capable of specifically binding to a second target (B′) that is FOLR1 on the surface of a target cell, comprising a VL region comprising a CDR-H1 as depicted in SEQ ID NO: 124, a CDR-H2 as depicted in SEQ ID NO: 125, a CDR-H3 as depicted in SEQ ID NO: 126, a CDR-L1 as depicted in SEQ ID NO: 127, a CDR-L2 as depicted in SEQ ID NO: 128, a CDR-L3 as depicted in SEQ ID NO: 129, wherein said second binding domain is a Fab; and (c) a third binding domain comprising two of the hinge domain-CH2 domain-CH3 domain elements, preferably as depicted in SEQ ID NOs: 53 and 67; wherein the first binding domain (A) is fused to the C terminus of a CH3 domain of the third domain and the second binding domain (B) is fused to the N terminus of a hinge region of the third domain.

In some embodiments the antibody construct of the present invention is preferably a bispecific antibody construct comprising (a) a first binding domain (A), which is capable of specifically binding to a first target (A′) that is CD16A on the surface of an immune effector cell comprising: (i) a VL region as depicted in SEQ ID NO: 8 or SEQ ID NO: 95, and a VH region as depicted in SEQ ID NO: 7 or SEQ ID NO: 94, wherein said first binding domain is a scFv; (b) a second binding domain which is capable of specifically binding to a second target (B′) that is FOLR1 on the surface of a target cell, comprising a VL region as depicted in SEQ ID NO: 121 and a VH region as depicted in SEQ ID NO: 120, wherein said second binding domain is a Fab; and (c) a third binding domain comprising two of the hinge domain-CH2 domain-CH3 domain elements, preferably as depicted in SEQ ID NOs: 53 and 67; wherein the first binding domain (A) is fused to the C terminus of a CH3 domain of the third domain and the second binding domain (B) is fused to the N terminus of a hinge region of the third domain. Alternatively, the second binding domain of said antibody construct which is capable of specifically binding to a second target (B′) that is FOLR1 on the surface of a target cell, comprises a VL region as depicted in SEQ ID NO: 131 and a VH region as depicted in SEQ ID NO: 130, wherein said second binding domain is a Fab.

An antibody construct of the invention is preferably an antibody construct selected from the group consisting of SEQ ID NOs: 142-143, and 138-139, i.e. an antibody construct having an amino acid sequence of SEQ ID NOs: 142-143 or SEQ ID NOs: 138-139, wherein SEQ ID NOs: 142-143 are preferred in the context of the present invention. In this respect is envisaged that the antibody comprises two of the recited heavy and light chains to form an IgAb.

An antibody construct of the invention is preferably an variant of an antibody construct selected from the group consisting of SEQ ID NOs: 142-143, and 138-139, wherein the variant has at least 90%, preferably at least 95%, more preferably at least 98%, even more preferably at least 99% sequence identity to any one of these aforementioned antibody constructs, provided that the CDR-L1-L3 sequences and the VH region of the first binding domain and the CDR sequences of the second binding domain comprised in these antibody constructs are not altered.

The present invention also relates to a nucleic acid molecule (DNA and RNA) that includes nucleotide sequences encoding an antibody construct disclosed herein. The present disclosure also encompasses a vector comprising a nucleic acid molecule of the invention. The present invention also encompasses a host cell containing said nucleic acid molecule or said vector. Since the degeneracy of the genetic code permits substitutions of certain codons by other codons specifying the same amino acid, the disclosure is not limited to a specific nucleic acid molecule encoding a antibody construct as described herein but encompasses all nucleic acid molecules that include nucleotide sequences encoding a functional polypeptide. In this regard, the present disclosure also relates to nucleotide sequences encoding the antibody constructs of the disclosure.

A nucleic acid molecule disclosed in this application may be “operably linked” to a regulatory sequence (or regulatory sequences) to allow expression of this nucleic acid molecule.

A nucleic acid molecule, such as DNA, is referred to as “capable of expressing a nucleic acid molecule” or capable “to allow expression of a nucleotide sequence” if it includes sequence elements which contain information regarding to transcriptional and/or translational regulation, and such sequences are “operably linked” to the nucleotide sequence encoding the polypeptide. An operable linkage is a linkage in which the regulatory sequence elements and the sequence to be expressed are connected in a way that enables gene expression. The precise nature of the regulatory regions necessary for gene expression may vary among species, but in general these regions include a promoter which, in prokaryotes, contains both the promoter per se, i.e. DNA elements directing the initiation of transcription, as well as DNA elements which, when transcribed into RNA, will signal the initiation of translation. Such promoter regions normally include 5′ non-coding sequences involved in initiation of transcription and translation, such as the −35/−10 boxes and the Shine-Dalgarno element in prokaryotes or the TATA box, CAAT sequences, and 5′-capping elements in eukaryotes. These regions can also include enhancer or repressor elements as well as translated signal and leader sequences for targeting the native polypeptide to a specific compartment of a host cell.

In addition, the 3′ non-coding sequences may contain regulatory elements involved in transcriptional termination, polyadenylation or the like. If, however, these termination sequences are not satisfactory functional in a particular host cell, then they may be substituted with signals functional in that cell.

Therefore, a nucleic acid molecule of the disclosure can include a regulatory sequence, such as a promoter sequence. In some embodiments a nucleic acid molecule of the disclosure includes a promoter sequence and a transcriptional termination sequence. Examples of promoters useful for expression in eukaryotic cells are the SV40 promoter or the CMV promoter.

The nucleic acid molecules of the disclosure can also be part of a vector or any other kind of cloning vehicle, such as a plasmid, a phagemid, a phage, a baculovirus, a cosmid or an artificial chromosome.

Such cloning vehicles can include, aside from the regulatory sequences described above and a nucleic acid sequence encoding an antibody construct as described herein, replication and control sequences derived from a species compatible with the host cell that is used for expression as well as selection markers conferring a selectable phenotype on transformed or transfected cells. Large numbers of suitable cloning vectors are known in the art, and are commercially available.

The present invention also relates to a method for the production of an antibody construct of the disclosure, wherein the antibody construct is produced starting from the nucleic acid coding for the antibody construct or any subunit therein. The method can be carried out in vivo, the polypeptide can, for example, be produced in a bacterial or eukaryotic host organism and then isolated from this host organism or its culture. It is also possible to produce an antibody construct of the disclosure in vitro, for example by use of an in vitro translation system.

When producing the antibody construct in vivo, a nucleic acid encoding such polypeptide is introduced into a suitable bacterial or eukaryotic host organism by means of recombinant DNA technology. For this purpose, the host cell may be transformed with a cloning vector that includes a nucleic acid molecule encoding an antibody construct as described herein using established standard methods. The host cell may then be cultured under conditions, which allow expression of the heterologous DNA and thus the synthesis of the corresponding polypeptide or antibody construct. Subsequently, the polypeptide or antibody construct is recovered either from the cell or from the cultivation medium.

Suitable host cells can be eukaryotic, such as immortalized mammalian cell lines (e.g., HeLa cells or CHO cells) or primary mammalian cells.

An antibody construct of the disclosure as described herein may be not necessarily generated or produced only by use of genetic engineering. Rather, such polypeptide can also be obtained by chemical synthesis such as Merrifield solid phase polypeptide synthesis or by in vitro transcription and translation. Methods for the solid phase and/or solution phase synthesis of proteins are well known in the art (see e.g. Bruckdorfer, T. et al. (2004) Curr. Pharm. Biotechnol. 5, 29-43).

An antibody construct of the disclosure may be produced by in vitro transcription/translation employing well-established methods known to those skilled in the art.

The invention also provides a composition, preferably a pharmaceutical composition comprising an antibody construct of the invention.

Certain embodiments provide pharmaceutical compositions comprising the antibody construct defined in the context of the invention and further one or more excipients such as those illustratively described in this section and elsewhere herein. Excipients can be used in the invention in this regard for a wide variety of purposes, such as adjusting physical, chemical, or biological properties of formulations, such as adjustment of viscosity, and or processes of one aspect of the invention to improve effectiveness and or to stabilize such formulations and processes against degradation and spoilage due to, for instance, stresses that occur during manufacturing, shipping, storage, pre-use preparation, administration, and thereafter.

In certain embodiments, the pharmaceutical composition may contain formulation materials for the purpose of modifying, maintaining or preserving, e.g., the pH, osmolarity, viscosity, clarity, color, isotonicity, odor, sterility, stability, rate of dissolution or release, adsorption or penetration of the composition (see, REMINGTON'S PHARMACEUTICAL SCIENCES, 18″ Edition, (A. R. Genrmo, ed.), 1990, Mack Publishing Company). In such embodiments, suitable formulation materials may include, but are not limited to:
amino acids such as glycine, alanine, glutamine, asparagine, threonine, proline, 2-phenylalanine, including charged amino acids, preferably lysine, lysine acetate, arginine, glutamate and/or histidine
antimicrobials such as antibacterial and antifungal agents
antioxidants such as ascorbic acid, methionine, sodium sulfite or sodium hydrogen-sulfite;
buffers, buffer systems and buffering agents which are used to maintain the composition at physiological pH or at a slightly lower pH; examples of buffers are borate, bicarbonate,
Tris-HCl, citrates, phosphates or other organic acids, succinate, phosphate, and histidine; for example Tris buffer of about pH 7.0-8.5;
non-aqueous solvents such as propylene glycol, polyethylene glycol, vegetable oils such as olive oil, and injectable organic esters such as ethyl oleate;
aqueous carriers including water, alcoholic/aqueous solutions, emulsions or suspensions, including saline and buffered media;
biodegradable polymers such as polyesters;
bulking agents such as mannitol or glycine;
chelating agents such as ethylenediamine tetraacetic acid (EDTA);
isotonic and absorption delaying agents;
complexing agents such as caffeine, polyvinylpyrrolidone, beta-cyclodextrin or hydroxypropyl-beta-cyclodextrin)
fillers;
monosaccharides; disaccharides; and other carbohydrates (such as glucose, mannose or dextrins); carbohydrates may be non-reducing sugars, preferably trehalose, sucrose, octasulfate, sorbitol or xylitol;
(low molecular weight) proteins, polypeptides or proteinaceous carriers such as human or bovine serum albumin, gelatin or immunoglobulins, preferably of human origin;
coloring and flavouring agents;
sulfur containing reducing agents, such as glutathione, thioctic acid, sodium thioglycolate, thioglycerol, [alpha]-monothioglycerol, and sodium thio sulfate
diluting agents;
emulsifying agents;
hydrophilic polymers such as polyvinylpyrrolidone)
salt-forming counter-ions such as sodium;
preservatives such as antimicrobials, anti-oxidants, chelating agents, inert gases and the like; examples are: benzalkonium chloride, benzoic acid, salicylic acid, thimerosal, phenethyl alcohol, methylparaben, propylparaben, chlorhexidine, sorbic acid or hydrogen peroxide);
metal complexes such as Zn-protein complexes;
solvents and co-solvents (such as glycerin, propylene glycol or polyethylene glycol);
sugars and sugar alcohols, such as trehalose, sucrose, octasulfate, mannitol, sorbitol or xylitol stachyose, mannose, sorbose, xylose, ribose, myoinisitose, galactose, lactitol, ribitol, myoinisitol, galactitol, glycerol, cyclitols (e.g., inositol), polyethylene glycol; and polyhydric sugar alcohols;
suspending agents;
surfactants or wetting agents such as pluronics, PEG, sorbitan esters, polysorbates such as polysorbate 20, polysorbate, triton, tromethamine, lecithin, cholesterol, tyloxapal; surfactants may be detergents, preferably with a molecular weight of >1.2 KD and/or a polyether, preferably with a molecular weight of >3 KD; non-limiting examples for preferred detergents are Tween 20, Tween 40, Tween 60, Tween 80 and Tween 85; non-limiting examples for preferred polyethers are PEG 3000, PEG 3350, PEG 4000 and PEG 5000;
stability enhancing agents such as sucrose or sorbitol;
tonicity enhancing agents such as alkali metal halides, preferably sodium or potassium chloride, mannitol sorbitol;
parenteral delivery vehicles including sodium chloride solution, Ringer's dextrose, dextrose and sodium chloride, lactated Ringer's, or fixed oils;
intravenous delivery vehicles including fluid and nutrient replenishers, electrolyte replenishers (such as those based on Ringer's dextrose).

It is evident to those skilled in the art that the different constituents of the pharmaceutical composition (e.g., those listed above) can have different effects, for example, and amino acid can act as a buffer, a stabilizer and/or an antioxidant; mannitol can act as a bulking agent and/or a tonicity enhancing agent; sodium chloride can act as delivery vehicle and/or tonicity enhancing agent; etc.

It is envisaged that the composition of the invention might comprise, in addition to the polypeptide of the invention defined herein, further biologically active agents, depending on the intended use of the composition.

In certain embodiments, the optimal pharmaceutical composition will be determined by one skilled in the art depending upon, for example, the intended route of administration, delivery format and desired dosage. See, for example, REMINGTON'S PHARMACEUTICAL SCIENCES, supra. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles.

Additional pharmaceutical compositions will be evident to those skilled in the art, including formulations involving the antibody construct of the invention in sustained- or controlled-delivery/release formulations. Techniques for formulating a variety of other sustained- or controlled-delivery means, such as liposome carriers, bio-erodible microparticles or porous beads and depot injections, are also known to those skilled in the art. See, for example, International Patent Application No. PCT/US93/00829, which describes controlled release of porous polymeric microparticles for delivery of pharmaceutical compositions. Sustained-release preparations may include semipermeable polymer matrices in the form of shaped articles, e.g., films, or microcapsules. Sustained release matrices may include polyesters, hydrogels, polylactides (as disclosed in U.S. Pat. No. 3,773,919 and European Patent Application Publication No. EP 058481), copolymers of L-glutamic acid and gamma ethyl-L-glutamate (Sidman et al., 1983, Biopolymers 2:547-556), poly (2-hydroxyethyl-methacrylate) (Langer et al., 1981, J. Biomed. Mater. Res. 15:167-277 and Langer, 1982, Chem. Tech. 12:98-105), ethylene vinyl acetate (Langer et al., 1981, supra) or poly-D(−)-3-hydroxybutyric acid (European Patent Application Publication No. EP 133,988). Sustained release compositions may also include liposomes that can be prepared by any of several methods known in the art. See, e.g., Eppstein et al., 1985, Proc. Natl. Acad. Sci. U.S.A. 82:3688-3692; European Patent Application Publication Nos. EP 036,676; EP 088,046 and EP 143,949.

The antibody construct may also be entrapped in microcapsules prepared, for example, by coacervation techniques or by interfacial polymerization (for example, hydroxymethylcellulose or gelatine-microcapsules and poly (methylmethacylate) microcapsules, respectively), in colloidal drug delivery systems (for example, liposomes, albumin microspheres, microemulsions, nanoparticles and nanocapsules), or in macroemulsions. Such techniques are disclosed in Remington's Pharmaceutical Sciences, 16th edition, Oslo, A., Ed., (1980).

Pharmaceutical compositions used for in vivo administration are typically provided as sterile preparations. Sterilization can be accomplished by filtration through sterile filtration membranes. When the composition is lyophilized, sterilization using this method may be conducted either prior to or following lyophilization and reconstitution. Compositions for parenteral administration can be stored in lyophilized form or in a solution. Parenteral compositions generally are placed into a container having a sterile access port, for example, an intravenous solution bag or vial having a stopper pierceable by a hypodermic injection needle.

In one embodiment of the pharmaceutical composition according to one aspect of the invention the composition is administered to a patient intravenously.

Methods and protocols for the intravenous (iv) administration of pharmaceutical compositions described herein are well known in the art.

The antibody construct of the invention and/or pharmaceutical composition of the invention is preferably used in the prevention, treatment or amelioration of a disease selected from a proliferative disease, a tumorous disease, a viral disease or an immunological disorder. In the context of the present invention, said diseases are preferably characterized by FOLR1 overexpression. Preferably, said tumorous disease is a malignant disease, preferably cancer.

The tumorous disease addressed by the present invention is a solid tumor. It is particularly envisaged that said solid tumor is selected from the group consisting of ovarian (e.g., high grade serous ovarian cancer or epithelial ovarian cancer), breast (particularly TNBC), renal, lung (e.g., non small cell lung cancer (NSCLC) or mesotheliomas), colorectal (CRC), kidney (particularly ccRCC), pancreatic (particularly PDAC), endometrial (e.g., non-malignant endometrial cancer) and brain cancer.

In preferred embodiments said tumorous disease is a metastatic tumor.

The present invention also provides a method for the treatment or amelioration of a disease, the method comprising the step of administering to a subject in need thereof an antibody construct according to the invention.

In one embodiment of said method for the treatment or amelioration of a disease the subject suffers from a proliferative disease, a tumorous disease, an infectious disease such as a viral disease, or an immunological disorder. It is preferred that said tumorous disease is a malignant disease, preferably cancer as defined elsewhere herein.

The antibody construct of the invention will generally be designed for specific routes and methods of administration, for specific dosages and frequencies of administration, for specific treatments of specific diseases, with ranges of bio-availability and persistence, among other things. The materials of the composition are preferably formulated in concentrations that are acceptable for the site of administration.

Formulations and compositions thus may be designed in accordance with the invention for delivery by any suitable route of administration. In the context of the present invention, the routes of administration include, but are not limited to
topical routes (such as epicutaneous, inhalational, nasal, opthalmic, auricular/aural, vaginal, mucosal);
enteral routes (such as oral, gastrointestinal, sublingual, sublabial, buccal, rectal); and
parenteral routes (such as intravenous, intraarterial, intraosseous, intramuscular, intracerebral, intracerebroventricular, epidural, intrathecal, subcutaneous, intraperitoneal, extra-amniotic, intraarticular, intracardiac, intradermal, intralesional, intrauterine, intravesical, intravitreal, transdermal, intranasal, transmucosal, intrasynovial, intraluminal).

The pharmaceutical compositions and the antibody construct of this invention are particularly useful for parenteral administration, e.g., subcutaneous or intravenous delivery, for example by injection such as bolus injection, or by infusion such as continuous infusion. Pharmaceutical compositions may be administered using a medical device. Examples of medical devices for administering pharmaceutical compositions are described in U.S. Pat. Nos. 4,475,196; 4,439,196; 4,447,224; 4,447, 233; 4,486,194; 4,487,603; 4,596,556; 4,790,824; 4,941,880; 5,064,413; 5,312,335; 5,312,335; 5,383,851; and 5,399,163. As described elsewhere herein, the pharmaceutical composition according to the invention is preferably administered intravenously.

In particular, the present invention provides for an uninterrupted administration of the suitable composition. As a non-limiting example, uninterrupted or substantially uninterrupted, i.e. continuous administration may be realized by a small pump system worn by the patient for metering the influx of therapeutic agent into the body of the patient. The pharmaceutical composition comprising the antibody construct of the invention can be administered by using said pump systems. Such pump systems are generally known in the art, and commonly rely on periodic exchange of cartridges containing the therapeutic agent to be infused. When exchanging the cartridge in such a pump system, a temporary interruption of the otherwise uninterrupted flow of therapeutic agent into the body of the patient may ensue. In such a case, the phase of administration prior to cartridge replacement and the phase of administration following cartridge replacement would still be considered within the meaning of the pharmaceutical means and methods of the invention together make up one “uninterrupted administration” of such therapeutic agent.

If the pharmaceutical composition has been lyophilized, the lyophilized material is first reconstituted in an appropriate liquid prior to administration. The lyophilized material may be reconstituted in, e.g., bacteriostatic water for injection (BWFI), physiological saline, phosphate buffered saline (PBS), or the same formulation the protein had been in prior to lyophilization.

The compositions of the present invention can be administered to the subject at a suitable dose. The dosage regimen will be determined by the attending physician and clinical factors. As is well known in the medical arts, therapeutically effective dosages for any one patient depend upon many factors, including the patient's size, body surface area, age, the particular compound to be administered, sex, time and route of administration, general health, and other drugs being administered concurrently.

A therapeutic effective amount or dosage of an antibody construct of the invention preferably results in a decrease in severity of disease symptoms, an increase in frequency or duration of disease symptom-free periods or a prevention of impairment or disability due to the disease affliction. For treating tumorous diseases, a therapeutically effective amount of the antibody construct of the invention preferably inhibits cell growth or tumor growth by at least about 20%, at least about 40%, at least about 50%, at least about 60%, at least about 70%, at least about 80%, or at least about 90% relative to untreated patients. The ability of a compound to inhibit tumor growth may be evaluated in an animal model predictive of efficacy in human tumors.

The present invention also relates to a kit comprising an antibody construct of the invention, a nucleic acid molecule of the invention, a vector of the invention or a host cell of the invention. The kit may comprise one or more recipients (such as vials, ampoules, containers, syringes, bottles, bags) of any appropriate shape, size and material (preferably waterproof, e.g. plastic or glass) containing the antibody construct or the pharmaceutical composition of the present invention in an appropriate dosage for administration. The kit may additionally contain instructions for use (e.g. in the form of a leaflet or instruction manual), means for administering the antibody construct of the present invention such as a syringe, pump, infuser or the like, means for reconstituting the antibody construct of the invention and/or means for diluting the antibody construct of the invention. The invention also provides kits for a single-dose administration unit. The kit of the invention may also contain a first recipient comprising a dried/lyophilized antibody construct and a second recipient comprising an aqueous formulation. In certain embodiments of this invention, kits containing single-chambered and multi-chambered pre-filled syringes (e.g., liquid syringes and lyosyringes) are provided. The kit of the invention may typically comprise a container comprising the antibody construct of the invention, the nucleic acid molecule of the invention, the vector of the invention, or the host cell of the invention, and optionally one or more other containers comprising materials desirable from a commercial and user standpoint, including buffers, diluents, filters, needles, syringes, and package inserts with instructions for use.

In some aspects, the invention can be further characterized by the following items.

Item 1. A bispecific antibody construct comprising (a) a first binding domain (A), which is capable of specifically binding to a first target (A′) that is CD16A on the surface of an immune effector cell, wherein the first binding domain comprises: (i) a VL region comprising CDR-L1 as depicted in SEQ ID NO: 4, a CDR-L2 as depicted in SEQ ID NO: 5, and a CDR-L3 as depicted in SEQ ID NO: 6; and (ii) a VH region as depicted in SEQ ID NO: 7 or SEQ ID NO: 94; and (b) a second binding domain (B), which is capable of specifically binding to a second target (B′) that is an antigen on the surface of a target cell, wherein the second target (B′) is FOLR1.

Item 2. The antibody construct of item 1, wherein the first binding domain (A) comprises a VL region as depicted in SEQ ID NO: 8 or SEQ ID NO: 95 and a VH region as depicted in SEQ ID NO: 7 or SEQ ID NO: 94.

Item 3. The antibody construct of item 1 or 2, wherein the first binding domain (A) is a variable domain (Fv), a single chain Fv (scFv), a Fab, a single chain diabody (scDb), a diabody (Db) or a double Fab, preferably a scFv.

Item 4. The antibody construct of any one of items 1 to 3, wherein the second binding domain (B) comprises a VH and a VL domain of an antibody.

Item 5. The antibody construct of any one of items 1 to 4, wherein the second binding domain (B) is a variable domain (Fv), a single chain Fv (scFv), a Fab, a single chain diabody (scDb), a diabody (Db) or a double Fab, preferably a double Fab.

Item 6. The antibody construct of any one of items 1 to 5, wherein the antibody construct binds to a target cell and an immune effector cell simultaneously.

Item 7. The antibody construct of any one of items 1 to 6, wherein the first binding domain binds to an epitope on CD16A which is C-terminal to the physiological Fcγ receptor binding domain, said epitope preferably comprising Y158 of SEQ ID NO: 50.

Item 8. The antibody construct of any one of items 1 to 7, further comprising a third domain (C) comprising a half-life extension domain.

Item 9. The antibody construct of any one of items 1 to 8, wherein said half-life extension domain comprises a CH2 domain, wherein the Fcγ receptor binding domain is silenced.

Item 10. The antibody construct of any one of items 1 to 8, wherein said half-life extension domain comprises a CH3 domain.

Item 11. The antibody construct of any one of items 1 to 10, wherein the antibody construct comprise at least one hinge domain and a CH3 domain fused to a CH2 domain in an amino to carboxyl order in the order hinge domain-CH2 domain-CH3 domain.

Item 12. The antibody construct of any one of items 1 to 11, wherein the antibody construct comprises at least two of the hinge domain-CH2 domain-CH3 domain elements.

Item 13. The antibody construct of any one of items 1 to 12, wherein the first binding domain (A) is fused to the C terminus of a CH3 domain and the second binding domain (B) is fused to the N terminus of a hinge region.

Item 14. The antibody construct of any one of items 1 to 13, wherein the antibody construct is monovalent for the first binding domain (A) and monovalent for the second binding domain (B).

Item 15. The antibody construct of any one of items 1 to 14, wherein the antibody construct is bivalent for the first binding domain (A) and bivalent for the second binding domain (B).

Item 16. The antibody construct of any one of items 1 to 13 or 15, wherein (a) the first binding domain (A), which is capable of specifically binding to a first target (A′) that is CD16A on the surface of an immune effector cell comprises: (i) a VL region comprising CDR-L1 as depicted in SEQ ID NO: 4, a CDR-L2 as depicted in SEQ ID NO: 5, and a CDR-L3 as depicted in SEQ ID NO: 6, and (ii) a VH region as depicted in SEQ ID NO: 7 or SEQ ID NO: 94, wherein said first binding domain is a scFv; (b) the second binding domain which is capable of specifically binding to a second target (B′) that is an antigen on the surface of a target cell that is FOLR1, comprises: (i) a VL region comprising a CDR-L1 as depicted in SEQ ID NO: 117, a CDR-L2 as depicted in SEQ ID NO: 118, a CDR-L3 as depicted in SEQ ID NO: 119, and (ii) a VH region comprising a CDR-H1 as depicted in SEQ ID NO: 114, a CDR-H2 as depicted in SEQ ID NO: 115, a CDR-H3 as depicted in SEQ ID NO: 116, wherein said second binding domain is a Fab; and (c) the third binding domain comprises two of the hinge domain-CH2 domain-CH3 domain elements, preferably as depicted in SEQ ID NOs: 53 and 67; wherein the first binding domain (A) is fused to the C terminus of a CH3 domain of the third domain and the second binding domain (B) is fused to the N terminus of a hinge region of the third domain.

Item 17. The antibody construct of any one of items 1 to 13 or 15, wherein (a) the first binding domain (A), which is capable of specifically binding to a first target (A′) that is CD16A on the surface of an immune effector cell comprises: (i) a VL region comprising CDR-L1 as depicted in SEQ ID NO: 4, a CDR-L2 as depicted in SEQ ID NO: 5, and a CDR-L3 as depicted in SEQ ID NO: 6, and (ii) a VH region as depicted in SEQ ID NO: 7 or SEQ ID NO: 94, wherein said first binding domain is a scFv; (b) the second binding domain which is capable of specifically binding to a second target (B′) that is an antigen on the surface of a target cell that is FOLR1, comprises: (i) a VL region comprising a CDR-L1 as depicted in SEQ ID NO: 127, a CDR-L2 as depicted in SEQ ID NO: 128, and a CDR-L3 as depicted in SEQ ID NO: 129, and (ii) a VH region comprising a CDR-H1 as depicted in SEQ ID NO: 124, a CDR-H2 as depicted in SEQ ID NO: 125, a CDR-H3 as depicted in SEQ ID NO: 126, wherein said second binding domain is a Fab; and (c) the third binding domain comprises two of the hinge domain-CH2 domain-CH3 domain elements, preferably as depicted in SEQ ID NOs: 53 and 67; wherein the first binding domain (A) is fused to the C terminus of a CH3 domain of the third domain and the second binding domain (B) is fused to the N terminus of a hinge region of the third domain.

Item 18. The antibody construct of any one of items 1 to 13, 15 or 16, wherein (a) the first binding domain (A), which is capable of specifically binding to a first target (A′) that is CD16A on the surface of an immune effector cell comprises (i) a VL region as depicted in SEQ ID NO: 8 or SEQ ID NO: 95, and (ii) a VH region as depicted in SEQ ID NO: 7 or SEQ ID NO: 94, wherein said first binding domain is a scFv; (b) the second binding domain which is capable of specifically binding to a second target (B′) that is FOLR1 on the surface of a target cell comprises: (i) a VL region as depicted in SEQ ID NO: 121 and (ii) a VH region as depicted in SEQ ID NO: 120, wherein said second binding domain is a Fab; and (c) the third binding domain comprises two of the hinge domain-CH2 domain-CH3 domain elements, preferably as depicted in SEQ ID NOs: 53 and 67; wherein the first binding domain (A) is fused to the C terminus of a CH3 domain of the third domain and the second binding domain (B) is fused to the N terminus of a hinge region of the third domain.

Item 19. The antibody construct of any one of items 1 to 13, 15 or 17, wherein (a) the first binding domain (A), which is capable of specifically binding to a first target (A′) that is CD16A on the surface of an immune effector cell comprises (i) a VL region as depicted in SEQ ID NO: 8 or SEQ ID NO: 95, and (ii) a VH region as depicted in SEQ ID NO: 7 or SEQ ID NO: 94, wherein said first binding domain is a scFv; (b) the second binding domain which is capable of specifically binding to a second target (B′) that is FOLR1 on the surface of a target cell comprises: (i) a VL region as depicted in SEQ ID NO: 131 and (ii) a VH region as depicted in SEQ ID NO: 130, wherein said second binding domain is a Fab; and (c) the third binding domain comprises two of the hinge domain-CH2 domain-CH3 domain elements, preferably as depicted in SEQ ID NOs: 53 and 67; wherein the first binding domain (A) is fused to the C terminus of a CH3 domain of the third domain and the second binding domain (B) is fused to the N terminus of a hinge region of the third domain.

Item 20A. The antibody construct of any one of items 1 to 17, wherein the second binding domain (B) specific for FOLR1 comprises a pair of VH- and VL-chains having a sequence as depicted in the pair of sequences having at least 85%, 90%, 95%, 96%, 97%, 98%, 99%, or 100% identity each to SEQ ID NOs: 120 and 121, respectively, or having at least 85%, 90%, 95%, 96%, 97%, 98%, 99%, or 100% identity each to SEQ ID NOs: 130 and 131, respectively.

Item 20B. The antibody construct of any one of items 1 to 13, 15, 16 or 18, having an amino acid sequence selected from the group consisting of SEQ ID NOs: 142-143 and 138-139, wherein SEQ ID NOs: 142-143 are preferred.

Item 21. The antibody construct of any one of items 1 to 20B, wherein the antibody construct induces less CD16A shedding as compared to a control construct having an amino acid sequence selected from the group consisting of SEQ ID NOs: 134-135, 136-137 and 140-142.

Item 22. A nucleic acid molecule comprising a sequence encoding an antibody construct of any one of items 1 to 21.

Item 23. A vector comprising a nucleic acid molecule of item 22.

Item 24. A host cell comprising a nucleic acid molecule of item 22 or a vector of item 23.

Item 25. A method of producing an antibody construct of any one of claims 1 to 21, said method comprising culturing a host cell of item 24 under conditions allowing the expression of the antibody construct of any one of claims 1 to 21 and recovering the produced antibody construct from the culture.

Item 26. A pharmaceutical composition comprising an antibody construct of any one of items 1 to 21, or produced by the method of item 25.

Item 27. The antibody construct of any one of items 1 to 21 for use in therapy.

Item 28. The antibody construct of any one of items 1 to 21, or produced by the method of item 25, for use in the prevention, treatment or amelioration of a disease selected from a proliferative disease, a tumorous disease, a viral disease or an immunological disorder.

Item 29. The antibody construct for use of item 28, wherein said tumorous disease is a solid tumor, preferably a malignant solid tumor.

Item 30. The antibody construct for use of item 29, wherein said solid tumor is selected from the group consisting of ovarian, breast (particularly TNBC), renal, lung, colorectal (CRC), kidney (particularly ccRCC), pancreatic (particularly PDAC), endometrial and brain cancer.

Item 31. A method of treatment or amelioration of a proliferative disease, a tumorous disease, a viral disease or an immunological disorder, comprising the step of administering to a subject in need thereof the antibody construct of any one of items 1 to 21, or produced by the method of item 25.

Item 32. A kit comprising an antibody construct of any one of items 1 to 21, or produced by the method of item 25, a nucleic acid of item 22, a vector of item 23, and/or a host cell of item 24.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 10%, preferably within 5%, more preferably within 2%, even more preferably within 1% of a given value or range (plus (+) or minus (−)). It includes, however, also the concrete number, e.g., about 20 includes 20.

The term “less than” or “greater than” includes the concrete number. For example, less than 20 means less than or equal to. Similarly, more than or greater than means more than or equal to, or greater than or equal to, respectively.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein, any of the terms “comprising”, “consisting essentially of” and “consisting of” may be replaced with either of the other two terms. For example, the disclosure of the term “comprising” includes the disclosure of the terms “consisting essentially of” as well as the disclosure of the term “consisting of”.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

A better understanding of the present invention and of its advantages will be obtained from the following examples, offered for illustrative purposes only. The examples are not intended to limit the scope of the present invention in any way.

### EXAMPLES

### Example 1: Detection of CD16A Interaction with CD16A Binding Domains

### Methods

Multivalent interaction kinetic of CD123×CD16A ICE to human CD16A^{158V}, CD16A^{158F} and cynomolgus CD16 was analyzed at 37° C. using a Biacore T200 instrument (GE Healthcare) equipped with a research-grade Sensor Chip CAP (Biotin CAPture Kit, GE Healthcare) pre-equilibrated in HBS-P+ running buffer. For multivalent interaction analysis, biotinylated-mFc.silenced/Avi-tagged antigens were captured (FC2, FC4) to a density of 120-200RU, before CD123×CD16A ICE were injected (concentration: 0-60 nM) for 240s at a flow rate of 40 μL/min and complex was left to dissociate for 300s at the same flow rate.

After each cycle, chip surfaces were regenerated with 6M guanidine-HCl, 0.25M NaOH and reloaded with Biotin Capture reagent. Interaction kinetics were determined by fitting data from multi-cycle kinetics experiments to a simple 1:1 interaction model using the local data analysis option (Rmax and RI) available within Biacore T200 Evaluation Software (v3.1). Referencing was done against a flow cell without captured ligand (Fc2-Fc1, Fc4-Fc3).

### Results

CD123×CD16A ICE binding to human CD16^{158V}, CD16A^{158F} and cynomolgus CD16 was measured by SPR using a multivalent multi-cycle kinetic set up at 37° C. (n=3; ²) n=1) with biotin captured recombinant CD16A^{158V}, CD16A^{158F} and cynomolgus CD16 (ligand) and scFv-IgAb_268 (CD16a1×CD123-1), scFv-IgAb_148 (CD16a2×CD123-1), scFv-IgAb_264 (CD16a1×CD123-2) (analyte). Affinity and kinetic parameters were evaluated for interaction with human CD16A and cynomolgus CD16 using a 1:1 Binding model. All molecules showed high interaction to human CD16A as well as to cynomolgus CD16 with apparent affinities in the range of K_{D} 0.195 nM-2.48 nM (FIG. 1).

### Example 2: Binding of CD123×CD16A Constructs to Cell Lines Expressing Human CD16A

### Methods

TABLE 2
Antibody constructs

| | Target | Target | Effector | Effector |
|---|---|---|---|---|
| Construct | specificity | domain | specificity | domain |
| scFv-IgAb_268 | CD123 | CD123-1 | CD16A | CD16a1 |
| scFv-IgAb_148 | CD123 | CD123-1 | CD16A | CD16a2 |
| scFv-IgAb_139 | CD123 | CD123-2 | RSV | NIST |
| | | | | RM8671 |

### Flp-In CHO Host Cell Culture

Flp-In CHO cells (Life Technologies, R75807), a derivative of CHO-K1 Chinese Hamster ovary cells, were adapted to growth in suspension in HyClone CDM4CHO medium supplemented (Cytiva, cat. SH30557.02) with L-Glutamine (Invitrogen, cat. 25030-024), HT Supplement (Thermo Fisher Scientific, cat. 41065012), Penicillin/Streptomycin (Invitrogen, cat. 1540-122) and 100 μg/mL Zeocin (Thermo Fisher Scientific, cat. R250-01). Single cell derived clonal lines were obtained by limiting dilution cloning in a medium mixture of standard culture medium with Ham's F-12 supplemented (Thermo Fisher Scientific, cat. 11500586) with InstiGRO CHO supplement (Solentim, cat. RS-1105), expanded, and cryopreserved in medium with 10% DMSO (Sigma, cat. D2650). Cultures were routinely subcultured after 2 or 3 days and diluted in fresh medium to 3E+5 viable cells/mL for a subsequent 2-day passage or 2E+5 viable cells/mL for a 3-day passage, cultured in shake flasks or tubes at 37° C., 5% CO2 and 120-200 rpm depending on the vessel type.

Generation of Stably Transfected Antigen Expressing Cells (cAg)

Suspension-adapted Flp-In CHO host cells were subcultured in standard medium without Zeocin one day prior to transfection. Recombinant CHO cells were generated by transfection of 2E+6 cells in 2 mL of CHO-S-SFMII medium (Thermo Fisher Scientific, cat. 12052-114), with expression plasmids encoding recombinant cell-anchored antigen sequences (cAgs) in a modified, version of pcDNA5/FRT vector, mediating Puromycin resistance or Hygromycin resistance and the Flp recombinase (pOG44, Thermo Fisher, V600520) using a total of 2.5 μg of DNA and Transporter 5 transfection reagent at a DNA:PEI ratio of 1:2.5 (μg/μg). DNA and transfection reagent were mixed in 100 μL NaCl solution (Sigma, cat. S8776), 0.9% and incubated for 20 minutes before addition to the cells. As a negative control (mock), cells were transfected with a control plasmid not mediating resistance. After 4 hours, transfected cells were diluted with 8 mL of a 1:1 medium mixture of standard culture medium with Ham's F-12. Selection of stably transfected cells was started on the following day by addition of 3.2 μg/mL of Puromycin Dihydrochloride (Thermo Fisher Scientific, cat. A1113803) as selection antibiotic and an increase to 6.3 μg/mL on day 2 or of 500 μg/ml of Hygromycin B (Thermo Fisher Scientific, cat. 10687010). Viable cell densities were measured twice per week, and cells were centrifuged and resuspended in fresh selection medium containing selection antibiotic at a maximal density of 2-4E+5 viable cells/mL. Concentration of Puromycin Dihydrochloride was increased to 7.0 μg/mL on day 10 after transfection. Stably transfected cell pools recovered in growth and viability after approximately 2-3 weeks, were expanded in standard culture medium and cryopreserved in freezing medium containing 7.5% DMSO. For analysis of antigen expression, cultures were propagated in shake flasks or tubes and subcultured after 2 or 3 days and diluted in fresh medium to 6E+5 viable cells/mL for a subsequent 2-day passage or 3E+5 viable cells/mL for a 3-day passage, cultured at 37° C., 5% CO2 and 120-200 rpm depending on the vessel type.

### Flow Cytometric Analysis

To analyze binding of different antibody constructs to CHO cells transfected with human CD16A (cAg_34), relative to CD16 expression by flow cytometry, 1-2×10⁵ were resuspended in 100 μL FACS buffer (PBS (Invitrogen, cat.: 14190-169) containing 2% heat-inactivated FCS (Invitrogen, cat.: 10270-106), and 0.1% sodium azide (Roth, Karlsruhe, Germany, cat.: A1430.0100)) in round-bottom 96-well microtiter plates. After washing in FACS buffer, cells were incubated in 50 μL FACS buffer without antibodies or with titrated antibodies starting at a concentration of 100 μg/mL followed by ten 5-fold serial dilutions for 30 min on ice in the dark. After washing twice, cells were incubated with APC-conjugated goat anti-human IgG (H+L)-APC (Dianova, cat. 109-136-088) for 30 min on ice in the dark. As controls, cells were only incubated with anti-human CD16-BV421 (clone 3G8, Biolegend, cat. 302038). After washing, binding was measured by flow cytometry and mean fluorescence intensities (MFI) of cell samples were calculated and corrected for background staining using control cells stained with secondary antibodies only.

### Statistical Analysis

Equilibrium dissociation constants (K_{D}) of antibody binding, mean and standard deviation (SD) were calculated by plotting MFI values and fitting a non-linear regression model for one-site binding to hyperbolic dose-response curves using GraphPad Prism for Windows (v9; GraphPad Software; La Jolla California USA).

### Results

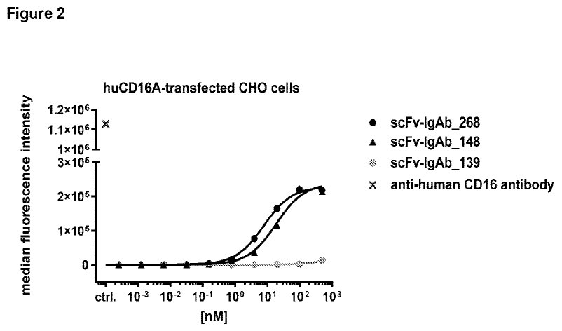

The apparent avidity of scFv-IgAb_268 (CD123×CD16A) and scFv-IgAb_148 (CD123×CD16A) to human (hu) CD16A was determined. CHO cells expressing recombinant huCD16A (cAg_34) were incubated with increasing concentrations of scFv-IgAb_268, scFv-IgAb_148 and binding was assessed relative to control molecules (scFv-IgAb_139) by flow cytometry. CD16 expression on huCD16A CHO cells was confirmed using anti-human CD16A antibody clone 3G8 (FIG. 2).

The antibody construct scFv-IgAb_268 exhibited a higher dose-dependent binding to huCD16A resulting in a mean K_{D} of 16.3 nM compared to scFv-IgAb_148 resulting in a mean K_{D} of 37.6 nM (FIG. 2, Table 3). No binding was detected by a negative control molecule (CD123×RSV, scFv-IgAb_139) comprising the same antibody scaffold and CD123-targeting domain as scFv-IgAb_268 but an irrelevant anti-RSV domain replacing CD16A. Hence these results corroborate higher binding specificity to human CD16A of scFv-IgAb_268 containing CD16a1 anti-CD16 effector domain compared to scFv-IgAb_148 containing CD16a2 anti-CD16 effector domain.

TABLE 3
Mean apparent avidity (KD) of scFv-IgAb_268,
scFv-IgAb_148 and control antibody to human CD16A expressed
on CHO cells. Binding of antibody constructs to huCD16A-transfected
CHO cells measured by flow cytometry of titrated scFv-IgAb_268
(CD123 × CD16A), scFv-IgAb_148 (CD123 × CD16A) and a negative
control molecule (scFv-IgAb_139, CD123 × RSV). Equilibrium
dissociation constants (KD) of antibody binding were calculated
by plotting MFI values and fitting a non-linear regression model
for one-site binding to hyperbolic dose-response curves
using GraphPad Prism.

| | K_{D} values [nM] | | |
|---|---|---|---|
| Experiment | scFv-IgAb_268 | scFv-IgAb_148 | scFv-IgAb_139 |
| 1 | 17.7 | 56.2 | n.a. |
| 2 | 7.8 | 18.7 | n.a. |
| 3 | 23.5 | 37.9 | n.a. |
| mean | 16.3 | 37.6 | n.a. |
| SD | 7.9 | 18.8 | n.a. |

SD, standard deviation; n.a., not applicable.

### Example 3: Assessment of Cell Surface Retention of Anti-CD123 Antibodies on NK Cells

### Methods

TABLE 4
Antibody constructs

| | Target | Target | Effector | Effector |
|---|---|---|---|---|
| Construct | specificity | domain | specificity | domain |
| scFv-IgAb_268 | CD123 | CD123-1 | CD16A | CD16a1 |
| IgAb_338 | CD123 | CD123-2 | IgG1 | Fc-enhanced |
| scFv-IgAb_148 | CD123 | CD123-1 | CD16A | CD16a2 |

Isolation of PBMC from Buffy Coats and Enrichment of Human NK Cells

PBMCs were isolated from buffy coats (German Red Cross, Mannheim, Germany) by density gradient centrifugation. The buffy coat samples were diluted with a two-to-threefold volume of PBS (Invitrogen, cat.: 14190-169), layered on a cushion of Lymphoprep (Stem Cell Technologies, cat.: 07861) and centrifuged at 800×g for 25 min at room temperature w/o brake. PBMC located in the interface were collected and washed 3 times with PBS before they were cultured in complete RPMI 1640 medium (RPMI 1640 medium supplemented 10% heat-inactivated FCS, 2 mM L-glutamine and 100 IU/mL penicillin G sodium and 100 μg/mL streptomycin sulfate (all components from Invitrogen)) overnight without stimulation. For the enrichment of NK cells PBMC were harvested from overnight cultures and used for one round of negative selection using the EasySep™ Human NK Cell Enrichment Kit (Stem Cell Technologies, cat.: 17055) for the immunomagnetic isolation of untouched human NK cells and the Big Easy EasySep™ Magnet (Stem Cell Technologies, cat.: 18001) according to the manufacturer's instructions.

### Flow Cytometric Detection of Cell Surface Retention on NK Cells

NK cells were suspended in a volume of 1 mL at a density of 10-15×10⁶ cells/mL in pre-chilled complete RPMI 1640 medium. Antibody constructs were added to a concentration of 100 μg/mL and incubated for 45 min on ice. Afterwards, 10 mL of complete RPMI 1640 medium were added and the cell suspension split into two equal volumes, washed twice with complete RPMI 1640 medium, and each NK cell suspension was resuspended in 10 mL complete RPMI 1640 medium. For each dissociation time aliquots of 1 mL NK cell suspension were then transferred into single tubes containing 9 mL pre-warmed complete RPMI 1640 medium. Diluted NK cell suspensions were then placed for the respective duration in a water bath at 37° C. to allow dissociation of bound antibodies and placed on ice to stop dissociation. The “0 min” sample was directly transferred on ice. Cell aliquots were washed once with FACS buffer (PBS (Invitrogen, cat.: 14190-169) containing 2% heat-inactivated FCS (Invitrogen, cat.: 10270-106), and 0.1% sodium azide (Roth, Karlsruhe, Germany, cat.: A1430.0100)) and transferred to a 96-well round-bottom plate for detection of cell surface-retained antibodies by flow cytometry. Cell surface bound scFv-IgAb_268, scFv-IgAb_148 and IgAb_338 were detected by staining with 10 μg/mL anti-anti-CD123 mAb (clone 8-1-1), followed by incubation with 15 μg/mL FITC goat anti-mouse IgG (Dianova, cat. 115-095-062) and staining with Fixable Viability Stain eFluor™ 780 (Fisher Scientific, cat.: 65-0865-14) to exclude dead cells. After the last washing step cells were resuspended in 0.2 mL of FACS buffer and the fluorescence of cells was measured using a flow cytometer, and median fluorescence intensities of the cell samples were calculated. After subtracting the fluorescence intensity values of the cells stained with the secondary and/or tertiary reagents alone, the MFI values at time-point 0 were taken to be 100%, and the percentages of remaining antibody were analyzed by non-linear regression using GraphPad Prism for Windows (v9; GraphPad Software; La Jolla California USA).

### Results

Primary human NK cells were preloaded with anti-CD123 antibody constructs containing different effector domains for CD16A to assess the retention of the constructs on the surface of NK cells. The Fc-enhanced anti-CD123 IgG1 antibody (IgAb_338) dissociated very rapidly form NK cells, reaching a lower plateau after the first 5-10 min. The CD123×CD16A scFv-IgAb_148 containing the CD16a2 effector domain exhibited a lower dissociation reaching a plateau of 20% remaining antibodies after 48 h (FIG. 3). In contrast to the Fc-enhanced IgG1 and scFv-IgAb_148 containing the CD16a2 anti-CD16A domain, AFM28 (CD123×CD16A scFv-IgAb_268) containing the CD16a1 anti-CD16A effector domain showed the substantial longer retention (˜60%) on NK cells after 24 h and 48 h dissociation at 37° C. (FIG. 3, Table 5).

TABLE 5
Remaining antibody in percentage [%] on NK cells after 24 h
Enriched primary human NK cells were preloaded with 100 μg/mL
CD123/CD16A scFv-IgAb_268, Fc-enhanced anti-CD123 IgG1
(IgAb_338), or CD123/CD16A scFv-IgAb_148 on ice, washed, and
then incubated at 37° C. for the indicated time periods in an excess
volume of complete RPMI 1640 medium to allow dissociation and to
prevent re-association. Residual antibodies after 24 h were
determined by flow cytometry, and median fluorescence intensity
(MFI) values at time-point 0 were taken to be 100%, and the percentages
of remaining antibody were analysed using GraphPad Prism.

| | Remaining antibody [%] | | |
|---|---|---|---|
| Experiment | scFv-IgAb_268 | scFv-IgAb_148 | IgAb_338 |
| 1 | 69.7 | 33.7 | 0.6 |
| 2 | 50.0 | 5.7 | 0.2 |
| mean | 59.9 | 19.7 | 0.4 |
| SD | 13.9 | 19.8 | 0.3 |

SD, standard deviation.

### Example 4: ADCC Against CD123+ EOL-1 Cells by Anti-CD123 Antibodies Methods

TABLE 6
Antibody constructs

| | Target | Target | Effector | Effector |
|---|---|---|---|---|
| Construct | specificity | domain | specificity | domain |
| scFv-IgAb_268 | CD123 | CD123-1 | CD16A | CD16a1 |
| scFv-IgAb_267 | CD123 | CD123-2 | CD16A | CD16a1 |
| scFv-IgAb_265 | CD123 | CD123-1 | CD16A | CD16a2 |
| scFv-IgAb_264 | CD123 | CD123-2 | CD16A | CD16a2 |

Isolation of PBMC from Buffy Coats and Enrichment of Human NK Cells

PBMCs were isolated from buffy coats (German Red Cross, Mannheim, Germany) by density gradient centrifugation. The buffy coat samples were diluted with a two-to-threefold volume of PBS (Invitrogen, cat.: 14190-169), layered on a cushion of Lymphoprep (Stem Cell Technologies, cat.: 07861) and centrifuged at 800×g for 25 min at room temperature w/o brake. PBMC located in the interface were collected and washed 3 times with PBS before they were cultured in complete RPMI 1640 medium (RPMI 1640 medium supplemented 10% heat-inactivated FCS, 2 mM L-glutamine and 100 IU/mL penicillin G sodium and 100 μg/mL streptomycin sulfate (all components from Invitrogen)) overnight without stimulation. For the enrichment of NK cells PBMC were harvested from overnight cultures and used for one round of negative selection using the EasySep™ Human NK Cell Enrichment Kit (Stem Cell Technologies, cat.: 17055) for the immunomagnetic isolation of untouched human NK cells and the Big Easy EasySep™ Magnet (Stem Cell Technologies, cat.: 18001) according to the manufacturer's instructions.

### Culture of EOL-1 Tumor Cell Line

The EOL-1 cell line was cultured under standard conditions as recommended by the supplier (DSMZ, cat.: ACC-386) at 37° C. and 5% CO2 in a humidified atmosphere in complete RPMI medium (RPMI 1640 medium supplemented with 10% h.i. FCS, 2 mM L glutamine, 100 U/mL penicillin G sodium, 100 μg/mL streptomycin sulfate).

### Calcein-Release Cytotoxicity Assays

Antibody-mediated target cell lysis by NK cells in vitro was assessed by quantifying the release of calcein into cell culture supernatants from calcein-labeled target cells. For this, target cells were labeled with 10 μM calcein AM for 30 min in RPMI 1640 medium without FCS at 37° C. After gentle washing, calcein-labeled cells were resuspended in complete RPMI medium at a density of 1×10⁵/mL. 1×10⁴ target cells were then seeded in individual wells of a round-bottom 96-well microtiter plate and, if not mentioned otherwise, mixed with enriched human NK cells at an effector-to-target cell (E:T) ratio of 5:1. The culture of NK cells with target cells was conducted in duplicate without antibody addition or in the presence of titrated antibodies starting at a concentration of 25 μg/mL followed by ten 2-fold serial dilutions. After centrifugation for 2 min at 200×g, microtiter plates were incubated for 4 h at 37° C. in a humidified atmosphere with 5% CO₂. Spontaneous calcein-release, maximal release and killing of targets by effectors in the absence of antibodies were determined in quadruplicate on each plate. Spontaneous release was determined by incubation of target cells in the absence of effector cells and in the absence of antibodies. Maximal release was achieved by adding Triton X-100 to a final concentration of 1% in the absence of effector cells and in the absence of antibodies. Following incubation, 100 μL cell-free cell culture supernatant was harvested from each well after centrifugation for 5 min at 500×g and transferred to black flat-bottom 96-well microtiter plates. Fluorescence counts of released calcein were measured at 520 nm using a multimode plate reader. Specific cell lysis was calculated according to the following formula: [fluorescence (sample)−fluorescence (spontaneous)]/[fluorescence (maximum)−fluorescence (spontaneous)]×100% wherein “Fluorescence (spontaneous)” and “Fluorescence (maximum)” are defined as fluorescence in absence of effector cells and antibodies and fluorescence induced by the addition of Triton X-100, respectively.

### Results

All four CD123×CD16A scFv-IgAb constructs induced NK cell-dependent lysis against EOL-1 cells at similar maximal efficacy in the low picomolar concentration range (FIG. 4).

### Example 5: Assessment of Shedding Inhibition of CD16A on Activated NK Cells in Presence of AFM28

### Methods

TABLE 7
Antibody constructs

| | Target | Target | Effector | Effector |
|---|---|---|---|---|
| Construct | specificity | domain | specificity | domain |
| scFv-IgAb_268 | CD123 | CD123-1 | CD16A | CD16a1 |
| IgAb_338 | CD123 | CD123-2 | IgG1 | Fc-enhanced |
| scFv-IgAb_148 | CD123 | CD123-1 | CD16A | CD16a2 |

Isolation of PBMC from Buffy Coats and Enrichment of Human NK Cells

PBMCs were isolated from buffy coats (German Red Cross, Mannheim, Germany) by density gradient centrifugation. The buffy coat samples were diluted with a two-to-threefold volume of PBS (Invitrogen, cat.: 14190-169), layered on a cushion of Lymphoprep (Stem Cell Technologies, cat.: 07861) and centrifuged at 800×g for 25 min at room temperature w/o brake. PBMC located in the interface were collected and washed 3 times with PBS before they were cultured in complete RPMI 1640 medium (RPMI 1640 medium supplemented 10% heat-inactivated FCS, 2 mM L-glutamine and 100 IU/mL penicillin G sodium and 100 μg/mL streptomycin sulfate (all components from Invitrogen)) overnight without stimulation. For the enrichment of NK cells PBMC were harvested from overnight cultures and used for one round of negative selection using the EasySep™ Human NK Cell Enrichment Kit (Stem Cell Technologies, cat.: 17055) for the immunomagnetic isolation of untouched human NK cells and the Big Easy EasySep™ Magnet (Stem Cell Technologies, cat.: 18001) according to the manufacturer's instructions.

### Flow Cytometric Detection of CD16A Expression on NK Cells

NK cells were suspended in a volume of 1 mL at a density of 10-15×10⁶ cells/mL in pre-chilled complete RPMI 1640 medium. Antibody constructs were added to a concentration of 100, 10, and 1 μg/mL and incubated for 45 min on ice. Afterwards cells were washed with in complete RPMI 1640 medium and transferred to 96-well round-bottom plate. NK cells were incubated with or without 50 ng/mL PMA and 0.5 μM Ionomycin for 4 h at 37° C. After the stimulation cells were washed with FACS buffer (PBS (Invitrogen, cat.: 14190-169) containing 2% heat-inactivated FCS (Invitrogen, cat.: 10270-106), and 0.1% sodium azide (Roth, Karlsruhe, Germany, cat.: A1430.0100)). To detect the CD16 level, cells were restained with 100 μg/mL scFv-IgAb_268, scFv-IgAb_148 or IgAb_338 followed by incubation with 15 μg/mL FITC-conjugated goat anti-mouse IgG (Dianova, cat. 115-095-062) and staining with Fixable Viability Stain eFluor™ 780 (Fisher Scientific, cat.: 65-0865-14) to exclude dead cells. After the last washing step cells were resuspended in 0.2 mL of FACS buffer and the fluorescence of cells was measured using a flow cytometer, and median fluorescence intensities of the cell samples were calculated. After subtracting the fluorescence intensity values of the cells stained with the secondary reagents alone, the MFI values were plotted using the GraphPad Prism software (v8.0/9.06.0/7.0; GraphPad Software; La Jolla California USA). Figures were generated using FlowJo Software (v10.6/10.8, FlowJo Software, BD Ashland USA).

### Statistical Analysis

The paired Student's t-test was used to compare quantitative variables. Statistical significance was assessed with GraphPad Prism software (v9.0). p values <0.05 were considered significant.

### Results

Primary human NK cells were preloaded with anti-CD123 constructs containing different effector domains for CD16A and were stimulated with PMA/Ionomycin. Expression levels of CD16 were assessed with flow cytometry. As described NK cells stimulated with PMA/Ionomycin show no expression of CD16 compared to unstimulated cells (FIGS. 5A-5C, FIGS. 6A-6C). This phenomenon was described in literature as shedding of CD16 in response of NK cell stimulation (Romee R. et al. 2013). NK cells incubated with different concentrations of Fc-enhanced anti-CD123 IgG1 antibody (IgAb_338) followed by PMA/Ionomycin stimulation showed a similar effect (FIG. 5C, FIG. 6C). Interestingly, high concentrations of CD123/CD16A scFv-IgAb_268 (100 μg/mL) containing the CD16a1 anti-CD16A effector domain exhibited a significant higher level of CD16 expression after stimulation compared to unstimulated NK cells (FIG. 5A, FIG. 6A). Furthermore, we could observe a concentration dependent shedding inhibition by scFv-IgAb_268 and to a lower extend by scFv-IgAb_148 (CD123/CD16A) containing CD16a2 anti-CD16A effector domain (FIG. 5A-5B, FIG. 6A-5B). However, the CD16 shedding inhibition effect on stimulated NK cells was stronger induced by scFv-IgAb_268 compared to the shedding inhibition induced by scFv-IgAb_148.

### Example 7: Target Cell-Independent Activation of NK Cells by Anti-CD123 Antibodies

### Methods

TABLE 8
Antibody constructs

| | Target | Target | Effector | Effector |
|---|---|---|---|---|
| Construct | specificity | domain | specificity | domain |
| scFv-IgAb_268 | CD123 | CD123-1 | CD16A | CD16a1 |
| scFv-IgAb_267 | CD123 | CD123-2 | CD16A | CD16a1 |
| scFv-IgAb_265 | CD123 | CD123-1 | CD16A | CD16a2 |
| scFv-IgAb_264 | CD123 | CD123-2 | CD16A | CD16a2 |

Isolation of PBMC from buffy coats and enrichment of human NK cells PBMCs were isolated from buffy coats (German Red Cross, Mannheim, Germany) by density gradient centrifugation. The buffy coat samples were diluted with a two-to-threefold volume of PBS (Invitrogen, cat.: 14190-169), layered on a cushion of Lymphoprep (Stem Cell Technologies, cat.: 07861) and centrifuged at 800×g for 25 min at room temperature w/o brake. PBMC located in the interface were collected and washed 3 times with PBS before they were cultured in complete RPMI 1640 medium (RPMI 1640 medium supplemented 10% heat-inactivated FCS, 2 mM L-glutamine and 100 IU/mL penicillin G sodium and 100 μg/mL streptomycin sulfate (all components from Invitrogen)) overnight without stimulation. For the enrichment of NK cells PBMC were harvested from overnight cultures and used for one round of negative selection using the EasySep™ Human NK Cell Enrichment Kit (Stem Cell Technologies, cat.: 17055) for the immunomagnetic isolation of untouched human NK cells and the Big Easy EasySep™ Magnet (Stem Cell Technologies, cat.: 18001) according to the manufacturer's instructions.

### Cultures and Flow Cytometric Analysis

Buffy coat-derived NK cells (5×10⁴) were cultured overnight in the presence titrated antibodies, starting at a concentration of 40 μg/mL followed by five 10-fold serial dilutions, or without antibodies in complete RPMI medium in 96-well microtiter plates. Afterwards, up-regulation of the NK cell activation marker CD137 on CD56+ CD45+ CD3− CD19− NK cells was assessed after extracellular staining with fluorescently conjugated mouse anti-human antibodies, diluted in 50 μL FACS buffer, by flow cytometry. The percentage of CD137-positive NK cells is indicated.

### Results

Of the four anti-CD123 antibodies, CD123×CD16A scFv-IgAb constructs constituting the anti-CD16A CD16a1 domain showed the lowest activity in up-regulation of the activation marker CD137 on NK cells in the absence of CD123+ target cells. At the highest tested concentration of 40 μg/mL, scFv-IgAb_268 appeared to have the lowest unspecific activity to activate NK cells, followed in sequence by scFv-IgAb_267, scFv-IgAb_265 and scFv-IgAb_264 (FIG. 7).

### Example 7: Target Cell-Dependent Activation of NK Cells by Anti-CD123 Antibodies

### Methods

TABLE 9
Antibody constructs

| | Target | Target | Effector | Effector |
|---|---|---|---|---|
| Construct | specificity | domain | specificity | domain |
| scFv-IgAb_268 | CD123 | CD123-1 | CD16A | CD16a1 |
| scFv-IgAb_267 | CD123 | CD123-2 | CD16A | CD16a1 |
| scFv-IgAb_265 | CD123 | CD123-1 | CD16A | CD16a2 |
| scFv-IgAb_264 | CD123 | CD123-2 | CD16A | CD16a2 |
| scFv-IgAb_239 | RSV | NIST | CD16A | CD16a1 |
| | | RM8671 | | |
| scFv-IgAb_238 | RSV | NIST | CD16A | CD16a2 |
| | | RM8671 | | |

### Culture of Tumor Cell Lines

The EOL-1 cell line was cultured under standard conditions as recommended by the supplier (DSMZ, cat.: ACC-386) at 37° C. and 5% CO₂ in a humidified atmosphere in complete RPMI medium (RPMI 1640 medium supplemented with 10% h.i. FCS, 2 mM L glutamine, 100 U/mL penicillin G sodium, 100 μg/mL streptomycin sulfate).

Isolation of PBMC from Buffy Coats and Enrichment of Human NK Cells

PBMCs were isolated from buffy coats (German Red Cross, Mannheim, Germany) by density gradient centrifugation. The buffy coat samples were diluted with a two-to-threefold volume of PBS (Invitrogen, cat.: 14190-169), layered on a cushion of Lymphoprep (Stem Cell Technologies, cat.: 07861) and centrifuged at 800×g for 25 min at room temperature w/o brake. PBMC located in the interface were collected and washed 3 times with PBS before they were cultured in complete RPMI 1640 medium (RPMI 1640 medium supplemented 10% heat-inactivated FCS, 2 mM L-glutamine and 100 IU/mL penicillin G sodium and 100 μg/mL streptomycin sulfate (all components from Invitrogen)) overnight without stimulation. For the enrichment of NK cells PBMC were harvested from overnight cultures and used for one round of negative selection using the EasySep™ Human NK Cell Enrichment Kit (Stem Cell Technologies, cat.: 17055) for the immunomagnetic isolation of untouched human NK cells and the Big Easy EasySep™ Magnet (Stem Cell Technologies, cat.: 18001) according to the manufacturer's instructions.

### Co-Cultures and Flow Cytometric Analysis

CMFDA-labelled EOL-1 cells (5×10⁴) were co-cultured with buffy coat-derived allogeneic NK cells (5×10⁴) at 1:1 cell ratio for 24 h in the presence titrated antibodies or control molecules, starting at a concentration of 50 μg/mL followed by six 10-fold serial dilutions, in complete RPMI medium in 96-well microtiter plates. Afterwards, up-regulation of the NK cell activation marker CD137 on CD56+ CD45+ CD3− CD19− NK cells was assessed after extracellular staining with fluorescently-conjugated mouse anti-human antibodies, diluted in 50 μL FACS buffer, by flow cytometry. The percentage of CD137-positive NK cells is indicated.

### Results

All four CD123×CD16A scFv-IgAb constructs specifically induced the up-regulation of the activation marker CD137 on NK cells in response to CD123+ EOL-1 cells (FIG. 8). Of note, antibody constructs constituting the anti-CD16A CD16a1 domain reached a peak in the percentages of CD137+ NK cells at 0.05 μg/mL, followed by decreasing percentages of CD137+ NK cells at higher concentrations. In contrast, antibody constructs constituting the anti-CD16A CD16a2 domain resulted in continuously increasing percentages of CD137+ NK cells up to the highest tested concentration of 50 μg/mL. Non-CD123-targeting RSV×CD16A control antibody constructs, replacing the CD123 by a non-binding RSV domain, failed to induce NK cell activation in response to EOL-1 cells.

### Example 8: Binding of CD123×CD16A Constructs to CD123+ and CD123− Tumor Cell Lines

### Methods

TABLE 10
Antibody constructs

| | Target | Target | Effector | Effector |
|---|---|---|---|---|
| Construct | specificity | domain | specificity | domain |
| scFv-IgAb_268 | CD123 | CD123-1 | CD16A | CD16a1 |
| scFv-IgAb_267 | CD123 | CD123-2 | CD16A | CD16a1 |
| scFv-IgAb_265 | CD123 | CD123-1 | CD16A | CD16a2 |
| scFv-IgAb_264 | CD123 | CD123-2 | CD16A | CD16a2 |

### Culture of Tumor Cell Lines

The EOL-1 (DSMZ, cat.: ACC-386) and Karpas-299 (DSMZ, cat.: ACC-31) cell lines were cultured under standard conditions as recommended by the supplier at 37° C. and 5% CO2 in a humidified atmosphere in complete RPMI medium (RPMI 1640 medium supplemented with 10% h.i. FCS, 2 mM L glutamine, 100 U/mL penicillin G sodium, 100 μg/mL streptomycin sulfate). Adherent A-431 cells were dislodged by accutase treatment and maintained under standard conditions as recommended by the supplier (DSMZ, cat.: ACC-91) at 37° C. and 5% CO2 in a humidified atmosphere in complete DMEM medium (Dulbecco's Modified Eagle's medium with 10% h.i. FCS, 2 mM L-glutamine, 100 U/mL penicillin G sodium, 100 μg/mL streptomycin sulfate).

### Flow Cytometric Analysis

To analyze binding of CD123×CD16A antibody constructs to CD123+ EOL-1 cells, CD123− A− 431 cells and CD123− Karpas-299 cells by flow cytometry, 1×10⁵ cells were resuspended in 100 μL FACS buffer in round-bottom 96-well microtiter plates. After washing in FACS buffer, cells were incubated in 100 μL FACS buffer without antibodies or with titrated antibodies starting at a concentration of 100 μg/mL followed by eight 10-fold serial dilutions for 45 min on ice in the dark. After washing twice, cells were incubated with APC-conjugated goat anti-human IgG (H+L)-APC (1/200 dilution) for 30 min on ice in the dark. After washing, binding was measured by flow cytometry and mean fluorescence intensities (MFI) of cell samples were calculated and corrected for background staining using control cells stained with secondary antibodies only.

### Results:

All four CD123×CD16A scFv-IgAb constructs showed comparable binding to CD123+ EOL-1 cells (FIG. 9A). In contrast, to CD123− A431 cells, scFv-IgAb_268 constituted of the CD123-1 domain and the CD16a1 binding domain showed lowest potential for unspecific binding. Overall, scFv-IgAb_268 showed least unspecific binding to CD123− A-431 cells, followed by scFv-IgAb_265, followed by scFv-IgAb_267, followed by scFv-IgAb_264 across different antibody construct batches tested (FIG. 9B).

### Example 9: NK Cell-Dependent Depletion of Primary Leukemic Blasts Mediated by Anti-CD123 Antibodies

### Methods

TABLE 11
Antibody constructs

| | Target | Target | Effector | Effector |
|---|---|---|---|---|
| Construct | specificity | domain | specificity | domain |
| scFv-IgAb_268 | CD123 | CD123-1 | CD16A | CD16a1 |
| scFv-IgAb_239 | RSV | NIST | CD16A | CD16a1 |
| | | RM8671 | | |
| IgAb_338 | CD123 | CD123-2 | IgG1 | Fc- |
| | | (Tala- | | enhanced |
| | | cotuzumab) | | |

TABLE 12
Primary samples from AML patients

| | | | | | | Identification | |
|---|---|---|---|---|---|---|---|
| | | | | Blast | % | of leukemic | |
| Patient | Patient | AML | | content | CD123⁺ | blasts by flow | |
| code | ID | subtype | Material | in PB | blasts | cytometry | Vendor |
| AML 1 | AML860L | M1 | PB | 86% | 69% | CD45⁺ CD34⁺ | Tissue |
| | | | | | | | Solutions |
| AML 2 | 202-2018- | M4 | PB + BM | 69% | 92% | CD45^{low} | Cureline |
| | 206- | | | | | CD34⁺ CD33⁻ | |
| | 31141/18 | | | | | | |
| AML 3 | 202-2018 | M2 | PB + BM | 60% | 99% | CD34⁺ CD33⁺ | Cureline |
| | 206- | | | | | | |
| | 2755/19 | | | | | | |
| AML 4 | 333-2019 | M2 | PB + BM | 49% | 99% | CD45^{med} | Cureline |
| | 206- | | | | | CD34⁺ CD33⁺ | |
| | 6258/19 | | | | | | |

Isolation of PBMC from Buffy Coats and Enrichment of Human NK Cells

PBMCs were isolated from buffy coats of healthy donors (German Red Cross, Mannheim, Germany) by density gradient centrifugation using SepMate-50 tubes. The buffy coat sample was diluted with a two-to-threefold volume of PBS (Invitrogen, cat.: 14190-169), layered on a cushion of Lymphoprep (Stem Cell Technologies, cat.: 07861) and centrifuged at 775×g for 20 min at ambient temperature with brake. PBMC located in the interface were collected and washed thrice with PBS. PBMC were maintained in RPMI 1640 medium supplemented with 10% h.i. FCS, 2 mM L-glutamine, 100 U/mL penicillin G sodium and 100 μg/mL streptomycin sulfate (referred to as complete RPMI medium, all components from Invitrogen) at 37° C. and 5% CO2 in a humidified atmosphere until use.

For the enrichment of NK cells PBMC were harvested from overnight cultures and used for one round of negative selection using the EasySep™ Human NK Cell Enrichment Kit (Stem Cell Technologies, cat.: 17055) for the immunomagnetic isolation of untouched human NK cells and the Big Easy EasySep™ Magnet (Stem Cell Technologies, cat.: 18001) according to the manufacturer's instructions. NK cells were resuspended in complete RPMI medium and immediately used.

### Thawing of Primary AML Patient's Material

Cryopreserved peripheral blood mononuclear cells (PBMC) and bone marrow mononuclear cells (BMMC) of AML patients were obtained from commercial biobanks (Cureline, USA; Tissue Solutions, UK) and thawed according to the manufacturer's instructions, briefly outlined as follows. Cureline: cells in cryovials were thawed for 1 to-2 minutes at 37° C., then swiftly transferred to pre-warmed (37° C.) complete RPMI medium, washed and immediately subjected to functional assays. Tissue Solutions: cells in cryovials were thawed for 1 to 2 minutes at 37° C., then swiftly transferred to chilled (4° C.) complete RPMI medium, washed and immediately subjected to functional assays. The number of viable cells was determined by trypan blue exclusion.

### Calcein-Release Cytotoxicity Assays

AML patient-derived PB (PBMC) or BM (BMMC), containing 49 to 86% of leukemic blasts (each 0.5×10⁵), were co-cultured with buffy coat-derived allogeneic NK cells (0.5×10⁵) at 1:1 cell ratio for 24 hours in the presence of titrated AFM28 (CD123×CD16A scFv-IgAb_268), control molecules or in the absence of antibody constructs in complete RPMI medium in 96-well microtiter plates. To support survival of leukemic blasts of the patient-derived AML samples, co-cultures were supplemented with 20 ng/mL GM-CSF (PeproTech, cat.: 300-03). In one experiments using the AML 1 sample, allogeneic NK cells were fluorescence-labelled with CMFDA prior to the assay to guide differentiation between tumor cells and NK cells. Afterwards, cell suspensions were subjected to extracellular staining of NK cell surface markers and markers to support determination of AML blasts within the AML patient-derived PBMC and BMMC, followed by Annexin V staining to distinguish live tumor cells from viable tumor cells from pre-apoptotic (Annexin V+ dead cell marker-) and dead cells (Annexin V+ dead cell marker+). The percentage of NK cell-dependent AFM28-mediated tumor cell depletion was assessed by flow cytometry and was compared to tumor cell depletion by NK cells in the absence of AFM28.

### Flow Cytometric Analysis

Extracellular staining of NK cell and tumor cell surface markers was performed with indicated fluorescence-labelled antibodies diluted in 50 μL FACS buffer for 30 minutes on ice in the dark in round-bottom 96-well microtiter plates. Afterwards, cells were washed once twice in FACS buffer followed by measurement on a CytoFlex S flow cytometer (Beckman Coulter) and analysis by CytExpert software (v2.4, Beckman Coulter). Leukemic blasts within AML PB and BM samples were identified using marker combinations of CD45 (Biolegend, cat.: 304048), CD33 (Biolegend, cat.: 366612), CD34 (Biolegend, cat.: 343534) as indicated in the table of primary AML samples above. To delineate the percentage of leukemic blasts positive for CD123 (BD Bioscience, cat.: 563599), the cut-off for CD123 negativity was inferred from the lymphocytic subpopulation (CD45^{high} CD33− CD34− SSC^{low} CD3+ (Biolegend, cat.: 300448) CD19+(Biolegend, cat.: 302242) cells) within AML PB and BM.

### Results

CD123 is overexpressed in many hematological malignancies and has been identified as one of the distinctive markers overexpressed on the surface of primary leukemic blasts and leukemic stem cells in AML patients, whereas in healthy tissue CD123 expression is rather restricted to, for instance, hematopoietic cell types such as basophils (Testa, 2019, Cancers, ncbi.nlm.nih.gov/pmc/articles/PMC6769702/). Here it was investigated whether AFM28 (CD123×CD16A scFv-IgAb_268) can induce NK cell-mediated depletion of primary leukemic blasts of peripheral blood and bone marrow matched from AML patients.

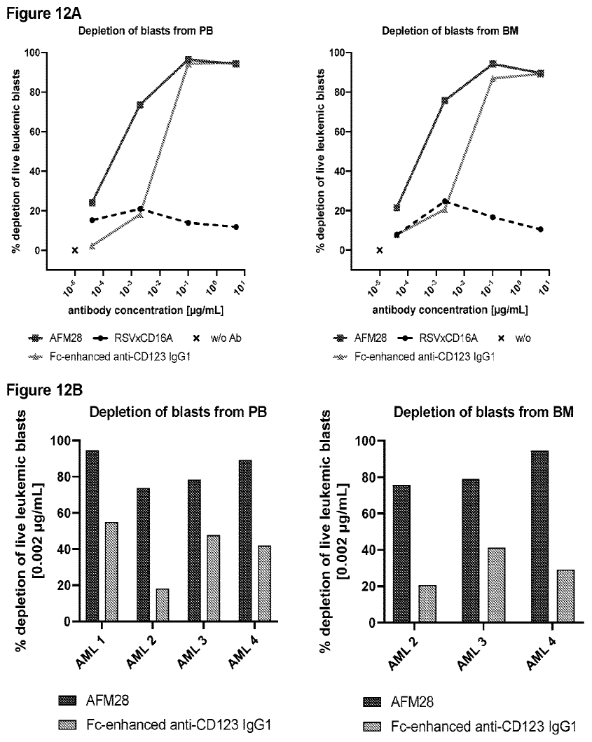

Buffy coat-derived NK cells were incubated with allogeneic AML patient-derived PBMC or BMMC in the presence of titrated increasing concentrations of AFM28 and control molecules in 24-hour flow cytometry-based tumor cell depletion assays. There was a marked dose-dependent increase in the depletion of primary leukemic blasts in the presence of AFM28 after co-culture with the allogeneic NK cells. Of note, the Fc-enhanced anti-CD123 IgG talacotuzumab (IgAb_338) showed a lower level of potency than AFM28, requiring higher concentrations to reach comparable anti-tumor activity against primary leukemic blasts (FIG. 12A-12B). In conclusion, AFM28 can induce the cytotoxic response of NK cells towards CD123-positive primary leukemic blasts from peripheral blood and bone marrow of AML patients.

### Example 10: scFv-IgAb_268 Depletion of CD123⁺ Primary AML Blasts and MDS Cells from Bone Marrow Samples, Sparing the CD34⁺/CD123⁻ Compartment

### Co-Cultures and Flow Cytometric Analysis

Freshly thawed bone marrow sample cells (5×10⁴) were co-cultured with buffy coat-derived allogeneic NK cells (5×10⁴) at 1:1 cell ratio for 24 h in triplicates in the presence of titrated antibody, starting at a concentration of 1000 pM followed by five dilutions (500 pM, 100 pM, 50 pM, 10 pM, 5 pM), in complete RPMI medium in 96-well microtiter plates. Afterwards, depletion of CD123⁺ target cells from the CD45⁺ mononuclear cell fraction was assessed after extracellular staining with fluorescently-conjugated mouse anti-human antibodies, diluted in 50 μL FACS buffer, by flow cytometry. Absolute cell numbers (viable, gated on CD45) are indicated in figure.

### Results

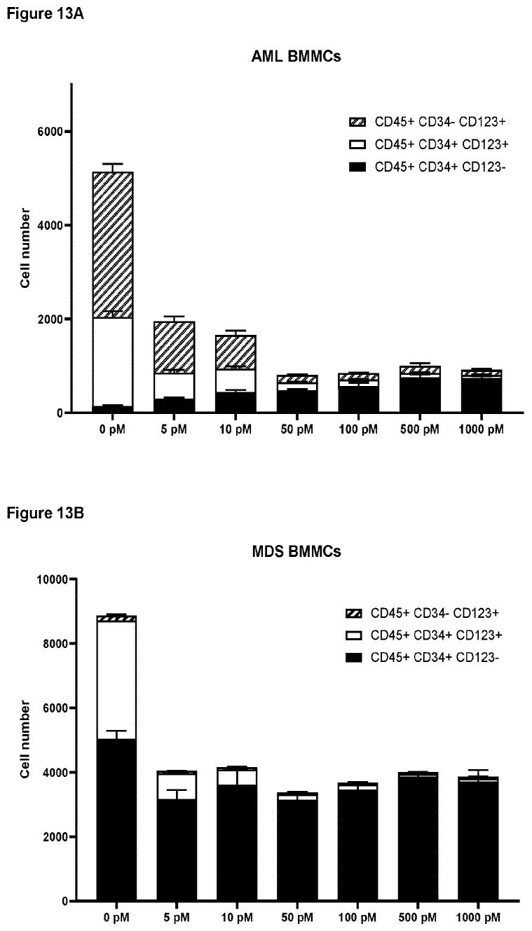

The CD123×CD16A scFv-IgAb_268 construct specifically induced the depletion of CD123+ BMMCs (FIG. 13A-13B). Of note, in the complex sample no lysis of CD123neg bystander cells, including normal hematopoetic stem cells (CD34⁺/CD123^{neg} HSC), was observed. NK cells alone did not induce bone marrow cell lysis in the absence of antibody.

The high specificity and affinity of binding to effector cells via CD16A and target cells via CD123 fosters efficacious depletion of tumor cells and potentially target-positive immunosuppressive cells in the tumor microenvironment (e.g. CD34^{neg}/CD33⁺/CD123⁺ BM-MDSC) and as a consequence restricts the unspecific lysis of other bystander cells and normal hematopoetic stem cells (CD34⁺/CD123^{neg} HSC) (FIG. 13A-13B).

Example 11: A Pre-Clinical Toxicology Model in Cynomolgus Monkey Suggested that scFv-IgAb_268 was Well Tolerated and Pharmacologically Active, as Demonstrated by Depletion of Peripheral Blood Basophils

### Methods

Ten naïve Cynomolgus monkeys of Mauritian origin were dosed weekly (q7d×28d) by a two-hour infusion for 4 weeks including a 2-week recovery phase. Animals were allocated to 4 groups summarized in Table 13.

TABLE 13

| | | Dose | Volume | | Necropsy | |
|---|---|---|---|---|---|---|
| | | level | of | Animals | after | |
| Group | Group | (mg/kg/ | infusion | per | 4 | 6 |
| number | description | day) | (mL/kg) | group | Weeks | Weeks |
| 1 | Vehicle | 0 | 10 | 1M + | 1M + | |
| | | | | 1F | 1F | |
| 2 | Low | 4 | 10 | 1M + | 1M + | |
| | | | | 1F | 1F | |
| 3 | Intermediate | 20 | 10 | 1M + | 1M + | |
| | | | | 1F | 1F | |
| 4 | High | 100 | 10 | 2M + | 1M + | 1M + |
| | | | | 2F | 1F | 1F |

Assessment of toxicity was based on clinical observations, body weights, body temperature, clinical and anatomic pathology. As additional endpoints, the determination of serum cytokine levels of IL-2, IL-6, IL-8, TNF-α, GM-CSF and INF-γ and a flow cytometric assessment of the lymphocyte subsets were included (CD45, CD3, CD4, CD8, CD20, CD16, CD159a). Furthermore, the quantification of the basophils and plasmacytoid dendritic cells (pDCs) in the peripheral blood was integrated as a pharmacodynamic endpoint (Busfield et al., 2014). Blood was collected for toxicokinetic evaluation of scFv-IgAb_268, and anti-drug antibodies were determined using electrochemiluminescence immuno-assays based on the MSD® platform. Full necropsies were performed on all animals, organ weights were determined followed by macroscopic and microscopic examinations for all tissues.

### Results

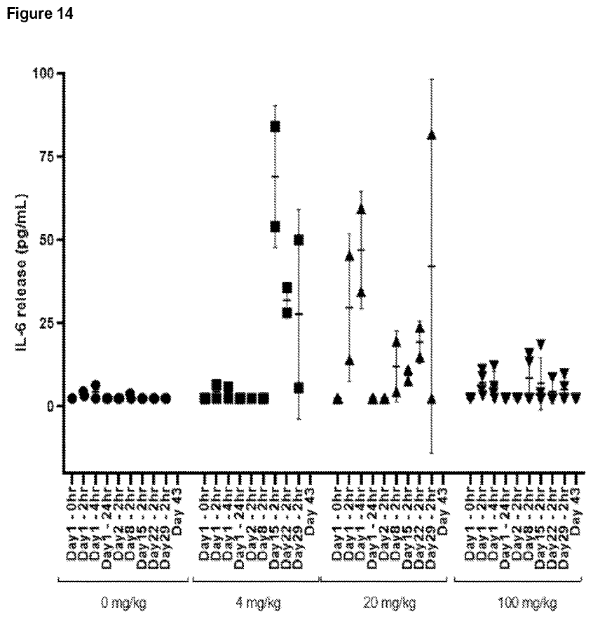

In this intravenous repeat dose range finder study, scFv-IgAb_268 did not induce systemic or local toxicity. All animals were clinically well and no effect on body weights, body temperature, or clinical pathology up to the maximum tested dose level of 100 mg/kg was observed. Findings of note were a transient non-dose dependent elevation of IL-6 levels 2-4 hours after commencement of infusion. IL-6 levels returned to normal after 24 hours (FIG. 14). scFv-IgAb_268 had no effect on IL-2, IL-8, IFN-γ, GM-CSF and TNF-α levels at any dose. Furthermore, at 100 mg/kg, scFv-IgAb_268 caused a transient reduction in absolute NK cell counts (CD3−CD20−CD159+ positive) after the first dose and a reduction in neutrophil counts on Days 22 and 29. Four out of eight scFv-IgAb_268 treated animals revealed marginal or marked spleen enlargement. The test item induced increased hematopoietic cellularity (slight or marked) in the sternal and femoral bone marrow in two female animals at 20 or 100 mg/kg as well as increased extramedullary hematopoiesis in the spleen.

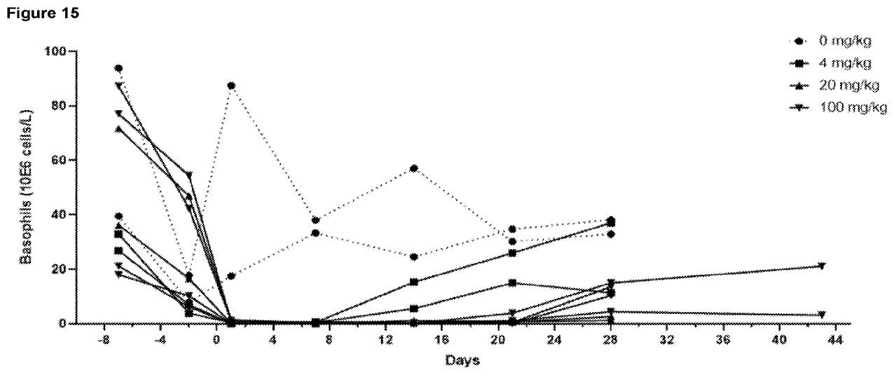

Depletion in absolute basophils and pDCs counts (CD123+) was observed in peripheral blood at all dose-levels 24 hours after the first administration demonstrating the expected pharmacodynamic effect of scFv-IgAb_268 (FIG. 15).

All animals treated with scFv-IgAb_268 were systemically exposed. TK parameters were determined after the first dose and serum t_{1/2} ranged from 27 to 78 hours. Half-lives are likely to be underestimated since β-elimination phase was not fully reached before the end of the dosing interval. As determined by area under the curve (AUC) more than dose proportional PK was observed as expected for an IgG like molecule. Five out of eight treated animals were tested positive for ADA with 4/5 revealing an effect on exposure.

### Example 12: Binding of FOLR1×CD16A Constructs to Cell Lines Expressing Human CD16A and Cynomolgus CD16

### Methods

TABLE 14
Antibody constructs

| | Target | Target | Effector | Effector |
|---|---|---|---|---|
| Construct | specificity | domain | specificity | domain |
| scFv-IgAb_381 | FOLR1 | FOLR1_1 | CD16A | CD16a4 |
| scFv-IgAb_387 | FOLR1 | FOLR1_1 | CD16A | CD16a3 |
| scFv-IgAb_162 | FOLR1 | FOLR1_1 | RSV | NIST RM8671 |

### Flp-In CHO Host Cell Culture

Flp-In CHO cells (Life Technologies, R75807), a derivative of CHO-K1 Chinese Hamster ovary cells, were adapted to growth in suspension in HyClone CDM4CHO medium supplemented (Cytiva, cat. SH30557.02) with L-Glutamine (Invitrogen, cat. 25030-024), HT Supplement (Thermo Fisher Scientific, cat. 41065012), Penicillin/Streptomycin (Invitrogen, cat. 1540-122) and 100 μg/mL Zeocin (Thermo Fisher Scientific, cat. R250-01). Single cell derived clonal lines were obtained by limiting dilution cloning in a medium mixture of standard culture medium with Ham's F-12 supplemented (Thermo Fisher Scientific, cat. 11500586) with InstiGRO CHO supplement (Solentim, cat. RS-1105), expanded, and cryopreserved in medium with 10% DMSO (Sigma, cat. D2650). Cultures were routinely subcultured after 2 or 3 days and diluted in fresh medium to 3E+5 viable cells/mL for a subsequent 2-day passage or 2E+5 viable cells/mL for a 3-day passage, cultured in shake flasks or tubes at 37° C., 5% CO2 and 120-200 rpm depending on the vessel type.

Generation of Stably Transfected Antigen Expressing Cells (cAg)

Suspension-adapted Flp-In CHO host cells were subcultured in standard medium without Zeocin one day prior to transfection. Recombinant CHO cells were generated by transfection of 2E+6 cells in 2 mL of CHO-S-SFMII medium (Thermo Fisher Scientific, cat. 12052-114), with expression plasmids encoding recombinant cell-anchored antigen sequences (cAgs) in a modified, version of pcDNA5/FRT vector, mediating Puromycin resistance or Hygromycin resistance and the Flp recombinase (pOG44, Thermo Fisher, V600520) using a total of 2.5 μg of DNA and Transporter 5 transfection reagent at a DNA:PEI ratio of 1:2.5 (μg/pg). DNA and transfection reagent were mixed in 100 μL NaCl solution (Sigma, cat. S8776), 0.9% and incubated for 20 minutes before addition to the cells. As a negative control (mock), cells were transfected with a control plasmid not mediating resistance. After 4 hours, transfected cells were diluted with 8 mL of a 1:1 medium mixture of standard culture medium with Ham's F-12. Selection of stably transfected cells was started on the following day by addition of 3.2 μg/mL of Puromycin Dihydrochloride (Thermo Fisher Scientific, cat. A1113803) as selection antibiotic and an increase to 6.3 μg/mL on day 2 or of 500 μg/ml of Hygromycin B (Thermo Fisher Scientific, cat. 10687010). Viable cell densities were measured twice per week, and cells were centrifuged and resuspended in fresh selection medium containing selection antibiotic at a maximal density of 2-4E+5 viable cells/mL. Concentration of Puromycin Dihydrochloride was increased to 7.0 μg/mL on day 10 after transfection. Stably transfected cell pools recovered in growth and viability after approximately 2-3 weeks, were expanded in standard culture medium and cryopreserved in freezing medium containing 7.5% DMSO. For analysis of antigen expression, cultures were propagated in shake flasks or tubes and subcultured after 2 or 3 days and diluted in fresh medium to 6E+5 viable cells/mL for a subsequent 2-day passage or 3E+5 viable cells/mL for a 3-day passage, cultured at 37° C., 5% CO2 and 120-200 rpm depending on the vessel type.

### Flow Cytometric Analysis

To analyze binding of different antibody constructs to CHO cells transfected with human CD16A (158F) (cAg_34), human CD16A (158V) (cAg_35) and cynomolgus CD16 (cAg_36), relative to CD16 expression by flow cytometry, 1-5×10⁵ were resuspended in 100 μL FACS buffer (PBS (BioWest, cat.: L0615-500) containing 2% heat-inactivated FCS (Invitrogen, cat.: 10500-064), and 0.1% sodium azide (Sigma, cat.: S8032_100G)) in round-bottom 96-well microtiter plates. After washing in FACS buffer, cells were incubated in 50 μL FACS buffer without antibodies or with titrated antibodies starting at a concentration of 1000 nM followed by elven 3-fold serial dilutions for 40-50 min on ice in the dark. After washing twice, cells were incubated with FITC-conjugated goat anti-human IgG (H+L) (Jackson Immunologies, cat.: 109-096-088) for 40-50 min on ice in the dark. As controls, cells were only incubated with anti-human CD16-FITC (clone 3G8, Biolegend, cat. 302006). After washing, binding was measured by flow cytometry and mean fluorescence intensities (MFI) of cell samples were calculated and corrected for background staining using control cells stained with secondary antibodies only.

### Statistical Analysis

Equilibrium dissociation constants (K_{D}) of antibody binding, mean and standard deviation (SD) were calculated by plotting MFI values and fitting a non-linear regression model for one-site binding to hyperbolic dose-response curves using GraphPad Prism for Windows (v9; GraphPad Software; La Jolla California USA).

### Results

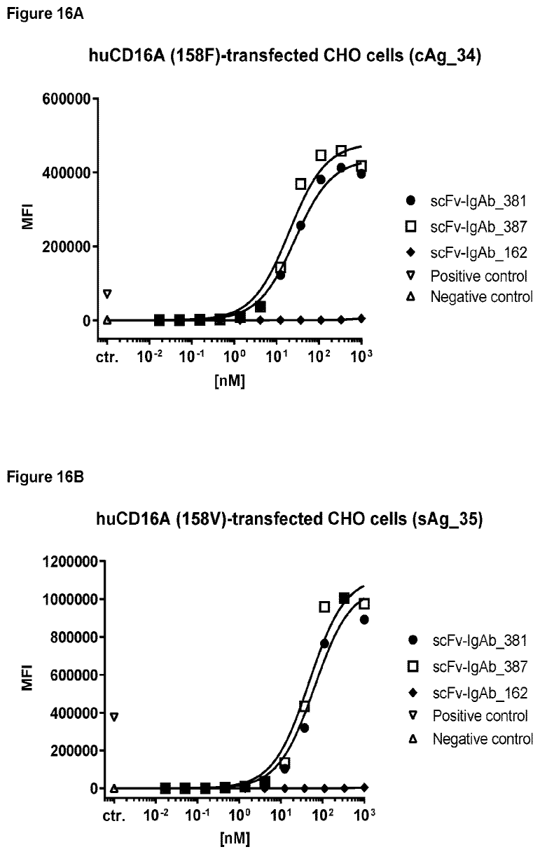

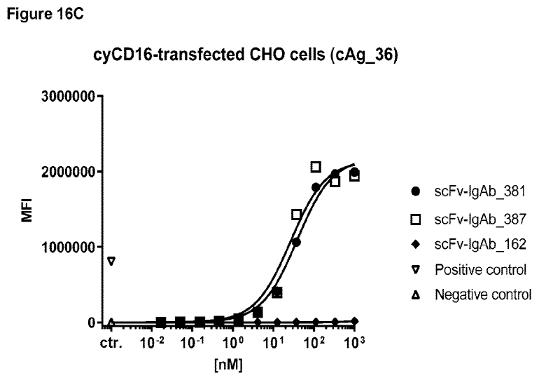

The apparent avidity of scFv-IgAb_381 and scFv-IgAb_387 to human (hu)CD16A-transfected CHO cells (both 158F and 158V allotypes) as well as cynomolgus (cy)CD16-transfected CHO cells was determined. CHO cells expressing recombinant huCD16A (158F) (cAg_34), huCD16A (158V) (cAg_35) and cyCD16 (cAg_36) were incubated with increasing concentrations of scFv-IgAb_381 and scFv-IgAb_387 and binding was assessed relative to control molecules (scFv-IgAb_162) by flow cytometry. Human CD16A and cynomolgus CD16 expression on transfected CHO cells was confirmed using anti-human CD16A-FITC antibody clone 302006 (Biolegend) (FIGS. 16A, 16B and 16C). The antibody construct scFv-IgAb_387 exhibited higher concentration-dependent binding to huCD16A(158F), huCD16A(158V) and cyCD16 than scFv-IgAb_381 (FIG. 16A, 16B, 16C and Table 15). No binding was detected by a negative control molecule (FOLR1×RSV, scFv-IgAb_162) comprising the same antibody scaffold and FOLR1-targeting domain as scFv-IgAb_381 and scFv-IgAb_387 but an irrelevant anti-RSV domain replacing CD16A. Hence these results corroborate higher binding specificity to human CD16A and cynomolgus CD16 of scFv-IgAb_387 containing CD16a3 anti-CD16A effector domain compared to scFv-IgAb_381 containing CD16a4 anti-CD16A effector domain.

TABLE 15
Mean apparent avidity (K_{D}) of scFv-IgAb_381, scFv-IgAb_387 and control antibody to human
CD16A (both 158F and 158V allotypes) and cynomolgus CD16 expressed on CHO cells.

| | | K_{D} [nM] | | | | |
|---|---|---|---|---|---|---|
| | ID | Exp. 1 | Exp. 2 | Exp. 3 | Mean | SD |
| cAg_34, | scFv-IgAb_381 | 22.06 | 29.34 | 27.15 | 26.2 | 3.05 |
| huCD16A | scFv-IgAb_387 | 16.02 | 28.48 | 19.95 | 21.5 | 5.20 |
| (158F) | scFv-IgAb_162 | n.a. | n.a. | n.a. | n.a. | n.a. |
| cAg_35, | scFv-IgAb_381 | 22.02 | 43.3 | 63.72 | 43.0 | 17.03 |
| huCD16A | scFv-IgAb_387 | 15.83 | 29.09 | 47.5 | 30.8 | 12.99 |
| (158V) | scFv-IgAb_162 | n.a. | n.a. | n.a. | n.a. | n.a. |
| cAg_36, | scFv-IgAb_381 | 38.56 | 31.7 | 28.22 | 32.8 | 4.30 |
| cyCD16 | scFv-IgAb_387 | 26.59 | 26.25 | 22.77 | 25.2 | 1.73 |
| | scFv-IgAb_162 | n.a | n.a | n.a | n.a. | n.a. |

Binding of antibody constructs to huCD16A (158F and 158V)-as well as cyCD16-transfected CHO cells was measured by flow cytometry. Equilibrium dissociation constants (KD) of antibody binding were calculated by plotting MFI values and fitting a non-linear regression model for one-site binding to hyperbolic dose-response curves using GraphPad Prism.
SD, standard deviation;
n.a., not applicable.

### Example 13: ADCC Against FOLR1+A2780 Cells by Anti-FOLR1 Antibodies

### Methods

TABLE 16
Antibody constructs

| | Target | Target | Effector | Effector |
|---|---|---|---|---|
| Construct | specificity | domain | specificity | domain |
| scFv-IgAb_273 | FOLR1 | FOLR1_1 | CD16A | CD16a4 |
| scFv-IgAb_274 | FOLR1 | FOLR1_1 | CD16A | CD16a4 |
| scFv-IgAb_275 | FOLR1 | FOLR1_1 | CD16A | CD16a3 |

Isolation of PBMC from buffy coats and enrichment of human NK cells PBMCs were isolated from buffy coats (German Red Cross, Mannheim, Germany) by density gradient centrifugation. The buffy coat samples were diluted with a two-to-threefold volume of PBS (Invitrogen, cat.: 14190-169), layered on a cushion of Lymphoprep (Stem Cell Technologies, cat.: 07861) and centrifuged at 800×g for 25 min at room temperature w/o brake. PBMC located in the interface were collected and washed 3 times with PBS before they were cultured in complete RPMI 1640 medium (RPMI 1640 medium supplemented 10% heat-inactivated FCS, 2 mM L-glutamine and 100 IU/mL penicillin G sodium and 100 μg/mL streptomycin sulfate (all components from Invitrogen)) overnight without stimulation. For the enrichment of NIK cells PBMC were harvested from overnight cultures and used for one round of negative selection using the EasySep™ Human NK Cell Enrichment Kit (Stem Cell Technologies, cat.: 17055) for the immunomagnetic isolation of untouched human NK cells and the Big Easy EasySep™ Magnet (Stem Cell Technologies, cat.: 18001) according to the manufacturer's instructions.

### Culture of A2780 Tumor Cell Line

The A2780 cell line was cultured under standard conditions as recommended by the supplier at 37° C. and 5% CO2 in a humidified atmosphere in complete RPMI medium (RPMI 1640 medium supplemented with 10% h.i. FCS, 2 mM L glutamine, 100 U/mL penicillin G sodium, 100 μg/mL streptomycin sulfate).

### Calcein-Release Cytotoxicity Assays

Antibody-mediated target cell lysis by NK cells in vitro was assessed by quantifying the release of calcein into cell culture supernatants from calcein-labeled target cells. For this, target cells were labeled with 10 μM calcein AM for 30 min in RPMI 1640 medium without FCS at 37° C. After gentle washing, calcein-labeled cells were resuspended in complete RPMI medium at a density of 1×10⁵/mL. 1×10⁴ target cells were then seeded in individual wells of a round-bottom 96-well microtiter plate and, if not mentioned otherwise, mixed with enriched human NK cells at an effector-to-target cell (E:T) ratio of 1.25:1. The culture of NK cells with target cells was conducted in duplicate without antibody addition or in the presence of increasing concentration of antibodies. After centrifugation for 2 min at 200×g, microtiter plates were incubated for 4 h at 37° C. in a humidified atmosphere with 5% CO2. Spontaneous calcein-release, maximal release and killing of targets by effectors in the absence of antibodies were determined in quadruplicate on each plate. Spontaneous release was determined by incubation of target cells in the absence of effector cells and in the absence of antibodies. Maximal release was achieved by adding Triton X-100 to a final concentration of 1% in the absence of effector cells and in the absence of antibodies. Following incubation, 100 μL cell-free cell culture supernatant was harvested from each well after centrifugation for 5 min at 500×g and transferred to black flat-bottom 96-well microtiter plates. Fluorescence counts of released calcein were measured at 520 nm using a multimode plate reader. Specific cell lysis was calculated according to the following formula: [fluorescence (sample)−fluorescence (spontaneous)]/[fluorescence (maximum)−fluorescence (spontaneous)]×100% wherein “Fluorescence (spontaneous)” and “Fluorescence (maximum)” are defined as fluorescence in absence of effector cells and antibodies and fluorescence induced by the addition of Triton X-100, respectively.

### Results

All three FOLR1×CD16A scFv-IgAb constructs induced NK cell-dependent lysis against A2780 cells at similar maximal efficacy (FIG. 17).

### Example 14: Target Cell-Independent Activation of NK Cells by Anti-FOLR1 Antibodies

### Methods

TABLE 17
Antibody constructs

| | Target | Target | Effector | Effector |
|---|---|---|---|---|
| Construct | specificity | domain | specificity | domain |
| scFv-IgAb_273 | FOLR1 | Farletuzumab | CD16A | CD16a4 |
| scFv-IgAb_274 | FOLR1 | Farletuzumab | CD16A | CD16a4 |
| scFv-IgAb_275 | FOLR1 | Farletuzumab | CD16A | CD16a3 |

Isolation of PBMC from Buffy Coats and Enrichment of Human NK Cells

PBMCs were isolated from buffy coats (German Red Cross, Mannheim, Germany) by density gradient centrifugation. The buffy coat samples were diluted with a two-to-threefold volume of PBS (Invitrogen, cat.: 14190-169), layered on a cushion of Lymphoprep (Stem Cell Technologies, cat.: 07861) and centrifuged at 800×g for 25 min at room temperature w/o brake. PBMC located in the interface were collected and washed 3 times with PBS before they were cultured in complete RPMI 1640 medium (RPMI 1640 medium supplemented 10% heat-inactivated FCS, 2 mM L-glutamine and 100 IU/mL penicillin G sodium and 100 μg/mL streptomycin sulfate (all components from Invitrogen)) overnight without stimulation. For the enrichment of NK cells PBMC were harvested from overnight cultures and used for one round of negative selection using the EasySep™ Human NK Cell Enrichment Kit (Stem Cell Technologies, cat.: 17055) for the immunomagnetic isolation of untouched human NK cells and the Big Easy EasySep™ Magnet (Stem Cell Technologies, cat.: 18001) according to the manufacturer's instructions.

### Cultures and Flow Cytometric Analysis

Buffy coat-derived NK cells (5×10⁴) were cultured for 24 h in the presence titrated antibodies, starting at a concentration of 660 nM followed by seven 10-fold serial dilutions, or without antibodies in complete RPMI 1640 medium in 96-well round-bottom microtiter plates. Afterwards, up-regulation of the NK cell activation marker CD137 and CD69 was assessed by extracellular staining with anti-CD16-FITC (Biolegend, cat: 302006), anti-CD69 PE (Biolegend, cat: 310906), anti-CD45 PerCP-Cy5.5 (Biolegend, cat: 304028), anti-CD56 PE-Cy7 (Biolegend, cat: 318318), anti-CD137 APC (Biolegend, cat: 309810) and viability dye (Thermo Fisher, cat:65-0865-18) diluted in 50 μL FACS buffer (PBS (Invitrogen, cat.: 14190-169) containing 2% heat-inactivated FCS (Invitrogen, cat.: 10270-106), and 0.1% sodium azide (Roth, Karlsruhe, Germany, cat.: A1430.0100)), followed by flow cytometric analysis. NK cells were gated as live, CD56+, and CD45+. The mean fluorescence intensity (MFI) of CD137 − and CD69 on NK cells is indicated. MFI values were analysed by non-linear regression using GraphPad Prism for Windows (v9; GraphPad Software; La Jolla California USA).

### Results

Of the three anti-FOLR1 antibodies, FOLR1/CD16A scFv-IgAb constructs constituting the anti-CD16A CD16a3 domain showed the lowest propensity to upregulate the activation markers CD69 and CD137 on NK cells in the absence of FOLR1⁺ target cells. At the highest tested concentration of 660 nM, scFv-IgAb_275 appeared to induce the lowest target-independent NK cell activation, followed in sequence by scFv-IgAb_274 and scFv-IgAb_273 (FIGS. 18A-18B).

### Example 15: Binding of FOLR1×CD16A Constructs to Cell Lines Expressing Human FOLR1 and Cynomolgus FOLR1

### Methods

TABLE 18
Antibody constructs

| | Target | Target | Effector | Effector |
|---|---|---|---|---|
| Construct | specificity | domain | specificity | domain |
| scFv-IgAb_381 | FOLR1 | FOLR1_1 | CD16A | CD16a4 |
| scFv-IgAb_387 | FOLR1 | FOLR1_1 | CD16A | CD16a3 |

### Flp-In CHO Host Cell Culture

Flp-In CHO cells (Life Technologies, R75807), a derivative of CHO-K1 Chinese Hamster ovary cells, were adapted to growth in suspension in HyClone CDM4CHO medium supplemented (Cytiva, cat. SH30557.02) with L-Glutamine (Invitrogen, cat. 25030-024), HT Supplement (Thermo Fisher Scientific, cat. 41065012), Penicillin/Streptomycin (Invitrogen, cat. 1540-122) and 100 μg/mL Zeocin (Thermo Fisher Scientific, cat. R250-01). Single cell derived clonal lines were obtained by limiting dilution cloning in a medium mixture of standard culture medium with Ham's F-12 supplemented (Thermo Fisher Scientific, cat. 11500586) with InstiGRO CHO supplement (Solentim, cat. RS-1105), expanded, and cryopreserved in medium with 10% DMSO (Sigma, cat. D2650). Cultures were routinely subcultured after 2 or 3 days and diluted in fresh medium to 3E+5 viable cells/mL for a subsequent 2-day passage or 2E+5 viable cells/mL for a 3-day passage, cultured in shake flasks or tubes at 37° C., 5% CO2 and 120-200 rpm depending on the vessel type.

Generation of Stably Transfected Antigen Expressing Cells (cAg)

Suspension-adapted Flp-In CHO host cells were subcultured in standard medium without Zeocin one day prior to transfection. Recombinant CHO cells were generated by transfection of 2E+6 cells in 2 mL of CHO-S-SFMII medium (Thermo Fisher Scientific, cat. 12052-114), with expression plasmids encoding recombinant cell-anchored antigen sequences (cAgs) in a modified, version of pcDNA5/FRT vector, mediating Puromycin resistance or Hygromycin resistance and the Flp recombinase (pOG44, Thermo Fisher, V600520) using a total of 2.5 μg of DNA and Transporter 5 transfection reagent at a DNA:PEI ratio of 1:2.5 (μg/μg). DNA and transfection reagent were mixed in 100 μL NaCl solution (Sigma, cat. S8776), 0.9% and incubated for 20 minutes before addition to the cells. As a negative control (mock), cells were transfected with a control plasmid not mediating resistance. After 4 hours, transfected cells were diluted with 8 mL of a 1:1 medium mixture of standard culture medium with Ham's F-12. Selection of stably transfected cells was started on the following day by addition of 3.2 μg/mL of Puromycin Dihydrochloride (Thermo Fisher Scientific, cat. A1113803) as selection antibiotic and an increase to 6.3 μg/mL on day 2 or of 500 μg/ml of Hygromycin B (Thermo Fisher Scientific, cat. 10687010). Viable cell densities were measured twice per week, and cells were centrifuged and resuspended in fresh selection medium containing selection antibiotic at a maximal density of 2-4E+5 viable cells/mL. Concentration of Puromycin Dihydrochloride was increased to 7.0 μg/mL on day 10 after transfection. Stably transfected cell pools recovered in growth and viability after approximately 2-3 weeks, were expanded in standard culture medium and cryopreserved in freezing medium containing 7.5% DMSO. For analysis of antigen expression, cultures were propagated in shake flasks or tubes and subcultured after 2 or 3 days and diluted in fresh medium to 6E+5 viable cells/mL for a subsequent 2-day passage or 3E+5 viable cells/mL for a 3-day passage, cultured at 37° C., 5% CO2 and 120-200 rpm depending on the vessel type.

### Flow Cytometric Analysis

To analyze binding of different antibody constructs to CHO cells transfected with human FOLR1 (cAg_125) and cynomolgus FOLR1 (cAg_126), 1-5×10⁵ were resuspended in 100 μL FACS buffer (PBS (BioWest, cat.: L0615-500) containing 2% heat-inactivated FCS (Invitrogen, cat.: 10500-064), and 0.1% sodium azide (Sigma, cat.: S8032_100G)) in round-bottom 96-well microtiter plates. After washing in FACS buffer, cells were incubated in 50 μL FACS buffer without antibodies or with titrated antibodies starting at a concentration of 1000 nM followed by eleven 3-fold serial dilutions for 40-50 min on ice in the dark. After washing twice, cells were incubated with FITC-conjugated goat anti-human IgG (H+L) (Jackson Immunologies, cat.: 109-096-088) for 40-50 min on ice in the dark. After washing, binding was measured by flow cytometry and mean fluorescence intensities (MFI) of cell samples were calculated and corrected for background staining using control cells stained with secondary antibodies only.

### Statistical Analysis

Equilibrium dissociation constants (K_{D}) of antibody binding, mean and standard deviation (SD) were calculated by plotting MFI values and fitting a non-linear regression model for one-site binding to hyperbolic dose-response curves using GraphPad Prism for Windows (v9; GraphPad Software; La Jolla California USA).

### Results

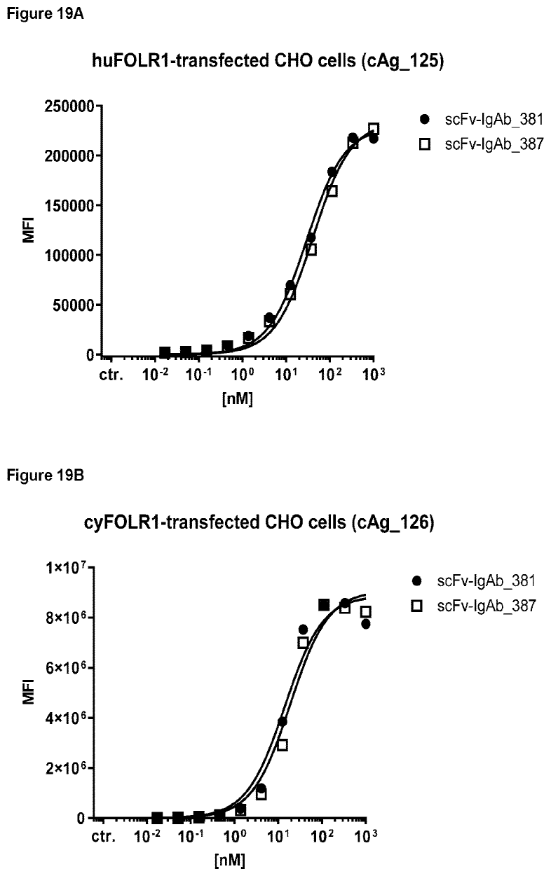

The apparent avidity of scFv-IgAb_381 and scFv-IgAb_387 to human (hu)FOLR1-transfected CHO cells as well as cynomolgus (cy)FOLR1-transfected CHO cells was determined. CHO cells expressing recombinant huFOLR1 (cAg_125) and cyFOLR1 (cAg_126) were incubated with increasing concentrations of scFv-IgAb_381 and scFv-IgAb_387 and binding was assessed by flow cytometry. Both scFv-IgAb_381 and scFv-IgAb_387 antibody constructs exhibited similar concentration-dependent binding to huFOLR1 and cyFOLR1 which can be attributed to the same anti-FOLR1 binding domain present in both antibody constructs (FIG. 19A, 19B and Table 19).

TABLE 19
Mean apparent avidity (K_{D}) of
scFv-IgAb_381 and scFv-IgAb_387 to human
FOLR1 and cynomolgus FOLR1 expressed on CHO cells.

| | | K_{D} [nM] | | | | |
|---|---|---|---|---|---|---|
| | ID | Exp. 1 | Exp. 2 | Exp. 3 | Mean | SD |
| cAg_125, | scFv-IgAb_381 | 20.32 | 24.37 | 29.27 | 24.7 | 3.66 |
| huFOLR1 | scFv-IgAb_387 | 35.06 | 31.61 | 40.65 | 35.8 | 3.72 |
| Ag_126, | scFv-IgAb_381 | 11.51 | 11.8 | 13.99 | 12.4 | 1.11 |
| cyFOLR1 | scFv-IgAb_387 | 14.93 | 15.31 | 18.79 | 16.3 | 1.74 |

Binding of antibody constructs to huFOLR1-as well as cyFOLR1-transfected CHO cells was measured by flow cytometry. Equilibrium dissociation constants (KD) of antibody binding were calculated by plotting MFI values and fitting a non-linear regression model for one-site binding to hyperbolic dose-response curves using GraphPad Prism.
SD, standard deviation;
n.a., not applicable.

### Example 16: FOLR1×CD16A Innate Immune-Cell Engager that Selectively Targets FOLR1 Expressing Tumor Cells to Safely Harness Potent Anti-Cancer Responses

### Background

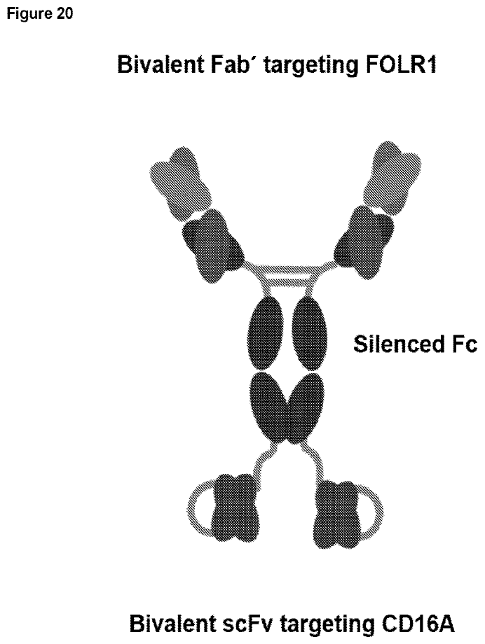

Innate Cell Engager (ICE®) molecules are designed to bivalently bind CD16A+ natural killer (NK) cells and macrophages and a tumor cell-surface antigen, inducing potent, tumor-directed cytotoxicity via antibody-dependent cellular cytotoxicity (ADCC) and antibody-dependent cellular phagocytosis (ADCP). scFv-IgAb_387 (FIG. 20) is a tetravalent, bispecific ICE® that bridges folate receptor alpha (FOLR1) on tumor cells with innate immune cells, to induce potent and selective targeted tumor cell killing. Herein we describe the structure, mechanism of action and preliminary safety data of scFv-IgAb_387.

TABLE 20
Antibody construct

| | Target | Target | Effector | Effector |
|---|---|---|---|---|
| Construct | specificity | domain | specificity | domain |
| scFv-IgAb_387 | FOLR1 | FOLR1_1 | CD16A | CD16a3 |

### Methods

Binding of scFv-IgAb_387 to CD16A and human FOLR1 was assessed by ELISA. Binding of scFv-IgAb_387 to NK cells was assessed by flow cytometry in the presence of physiological levels of IgG (10 mg/mL). ADCC was assessed using a 4h-calcein release assay with purified NK cells from healthy donors against a panel of tumor cell lines with different expression levels of FOLR1. ADCP was assessed using a flow cytometry-based method with macrophages derived from healthy donor monocytes and target cell lines with different levels of FORL1 expression. Quantification of secreted cytokines by healthy donor PBMC cultures in the presence of scFv-IgAb_387 and target cells was assessed after a 24 h incubation using multiplex cytokine quantification.

### Results

scFv-IgAb_387 binds to CD16A (both 158V and 158F variants) with an apparent avidity of 0.1 nM and to hFOLR1 with an apparent avidity of 0.05 nM. Physiological levels of competing IgG do not alter binding efficacy. scFv-IgAb 387 induces potent and selective ADCC, even on FOLR1 low cells. scFv-IgAb_387 is also shown to induce efficient ADCP in vitro. Further, scFv-IgAb_387 is demonstrated to be more efficacious and potent in both ADCC and ADCP assays than farletuzumab, an Fc competent monoclonal antibody targeting FOLR1 which shares the same VH/VL sequence as scFv-IgAb_387. Co-culture of PBMCs, tumor cells and scFv-IgAb_387 showed concentration-dependent release of pro-inflammatory cytokines (IFNγ, IP-10, TNFα) and minimal off-target cytokine release.

### CONCLUSIONS

We demonstrate that scFv-IgAb_387 selectively and potently kills tumor cells with a range of expression levels of FOLR1 by two complimentary mechanisms: ADCC and ADCP. The high avidity for CD16A imparts increased potency and efficacy compared to an Fc competent, FOLR1-targeting antibody with the same VH/VL sequence. As scFv-IgAb_387 binds selectively to CD16A outside the IgG binding epitope, physiological levels of IgG do not compete for binding. We show that scFv-IgAb_387 induces moderate concentration-dependent pro-inflammatory cytokine release in a target-restricted manner, confirming a potent but safe in vitro profile of scFv-IgAb_387.

### Example 17: Assessment of FOLR1×CD16A scFv-IgAb-Induced ADCC in the Presence of Soluble FOLR1

To assess the impact of soluble FOLR1 (sFOLR1) on FOLR1×CD16A scFv-IgAb_387-induced lysis of target cells by NK cells, 4 h calcein-release cytotoxicity assays were be performed on A2780 and A-549 target cells with enriched primary human NK cells as effector cells at an E:T ratio of 2.5:1 and a fixed, non-saturating concentration of scFv-IgAb_387 in the presence of a 2—, 25-, and 50-fold molar excess of sFOLR1 and soluble mesothelin (sMSLN) as a negative control.

### Methods

Isolation of PBMC from buffy coats and enrichment of NK cells PBMCs were isolated from buffy coats (German Red Cross, Mannheim, Germany) by density gradient centrifugation. The buffy coat samples were diluted with a two-to-threefold volume of PBS (Invitrogen, cat.: 14190-169), layered on a cushion of Lymphoprep (Stem Cell Technologies, cat.: 07861) and centrifuged at 800×g for 25 min at room temperature w/o brake. PBMC located in the interface were collected and washed 3 times with PBS before they were cultured in RPMI 1640 medium (Invitrogen, cat.: 21875-091) supplemented with 10% heat-inactivated (h.i.) FCS (Invitrogen, cat.: 10270-106), 2 mM L-glutamine (Invitrogen, cat.: 25030-024), and 100 U/mL penicillin G sodium/100 μg/mL streptomycin sulfate (Invitrogen, cat.: 15140-122) (herein referred to as complete RPMI 1640 medium) overnight without stimulation. For the enrichment of NK cells, PBMC were harvested from overnight cultures and used for one round of negative selection using the EasySep™ Human NK Cell Enrichment Kit (Stem Cell Technologies, cat.: 17055) for the immunomagnetic isolation of untouched human NK cells and the Big Easy EasySep™ Magnet (Stem Cell Technologies, cat.: 18001) according to the manufacturer's instructions.

Culture of Tumor Cell Lines A-549 cells (CLS, cat.: 30014) were cultured in advanced DMEM/F12 (Invitrogen, cat.: 12634010) supplemented with 5% heat-inactivated (h.i.) FCS (Invitrogen, cat.: 10270-106), 2 mM L-glutamine (Invitrogen, cat.: 25030-024), and 100 U/mL penicillin G sodium/100 μg/mL streptomycin sulfate (Invitrogen, cat.: 15140-122). The ovarian cancer cell line A2780 was purchased from Sigma (cat.: 93112519-1VL) and cultured in complete RPMI 1640 medium. All cell lines were cultured under standard conditions at 37° C. and 5% CO₂ in a humidified atmosphere.

### 4 h Calcein-Release Cytotoxicity Assays

For the calcein-release cytotoxicity assays, target cells were labeled with 10 μM of the fluorescent dye calcein AM (Invitrogen, cat.: cat.: C3100MP) for 30 min in RPMI 1640 medium without FCS, and aliquots of 1×10⁴ calcein-labeled target cells were seeded in individual wells of a round-bottom 96-well micro plate together with freshly isolated and enriched primary human NK cells at an effector:target (E:T) ratio of 2.5:1. 10 pM scFv-IgAb_387 were added in assays on A2780 target cells, and 50 pM scFv-IgAb_387 were added in assays on A-549 target cells in the presence of 2-, 25-, or 50-fold molar excess of soluble FOLR1 (R&D Systems, cat.: 5646-FR-050), soluble mesothelin (R&D Systems, cat.: 3265-MS-050), or without soluble antigens in triplicates. scFv-IgAb_387 was added after soluble antigens to the wells.

Spontaneous calcein-release, maximal release and killing of targets by effectors in the absence of antibodies were determined in quadruplicate on each plate. Spontaneous release was determined by incubation of target cells in the absence of effector cells and in the absence of antibodies. Maximal release was achieved by adding Triton X-100 (Roth, cat.: 3051.2) to a final concentration of 1% in the absence of effector cells and in the absence of antibodies. After centrifugation for 2 min at 200×g, microtiter plates were incubated for 4 h at 37° C. in a humidified atmosphere with 5% CO₂. Following incubation, 100 μL cell-free cell culture supernatant was harvested from each well after centrifugation for 5 min at 500×g and transferred to black flat-bottom 96-well micro plates. Fluorescence counts of released calcein were measured at 520 nm using a multimode plate reader (EnSight, Perkin Elmer). Specific cell lysis was calculated according to the following formula: [fluorescence (sample)−fluorescence (spontaneous)]/[fluorescence (maximum)-fluorescence (spontaneous)]×100% wherein “fluorescence (spontaneous)” and “fluorescence (maximum)” are defined as fluorescence in absence of effector cells and antibodies and fluorescence induced by the addition of Triton X-100, respectively. Mean values of specific target cell lysis (%) and standard deviations (SD) were plotted using GraphPad Prism (v9; GraphPad Software, La Jolla California USA).

### Results

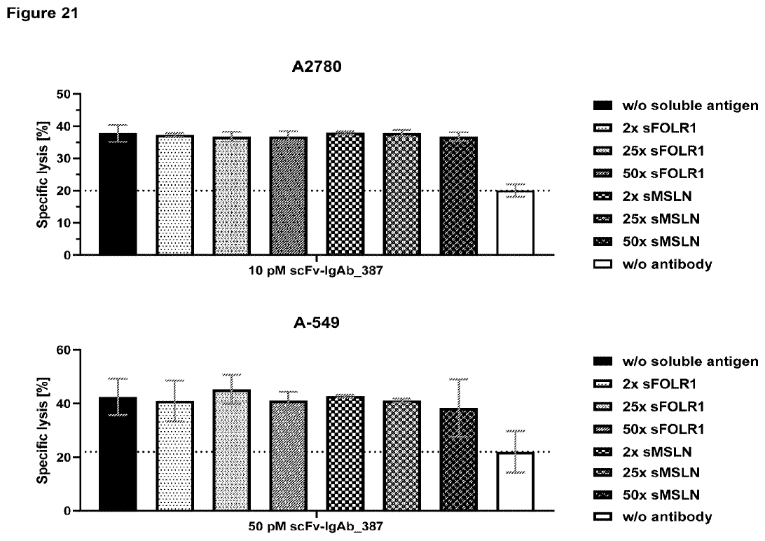

FOLR1×CD16A scFv-IgAb_387 induced lysis of A2780 and A-549 target cells by NK cells at an E:T ratio of 2.5:1 in 4 h calcein-release cytotoxicity assays that was clearly above the lysis induced by NK cells in the absence of antibodies. Addition of soluble FOLR1 at a 2-fold, 25-fold, or 50-fold molar excess relative to the scFv-IgAb_387 concentration did not reduce antibody-mediated cytotoxicity. As expected, soluble mesothelin that was used as a negative control, did also not change the efficacy of scFv-IgAb_387-mediated target cell lysis (FIG. 21).

### Example 18: Assessment of FOLR1×CD16A scFv-IgAb-Induced ADCP′

To assess the potency and efficacy of FOLR1×CD16A scFv-IgAb_387 to induce antibody-dependent cellular phagocytosis (ADCP) macrophages were differentiated from enriched primary human monocytes and used as effector cells in 4 h flow-based ADCP assays with CMFDA-labeled HeLa, HCC-78, and DK-MG target cells in the presence of serial dilutions of scFv-IgAb_387, anti-FOLR1 IgG1 IgAb_355 (SEQ ID NOs: 158+159), RSV/CD16A scFv-IgAb_444 (SEQ ID NO: 156+157), and FOLR1/RSV scFv-IgAb_162.

### Methods

Isolation of PBMC from Buffy Coats and Enrichment of Monocytes

PBMCs were isolated from buffy coats (Transfusion department, University Hospital Pilsen, Czech Republic) by density gradient centrifugation. The buffy coat sample was diluted with a two-to-threefold volume of PBS (BioWest, cat.: L0615-500), layered on a cushion of Lymphoprep (Scintila, cat.: 07811) and centrifuged at 800×g for 25 min at room temperature without brake. PBMC located in the interface were collected and washed 3 times with PBS. PBMC were then used for the enrichment of monocytes by positive selection using the EasySep™ Human CD14 Positive Selection KIT II (Stem Cell Technologies, cat.: 17858) and the Big Easy EasySep™ Magnet (Stem Cell Technologies, cat.: 18001) according to the manufacturer's instructions.

Differentiation of Macrophages from Monocytes

Enriched monocytes were cultured in RPMI 1640 medium (Life Technologies, cat.: 21875-034) supplemented with 10% heat-inactivated (h.i.) FCS (Invitrogen, cat.: 10500-064), 2 mM L-glutamine (Invitrogen, cat.: 25030-024), and 100 U/mL penicillin G sodium and 100 μg/mL streptomycin sulfate (BioWest, cat.: L0022-100) (herein referred to as complete RPMI 1640 medium) in the presence of 50 ng/mL M-CSF (ThermoFisher, cat.: PHC9501) for 5 days 37° C. and 5% CO₂ in a humidified atmosphere. After medium exchange, cells were cultured for additional 2 days in complete RPMI 1640 medium supplemented with M-CSF.

### Culture of Tumor Cell Lines

HeLa cells were cultured in DMEM medium (Fisher Scientific, cat.: 41965062) supplemented with 10% h.i. FCS, 2 mM L-glutamine, and 100 U/mL penicillin G sodium/100 μg/mL streptomycin sulfate. The HCC-78 (DSMZ, cat.: ACC 563) and DK-MG (DSMZ, cat.: ACC 277) cell lines were cultured in RPMI 1640 medium supplemented with 10% h.i. FCS, 2 mM L-glutamine, and 100 U/mL penicillin G sodium/100 μg/mL streptomycin sulfate. All cell lines were cultured under standard conditions at 37° C. and 5% CO₂ in a humidified atmosphere.

### 4 h ADCP Assays

For the ADCP assays aliquots of 5×10⁴ in vitro differentiated macrophages were seeded in individual wells of 96-well UpCell micro plates (ThermoFisher, cat.: 174897) in 100 μL/well complete RPMI 1640 medium and cultured overnight 37° C. and 5% CO₂ in a humidified atmosphere.

On the same day target cells were labeled with 0.5 μM CMFDA (Invitrogen, cat.: C7025) in RPMI 1640 medium without FCS at 37° C. for 30 min, washed once with RPMI 1640 medium without FCS, and then resuspended in complete RPMI 1640 medium and cultured overnight 37° C. and 5% CO₂ in a humidified atmosphere.

On the day of the ADCP assay, CMFDA-labeled target cells were harvested, counted, and aliquots of 1×10⁴ target cells per well were then seeded on top of the pre-cultured adherent macrophages resulting in an E:T ratio of 5:1. The indicated antibodies were added at 10 serial 1:10 dilutions starting at 500 nM to the co-cultures. As controls, macrophages alone, target cells alone, and macrophages with target cells were seeded in the absence of antibodies. After centrifugation at 300×g for 1 min at room temperature, plates were incubated for 4 h at 37° C. and 5% CO₂ in a humidified atmosphere,

After incubation, cultures were washed twice with chilled PBS (Invitrogen, cat.: 392-0434) containing 2% h.i. FCS (Invitrogen, cat.: 10500-064), and 0.1% sodium azide (Sigma, cat.: S8032) (herein referred to as FACS buffer), and then stained for 30 min in the dark with AlexaFluor700-conjugated mAb anti-CD11b (Biolegend, cat.: 101222) and eFluor780 viability dye (ThermoFisher, cat.: 65-0865-14) in FACS buffer supplemented with 1 mg/mL polyclonal human IgG (Cutaquig, Octapharma, cat.: K939D8143). After an additional washing step, cells were resuspended in FACS buffer, and the number of viable CMFDA⁺ and CD11b⁺ events in a defined sample volume was quantified using a flow cytometer (MACSQuant X, Miltenyi Biotec). The percentage of phagocytosis was calculated using the formula: (number of viable, CMFDA⁺/CD11b⁺ target cells)/(number of viable, CMFDA⁺ target cells)×100%.

Phagocytosis values (%) were plotted and, if applicable, in vitro potency (EC₅₀) and efficacy (Eₜₒₚ) were determined by fitting the non-linear regression model to sigmoidal dose-response curves (variable slope) using GraphPad Prism (v9; GraphPad Software, La Jolla California USA).

### Results

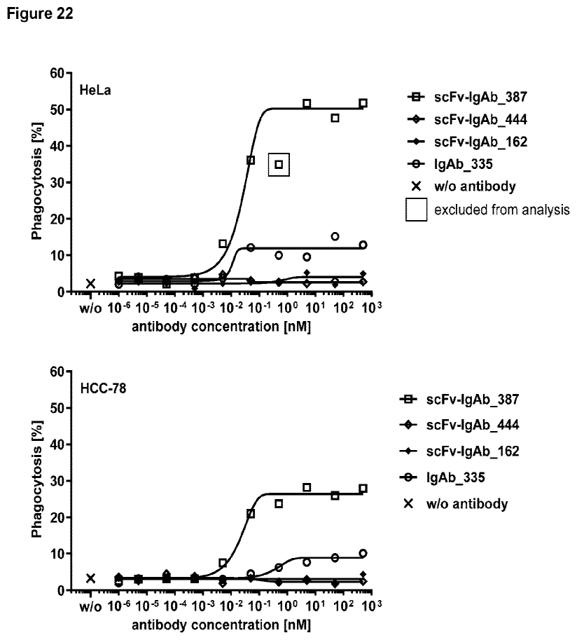

The results of independent ADCP assays demonstrated efficacious phagocytosis of HeLa cells expressing high levels of FOLR1 (mean SABC: 354,206) and HCC-78 target cells expressing medium levels of FOLR1 (mean SABC: 96,969) induced by FOLR1/CD16A scFv-IgAb_387 in the presence of human macrophages (FIG. 22). Anti-FOLR1 IgG1 IgAb_355 induced phagocytosis with lower efficacy (Table 21), and no measurable phagocytosis of FOLR1^{low} DK-MG target cells, which were still phagocytosed in the presence of scFv-IgAb 387. The two control antibody constructs RSV/CD16A scFv-SgAb444 and FOLR1/RSV scFv-DgAb_162, in which either the anti-FOLR1 or the anti-CD16A Fv domain is replaced by an anti-RSV Fv domain, induced no ADCP under the same experimental conditions demonstrating specificity of scFv-IgAb_387-mediated ADCP and the requirement of both, anti-FOLR1 Fabs and anti-CD16A scFv for efficacious and potent ADCP.

TABLE 21
Mean efficacies (Phagocytosis [%]) and potencies
(EC₅₀) determined in 3 independent ADCP assays
on HeLa, HCC-78, and DK-MG target cells

| | | Phagocytosis [%] | | EC50 [nM] | |
|---|---|---|---|---|---|
| | Antibody | Mean | SD | Mean | SD |
| HeLa | scFv-IgAb_387 | 42.9 | 7.0 | 0.16 | 0.13 |
| | IgAb_335 | 16.4 | 4.3 | 0.78 | 1.06 |
| | scFv-IgAb_444 | 0 | 0 | n.a. | n.a. |
| | scFv-IgAb_162 | 0 | 0 | n.a. | n.a. |
| HCC-78 | scFv-IgAb_387 | 26.0 | 3.9 | 17.78 | 24.99 |
| | IgAb_335 | 10.7 | 0.1 | 0.81 | 0.74 |
| | scFv-IgAb_444 | 0 | 0 | n.a. | n.a. |
| | scFv-IgAb_162 | 0 | 0 | n.a. | n.a. |
| DK-MG | scFv-IgAb_387 | 17.6 | 4.5 | 0.10 | 0.10 |
| | IgAb_335 | 0 | 0 | n.a. | n.a. |
| | scFv-IgAb_444 | 0 | 0 | n.a. | n.a. |
| | scFv-IgAb_162 | 0 | 0 | n.a. | n.a. |

### Example 19: ScFv-IgAb_387 Displays Antitumor Efficacy In Vivo in a Xenograft Model of Ovarian Cancer

### Methods

### Cell Culture

Tumor IGROV-1 cell line was maintained in vitro as monolayer culture in the appropriate culture media supplemented with 10% fetal bovine serum and incubated at 37° C. in a humidified atmosphere with 5% CO2. Tumor cells were routinely sub-cultured by trypsin-EDTA treatment 2˜3 times a week, depending on the growth rate. The cells in an exponential growth phase were harvested and counted for inoculation. Cells were tested to be pathogens- and mycoplasma-free.

### Mice

CB-17 SCID-Fcgr4tm2 (FCGR3A)/Bcgen mice were used for this study. Briefly, the regulatory region and exons 1˜5 of mouse Fcgr4 gene that encode the full-length protein CD16 was replaced by human FCGR3A exons 1˜4 and regulatory region in B-hCD16A mice (CB-17 SCID).

### Tumor Inoculation

IGROV-1 cells were harvested in an exponential growth phase and centrifuged at 335×g in a refrigerated centrifuge and the medium aspirated. Cell pellet was re-suspended in 10× volume serum-free medium, filtered through a 70 μm nylon mesh cell strainer and counted using NucleoCounter®NC-200™. The cell suspension was centrifuged again as above and re-suspended in serum-free medium to obtain double the final concentration and then diluted 1:1 with Matrigel, so each 100 μL delivered the number of cells needed per inoculation. Each mouse was inoculated subcutaneously at the right lower flank (near the dorsal thigh region). Mice were lightly anesthetized during the implantation procedure.

### Groups

The study groups and number of animals per group are shown in Table 22.

In the prophylactic group, the treatment was initiated a day after the inoculation (day 1) where the day of inoculation was represented as day 0. In the therapeutic group, the treatment started when the tumor volume (TV) had reached about 50 mm³ (approximately 10-11 days post-inoculation). Based on the TV, mice were grouped into respective groups using Matched Distribution randomization method of the Studylog software (Studylog Systems, San Francisco, USA).

TABLE 22
The different groups with their number of mice per group (N), dose, route
of administration (ROA), frequency and dosing schedule are shown.

| | | | | Dose | | | Dosing Schedule [symbol] |
|---|---|---|---|---|---|---|---|
| G | Cohort | Treatment | N | (mg/kg) | ROA | Frequency | (Days post-inoculation) |
| 1 | Therapeutic | Vehicle- | 10 | 0 | IV | BIW [symbol] | 11 [symbol] , 15, 18, 22, 25, 29, 32, 36 |
| | | Tx | | | | | |
| 2 | Therapeutic | AFVT- | 10 | 30 | IV | BIW [symbol] | 11 [symbol] , 15, 18, 22, 25, 29, 32, 36 |
| | | 2101-Tx | | | | | |
| 3 | Prophylactic | AFVT- | 10 | 30 | IV | BIW [symbol] | 1, 4, 8, 11, 15, 18, 22, 25, 29, |
| | | 2101-Pro | | | | | 32, 36 |

Dosing vol is 5 mL/kg for IV. [symbol]
For BIW, the 2^{nd} dose may be either on 3 or 4 days from the 1^{st}. The actual dosing schedule may change depending on the treatment initiation date. [symbol]
Treatment in Tx cohorts is initiated when TV is −50 mm³ [symbol] approximately 10 ± 1 days post-inoculation.
IV, intravenous;
BIW, twice per week. [symbol]
indicates data missing or illegible when filed

### Flow Cytometric Analysis

Cells were resuspended in FcR-blocking solution, stained with FACS antibodies, resuspended in Live/Dead staining, fixed using fixation solution, and analyzed by flow cytometer. Raw data were analyzed using FlowJo software. Cells were gated on live and single cells (FSC-A vs. FSC-H) using Live-Dead dye followed by gating on lineage markers. The median fluorescence intensity (MFI) and percentage of cells positive for specific markers were exported and plotted using GraphPad prism software. Fluorescence minus one (FMO) controls were used as reference for setting the gates.

### Statistical Analysis

All the data were analyzed using GraphPad software Prism 9 and P<0.05 was considered statistically significant.

### Results

### Tumor Growth Analysis

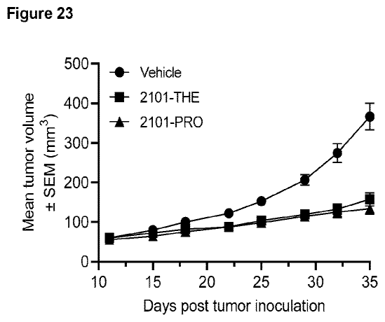

Mice treated with scFv-IgAb_387, both in the prophylactic and therapeutic setting, showed a reduction of tumor growth compared to vehicle-treated mice. After 35 days of tumor growth, the mean tumor volume of the vehicle-treated group was 366 mm³, while the groups treated therapeutically or prophylactically had a significantly smaller neoplastic lesion of 157 mm³ and 133 mm³, respectively (FIG. 23).

### Flow Cytometry Analysis

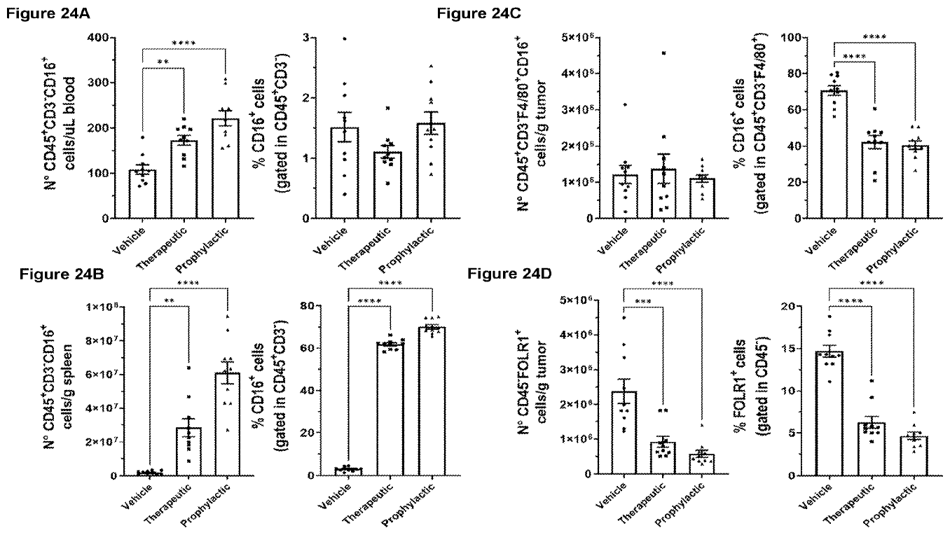

Mice were sacrificed at the termination of the experiment, 35 days after the tumor inoculation. Blood, spleen, and tumor were harvested and analyzed by flow cytometry. Signs of pharmacodynamic activity in periphery of scFv-IgAb_387 were observed in blood where the number, but not the frequency, of CD16⁺ cells increased in treated mice (FIG. 24A). A similar phenomenon was observed in spleen as well. Specifically, both therapeutic and prophylactic scFv-IgAb_387 treatments increased remarkably the absolute number and the frequency of CD16⁺ cells (FIG. 24B). Differently, in tumor a reduction of the percentage of CD16⁺ tumor associated macrophages (TAM) was observed suggesting an ongoing process of repolarization of the tumor microenvironment (FIG. 24C). In line with the mechanism of action of scFv-IgAb_387, there was a significant reduction of the number and the frequency of FOLR1⁺ tumor cells, suggesting an elimination of the FOLR1⁺ tumor clones induced by the ICE (FIG. 24D).

### Example 20: Induction of Cytokine Release by FOLR1×CD16A scFv-IgAb in Cultures of Human PBMC in the Absence and Presence of Target Cells

To assess the on- and off-target cytokine release induced by FOLR1×CD16A scFv-IgAb_387 in vitro PBMC from healthy blood donors were cultured in the absence of target cells, or co-cultured together with FOLR1^{high} OVCAR-3, FOLR1^{low} A-549 target cells, or FOLR1⁻ Raji cells in the presence of serial dilutions of FOLR1×CD16A scFv-IgAb_387 or without antibody. As a positive control for induction of strong cytokine release, CD3/CD28 activator beads were added. Cytokine were quantified in the cell culture supernatant after 24 h incubation.

### Methods

Isolation of PBMC from Buffy Coats

PBMCs were isolated from buffy coats (German Red Cross, Mannheim, Germany) by density gradient centrifugation. The buffy coat samples were diluted with a two-to-threefold volume of PBS (Invitrogen, cat.: 14190-169), layered on a cushion of Lymphoprep (Stem Cell Technologies, cat.: 07861) and centrifuged at 800×g for 25 min at room temperature w/o brake. PBMC located in the interface were collected and washed 3 times with PBS before they were used in the cytokine release assay.

### Culture of Tumor Cell Lines

A-549 cells (CLS, cat.: 30014) were cultured in advanced DMEM/F12 (Invitrogen, cat.: 12634010) supplemented with 5% heat-inactivated (h.i.) FCS (Invitrogen, cat.: 10270-106), 2 mM L-glutamine (Invitrogen, cat.: 25030-024), and 100 U/mL penicillin G sodium/100 μg/mL streptomycin sulfate (Invitrogen, cat.: 15140-122). OVCAR-3 cells (ATCC, cat.: HTB-161) were cultured in RPMI 1640 (Invitrogen, cat.: 21875-091) supplemented with 20% h.i. FCS, 2 mM L-glutamine, 100 U/mL penicillin G sodium/100 μg/mL streptomycin sulfate, and 10 μg/mL bovine insulin (Sigma, cat.: I0516-5ML). Raji cells (DSMZ, cat.: ACC 319) were cultured in RPMI 1640 medium supplemented with 10% h.i. FCS, 2 mM L glutamine, and 100 U/mL penicillin G sodium/100 μg/mL streptomycin sulfate (herein referred to as complete RPMI 1640 medium). All cell lines were cultured under standard conditions at 37° C. and 5% CO₂ in a humidified atmosphere.

### Cytokine Release Assay

Aliquots of 5×10⁵ freshly isolated PBMC were seeded in the presence or absence of FOLR1+A-549, FOLR1⁺ OVCAR-3, and FOLR1⁻ Raji cells at an effector-to-target (E:T) ratio of 20:1 in individual wells of 96-well, flat-bottom micro plates that were blocked with complete RPMI medium overnight. Antibodies were added at 8 serial 1:10 dilutions starting at 100 μg/mL. As a positive control for cytokine release, CD3/CD28 Dynabeads (4×10⁵/well; ThermoFisher, cat.: 11131D) were added. After 24 h culture in a total volume of 200 μL/well in complete RPMI medium, aliquots of cell-free supernatant were harvest and stored at −80° C. until quantification of MCP-1, MIP-1α, MIP-1β, CD40 Ligand, IP-10, GM-CSF, Granzyme B, IFN-α, IFN-γ, IL-1α, IL-1β, IL-1ra, IL-2, IL-4, IL-6, IL-8, IL-10, IL-12p70, IL-13, IL-15, IL-17, IL-33, PD-L1, and TNF-α by bead-based multiplex methodology at Bioassay GmbH (Heidelberg, Germany) using Human Immunotherapy Luminex Performance Assay 24-plex Fixed Panel (R&D Systems, cat.: LKTM010). Three experiments with PBMC from independent blood donors were performed. The quantified cytokine concentrations were plotted against the antibody concentration by non-linear regression/sigmoidal dose-response (variable slope) using GraphPad Prism for Windows (v9; GraphPad Software; La Jolla California USA).

### Results

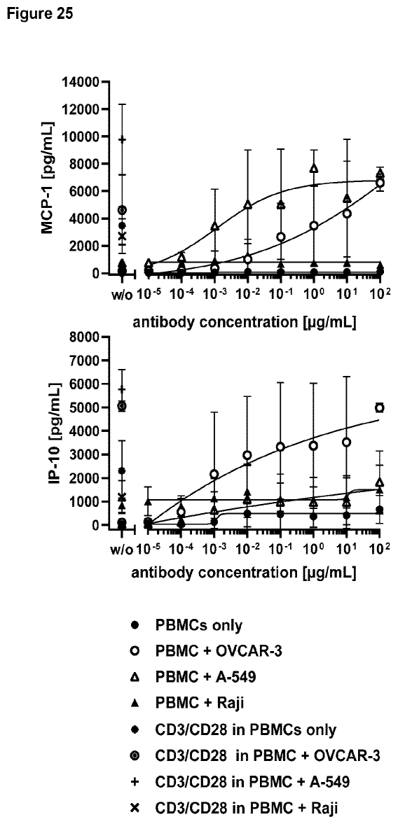

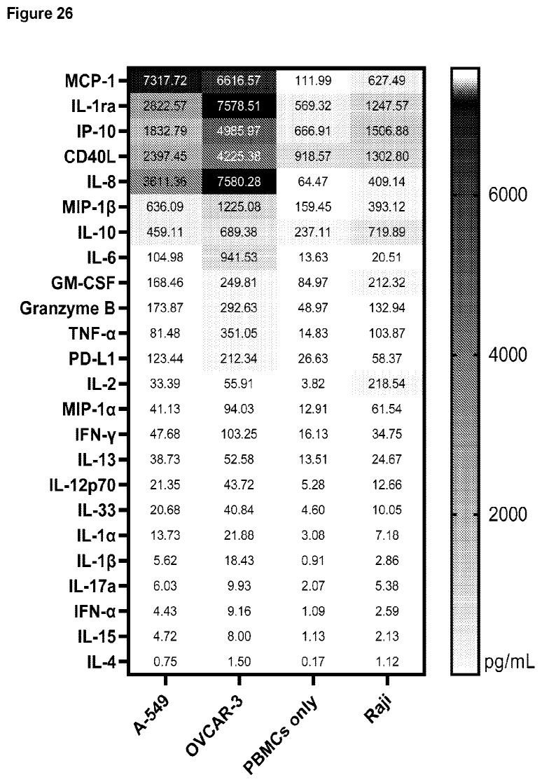

The addition of increasing concentrations of FOLR1×CD16A scFv-IgAb_387 did not induce substantial release of the tested cytokines in 24 h cultures of human PBMC, suggesting no off-target immune cell activation by scFv-IgAb_387. The results of the measured MCP-1 and IP-10 concentrations are presented in FIG. 25 as an example. The addition of tumor cell lines to the PBMC cultures in the absence of antibodies induced in most cases no substantial cytokine release. The addition of Raji cells to PBMC was associated with elevated background levels of some cytokines, e.g. IP-10 (see FIG. 26), MIP-1α, MIP-1β, IFN-γ, IL-10, TNF-α, IL-2, GM-CSF, Granzyme B (data not shown).

In the presence of FOLR1⁻ Raji cells FOLR1×CD16A scFv-IgAb_387 did not induce a concentration-dependent secretion of the tested cytokines. In contrast, in the presence of FOLR1⁺ A-549 or FOLR1⁺ OVCAR-3 target cells, scFv-IgAb_387 stimulated the release of several cytokines in a concentration manner. Among the induced cytokines MCP-1, IL-1ra, IP-10, CD40L, IL-8 and MIP-lb were the ones with the highest concentrations in the supernatant (see FIG. 25 and FIG. 26). The levels of cytokines induced by FOLR1×CD16A scFv-IgAb_387 were mostly higher in co-cultures with OVCAR-3 target cells expressing high levels of cell surface FOLR1 relative to the co-cultures with A-549 target cells expressing lower levels of FOLR1 (FIG. 26). In conclusion, the results demonstrate a moderate, concentration-dependent, and target-restricted induction of pro-inflammatory cytokines by FOLR1×CD16A scFv-IgAb_387 in cultures of human PBMC, but not in the absence of FOLR1⁺ target cells, suggesting a potent but safe in vitro profile of scFv-IgAb_387.

### Example 21: scFv-IgAb_387-Mediated ADCC Tumor Cell Lines Expressing Different Levels of FOLR1

The objective of this study was to assess the efficacy and potency of scFv-IgAb_275 and scFv-IgAb_387 in mediating antibody-dependent cellular cytotoxicity (ADCC) towards tumor target cell lines in vitro. For this, a broad panel of tumor cell lines of different cellular backgrounds and origins expressing different levels of FOLR1 were included in the study.

### Methods

TABLE 23
Antibody constructs

| | Target | Target | Effector | Effector |
|---|---|---|---|---|
| Construct | specificity | domain | specificity | domain |
| scFv-IgAb_275 | FOLR1 | FOLR1_1 | CD16A | CD16a3 |
| scFv-IgAb_387 | FOLR1 | FOLR1_1 | CD16A | CD16a3 |
| scFv-IgAb_444 | RSV | NIST | CD16A | CD16a3 |
| | | RM8671 | | |
| scFv-IgAb_162 | FOLR1 | FOLR1_1 | RSV | NIST |
| | | | | RM8671 |
| IgAb_335 | FOLR1 | FOLR1_1 | IgG1 | Fc |

### Culture of Cell Lines

KARPAS-299 (DSMZ, cat.: ACC-31), NCI-H2110 (ATCC, cat.: CRL-5924), HCC-78 (DSMZ, cat.: ACC 563), HCC-1954 (ATCC, cat.: CRL-2338), HCC-15 (DSMZ, cat.: ACC 496), HCC-1187 (ATCC, cat.: CRL-2322), HCC-44 (DSMZ, cat.: ACC 534), Raji (DSMZ, cat.: ACC 319), and A2780 (Sigma, cat.: 93112519) were cultured in complete RPMI 1640 medium (RPMI 1640 medium (Life Technologies, cat.: 21875-034) supplemented with 10% heat-inactivated (h.i.) FCS (Invitrogen, cat.: 10500-064), 2 mM L-glutamine (Invitrogen, cat.: 25030-024), 100 U/mL penicillin G sodium, 100 μg/mL streptomycin sulfate (BioWest, cat.: L0022-100)). HCC-827 (DSMZ, cat.: ACC-566) was cultured in RPMI 1640 medium supplemented with 20% h.i. FCS, 2 mM L glutamine, 100 U/mL penicillin G sodium, 100 μg/mL streptomycin sulfate. SKBR-3 (DSMZ, cat.: ACC-736) was cultured in McCoys 5a medium supplemented with 20% h.i. FCS, 2 mM L-glutamine, 100 U/mL penicillin G sodium, 100 μg/mL streptomycin sulfate. MDA-MB-231 (DSMZ, cat.: ACC-732) was cultured in Leibovitz′ L15 medium supplemented with 10% h.i. FCS, 2 mM L-glutamine, 100 U/mL penicillin G sodium, 100 μg/mL streptomycin sulfate. IGROV-1 (Millipore, cat.: SCC203) was cultured in DMEM High Glucose medium (Fisher Scientific, cat.: 41965062) supplemented with 10% h.i. FCS, 100 U/mL penicillin G sodium, 100 μg/mL streptomycin sulfate. T47-D (DSMZ, cat.: ACC 739) was cultured in RPMI 1640 medium supplemented with 15% h.i. FCS, 2 mM L-glutamine, 100 U/mL penicillin G sodium, 100 μg/mL streptomycin sulfate, and 10 μg/mL human Insulin (Sigma, cat.: I9278). OVCAR-3 (ATCC, cat.: HTB-161) was cultured in RPMI 1640 medium supplemented with 20% h.i. FCS, 2 mM L-glutamine, 100 U/mL penicillin G sodium, 100 μg/mL streptomycin sulfate, and 10 μg/mL bovine Insulin (Sigma, cat.: I0516). SCOV-3 (ATCC, cat.: HTB-77) was cultured in McCoys 5a medium (LGC Standards, cat.: ATCC-30-2007) supplemented with 10% h.i. FCS, 2 mM L-glutamine, 100 U/mL penicillin G sodium, 100 μg/mL streptomycin sulfate. All cell lines, except MDA-MB-231, were cultured under standard conditions as recommended by the supplier at 37° C. and 5% CO2 in a humidified atmosphere. MDA-MB-231 cultured at 37° C. and without CO₂ in a humidified atmosphere.

### Quantification of FOLR1 Surface Expression Levels

FOLR1 cell surface expression was determined on all tested tumor cell lines using QIFIKIT (Agilent, cat.: DAK007811-8) and anti-FOLR1 mAb LK26 (Abcam, cat.: ab3361) according to manufacturer's instructions. In brief, aliquots of 0.5×10⁶ cells were stained with 40 μg/mL mAb LK26 followed by F(ab′)2 fragment of FITC-conjugate goat anti-mouse IgG. As negative control, aliquots of 0.5×10⁶ cells were stained with 40 μg/mL mAb MG2a-53 (Biolegend, cat.: 401501) followed by F(ab′)₂ fragment of FITC conjugate goat anti-mouse IgG. For the generation of a calibration curve, calibration beads containing 5 populations of beads bearing different distinct numbers of mAb molecules were stained with F(ab′)₂ fragment of FITC-conjugate goat anti-mouse IgG. From the resulting mean fluorescence intensities determined by flow cytometry, a calibration curve was calculated using non-linear regression according to manufacturer's instructions. The calibration curve was used to interpolate the antibody binding capacity (ABC) for mAb LK26 and mAb MG2a-53 used to assess background binding. Specific antibody binding capacity (SABC) was calculated by subtracting ABC from MG2a-53 from the ABC from mAb LK26. If not otherwise indicated, SABC numbers were determined in at least 3 independent experiments and mean values were calculated.

Isolation of Human PBMC from Buffy Coats

PBMCs were isolated from buffy coats (Transfusion department, University Hospital Pilsen, Czech Republic) by density gradient centrifugation. The buffy coat sample was diluted with a two-to-threefold volume of PBS (BioWest, cat.: L0615-500), layered on a cushion of Lymphoprep (Scintila, cat.: 07811) and centrifuged at 800×g for 25 min at room temperature without brake. PBMC located in the interface were collected and washed 3 times with PBS before they were cultured overnight in RPMI 1640 medium (Life Technologies, cat.: 21875-034) supplemented with 10% heat-inactivated FCS (Invitrogen, cat.: 10500-064), 2 mM L-glutamine (Invitrogen, cat.: 25030-024), 100 U/mL penicillin G sodium and 100 μg/mL streptomycin sulfate (BioWest, cat.: L0022-100) at 37° C. and 5% CO2 in a humidified atmosphere without stimulation.

Enrichment of Human NK Cells from PBMC

For the enrichment of NK cells, PBMCs were harvested from overnight cultures and for one round of negative selection using the EasySep™ Human NK Cell Enrichment kit (Stem Cell Technologies, cat.: 17955) for the immunomagnetic isolation of human NK cells and the Big Easy SepMate™ Magnet (Stem Cell Technologies, cat.: 18001) according to the manufacturer's instructions.

### Freezing of Isolated NK Cells

Isolated NK cells have been centrifuged at 400×g, 5 min, 4° C. Cell pellet has been resuspended in freezing media (90% h.i. FCS and 10% DMSO) at a density 1×10⁷ cells/mL. Cells were frozen overnight at −80° C. and then have been transferred to liquid nitrogen for long term storage.

### Calcein-Release Cytotoxicity Assays

Antibody-mediated target cell lysis by NK cells in vitro was assessed by quantifying the release of calcein into cell culture supernatants from calcein-labeled target cells. For this, target cells were labeled with 10 μM calcein AM (Fisher Scientific, cat.: C3100MP) for 30 min in RPMI 1640 medium without FCS at 37° C. After gentle washing, calcein-labeled cells were resuspended in complete RPMI medium at a density of 1×10⁵/mL. 1×10⁴ target cells were then seeded in individual wells of a round-bottom 96-well microtiter plate and, if not mentioned otherwise, mixed with enriched human NK cells at an effector-to-target cell (E:T) ratio of 5:1. The culture of NK cells with target cells was conducted in duplicate without antibody addition or in the presence of titrated antibodies starting at a concentration of 100 nM followed by ten 6-fold serial dilutions. After centrifugation for 2 min at 200×g, microtiter plates were incubated for 4 h at 37° C. in a humidified atmosphere with 5% CO2. Spontaneous calcein-release, maximal release and killing of targets by effectors in the absence of antibodies were determined in quadruplicate on each plate. Spontaneous release was determined by incubation of target cells in the absence of effector cells and in the absence of antibodies. Maximal release was achieved by adding Triton X-100 (Carl Roth, cat.: 3051.2) to a final concentration of 1% in the absence of effector cells and in the absence of antibodies. Following incubation, 100 μL cell-free cell culture supernatant was harvested from each well after centrifugation for 5 min at 500×g and transferred to black flat-bottom 96-well microtiter plates. Fluorescence counts of released calcein were measured at 520 nm using a multimode plate reader. Specific cell lysis was calculated according to the following formula: [fluorescence (sample)−fluorescence (spontaneous)]/[fluorescence (maximum)−fluorescence (spontaneous)]×100% wherein “Fluorescence (spontaneous)” and “Fluorescence (maximum)” are defined as fluorescence in absence of effector cells and antibodies and fluorescence induced by the addition of Triton X-100, respectively.

### Statistical Analysis

Mean values of specific target cell lysis (%) and standard deviations (SD) were plotted and in vitro potency (EC₅₀) and efficacy (Eₘₐₓ) were determined by fitting the non-linear regression model to sigmoidal dose-response curves (variable slope) using GraphPad Prism (v9; GraphPad Software, La Jolla California USA). A nonlinear-regression model was considered suitable if R² was bigger or equal to 0.8.

### Results

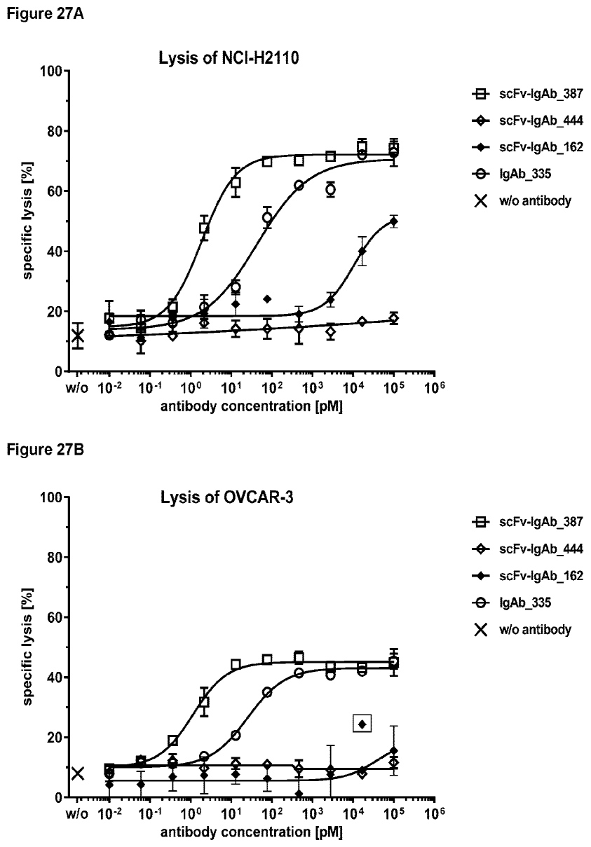

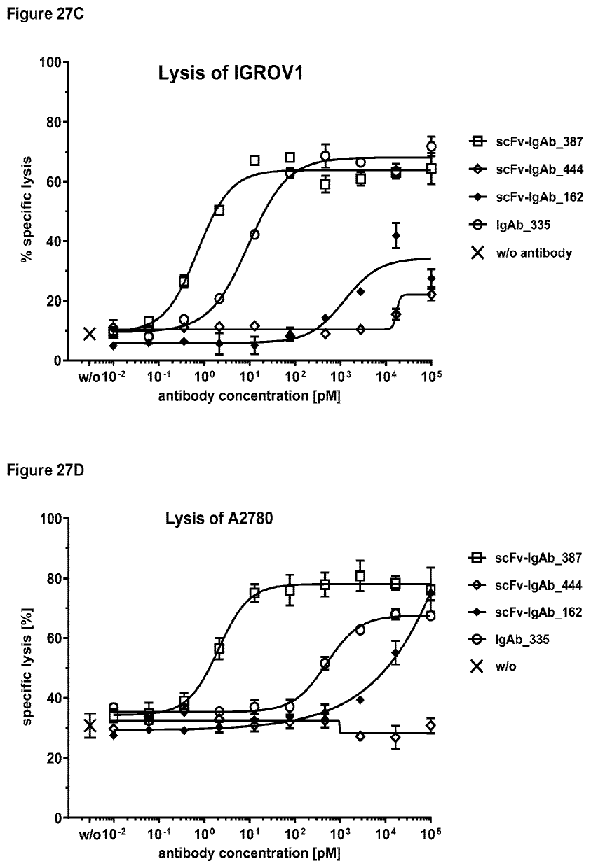

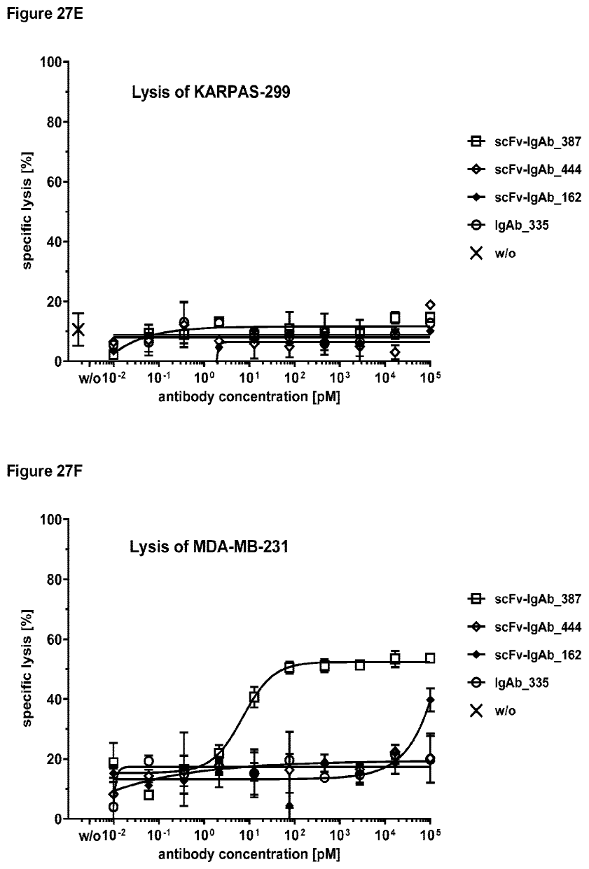

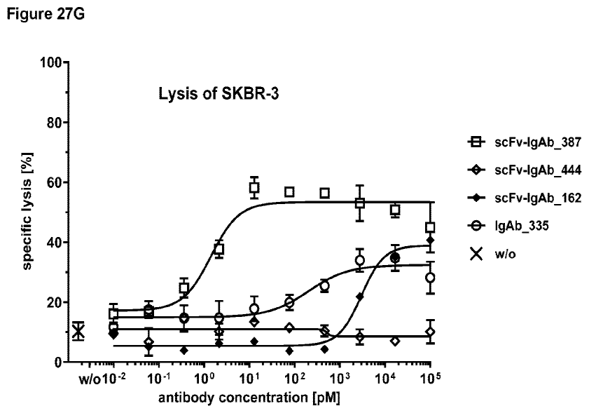

FOLR1×CD16A scFv-IgAb_275 and scFv-IgAb_387 induced potent and efficient lysis of all tested FOLR1-positive tumor cell lines in the presence of NK cells in a concentration-dependent manner (FIGS. 27A-G), but not on FOLR1-negative KARPAS-299 or Raji cells, with mean EC₅₀ values ranging from 0.8 pM on OVCAR-3 cells to 6.0 pM on HCC-827 cells, and mean Eₘₐₓ values in the range between 87.7% (NCI-H2110) and 32.0% (HCC-827) (Table 24). In vitro ADCC mediated by scFv-IgAb_275 and scFv-IgAb_387 was independent of the FOLR1-expression level. In contrast, anti-FOLR1 IgG1 with farletuzumab-derived Fv domains (IgAb_335) exhibited clearly lower potencies on target cells with low FOLR1 expression levels and induced no lysis of cells with very low surface levels of FOLR1. The control antibody constructs scFv-IgAb_444 (RSV×CD16A), and scFv-IgAb_162 (FOLR1×NIST) where either the CD16A- or the FOLR1-targeting domains were replaced by RSV-targeting domains or harboring a Fc-silenced IgG1, induced no or only at very high antibody concentrations cytotoxicity, indicating that both, the anti-FOLR1 and the anti-CD16A targeting domains are required for potent and efficacious induction of ADCC as it was observed for scFv-IgAb_387. The FOLR1×RSV scFv-IgAb_162 induced on various FOLR1⁺ tumor cell lines at higher antibody concentrations (>1 nM) target cell lysis, but with substantial lower potency and efficacy than scFv-IgAb_387, it is most likely not due to a direct cytotoxic effect induced by binding to FOLR1 on the tumor target cells, but rather due to the anti-RSV domains that were introduced to replace the anti-CD16A domains in this control antibody construct. Target-specificity of scFv-IgAb_387 was further demonstrated using KARPAS-299 target cells, a FOLR1-negative cell line that expresses CD30. Neither scFv-IgAb_387 nor the control antibodies IgAb_335 (anti-FOLR1 IgG1), scFv-IgAb_444 (RSV×CD16A), or scFv-IgAb_162 (FOLR1×RSV) induced cell lysis.

Table 24: Tabulated summary of potency (EC₅₀) and efficacy (Eₘₐₓ) determined for FOLR1×CD16A scFv-IgAb_275, scFv-IgAb_387, and anti-FOLR1 IgG1 IgAb_335 on various target cell lines. Potency (EC₅₀) and efficacy (Eₘₐₓ) were determined in 4 h calcein-release assays using enriched primary human NK cells as effector cells at an E:T ratio of 5:1. Specific antibody binding capacity (SABC) as a marker for FOLR1 expression level on cells was determined on all tumor cell lines using QIFIKIT. Mean SABC, EC₅₀, and Eₘₐₓ values from ≥3 experiments are presented; n.a., not applicable.

TABLE 24

| | | | | | scFv- | | | | scFv- |
|---|---|---|---|---|---|---|---|---|---|
| | | | | | IgAb fold- | | | | IgAb fold- |
| | | FOLR1 | scFv- | scFv- | improvement | scFv- | scFv- | | improvement |
| | | expression | IgAb_275 | IgAb_387 | in potency vs | IgAb_275 | IgAb_387 | IgAb_335 | in efficacy vs |
| Cell Line | Indication | [SABC] | EC₅₀ [pM] | EC₅₀ [pM] | IgAb_335 | Eₘₐₓ [%] | Eₘₐₓ [%] | Eₘₐₓ [%] | IgAb_335 |
| IGROV-1 | OC | 209,884 | | 1.0 | 15.1 | | 39.1 | 43.9 | 0.9 |
| NCI-H2110 | NSCLC | 116,216 | | 0.8 | 34.4 | | 87.7 | 71.1 | 1.2 |
| HCC-78 | NSCLC | 96,969 | 24.5 | | 25.7 | 38.7 | | 32.6 | 1.2 |
| T47-D | BC | 51,099 | 0.5 | | 33.8 | 49.7 | | 56.4 | 0.9 |
| HCC-1954 | BC | 41,543 | 13.8 | | 70.3 | 41.7 | | 37.1 | 1.1 |
| OVCAR-3 | OC | 36,094 | | 1.1 | 40.4 | | 47.5 | 34.5 | 1.4 |
| HCC-15 | LC | 27,520 | 49.8 | | 3.4 | 37.5 | | 29.0 | 1.3 |
| SKOV-3 | OC | 25,244 | 4.5 | | 139.5 | 69.6 | | 46.7 | 1.5 |
| HCC-827 | NSCLC | 24,267 | | 2.8 | 30.8 | | 32.0 | 16.6 | 1.9 |
| SK-BR-3 | BC | 4,570 | 3.6 | | 143.6 | 49.7 | | 33.2 | 1.5 |
| HCC-1187 | BC | 4,189 | 4.5 | | 51.5 | 71.4 | | 72.0 | 1.0 |
| HCC-44 | NSCLC | 2,323 | 18.3 | | 109.1 | 40.0 | | 20.2 | 2.0 |
| MDA-MB-231 | TNBC | 1,976 | | 6.5 | 54.0 | | 46.8 | 22.3 | 2.1 |
| A2780 | OC | 1,662 | | 5.5 | 592.6 | | 75.4 | 25.9 | 2.9 |
| KARPAS-299 | ALCL | 0 | | n.a. | n.a. | | 0.0 | 0.0 | n.a. |
| Raji | BCL | 0 | | n.a. | n.a. | | 0.0 | 0.0 | n.a. |

### Example 22: Binding of FOLR1×CD16A Constructs to Primary Human NK Cells in the Presence or Absence of 10 mg/mL Polyclonal Human IgG

The objective of this study was the assessment of scFv-IgAb_387 binding to endogenously expressed CD16A on primary human NK cells in the presence and absence of physiological concentration of polyclonal human IgG. scFv-IgAb_387 demonstrated concentration-dependent binding to primary human NK cells with apparent KD value of 4.3 nM. Importantly, under physiological conditions (in the presence of 10 mg/mL polyclonal human IgG) scFv-IgAb_387 retained high affinity interaction with CD16A exhibiting only a marginal decrease of avidity (5.3-fold loss in K_{D}).

### Methods

TABLE 25
Antibody constructs

| | Target | Target | Effector | Effector |
|---|---|---|---|---|
| Construct | specificity | domain | specificity | domain |
| scFv-IgAb_387 | FOLR1 | FOLR1_1 | CD16A | CD16a3 |
| scFv-IgAb_444 | RSV | NIST RM8671 | CD16A | CD16a3 |
| scFv-IgAb_162 | FOLR1 | FOLR1_1 | RSV | NIST RM8671 |
| IgAb_335 | FOLR1 | FOLR1_1 | IgG1 | Fc |

### Biotinylation of Antibodies

Antibodies were chemically biotinylated using EZ-Link™ NHS-PEG4-Biotin Kit (Thermo Scientific, cat.: A39259) in 1×PBS buffer pH 7.4 (BioWest, cat.: L0615-500). Before and after biotinylation antibodies were re-buffered using Zeba™ spin-desalting columns (Thermo Scientific, cat.: 89892). Concentration of biotinylated antibodies has been quantified using UV-Spectroscopy. Biotinylated proteins were analyzed by reduced SDS-PAGE (Bio-Rad, cat.: 4561086) and reduced WB (hFc detection) and completeness of biotinylation has been evaluated by ELISA with and without pre-treatment of streptavidin microbeads (Fisher Scientific, cat.: 11206D).

Isolation of Human PBMC from Buffy Coats

PBMCs were isolated from buffy coats (Transfusion department, University Hospital Pilsen, Czech Republic) by density gradient centrifugation. The buffy coat sample was diluted with a two-to-threefold volume of PBS, layered on a cushion of Lymphoprep (Scintila, cat.: 07811) and centrifuged at 800×g for 25 min at room temperature without brake. PBMC located in the interface were collected and washed 3 times with PBS before they were cultured overnight in RPMI 1640 medium (Life Technologies, cat.: 21875-034) supplemented with 10% heat-inactivated FCS (Invitrogen, cat.: 10500-064), 2 mM L-glutamine (Invitrogen, cat.: 25030-024), 100 U/mL penicillin G sodium and 100 μg/mL streptomycin sulfate (BioWest, cat.: L0022-100) at 37° C. and 5% CO₂ in a humidified atmosphere without stimulation.

Enrichment of Human NK Cells from PBMC

For the enrichment of NK cells, PBMCs were harvested from overnight cultures and for one round of negative selection using the EasySep™ Human NK Cell Enrichment kit (Stem Cell Technologies, cat.: 17955) for the immunomagnetic isolation of human NK cells and the Big Easy SepMate™ Magnet (Stem Cell Technologies, cat.: 18001) according to the manufacturer's instructions.

### Freezing of Isolated NK Cells

Isolated NK cells have been centrifuged at 400×g, 5 min, 4° C. Cell pellet has been resuspended in freezing media (90% FCS plus 10% DMSO (Sigma, cat.: D2650) at a density of 1×10⁷ cells/mL. Cells were frozen overnight at −80° C. and then have been transferred to liquid nitrogen for long-term storage.

### Flow Cytometry

Frozen NK cells were thawed, and viability was determined with trypan blue (Sigma-Aldrich, cat.: T8154). Cells have been centrifuged at 400×g, 5 min, 4° C. Cell pellets were resuspended in FACS buffer PBS (Invitrogen, cat.: 392-0434) containing 2% h.i. FCS (Invitrogen, cat.: 10500-064), and 0.1% sodium azide (Sigma, cat.: S8032) at 2×10⁶ cells/mL. 100 μL/well of the cell suspension were transferred into U-shaped 96 well plates, cells were pelleted at 400×g, 5 min. 4° C., and resuspended in 50 μL/well of diluted antibody constructs. For assays with human polyclonal IgG either 50 μL/well of diluted Cutaquig (Octapharma, cat.: K939D8143) or FACS buffer has been added. After 40-50 minutes' incubation at 37° C., cells were washed 3-times with ice-cold FACS buffer. Cell pellets were resuspended in 25 μL of 25-fold diluted FITC-conjugated secondary antibody (Jackson Immuno Research, cat.: 109-096-088) or 100-fold diluted streptavidin-FITC (Fisher Scientific, cat.: 11-4317-87) and 25 μL 500-fold diluted viability dye (Thermo Fisher, cat.: 65-0865-14) and incubated for 40-50 minutes on ice in the dark. After the final incubation step, cells were washed 2-times with ice-cold FACS buffer, and the cell pellet was resuspended in 50 μL FACS buffer. Fluorescence intensity of >1×10⁴ viable cells was analyzed by flow cytometry and the median fluorescence intensity (MFI) was determined for each sample.

### Statistical Analysis

Equilibrium dissociation constants (KD) of antibody binding, mean and standard deviation (SD) were calculated by plotting MFI values and fitting a non-linear regression model for one-site binding to hyperbolic dose-response curves using GraphPad Prism for Windows (v9; GraphPad Software; La Jolla California USA).

### Results

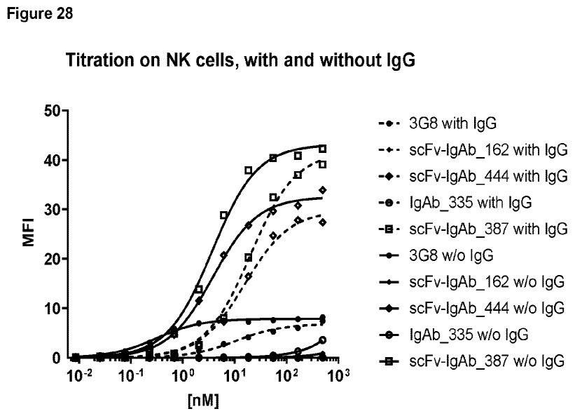

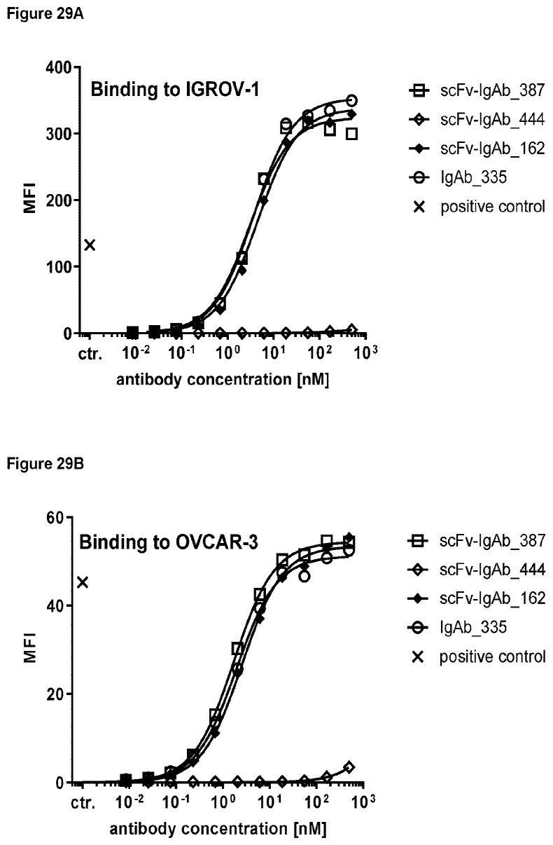

ScFv-IgAb_387 binding to primary human NK cells was investigated by flow cytometry. To investigate the influence of physiological CD16A ligand on antibody binding, biotinylated antibodies were titrated on human NK cells in the presence or absence of 10 mg/mL polyclonal human IgG (FIG. 28). Analysis of antibody binding in four independent experiments demonstrated high avidity binding of scFv-IgAb_387 to NK cells with mean apparent K_{D} of 4.3 nM without IgG (range: 3.7 nM-5.4 nM) and 23.1 nM with IgG: range: 14.3 nM-36.4 nM), respectively, resulting in a mean 5.3-fold loss of avidity when polyclonal IgG was added (Table 26). 3G8, a murine IgG anti-human CD16 showed NK cell binding with a mean apparent avidity (K_{D}) of 0.3 nM in the absence of polyclonal IgG, which was substantially reduced 185-fold in the presence of IgG. Weak binding to NK cells in the absence of polyclonal IgG was also detected for an anti-FOLR1 IgG1 antibody comprising wild-type Fc (IgAb_335). In this case, 10 mg/mL competing IgG during antibody incubation fully abrogated antibody binding.

TABLE 26
Mean apparent avidity (K_{D}) of scFv-IgAb_387 and control antibodies
on NK cells in the presence or absence of polyclonal human IgG.

| | | | | with 10 mg/ml | | | |
|---|---|---|---|---|---|---|---|
| | | | | polyclonal | | | |
| | without IgG | | | human IgG | | | fold loss in |
| antibody | K_{D} [nM] | | | K_{D} [nM] | | | K_{D} induced |
| construct | mean | SD | n | mean | SD | n | by IgG |
| scFv-IgAb_387 | 4.3 | 0.7 | 4 | 23.1 | 8.2 | 4 | 5.3 |
| scFv-IgAb_444 | 3.8 | 0.9 | 4 | 19.9 | 9.5 | 4 | 5.0 |
| scFv-IgAb_162 | n.b. | n.b | 4 | n.b. | n.b. | 4 | n.a. |
| IgAb_335 | n.a. | n.a | 4 | n.b. | n.b. | 4 | n.a. |
| anti-CD16 (3G8) | 0.3 | 0.07 | 4 | 54.6 | 46.3 | 4 | 185 |

Binding of antibody constructs to enriched human NK cells in the presence or absence of 10 mg/mL polyclonal human IgG was measured by flow cytometry. Equilibrium dissociation constants (K_{D}) of antibody binding were calculated by plotting MFI values and fitting a non-linear regression model for one-site binding to hyperbolic dose-response curves using GraphPad Prism.
SD, standard deviation; n, number of experiments;
n.a., not applicable;
n.b., no binding.

### Example 23: Assessment of scFv-IgAb_387 Avidity and Binding Specificity to FOLR1-Positive Target Cells by Flow Cytometry

The objective of this study was to assess the apparent binding avidity and specificity of scFv-IgAb_387 to various tumor cell lines of different origins with different FOLR1 expression levels. scFv-IgAb_387 showed high avidity binding on high-to-mid FOLR1 expressing cell lines resulting in apparent K_{D} values in the range of 1.7 to 3.7 nM. Using cell lines with low cell-surface FOLR1 expression showed specific concentration-dependent binding as well, but no calculation of apparent K_{D} was possible due to low signal intensity. In contrast, no concentration-dependent or specific binding of scFv-IgAb_387 to FOLR1-negative tumor cells was observed.

### Methods

TABLE 27
Antibody constructs

| | Target | Target | Effector | Effector |
|---|---|---|---|---|
| Construct | specificity | domain | specificity | domain |
| scFv-IgAb_387 | FOLR1 | FOLR1_1 | CD16A | CD16a3 |
| scFv-IgAb_444 | RSV | NIST RM8671 | CD16A | CD16a3 |
| scFv-IgAb_162 | FOLR1 | FOLR1_1 | RSV | NIST RM8671 |
| IgAb_335 | FOLR1 | FOLR1_1 | IgG1 | Fc |

### Culture of Cells Lines

KARPAS-299 (DSMZ, cat.: ACC-31), NCI-H2110 (ATCC, cat.: CRL-5924), A2780 (Sigma, cat.: 93112519) were cultured in complete RPMI 1640 medium (RPMI 1640 medium (Life Technologies, cat.: 21875-034) supplemented with 10% heat-inactivated (h.i.) FCS (Invitrogen, cat.: 10500-064), 2 mM L-glutamine (Invitrogen, cat.: 25030-024), 100 U/mL penicillin G sodium, 100 μg/mL streptomycin sulfate (BioWest, cat.: L0022-100)). HCC-827 (DSMZ, cat.: ACC-566) was cultured in RPMI 1640 medium supplemented with 20% h.i. FCS, 2 mM L-glutamine, 100 U/mL penicillin G sodium, 100 μg/mL streptomycin sulfate. SKBR-3 (DSMZ, cat.: ACC-736) was cultured in McCoys 5a medium supplemented with 20% h.i. FCS, 2 mM L-glutamine, 100 U/mL penicillin G sodium, 100 μg/mL streptomycin sulfate. IGROV-1 (Millipore, cat.: SCC203) was cultured in DMEM High Glucose medium (Fisher Scientific, cat.: 41965062) supplemented with 10% h.i. FCS, 100 U/mL penicillin G sodium, 100 μg/mL streptomycin sulfate. OVCAR-3 (ATCC, cat.: HTB-161) was cultured in RPMI 1640 medium supplemented with 20% h.i. FCS, 2 mM L-glutamine, 100 U/mL penicillin G sodium, 100 μg/mL streptomycin sulfate, and 10 μg/mL bovine Insulin (Sigma, cat.: I0516). All cell lines were cultured under standard conditions as recommended by the supplier at 37° C. and 5% CO₂ in a humidified atmosphere.

### Quantification of FOLR1 Surface Expression Levels

FOLR1 cell surface expression was determined on all tested tumor cell lines using QIFIKIT (Agilent, cat.: DAK007811-8) and anti-FOLR1 mAb LK26 (Abcam, cat.: ab3361) according to manufacturer's instructions. In brief, aliquots of 0.5×10⁶ cells were stained with 40 μmL mAb LK26 followed by F(ab′)₂ fragment of FITC-conjugate goat anti-mouse IgG. As negative control, aliquots of 0.5×10⁶ cells were stained with 40 μg/mL mAb MG2a-53 (Biolegend, cat.: 401501) followed by F(ab′)₂ fragment of FITC conjugate goat anti-mouse IgG. For the generation of a calibration curve, calibration beads containing 5 populations of beads bearing different distinct numbers of mAb molecules were stained with F(ab′)₂ fragment of FITC-conjugate goat anti-mouse IgG. From the resulting mean fluorescence intensities determined by flow cytometry, a calibration curve was calculated using non-linear regression according to manufacturer's instructions. The calibration curve was used to interpolate the antibody binding capacity (ABC) for mAb LK26 and mAb MG2a-53 used to assess background binding. Specific antibody binding capacity (SABC) was calculated by subtracting ABC from MG2a-53 from the ABC from mAb LK26. If not otherwise indicated, SABC numbers were determined in at least 3 independent experiments and mean values were calculated.

### Assessment of Apparent Avidity on Tumor Cells by Flow Cytometry

Cells were collected and centrifuged at 400×g, 5 min, 4° C. Cell pellets were resuspended in FACS buffer (PBS (Invitrogen, cat.: 392-0434) containing 2% h.i. FCS (Invitrogen, cat.: 10500-064), and 0.1% sodium azide (Sigma, cat.: S8032)) at 1-5×10⁶ cells/mL. 100 μL/well of the cell suspension were transferred into U-shaped, 96-well micro plates (0.1-0.5×10⁶ cells per well), cells were pelleted at 400×g, 5 min, 4° C., and resuspended in 100 μL/well of serial dilutions of the indicated antibodies in FACS buffer. After 40-50 min incubation at 37° C., cells were washed 3-times with ice-cold FACS buffer. Cell pellets were resuspended in 50 μL of 25-fold diluted FITC-conjugated goat (Fab′)₂ anti-human IgG secondary antibody (Jackson Immuno Research, cat.: 109-096-088) and 50 μL 500-fold diluted viability dye eFluor™ 780 (Thermo Fisher, cat.: 65-0865-14) and incubated for 40-50 min on ice in the dark. After the final incubation step, cells were washed 2-times with ice-cold FACS buffer, and the cell pellet was resuspended in 100 μL FACS buffer. Fluorescence intensities of >1×10⁴ viable cells were analyzed by flow cytometry and the median fluorescence intensity (MFI) was determined for each sample.

### Statistical Analysis

Equilibrium dissociation constants (K_{D}) of antibody binding, mean and standard deviation (SD) were calculated by plotting MFI values and fitting a non-linear regression model for one-site binding to hyperbolic dose-response curves using GraphPad Prism for Windows (v9; GraphPad Software; La Jolla California USA).

### Results

The specificity of scFv-IgAb_387 binding to the panel of tumor cell lines was investigated by flow cytometry using a FITC-conjugated goat anti-human IgG secondary antibody for scFv-IgAb_387 detection. scFv-IgAb_387 demonstrated concentration-dependent binding to FOLR1 high-to-mid expressing cell lines IGROV-1, NCI-H2110, OVCAR-3, and HCC-827 with apparent affinities in the range of 1.7 nM to 3.7 nM (FIGS. 29A and 29B, Table 28). Similar results were obtained for the control molecules scFv-IgAb_162 (2.5 nM to 5.2 nM), and IgAb_335 (1.8 nM to 4.4 nM), which contain the same FOLR1 binding domain as scFv-IgAb_387. Titration of scFv-IgAb_387 on tumor cell lines with low-expressing FOLR1 levels such as SKBR-3, and A2780, yielded a concentration-dependent binding signal. However, no apparent K_{D} could be determined due to the low intensity of the fluorescence binding signal. No binding of scFv-IgAb_387 could be measured on the FOLR1-negative cell line KARPAS-299.

TABLE 28
Apparent avidities (K_{D}) determined for scFv-IgAb_387 and control
antibodies on tumor cell lines expressing different levels of FOLR1.

| | FOLR1 | scFv-IgAb_387 | | scFv-IgAb_162 | | IgAb_335 | | scFv-IgAb_444 | |
|---|---|---|---|---|---|---|---|---|---|
| | expression | K_{D} [nM] | | K_{D} [nM] | | K_{D} [nM] | | K_{D} [nM] | |
| Cell line | [SABC] | Mean | SD | Mean | SD | Mean | SD | Mean | SD |
| IGROV-1 | 209884 | 3.7 | 0.5 | 5.2 | 0.5 | 4.4 | 0.5 | n.b. | n.a. |
| NCI-H2110 | 116216 | 2.5 | 0.1 | 3.0 | 0.0 | 2.3 | 0.1 | n.b. | n.a. |
| OVCAR-3 | 34578 | 1.7 | 0.1 | 2.5 | 0.2 | 1.8 | 0.2 | n.b. | n.a. |
| HCC-827 | 24267 | 2.7 | 0.4 | 3.7 | 0.7 | 3.2 | 0.5 | n.b. | n.a. |

Apparent avidities (K_{D}) for the indicated antibody constructs were determined by staining of the tumor cell lines with serial dilutions of the antibody constructs at 37° C. and flow cytometric analysis. Specific antibody binding capacity (SABC) as a marker for FOLR1 expression level on cells was determined on all tumor cell lines using QIFIKIT.
Mean SABC, K_{D} values and standard deviations (SD) from ≥3 experiments are presented.
n.b. no binding,
n.a. not available.

### Example 24: High Avidity Interaction of FOLR1×CD16A scFv-IgAb_387 with Recombinant CD16A

The binding of FOLR1/CD16A scFv-IgAb_387 to recombinant human CD16A was assessed in ELISA.

### Methods

TABLE 28
Antibody constructs

| | Target | Target | Effector | Effector |
|---|---|---|---|---|
| Construct | specificity | domain | specificity | domain |
| scFv-IgAb_387 | FOLR1 | FOLR1_1 | CD16A | CD16a3 |
| scFv-IgAb_444 | RSV | NIST | CD16A | CD16a3 |
| | | RM8671 | | |
| scFv-IgAb_162 | FOLR1 | FOLR1_1 | RSV | NIST |
| | | | | RM8671 |
| IgAb_335 | FOLR1 | FOLR1_1 | IgG1 | Fc |

### ELISA Assay

96-well ELISA plates (F96 Maxisorp Immuno Plate, Nunc, cat: 442404) were coated overnight at 4° C. with 50 μL/well of 10 μg/mL human CD16A-mFc (158V) or human CD16A-mFc (158F) (K. Ellwanger et al. (2019) mAbs, 11:5, 899-918) in DPBS (Gibco, 14190). After overnight incubation plates were washed three times with 1×PBS/0.1% Tween20 (Sigma, P9416-100ML) and blocked with Candor Blocking (Candor, cat.: 110125) solution for 2 h at RT under mild agitation. Plates were washed again three times with PBST and subsequently incubated with serial dilutions of scFv-IgAb_387 (FOLR1×CD16A) or control antibodies (scFv-IgAb_444 (RSV(NIST)×CD16A, scFv-IgAb_162 (FOLR1×RSV(NIST), IgAb_335 (FOLR1×Farletuzumab), and the control mouse IgG1 anti-human CD16 (clone 3G8, Biolegend, cat: 302050)) in LowCross buffer (Candor, cat.: 100125). After 1 h incubation at RT under mild agitation, plates were washed five times with PBST. The plate was incubated for 1 h at RT under mild agitation with respective secondary antibodies. Anti-human Fab-HRP (Jackson Immuno, cat: 109-035-097) and anti-mouse HRP (Jackson Immuno, cat: 115-035-071) were applied at appropriate concentrations in LowCross buffer. After incubation with the secondary antibodies, the plate was washed five times with PBST and one time with PBS. Chromogenic substrate (1:1 mixture of TMB:TMBB, SeraCare cat: 5120-0048 and 5120-0037) was added and the reaction was stopped by addition of an equal volume of 0.5 M H₂SO₄ after sufficient color development. Absorbance at 450 nm was measured in an ELISA plate reader (Sunrise Absorbance Reader 901000833, Schoeller Instruments). The absorbance values were corrected by subtracting the background of the respective secondary antibody and fitted with a one site binding function (hyperbola) using GraphPad Prism (version 9.3.1. GraphPad Software, La Jolla California USA). K_{D} is the concentration of ligand required to reach half-maximal binding.

### Results

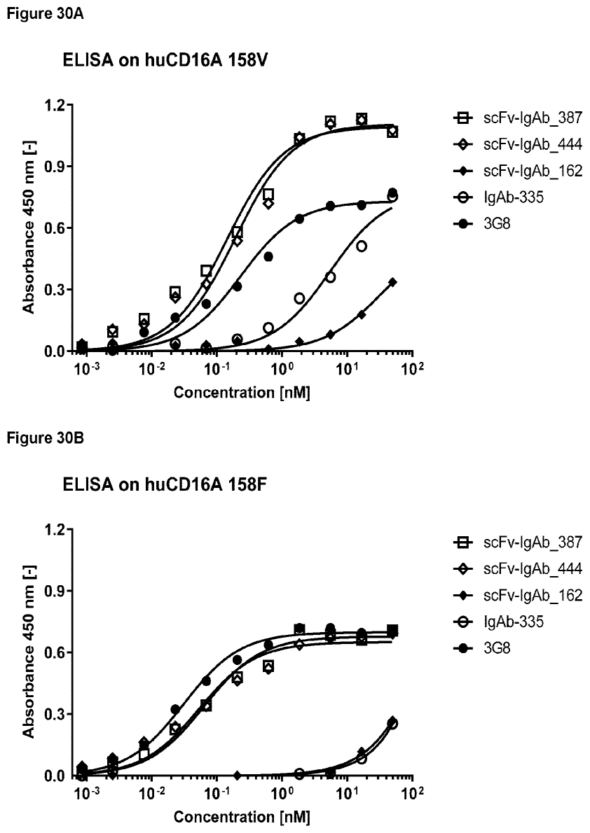

ScFv-IgAb_387 (FOLR1/CD16A) and scFv-IgAb_444 (RSV(NIST)×CD16A) harbor the same CD16A binding domain (CD16a3) and showed identical, concentration-dependent binding to human CD16A (both 158F and 158V allotypes). Apparent K_{D} for scFv-IgAb_387 was 0.09 nM (CD16A 158V) and 0.04 nM (CD16A 158F), respectively. IgAb_335 showed a weaker binding to human CD16A 158V as compared to scFv-IgAb_387 with an apparent K_{D} of 3.53 nM, and binding to human CD16A 158F was hardly detectable. scFv-IgAb_162 (FOLR1×RSV(NIST)) showed unspecific binding to CD16A (both allotypes) at higher concentrations (FIGS. 30A and 30B and Tables 29 and 30).

TABLE 29
Mean apparent avidity (K_{D}) of scFv-IgAb_387 and control antibodies
to recombinant human CD16A 158V as determined in ELISA

| | Antigen | | |
|---|---|---|---|
| | CD16A 158V (sAg_149) | | |
| | KD (nM) | | |
| Construct | mean | SD | n |
| scFv-IgAb_387 | 0.09 | 0.04 | 3 |
| scFv-IgAb_444 | 0.12 | 0.06 | 3 |
| scFv-IgAb_162 | n.a. | n.a. | 3 |
| IgAb_335 | 3.53 | 1.40 | 3 |
| anti-CD16 (3G8) | 0.11 | 0.09 | 3 |

n.a., not applicable

TABLE 30
Mean apparent avidity (K_{D}) of scFv-IgAb_387 and control antibodies
to recombinant human CD16A 158F as determined in ELISA

| | Antigen | | |
|---|---|---|---|
| | CD16A 158F (sAg_107) | | |
| | KD (nM) | | |
| Construct | mean | SD | n |
| scFv-IgAb_387 | 0.04 | 0.02 | 3 |
| scFv-IgAb_444 | 0.06 | 0.06 | 3 |
| scFv-IgAb_162 | n.a. | n.a. | 3 |
| IgAb_335 | n.a. | n.a. | 3 |
| anti-CD16 (3G8) | 0.02 | 0.01 | 3 |

n.a., not applicable

### Example 25: High Avidity Interaction of FOLR1/CD16A scFv-IgAb with Recombinant FOLR1

The binding of FOLR1×CD16A scFv-IgAb_387 to recombinant human FOLR1 was assessed in ELISA.

### Methods

TABLE 31
Antibody constructs

| | Target | Target | Effector | Effector |
|---|---|---|---|---|
| Construct | specificity | domain | specificity | domain |
| scFv-IgAb_387 | FOLR1 | FOLR1_1 | CD16A | CD16a3 |
| scFv-IgAb_444 | RSV | NIST | CD16A | CD16a3 |
| | | RM8671 | | |
| scFv-IgAb_162 | FOLR1 | FOLR1_1 | RSV | NIST |
| | | | | RM8671 |
| IgAb_335 | FOLR1 | FOLR1_1 | IgG1 | Fc |

### ELISA Assay

96 well ELISA plates (F96 Maxisorp Immuno Plate, Nunc, cat: 442404) were coated overnight at 4° C. with 50 μL/well of 10 μg/mL human FOLR1-mFc_Avi-tag in DPBS. After overnight incubation plates were washed three times with PBST and blocked with Candor Blocking solution for 2 h at RT under mild agitation. Plates were washed again three times with PBST and subsequently incubated with serial dilutions of FOLR1/CD16A scFv-IgAb_387 or control antibodies (scFv-IgAb_444 (RSV(NIST)×CD16A, scFv-IgAb_162 (FOLR1×RSV(NIST), IgAb_335 (FOLR1×Farletuzumab), and the control sheep anti-human FOLR1 antibody (R&D Systems, cat: AF5319)) in LowCross buffer. After 1 h incubation at RT under mild agitation, plates were washed five times with PBST. The plate was incubated for 1 h at RT under mild agitation with respective secondary antibodies. Anti-human Fab-HRP (Jackson Immuno, cat: 109-035-097) and anti-mouse HRP (Jackson Immuno, cat: 115-035-071) were applied at appropriate concentrations in LowCross buffer. After incubation with the secondary antibodies, the plate was washed five times with PBST and one time with PBS. Chromogenic substrate (1:1 mixture of TMB:TMBB, SeraCare cat: 5120-0048 and 5120-0037) was added and the reaction was stopped by addition of an equal volume of 0.5 M H₂SO₄ after sufficient color development. Absorbance at 450 nm was measured in an ELISA plate reader (Sunrise Absorbance Reader 901000833, Schoeller Instruments). The absorbance values were corrected by subtracting the background of the respective secondary antibody and fitted with a one site binding function (hyperbola) using GraphPad Prism (version 9.3.1. GraphPad Software, La Jolla California USA). K_{D} is the concentration of ligand required to reach half-maximal binding.

### Results

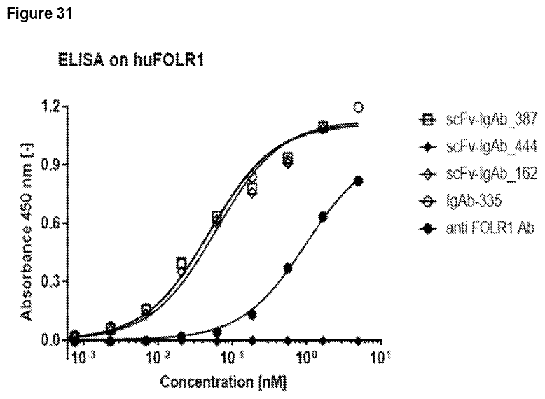

ScFv-IgAb_387 (FOLR1/CD16A), scFv-IgAb_162 (FOLR1×RSV) and IgAb_335 (FOLR1×IgG1) demonstrated dose-dependent binding to human FOLR1 with an apparent K_{D} of 0.05 nM (scFv-IgAb_387 and IgAb_335) and 0.06 nM (scFv-IgAb_162). No difference was detected since all three antibody constructs harbor the same folate receptor domain. The commercial antibody clone used as a positive control (sheep anti-human FOLR1 antibody) showed a weaker binding to recombinant human FOLR1. As expected, binding of the negative control sc-Fv-IgAb 444 (RSV×CD16A) to huFOLR1 was not detectable. (FIG. 31 and Table 32)

TABLE 32
Mean apparent avidity (K_{D}) of scFv-IgAb_387 and control
antibodies to recombinant human FOLR1 as determined in ELISA

| | Antigen | | |
|---|---|---|---|
| | human FOLR1 (sAg_303) | | |
| | KD (nM) | | |
| Construct | mean | SD | n |
| scFv-IgAb_387 | 0.05 | 0.00 | 3 |
| scFv-IgAb_444 | n.b. | n.b. | 3 |
| scFv-IgAb_162 | 0.06 | 0.01 | 3 |
| IgAb_335 | 0.05 | 0.00 | 3 |

n.b., no binding

1. A bispecific antibody construct comprising (a) a first binding domain (A), which is capable of specifically binding to a first target (A′) that is CD16A on the surface of an immune effector cell, wherein the first binding domain comprises: (i) a VL region comprising CDR-L1 as depicted in SEQ ID NO: 4, a CDR-L2 as depicted in SEQ ID NO: 5, and a CDR-L3 as depicted in SEQ ID NO: 6; and (ii) a VH region as depicted in SEQ ID NO: 7 or SEQ ID NO: 94; and (b) a second binding domain (B), which is capable of specifically binding to a second target (B′) that is an antigen on the surface of a target cell, wherein the second target (B′) is FOLR1.

2. The antibody construct of claim 1, wherein the first binding domain (A) comprises a VL region as depicted in SEQ ID NO: 8 or SEQ ID NO: 95 and a VH region as depicted in SEQ ID NO: 7 or SEQ ID NO: 94.

3. The antibody construct of claim 1 or 2, wherein the first binding domain (A) is a variable domain (Fv), a single chain Fv (scFv), a Fab, a single chain diabody (scDb), a diabody (Db) or a double Fab, preferably a scFv.

4. The antibody construct of any one of claims 1 to 3, wherein the second binding domain (B) comprises a VH and a VL domain of an antibody.

5. The antibody construct of any one of claims 1 to 4, wherein the second binding domain (B) is a variable domain (Fv), a single chain Fv (scFv), a Fab, a single chain diabody (scDb), a diabody (Db) or a double Fab, preferably a double Fab.

6. The antibody construct of any one of claims 1 to 5, wherein the antibody construct binds to a target cell and an immune effector cell simultaneously.

7. The antibody construct of any one of claims 1 to 6, wherein the first binding domain binds to an epitope on CD16A which is C-terminal to the physiological Fcγ receptor binding domain, said epitope preferably comprising Y158 of SEQ ID NO: 50.

8. The antibody construct of any one of claims 1 to 7, further comprising a third domain (C) comprising a half-life extension domain.

9. The antibody construct of any one of claims 1 to 8, wherein said half-life extension domain comprises a CH2 domain, wherein the Fc receptor binding domain is silenced.

10. The antibody construct of any one of claims 1 to 8, wherein said half-life extension domain comprises a CH3 domain.

11. The antibody construct of any one of claims 1 to 10, wherein the antibody construct comprise at least one hinge domain and a CH3 domain fused to a CH2 domain in an amino to carboxyl order in the order hinge domain-CH2 domain-CH3 domain.

12. The antibody construct of any one of claims 1 to 11, wherein the antibody construct comprises at least two of the hinge domain-CH2 domain-CH3 domain elements.

13. The antibody construct of any one of claims 1 to 12, wherein the first binding domain (A) is fused to the C terminus of a CH3 domain and the second binding domain (B) is fused to the N terminus of a hinge region.

14. The antibody construct of any one of claims 1 to 13, wherein the antibody construct is monovalent for the first binding domain (A) and monovalent for the second binding domain (B).

15. The antibody construct of any one of claims 1 to 14, wherein the antibody construct is bivalent for the first binding domain (A) and bivalent for the second binding domain (B).

16. The antibody construct of any one of claims 1 to 13 or 15, wherein (a) the first binding domain (A), which is capable of specifically binding to a first target (A′) that is CD16A on the surface of an immune effector cell comprises: (i) a VL region comprising CDR-L1 as depicted in SEQ ID NO: 4, a CDR-L2 as depicted in SEQ ID NO: 5, and a CDR-L3 as depicted in SEQ ID NO: 6, and (ii) a VH region as depicted in SEQ ID NO: 7 or SEQ ID NO: 94, wherein said first binding domain is a scFv; (b) the second binding domain which is capable of specifically binding to a second target (B′) that is an antigen on the surface of a target cell that is FOLR1, comprises: (i) a VL region comprising a CDR-L1 as depicted in SEQ ID NO: 117, a CDR-L2 as depicted in SEQ ID NO: 118, a CDR-L3 as depicted in SEQ ID NO: 119, and (ii) a VH region comprising a CDR-H1 as depicted in SEQ ID NO: 114, a CDR-H2 as depicted in SEQ ID NO: 115, a CDR-H3 as depicted in SEQ ID NO: 116, wherein said second binding domain is a Fab; and (c) the third binding domain comprises two of the hinge domain-CH2 domain-CH3 domain elements, preferably as depicted in SEQ ID NOs: 53 and 67; wherein the first binding domain (A) is fused to the C terminus of a CH3 domain of the third domain and the second binding domain (B) is fused to the N terminus of a hinge region of the third domain.

17. The antibody construct of any one of claims 1 to 13 or 15, wherein (a) the first binding domain (A), which is capable of specifically binding to a first target (A′) that is CD16A on the surface of an immune effector cell comprises: (i) a VL region comprising CDR-L1 as depicted in SEQ ID NO: 4, a CDR-L2 as depicted in SEQ ID NO: 5, and a CDR-L3 as depicted in SEQ ID NO: 6, and (ii) a VH region as depicted in SEQ ID NO: 7 or SEQ ID NO: 94, wherein said first binding domain is a scFv; (b) the second binding domain which is capable of specifically binding to a second target (B′) that is an antigen on the surface of a target cell that is FOLR1, comprises: (i) a VL region comprising a CDR-L1 as depicted in SEQ ID NO: 127, a CDR-L2 as depicted in SEQ ID NO: 128, and a CDR-L3 as depicted in SEQ ID NO: 129, and (ii) a VH region comprising a CDR-H1 as depicted in SEQ ID NO: 124, a CDR-H2 as depicted in SEQ ID NO: 125, a CDR-H3 as depicted in SEQ ID NO: 126, wherein said second binding domain is a Fab; and (c) the third binding domain comprises two of the hinge domain-CH2 domain-CH3 domain elements, preferably as depicted in SEQ ID NOs: 53 and 67; wherein the first binding domain (A) is fused to the C terminus of a CH3 domain of the third domain and the second binding domain (B) is fused to the N terminus of a hinge region of the third domain.

18. The antibody construct of any one of claims 1 to 13, 15 or 16, wherein (a) the first binding domain (A), which is capable of specifically binding to a first target (A′) that is CD16A on the surface of an immune effector cell comprises (i) a VL region as depicted in SEQ ID NO: 8 or SEQ ID NO: 95, and (ii) a VH region as depicted in SEQ ID NO: 7 or SEQ ID NO: 94, wherein said first binding domain is a scFv; (b) the second binding domain which is capable of specifically binding to a second target (B′) that is FOLR1 on the surface of a target cell comprises: (i) a VL region as depicted in SEQ ID NO: 121 and (ii) a VH region as depicted in SEQ ID NO: 120, wherein said second binding domain is a Fab; and (c) the third binding domain comprises two of the hinge domain-CH2 domain-CH3 domain elements, preferably as depicted in SEQ ID NOs: 53 and 67; wherein the first binding domain (A) is fused to the C terminus of a CH3 domain of the third domain and the second binding domain (B) is fused to the N terminus of a hinge region of the third domain.

19. The antibody construct of any one of claims 1 to 13, 15 or 17, wherein (a) the first binding domain (A), which is capable of specifically binding to a first target (A′) that is CD16A on the surface of an immune effector cell comprises (i) a VL region as depicted in SEQ ID NO: 8 or SEQ ID NO: 95, and (ii) a VH region as depicted in SEQ ID NO: 7 or SEQ ID NO: 94, wherein said first binding domain is a scFv; (b) the second binding domain which is capable of specifically binding to a second target (B′) that is FOLR1 on the surface of a target cell comprises: (i) a VL region as depicted in SEQ ID NO: 131 and (ii) a VH region as depicted in SEQ ID NO: 130, wherein said second binding domain is a Fab; and (c) the third binding domain comprises two of the hinge domain-CH2 domain-CH3 domain elements, preferably as depicted in SEQ ID NOs: 53 and 67; wherein the first binding domain (A) is fused to the C terminus of a CH3 domain of the third domain and the second binding domain (B) is fused to the N terminus of a hinge region of the third domain.

20. The antibody construct of any one of claims 1 to 17, wherein the second binding domain (B) specific for FOLR1 comprises a pair of VH- and VL-chains having a sequence as depicted in the pair of sequences having at least 85%, 90%, 95%, 96%, 97%, 98%, 99%, or 100% identity each to SEQ ID NOs: 120 and 121, respectively, or having at least 85%, 90%, 95%, 96%, 97%, 98%, 99%, or 100% identity each to SEQ ID NOs: 130 and 131, respectively.

21. The antibody construct of any one of claims 1 to 13, 15, 16 or 18, having an amino acid sequence selected from the group consisting of SEQ ID NOs: 142-143 and 138-139, wherein SEQ ID NOs: 142-143 are preferred.

22. The antibody construct of any one of claims 1 to 20, wherein the antibody construct induces less CD16A shedding as compared to a control construct having an amino acid sequence selected from the group consisting of SEQ ID NOs: 134-135, 136-137 and 140-142.

23. A nucleic acid molecule comprising a sequence encoding an antibody construct of any one of claims 1 to 22.

24. A vector comprising a nucleic acid molecule of claim 23.

25. A host cell comprising a nucleic acid molecule of claim 23 or a vector of claim 24.

26. A method of producing an antibody construct of any one of claims 1 to 22, said method comprising culturing a host cell of claim 25 under conditions allowing the expression of the antibody construct of any one of claims 1 to 22 and recovering the produced antibody construct from the culture.

27. A pharmaceutical composition comprising an antibody construct of any one of claims 1 to 22, or produced by the method of claim 26.

28. The antibody construct of any one of claims 1 to 22 for use in therapy.

29. The antibody construct of any one of claims 1 to 22, or produced by the method of claim 26, for use in the prevention, treatment or amelioration of a disease selected from a proliferative disease, a tumorous disease, a viral disease or an immunological disorder.

30. The antibody construct for use of claim 29, wherein said tumorous disease is a solid tumor, preferably a malignant solid tumor.

31. The antibody construct for use of claim 30, wherein said solid tumor is selected from the group consisting of ovarian (optionally high grade serous ovarian cancer or epithelial ovarian cancer), breast (optionally TNBC), renal, lung (optionally non small cell lung cancer (NSCLC) or mesotheliomas), colorectal (optionally CRC), kidney (optionally ccRCC), pancreatic (optionally PDAC), endometrial (optionally non-malignant endometrial cancer) and brain cancer.

32. A method of treatment or amelioration of a proliferative disease, a tumorous disease, a viral disease or an immunological disorder, comprising the step of administering to a subject in need thereof the antibody construct of any one of claims 1 to 22, or produced by the method of claim 26.

33. A kit comprising an antibody construct of any one of claims 1 to 22, or produced by the method of claim 26, a nucleic acid of claim 23, a vector of claim 24, and/or a host cell of claim 25.


SEQUENCE LISTING (163 sequences)

SEQ ID NO: 1 - protein, 5 aa, synthetic construct
  source 1..5: note = antibody CDR-H1 sequence
NYYMQ

SEQ ID NO: 2 - protein, 17 aa, synthetic construct
  source 1..17: note = antibody CDR-H2 sequence
IINPSGGVTSYAQKFQG

SEQ ID NO: 3 - protein, 11 aa, synthetic construct
  source 1..11: note = antibody CDR-H3 sequence
GSAYYYDFADY

SEQ ID NO: 4 - protein, 11 aa, synthetic construct
  source 1..11: note = antibody CDR-L1 sequence
GGNNIGSKSVH

SEQ ID NO: 5 - protein, 7 aa, synthetic construct
  source 1..7: note = antibody CDR-L2 sequence
QDKKRPS

SEQ ID NO: 6 - protein, 9 aa, synthetic construct
  source 1..9: note = antibody CDR-L3 sequence
QVWDDYIVL

SEQ ID NO: 7 - protein, 120 aa, synthetic construct
  source 1..120: note = antibody VH domain
QVQLVQSGAEVKKPGASVKVSCKASGYTFTNYYMQWVRQAPGQCLEWMGIINPSGGVTSYAQKFQGRVTMTRDTSTSTVYMELSSLRSEDTAVYYCARGSAYYYDFADYWGQGTLVTVSS

SEQ ID NO: 8 - protein, 106 aa, synthetic construct
  source 1..106: note = antibody VL domain
SYELTQPLSVSVALGQTARITCGGNNIGSKSVHWYQQKPGQAPVLVIYQDKKRPSGIPERFSGSNSGNTATLTISRAQAGDEADYYCQVWDDYIVLFGCGTKLTVL

SEQ ID NO: 9 - protein, 247 aa, synthetic construct
  source 1..247: note = antibody scFv
SYELTQPLSVSVALGQTARITCGGNNIGSKSVHWYQQKPGQAPVLVIYQDKKRPSGIPERFSGSNSGNTATLTISRAQAGDEADYYCQVWDDYIVLFGCGTKLTVLGGSGGSGGSGGSGGSGGSGGSQVQLVQSGAEVKKPGASVKVSCKASGYTFTNYYMQWVRQAPGQCLEWMGIINPSGGVTSYAQKFQGRVTMTRDTSTSTVYMELSSLRSEDTAVYYCARGSAYYYDFADYWGQGTLVTVSS

SEQ ID NO: 10 - protein, 482 aa, synthetic construct
  source 1..482: note = antibody scDb
SYELTQPLSVSVALGQTARITCGGNNIGSKSVHWYQQKPGQAPVLVIYQDKKRPSGIPERFSGSNSGNTATLTISRAQAGDEADYYCQVWDDYIVLFGCGTKLTVLGGSGGSQVQLVQSGAEVKKPGASVKVSCKASGYTFTNYYMQWVRQAPGQCLEWMGIINPSGGVTSYAQKFQGRVTMTRDTSTSTVYMELSSLRSEDTAVYYCARGSAYYYDFADYWGQGTLVTVSSGGSGGSGGSGGSGGSGGSSYELTQPLSVSVALGQTARITCGGNNIGSKSVHWYQQKPGQAPVLVIYQDKKRPSGIPERFSGSNSGNTATLTISRAQAGDEADYYCQVWDDYIVLFGCGTKLTVLGGSGGSQVQLVQSGAEVKKPGASVKVSCKASGYTFTNYYMQWVRQAPGQCLEWMGIINPSGGVTSYAQKFQGRVTMTRDTSTSTVYMELSSLRSEDTAVYYCARGSAYYYDFADYWGQGTLVTVSS

SEQ ID NO: 11 - protein, 5 aa, synthetic construct
  source 1..5: note = antibody CDR-H1 sequence
SYYMH

SEQ ID NO: 12 - protein, 17 aa, synthetic construct
  source 1..17: note = antibody CDR-H2 sequence
AIEPRYGSTSYAQKFQG

SEQ ID NO: 13 - protein, 11 aa, synthetic construct
  source 1..11: note = antibody CDR-H3 sequence
GSAYYYDFADY

SEQ ID NO: 14 - protein, 11 aa, synthetic construct
  source 1..11: note = antibody CDR-L1 sequence
GGHNIGSKNVH

SEQ ID NO: 15 - protein, 7 aa, synthetic construct
  source 1..7: note = antibody CDR-L2 sequence
QDNKRPS

SEQ ID NO: 16 - protein, 9 aa, synthetic construct
  source 1..9: note = antibody CDR-L3 sequence
QVWDNYNVL

SEQ ID NO: 17 - protein, 120 aa, synthetic construct
  source 1..120: note = antibody VH domain
QVQLVQSGAEVKKPGASVKVSCKASGYTFTSYYMHWVRQAPGQCLEWMGAIEPRYGSTSYAQKFQGRVTMTRDTSTSTVYMELSSLRSEDTAVYYCARGSAYYYDFADYWGQGTLVTVSS

SEQ ID NO: 18 - protein, 106 aa, synthetic construct
  source 1..106: note = antibody VL domain
SYELTQPLSVSVALGQTARITCGGHNIGSKNVHWYQQKPGQAPVLVIYQDNKRPSGIPERFSGSNSGNTATLTISRAQAGDEADYYCQVWDNYNVLFGCGTKLTVL

SEQ ID NO: 19 - protein, 247 aa, synthetic construct
  source 1..247: note = antibody scFv
SYELTQPLSVSVALGQTARITCGGHNIGSKNVHWYQQKPGQAPVLVIYQDNKRPSGIPERFSGSNSGNTATLTISRAQAGDEADYYCQVWDNYNVLFGCGTKLTVLGGSGGSGGSGGSGGSGGSGGSQVQLVQSGAEVKKPGASVKVSCKASGYTFTSYYMHWVRQAPGQCLEWMGAIEPRYGSTSYAQKFQGRVTMTRDTSTSTVYMELSSLRSEDTAVYYCARGSAYYYDFADYWGQGTLVTVSS

SEQ ID NO: 20 - protein, 482 aa, synthetic construct
  source 1..482: note = antibody scDb
SYELTQPLSVSVALGQTARITCGGHNIGSKNVHWYQQKPGQAPVLVIYQDNKRPSGIPERFSGSNSGNTATLTISRAQAGDEADYYCQVWDNYNVLFGCGTKLTVLGGSGGSQVQLVQSGAEVKKPGASVKVSCKASGYTFTSYYMHWVRQAPGQCLEWMGAIEPRYGSTSYAQKFQGRVTMTRDTSTSTVYMELSSLRSEDTAVYYCARGSAYYYDFADYWGQGTLVTVSSGGSGGSGGSGGSGGSGGSSYELTQPLSVSVALGQTARITCGGHNIGSKNVHWYQQKPGQAPVLVIYQDNKRPSGIPERFSGSNSGNTATLTISRAQAGDEADYYCQVWDNYNVLFGCGTKLTVLGGSGGSQVQLVQSGAEVKKPGASVKVSCKASGYTFTSYYMHWVRQAPGQCLEWMGAIEPRYGSTSYAQKFQGRVTMTRDTSTSTVYMELSSLRSEDTAVYYCARGSAYYYDFADYWGQGTLVTVSS

SEQ ID NO: 21 - protein, 5 aa, synthetic construct
  source 1..5: note = antibody CDR-H1 sequence
DYYMK

SEQ ID NO: 22 - protein, 17 aa, synthetic construct
  source 1..17: note = antibody CDR-H2 sequence
DIIPSNGATFYNQKFKG

SEQ ID NO: 23 - protein, 11 aa, synthetic construct
  source 1..11: note = antibody CDR-H3 sequence
SHLLRASWFAY

SEQ ID NO: 24 - protein, 17 aa, synthetic construct
  source 1..17: note = antibody CDR-L1 sequence
KSSQSLLNTGNQKNYLT

SEQ ID NO: 25 - protein, 7 aa, synthetic construct
  source 1..7: note = antibody CDR-L2 sequence
WASTRES

SEQ ID NO: 26 - protein, 9 aa, synthetic construct
  source 1..9: note = antibody CDR-L3 sequence
QNDYSYPYT

SEQ ID NO: 27 - protein, 120 aa, synthetic construct
  source 1..120: note = antibody VH domain
QVQLQQSGAEVKKPGASVKVSCKASGYTFTDYYMKWVKQSHGKSLEWMGDIIPSNGATFYNQKFKGKATLTVDRSTSTAYMELSSLRSEDTAVYYCARSHLLRASWFAYWGQGTLVTVSS

SEQ ID NO: 28 - protein, 113 aa, synthetic construct
  source 1..113: note = antibody VL domain
DFVMTQSPDSLAVSLGERATINCKSSQSLLNTGNQKNYLTWYQQKPGQPPKLLIYWASTRESGVPDRFTGSGSGTDFTLTISSLQAEDVAVYYCQNDYSYPYTFGGGTKLEIK

SEQ ID NO: 29 - protein, 251 aa, synthetic construct
  source 1..251: note = antibody scFv
QVQLQQSGAEVKKPGASVKVSCKASGYTFTDYYMKWVKQSHGKSLEWMGDIIPSNGATFYNQKFKGKATLTVDRSTSTAYMELSSLRSEDTAVYYCARSHLLRASWFAYWGQGTLVTVSSGGSGGSGGSGGSGGSGGSDFVMTQSPDSLAVSLGERATINCKSSQSLLNTGNQKNYLTWYQQKPGQPPKLLIYWASTRESGVPDRFTGSGSGTDFTLTISSLQAEDVAVYYCQNDYSYPYTFGGGTKLEIK

SEQ ID NO: 30 - protein, 499 aa, synthetic construct
  source 1..499: note = antibody scDb
QVQLQQSGAEVKKPGASVKVSCKASGYTFTDYYMKWVKQSHGKSLEWMGDIIPSNGATFYNQKFKGKATLTVDRSTSTAYMELSSLRSEDTAVYYCARSHLLRASWFAYWGQGTLVTVSSGGSGGSDFVMTQSPDSLAVSLGERATINCKSSQSLLNTGNQKNYLTWYQQKPGQPPKLLIYWASTRESGVPDRFTGSGSGTDFTLTISSLQAEDVAVYYCQNDYSYPYTFGGGTKLEIKGGSGGSGGSGGSGGSGGSGGSQVQLQQSGAEVKKPGASVKVSCKASGYTFTDYYMKWVKQSHGKSLEWMGDIIPSNGATFYNQKFKGKATLTVDRSTSTAYMELSSLRSEDTAVYYCARSHLLRASWFAYWGQGTLVTVSSGGSGGSDFVMTQSPDSLAVSLGERATINCKSSQSLLNTGNQKNYLTWYQQKPGQPPKLLIYWASTRESGVPDRFTGSGSGTDFTLTISSLQAEDVAVYYCQNDYSYPYTFGGGTKLEIK

SEQ ID NO: 31 - protein, 5 aa, synthetic construct
  source 1..5: note = antibody CDR-H1 sequence
DYYMK

SEQ ID NO: 32 - protein, 17 aa, synthetic construct
  source 1..17: note = antibody CDR-H2 sequence
DIIPSNGATFYNQKFKG

SEQ ID NO: 33 - protein, 11 aa, synthetic construct
  source 1..11: note = antibody CDR-H3 sequence
SHLLRASWFAY

SEQ ID NO: 34 - protein, 17 aa, synthetic construct
  source 1..17: note = antibody CDR-L1 sequence
KSSQSLLNSGNQKNYLT

SEQ ID NO: 35 - protein, 7 aa, synthetic construct
  source 1..7: note = antibody CDR-L2 sequence
WASTRES

SEQ ID NO: 36 - protein, 9 aa, synthetic construct
  source 1..9: note = antibody CDR-L3 sequence
QNDYSYPYT

SEQ ID NO: 37 - protein, 120 aa, synthetic construct
  source 1..120: note = antibody VH domain
QVQLVQSGAEVKKPGASVKMSCKASGYTFTDYYMKWVKQAPGQGLEWIGDIIPSNGATFYNQKFKGKATLTVDRSISTAYMHLNRLRSDDTAVYYCTRSHLLRASWFAYWGQGTLVTVSS

SEQ ID NO: 38 - protein, 113 aa, synthetic construct
  source 1..113: note = antibody VL domain
DFVMTQSPDSLAVSLGERATINCKSSQSLLNSGNQKNYLTWYLQKPGQPPKLLIYWASTRESGVPDRFSGSGSGTDFTLTISSLQAEDVAVYYCQNDYSYPYTFGQGTKLEIK

SEQ ID NO: 39 - protein, 251 aa, synthetic construct
  source 1..251: note = antibody scFv
QVQLVQSGAEVKKPGASVKMSCKASGYTFTDYYMKWVKQAPGQGLEWIGDIIPSNGATFYNQKFKGKATLTVDRSISTAYMHLNRLRSDDTAVYYCTRSHLLRASWFAYWGQGTLVTVSSGGSGGSGGSGGSGGSGGSDFVMTQSPDSLAVSLGERATINCKSSQSLLNSGNQKNYLTWYLQKPGQPPKLLIYWASTRESGVPDRFSGSGSGTDFTLTISSLQAEDVAVYYCQNDYSYPYTFGQGTKLEIK

SEQ ID NO: 40 - protein, 499 aa, synthetic construct
  source 1..499: note = antibody scDb
QVQLVQSGAEVKKPGASVKMSCKASGYTFTDYYMKWVKQAPGQGLEWIGDIIPSNGATFYNQKFKGKATLTVDRSISTAYMHLNRLRSDDTAVYYCTRSHLLRASWFAYWGQGTLVTVSSGGSGGSDFVMTQSPDSLAVSLGERATINCKSSQSLLNSGNQKNYLTWYLQKPGQPPKLLIYWASTRESGVPDRFSGSGSGTDFTLTISSLQAEDVAVYYCQNDYSYPYTFGQGTKLEIKGGSGGSGGSGGSGGSGGSGGSQVQLVQSGAEVKKPGASVKMSCKASGYTFTDYYMKWVKQAPGQGLEWIGDIIPSNGATFYNQKFKGKATLTVDRSISTAYMHLNRLRSDDTAVYYCTRSHLLRASWFAYWGQGTLVTVSSGGSGGSDFVMTQSPDSLAVSLGERATINCKSSQSLLNSGNQKNYLTWYLQKPGQPPKLLIYWASTRESGVPDRFSGSGSGTDFTLTISSLQAEDVAVYYCQNDYSYPYTFGQGTKLEIK

SEQ ID NO: 41 - protein, 4 aa, synthetic construct
  source 1..4: note = linker
GGGS

SEQ ID NO: 42 - protein, 6 aa, synthetic construct
  source 1..6: note = linker
GGSGGS

SEQ ID NO: 43 - protein, 9 aa, synthetic construct
  source 1..9: note = linker
GGSGGSGGS

SEQ ID NO: 44 - protein, 18 aa, synthetic construct
  source 1..18: note = linker
GGSGGSGGSGGSGGSGGS

SEQ ID NO: 45 - protein, 21 aa, synthetic construct
  source 1..21: note = linker
GGSGGSGGSGGSGGSGGSGGS

SEQ ID NO: 46 - protein, 5 aa, synthetic construct
  source 1..5: note = linker
GGGGS

SEQ ID NO: 47 - protein, 10 aa, synthetic construct
  source 1..10: note = linker
GGGGSGGGGS

SEQ ID NO: 48 - protein, 20 aa, synthetic construct
  source 1..20: note = linker
GGGGSGGGGSGGGGSGGGGS

SEQ ID NO: 49 - protein, 30 aa, synthetic construct
  source 1..30: note = linker
GGGGSGGGGSGGGGSGGGGSGGGGSGGGGS

SEQ ID NO: 50 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 51 - protein, 254 aa, Macaca fascicularis
MWQLLLPTALLLLVSAGMRAEDLPKAVVFLEPQWYRVLEKDRVTLKCQGAYSPEDNSTRWFHNESLISSQTSSYFIAAARVNNSGEYRCQTSLSTLSDPVQLEVHIGWLLLQAPRWVFKEEESIHLRCHSWKNTLLHKVTYLQNGKGRKYFHQNSDFYIPKATLKDSGSYFCRGLIGSKNVSSETVNITITQDLAVSSISSFFPPGYQVSFCLVMVLLFAVDTGLYFSMKKSIPSSTRDWEDHKFKWSKDPQDK

SEQ ID NO: 52 - protein, 233 aa, Homo sapiens
MWQLLLPTALLLLVSAGMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 53 - protein, 15 aa, Homo sapiens
EPKSCDKTHTCPPCP

SEQ ID NO: 54 - protein, 10 aa, Homo sapiens
EPKSCDKTHT

SEQ ID NO: 55 - protein, 10 aa, Homo sapiens
DKTHTCPPCP

SEQ ID NO: 56 - protein, 12 aa, Homo sapiens
ERKCCVECPPCP

SEQ ID NO: 57 - protein, 16 aa, Homo sapiens
ELKTPLDTTHTCPRCP

SEQ ID NO: 58 - protein, 17 aa, Homo sapiens
ELKTPLGDTTHTCPRCP

SEQ ID NO: 59 - protein, 12 aa, Homo sapiens
ESKYGPPCPSCP

SEQ ID NO: 60 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 61 - protein, 329 aa, synthetic construct
  source 1..329: note = artificial heavy chain constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 62 - protein, 329 aa, synthetic construct
  source 1..329: note = artificial heavy chain constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 63 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 64 - protein, 107 aa, Homo sapiens [sequence omitted]

SEQ ID NO: 65 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 66 - protein, 216 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 67 - protein, 216 aa, synthetic construct
  source 1..216: note = artificial heavy chain constant domain
APEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 68 - protein, 216 aa, synthetic construct
  source 1..216: note = artificial heavy chain constant domain
APEFEGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 69 - protein, 216 aa, synthetic construct
  source 1..216: note = artificial heavy chain constant domain
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 70 - protein, 216 aa, synthetic construct
  source 1..216: note = artificial heavy chain constant domain
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPG

SEQ ID NO: 71 - protein, 216 aa, synthetic construct
  source 1..216: note = artificial heavy chain constant domain
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPG

SEQ ID NO: 72 - protein, 216 aa, synthetic construct
  source 1..216: note = artificial heavy chain constant domain
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 73 - protein, 216 aa, synthetic construct
  source 1..216: note = artificial heavy chain constant domain
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - protein, 216 aa, synthetic construct
  source 1..216: note = artificial heavy chain constant domain
APEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPG

SEQ ID NO: 75 - protein, 216 aa, synthetic construct
  source 1..216: note = artificial heavy chain constant domain
APEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPG

SEQ ID NO: 76 - protein, 216 aa, synthetic construct
  source 1..216: note = artificial heavy chain constant domain
APEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 77 - protein, 216 aa, synthetic construct
  source 1..216: note = artificial heavy chain constant domain
APEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 78 - protein, 216 aa, synthetic construct
  source 1..216: note = artificial heavy chain constant domain
APEFEGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPG

SEQ ID NO: 79 - protein, 216 aa, synthetic construct
  source 1..216: note = artificial heavy chain constant domain
APEFEGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPG

SEQ ID NO: 80 - protein, 216 aa, synthetic construct
  source 1..216: note = artificial heavy chain constant domain
APEFEGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 81 - protein, 216 aa, synthetic construct
  source 1..216: note = artificial heavy chain constant domain
APEFEGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 82 - protein, 726 aa, synthetic construct
  source 1..726: note = artificial antibody construct heavy chain
QVQLVQSGAEVKKPGASVKMSCKASGYTFTDYYMKWVKQAPGQGLEWIGDIIPSNGATFYNQKFKGKATLTVDRSISTAYMHLNRLRSDDTAVYYCTRSHLLRASWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSSYELTQPLSVSVALGQTARITCGGHNIGSKNVHWYQQKPGQAPVLVIYQDNKRPSGIPERFSGSNSGNTATLTISRAQAGDEADYYCQVWDNYNVLFGCGTKLTVLGGSGGSGGSGGSGGSGGSGGSQVQLVQSGAEVKKPGASVKVSCKASGYTFTSYYMHWVRQAPGQCLEWMGAIEPRYGSTSYAQKFQGRVTMTRDTSTSTVYMELSSLRSEDTAVYYCARGSAYYYDFADYWGQGTLVTVSS

SEQ ID NO: 83 - protein, 220 aa, synthetic construct
  source 1..220: note = artificial antibody construct light chain
DFVMTQSPDSLAVSLGERATINCKSSQSLLNSGNQKNYLTWYLQKPGQPPKLLIYWASTRESGVPDRFSGSGSGTDFTLTISSLQAEDVAVYYCQNDYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 726 aa, synthetic construct
  source 1..726: note = artificial antibody construct heavy chain
QVQLQQSGAEVKKPGASVKVSCKASGYTFTDYYMKWVKQSHGKSLEWMGDIIPSNGATFYNQKFKGKATLTVDRSTSTAYMELSSLRSEDTAVYYCARSHLLRASWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSSYELTQPLSVSVALGQTARITCGGHNIGSKNVHWYQQKPGQAPVLVIYQDNKRPSGIPERFSGSNSGNTATLTISRAQAGDEADYYCQVWDNYNVLFGCGTKLTVLGGSGGSGGSGGSGGSGGSGGSQVQLVQSGAEVKKPGASVKVSCKASGYTFTSYYMHWVRQAPGQCLEWMGAIEPRYGSTSYAQKFQGRVTMTRDTSTSTVYMELSSLRSEDTAVYYCARGSAYYYDFADYWGQGTLVTVSS

SEQ ID NO: 85 - protein, 220 aa, synthetic construct
  source 1..220: note = artificial antibody construct light chain
DFVMTQSPDSLAVSLGERATINCKSSQSLLNTGNQKNYLTWYQQKPGQPPKLLIYWASTRESGVPDRFTGSGSGTDFTLTISSLQAEDVAVYYCQNDYSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 726 aa, synthetic construct
  source 1..726: note = artificial antibody construct heavy chain
QVQLVQSGAEVKKPGASVKMSCKASGYTFTDYYMKWVKQAPGQGLEWIGDIIPSNGATFYNQKFKGKATLTVDRSISTAYMHLNRLRSDDTAVYYCTRSHLLRASWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSSYELTQPLSVSVALGQTARITCGGNNIGSKSVHWYQQKPGQAPVLVIYQDKKRPSGIPERFSGSNSGNTATLTISRAQAGDEADYYCQVWDDYIVLFGCGTKLTVLGGSGGSGGSGGSGGSGGSGGSQVQLVQSGAEVKKPGASVKVSCKASGYTFTNYYMQWVRQAPGQCLEWMGIINPSGGVTSYAQKFQGRVTMTRDTSTSTVYMELSSLRSEDTAVYYCARGSAYYYDFADYWGQGTLVTVSS

SEQ ID NO: 87 - protein, 220 aa, synthetic construct; identical to SEQ ID NO: 83
  source 1..220: note = artificial antibody construct light chain

SEQ ID NO: 88 - protein, 726 aa, synthetic construct
  source 1..726: note = artificial antibody construct heavy chain
QVQLQQSGAEVKKPGASVKVSCKASGYTFTDYYMKWVKQSHGKSLEWMGDIIPSNGATFYNQKFKGKATLTVDRSTSTAYMELSSLRSEDTAVYYCARSHLLRASWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSSYELTQPLSVSVALGQTARITCGGNNIGSKSVHWYQQKPGQAPVLVIYQDKKRPSGIPERFSGSNSGNTATLTISRAQAGDEADYYCQVWDDYIVLFGCGTKLTVLGGSGGSGGSGGSGGSGGSGGSQVQLVQSGAEVKKPGASVKVSCKASGYTFTNYYMQWVRQAPGQCLEWMGIINPSGGVTSYAQKFQGRVTMTRDTSTSTVYMELSSLRSEDTAVYYCARGSAYYYDFADYWGQGTLVTVSS

SEQ ID NO: 89 - protein, 220 aa, synthetic construct; identical to SEQ ID NO: 85
  source 1..220: note = artificial antibody construct light chain

SEQ ID NO: 90 - protein, 449 aa, synthetic construct
  source 1..449: note = artificial antibody construct heavy chain
EVQLVQSGAEVKKPGESLKISCKGSGYSFTDYYMKWARQMPGKGLEWMGDIIPSNGATFYNQKFKGQVTISADKSISTTYLQWSSLKASDTAMYYCARSHLLRASWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 91 - protein, 220 aa, synthetic construct
  source 1..220: note = artificial antibody construct light chain
DIVMTQSPDSLAVSLGERATINCESSQSLLNSGNQKNYLTWYQQKPGQPPKPLIYWASTRESGVPDRFSGSGSGTDFTLTISSLQAEDVAVYYCQNDYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 726 aa, synthetic construct
  source 1..726: note = artificial antibody construct heavy chain
QVQLQQSGAEVKKPGASVKVSCKASGYTFTDYYMKWVKQSHGKSLEWMGDIIPSNGATFYNQKFKGKATLTVDRSTSTAYMELSSLRSEDTAVYYCARSHLLRASWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGSGGSGGSGGSGGSGGSGGSQVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSS

SEQ ID NO: 93 - protein, 220 aa, synthetic construct; identical to SEQ ID NO: 85
  source 1..220: note = artificial antibody construct light chain

SEQ ID NO: 94 - protein, 121 aa, synthetic construct
  source 1..121: note = antibody VH domain
QVQLVQSGAEVKKPGASVKVSCKASGYTFTNYYMQWVRQAPGQCLEWMGIINPSGGVTSYAQKFQGRVTMTRDTSTSTVYMELSSLRSEDTAVYYCARGSAYYYDFADYWGQGTLVTVSSG

SEQ ID NO: 95 - protein, 106 aa, synthetic construct; identical to SEQ ID NO: 8
  source 1..106: note = antibody VL domain

SEQ ID NO: 96 - protein, 248 aa, synthetic construct
  source 1..248: note = antibody scFv
SYELTQPLSVSVALGQTARITCGGNNIGSKSVHWYQQKPGQAPVLVIYQDKKRPSGIPERFSGSNSGNTATLTISRAQAGDEADYYCQVWDDYIVLFGCGTKLTVLGGSGGSGGSGGSGGSGGSGGSQVQLVQSGAEVKKPGASVKVSCKASGYTFTNYYMQWVRQAPGQCLEWMGIINPSGGVTSYAQKFQGRVTMTRDTSTSTVYMELSSLRSEDTAVYYCARGSAYYYDFADYWGQGTLVTVSSG

SEQ ID NO: 97 - protein, 483 aa, synthetic construct
  source 1..483: note = antibody scDb
SYELTQPLSVSVALGQTARITCGGNNIGSKSVHWYQQKPGQAPVLVIYQDKKRPSGIPERFSGSNSGNTATLTISRAQAGDEADYYCQVWDDYIVLFGCGTKLTVLGGSGGSQVQLVQSGAEVKKPGASVKVSCKASGYTFTNYYMQWVRQAPGQCLEWMGIINPSGGVTSYAQKFQGRVTMTRDTSTSTVYMELSSLRSEDTAVYYCARGSAYYYDFADYWGQGTLVTVSSGGSGGSGGSGGSGGSGGSSYELTQPLSVSVALGQTARITCGGNNIGSKSVHWYQQKPGQAPVLVIYQDKKRPSGIPERFSGSNSGNTATLTISRAQAGDEADYYCQVWDDYIVLFGCGTKLTVLGGSGGSQVQLVQSGAEVKKPGASVKVSCKASGYTFTNYYMQWVRQAPGQCLEWMGIINPSGGVTSYAQKFQGRVTMTRDTSTSTVYMELSSLRSEDTAVYYCARGSAYYYDFADYWGQGTLVTVSSG

SEQ ID NO: 98 - protein, 5 aa, synthetic construct
  source 1..5: note = antibody CDR-H1 sequence
NYYMQ

SEQ ID NO: 99 - protein, 17 aa, synthetic construct
  source 1..17: note = antibody CDR-H2 sequence
IINPSGGVTSYAQKFQG

SEQ ID NO: 100 - protein, 11 aa, synthetic construct
  source 1..11: note = antibody CDR-H3 sequence
GSAYYYDFADY

SEQ ID NO: 101 - protein, 11 aa, synthetic construct
  source 1..11: note = antibody CDR-L1 sequence
GGNNIGSKSVH

SEQ ID NO: 102 - protein, 7 aa, synthetic construct
  source 1..7: note = antibody CDR-L2 sequence
QDKKRPS

SEQ ID NO: 103 - protein, 9 aa, synthetic construct
  source 1..9: note = antibody CDR-L3 sequence
QVWDDYIVL

SEQ ID NO: 104 - protein, 119 aa, synthetic construct
  source 1..119: note = antibody VH domain
QVQLVQSGAEVKKPGASVKVSCKASGYTFTSYYMHWVRQAPGQCLEWMGAIEPTYGSTSYAQKFQGRVTMTRDTSTSTVYMELSSLRSEDTAVYYCARGSAYYYDFADYWGQGTLVTVS

SEQ ID NO: 105 - protein, 106 aa, synthetic construct; identical to SEQ ID NO: 18
  source 1..106: note = antibody VL domain

SEQ ID NO: 106 - protein, 246 aa, synthetic construct
  source 1..246: note = antibody scFv
SYELTQPLSVSVALGQTARITCGGHNIGSKNVHWYQQKPGQAPVLVIYQDNKRPSGIPERFSGSNSGNTATLTISRAQAGDEADYYCQVWDNYNVLFGCGTKLTVLGGSGGSGGSGGSGGSGGSGGSQVQLVQSGAEVKKPGASVKVSCKASGYTFTSYYMHWVRQAPGQCLEWMGAIEPTYGSTSYAQKFQGRVTMTRDTSTSTVYMELSSLRSEDTAVYYCARGSAYYYDFADYWGQGTLVTVS

SEQ ID NO: 107 - protein, 481 aa, synthetic construct
  source 1..481: note = antibody scDb
SYELTQPLSVSVALGQTARITCGGHNIGSKNVHWYQQKPGQAPVLVIYQDNKRPSGIPERFSGSNSGNTATLTISRAQAGDEADYYCQVWDNYNVLFGCGTKLTVLGGSGGSQVQLVQSGAEVKKPGASVKVSCKASGYTFTSYYMHWVRQAPGQCLEWMGAIEPTYGSTSYAQKFQGRVTMTRDTSTSTVYMELSSLRSEDTAVYYCARGSAYYYDFADYWGQGTLVTVSSGGSGGSGGSGGSGGSGGSSYELTQPLSVSVALGQTARITCGGHNIGSKNVHWYQQKPGQAPVLVIYQDNKRPSGIPERFSGSNSGNTATLTISRAQAGDEADYYCQVWDNYNVLFGCGTKLTVLGGSGGSQVQLVQSGAEVKKPGASVKVSCKASGYTFTSYYMHWVRQAPGQCLEWMGAIEPTYGSTSYAQKFQGRVTMTRDTSTSTVYMELSSLRSEDTAVYYCARGSAYYYDFADYWGQGTLVTVS

SEQ ID NO: 108 - protein, 5 aa, synthetic construct
  source 1..5: note = antibody CDR-H1 sequence
SYYMH

SEQ ID NO: 109 - protein, 17 aa, synthetic construct
  source 1..17: note = antibody CDR-H2 sequence
AIEPRYGSTSYAQKFQG

SEQ ID NO: 110 - protein, 11 aa, synthetic construct
  source 1..11: note = antibody CDR-H3 sequence
GSAYYYDFADY

SEQ ID NO: 111 - protein, 11 aa, synthetic construct
  source 1..11: note = antibody CDR-L1 sequence
GGHNIGSKNVH

SEQ ID NO: 112 - protein, 7 aa, synthetic construct
  source 1..7: note = antibody CDR-L2 sequence
QDNKRPS

SEQ ID NO: 113 - protein, 9 aa, synthetic construct
  source 1..9: note = antibody CDR-L3 sequence
QVWDNYNVL

SEQ ID NO: 114 - protein, 5 aa, synthetic construct
  source 1..5: note = antibody CDR-H1 sequence
GYGLS

SEQ ID NO: 115 - protein, 17 aa, synthetic construct
  source 1..17: note = antibody CDR-H2 sequence
MISSGGSYTYYADSVKG

SEQ ID NO: 116 - protein, 10 aa, synthetic construct
  source 1..10: note = antibody CDR-H3 sequence
HGDDPAWFAY

SEQ ID NO: 117 - protein, 12 aa, synthetic construct
  source 1..12: note = antibody CDR-L1 sequence
SVSSSISSNNLH

SEQ ID NO: 118 - protein, 7 aa, synthetic construct
  source 1..7: note = antibody CDR-L2 sequence
GTSNLAS

SEQ ID NO: 119 - protein, 11 aa, synthetic construct
  source 1..11: note = antibody CDR-L3 sequence
QQWSSYPYMYT

SEQ ID NO: 120 - protein, 119 aa, synthetic construct
  source 1..119: note = antibody VH domain
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSS

SEQ ID NO: 121 - protein, 110 aa, synthetic construct
  source 1..110: note = antibody VL domain
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIK

SEQ ID NO: 122 - protein, 247 aa, synthetic construct
  source 1..247: note = antibody scFv
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSGGSGGSGGSGGSGGSGGSDIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIK

SEQ ID NO: 123 - protein, 491 aa, synthetic construct
  source 1..491: note = antibody scDb
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSGGSGGSDIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKGGSGGSGGSGGSGGSGGSGGSEVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSGGSGGSDIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIK

SEQ ID NO: 124 - protein, 5 aa, synthetic construct
  source 1..5: note = antibody CDR-H1 sequence
GYFMN

SEQ ID NO: 125 - protein, 17 aa, synthetic construct
  source 1..17: note = antibody CDR-H2 sequence
RIHPYDGDTFYNQKFQG

SEQ ID NO: 126 - protein, 9 aa, synthetic construct
  source 1..9: note = antibody CDR-H3 sequence
YDGSRAMDY

SEQ ID NO: 127 - protein, 15 aa, synthetic construct
  source 1..15: note = antibody CDR-L1 sequence
KASQSVSFAGTSLMH

SEQ ID NO: 128 - protein, 7 aa, synthetic construct
  source 1..7: note = antibody CDR-L2 sequence
RASNLEA

SEQ ID NO: 129 - protein, 9 aa, synthetic construct
  source 1..9: note = antibody CDR-L3 sequence
QQSREYPYT

SEQ ID NO: 130 - protein, 118 aa, synthetic construct
  source 1..118: note = antibody VH domain
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSS

SEQ ID NO: 131 - protein, 111 aa, synthetic construct
  source 1..111: note = antibody VL domain
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIK

SEQ ID NO: 132 - protein, 247 aa, synthetic construct
  source 1..247: note = antibody scFv
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSSGGSGGSGGSGGSGGSGGSDIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIK

SEQ ID NO: 133 - protein, 491 aa, synthetic construct
  source 1..491: note = antibody scDb
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSSGGSGGSDIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIKGGSGGSGGSGGSGGSGGSGGSQVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSSGGSGGSDIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIK

SEQ ID NO: 134 - protein, 725 aa, synthetic construct
  source 1..725: note = artificial antibody construct heavy chain
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSSYELTQPLSVSVALGQTARITCGGHNIGSKNVHWYQQKPGQAPVLVIYQDNKRPSGIPERFSGSNSGNTATLTISRAQAGDEADYYCQVWDNYNVLFGCGTKLTVLGGSGGSGGSGGSGGSGGSGGSQVQLVQSGAEVKKPGASVKVSCKASGYTFTSYYMHWVRQAPGQCLEWMGAIEPTYGSTSYAQKFQGRVTMTRDTSTSTVYMELSSLRSEDTAVYYCARGSAYYYDFADYWGQGTLVTVSS

SEQ ID NO: 135 - protein, 217 aa, synthetic construct
  source 1..217: note = artificial antibody construct light chain
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 725 aa, synthetic construct
  source 1..725: note = artificial antibody construct heavy chain
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSSYELTQPLSVSVALGQTARITCGGHNIGSKNVHWYQQKPGQAPVLVIYQDNKRPSGIPERFSGSNSGNTATLTISRAQAGDEADYYCQVWDNYNVLFGCGTKLTVLGGSGGSGGSGGSGGSGGSGGSQVQLVQSGAEVKKPGASVKVSCKASGYTFTSYYMHWVRQAPGQCLEWMGAIEPRYGSTSYAQKFQGRVTMTRDTSTSTVYMELSSLRSEDTAVYYCARGSAYYYDFADYWGQGTLVTVSS

SEQ ID NO: 137 - protein, 217 aa, synthetic construct; identical to SEQ ID NO: 135
  source 1..217: note = artificial antibody construct light chain

SEQ ID NO: 138 - protein, 725 aa, synthetic construct
  source 1..725: note = artificial antibody construct heavy chain
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSSYELTQPLSVSVALGQTARITCGGNNIGSKSVHWYQQKPGQAPVLVIYQDKKRPSGIPERFSGSNSGNTATLTISRAQAGDEADYYCQVWDDYIVLFGCGTKLTVLGGSGGSGGSGGSGGSGGSGGSQVQLVQSGAEVKKPGASVKVSCKASGYTFTNYYMQWVRQAPGQCLEWMGIINPSGGVTSYAQKFQGRVTMTRDTSTSTVYMELSSLRSEDTAVYYCARGSAYYYDFADYWGQGTLVTVSS

SEQ ID NO: 139 - protein, 217 aa, synthetic construct; identical to SEQ ID NO: 135
  source 1..217: note = artificial antibody construct light chain

SEQ ID NO: 140 - protein, 724 aa, synthetic construct
  source 1..724: note = artificial antibody construct heavy chain
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSSYELTQPLSVSVALGQTARITCGGHNIGSKNVHWYQQKPGQAPVLVIYQDNKRPSGIPERFSGSNSGNTATLTISRAQAGDEADYYCQVWDNYNVLFGCGTKLTVLGGSGGSGGSGGSGGSGGSGGSQVQLVQSGAEVKKPGASVKVSCKASGYTFTSYYMHWVRQAPGQCLEWMGAIEPTYGSTSYAQKFQGRVTMTRDTSTSTVYMELSSLRSEDTAVYYCARGSAYYYDFADYWGQGTLVTVS

SEQ ID NO: 141 - protein, 217 aa, synthetic construct; identical to SEQ ID NO: 135
  source 1..217: note = artificial antibody construct light chain

SEQ ID NO: 142 - protein, 726 aa, synthetic construct
  source 1..726: note = artificial antibody construct heavy chain
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSSYELTQPLSVSVALGQTARITCGGNNIGSKSVHWYQQKPGQAPVLVIYQDKKRPSGIPERFSGSNSGNTATLTISRAQAGDEADYYCQVWDDYIVLFGCGTKLTVLGGSGGSGGSGGSGGSGGSGGSQVQLVQSGAEVKKPGASVKVSCKASGYTFTNYYMQWVRQAPGQCLEWMGIINPSGGVTSYAQKFQGRVTMTRDTSTSTVYMELSSLRSEDTAVYYCARGSAYYYDFADYWGQGTLVTVSSG

SEQ ID NO: 143 - protein, 217 aa, synthetic construct; identical to SEQ ID NO: 135
  source 1..217: note = artificial antibody construct light chain

SEQ ID NO: 144 - protein, 702 aa, synthetic construct
  source 1..702: note = artificial antibody construct heavy chain
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSGGSGGSGGSGGSGGSGGSDIQMTQSPSTLSASVGDRVTITCSASSRVGYMHWYQQKPGKAPKLLIYDTSKLASGVPSRFSGSGSGTEFTLTISSLQPDDFATYYCFQGSGYPFTFGGGTKVEIK

SEQ ID NO: 145 - protein, 217 aa, synthetic construct; identical to SEQ ID NO: 135
  source 1..217: note = artificial antibody construct light chain

SEQ ID NO: 146 - protein, 7 aa, synthetic construct
  source 1..7: note = antibody CDR-H1 sequence
TAGMSVG

SEQ ID NO: 147 - protein, 16 aa, synthetic construct
  source 1..16: note = antibody CDR-H2 sequence
DIWWDDKKHYNPSLKD

SEQ ID NO: 148 - protein, 10 aa, synthetic construct
  source 1..10: note = antibody CDR-H3 sequence
DMIFNFYFDV

SEQ ID NO: 149 - protein, 10 aa, synthetic construct
  source 1..10: note = antibody CDR-L1 sequence
SASSRVGYMH

SEQ ID NO: 150 - protein, 7 aa, synthetic construct
  source 1..7: note = antibody CDR-L2 sequence
DTSKLAS

SEQ ID NO: 151 - protein, 9 aa, synthetic construct
  source 1..9: note = antibody CDR-L3 sequence
FQGSGYPFT

SEQ ID NO: 152 - protein, 120 aa, synthetic construct [sequence omitted]

SEQ ID NO: 153 - protein, 106 aa, synthetic construct [sequence omitted]

SEQ ID NO: 154 - protein, 244 aa, synthetic construct
  source 1..244: note = antibody scFv
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSGGSGGSGGSGGSGGSGGSDIQMTQSPSTLSASVGDRVTITCSASSRVGYMHWYQQKPGKAPKLLIYDTSKLASGVPSRFSGSGSGTEFTLTISSLQPDDFATYYCFQGSGYPFTFGGGTKVEIK

SEQ ID NO: 155 - protein, 485 aa, synthetic construct
  source 1..485: note = antibody scDb
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSGGSGGSDIQMTQSPSTLSASVGDRVTITCSASSRVGYMHWYQQKPGKAPKLLIYDTSKLASGVPSRFSGSGSGTEFTLTISSLQPDDFATYYCFQGSGYPFTFGGGTKVEIKGGSGGSGGSGGSGGSGGSGGSQVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSGGSGGSDIQMTQSPSTLSASVGDRVTITCSASSRVGYMHWYQQKPGKAPKLLIYDTSKLASGVPSRFSGSGSGTEFTLTISSLQPDDFATYYCFQGSGYPFTFGGGTKVEIK

SEQ ID NO: 156 - protein, 727 aa, synthetic construct
  source 1..727: note = scFv-IgAb_444_HC
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSSYELTQPLSVSVALGQTARITCGGNNIGSKSVHWYQQKPGQAPVLVIYQDKKRPSGIPERFSGSNSGNTATLTISRAQAGDEADYYCQVWDDYIVLFGCGTKLTVLGGSGGSGGSGGSGGSGGSGGSQVQLVQSGAEVKKPGASVKVSCKASGYTFTNYYMQWVRQAPGQCLEWMGIINPSGGVTSYAQKFQGRVTMTRDTSTSTVYMELSSLRSEDTAVYYCARGSAYYYDFADYWGQGTLVTVSSG

SEQ ID NO: 157 - protein, 213 aa, synthetic construct
  source 1..213: note = scFv-IgAb_444_LC
DIQMTQSPSTLSASVGDRVTITCSASSRVGYMHWYQQKPGKAPKLLIYDTSKLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 158 - protein, 449 aa, synthetic construct
  source 1..449: note = IgAb_335_HC
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 217 aa, synthetic construct; identical to SEQ ID NO: 135
  source 1..217: note = IgAb_335_LC

SEQ ID NO: 160 - protein, 726 aa, synthetic construct
  source 1..726: note = scFv-IgAb_238_HC
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSSYELTQPLSVSVALGQTARITCGGHNIGSKNVHWYQQKPGQAPVLVIYQDNKRPSGIPERFSGSNSGNTATLTISRAQAGDEADYYCQVWDNYNVLFGCGTKLTVLGGSGGSGGSGGSGGSGGSGGSEVQLVQSGAEVKKPGASVKVSCKASGYTFTSYYMHWVRQAPGQCLEWMGAIEPRYGSTSYAQKFQGRVTMTRDTSTSTVYMELSSLRSEDTAVYYCARGSAYYYDFADYWGQGTLVTVSS

SEQ ID NO: 161 - protein, 213 aa, synthetic construct; identical to SEQ ID NO: 157
  source 1..213: note = scFv-IgAb_238_LC

SEQ ID NO: 162 - protein, 726 aa, synthetic construct
  source 1..726: note = scFv-IgAb_239_HC
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSSYELTQPLSVSVALGQTARITCGGNNIGSKSVHWYQQKPGQAPVLVIYQDKKRPSGIPERFSGSNSGNTATLTISRAQAGDEADYYCQVWDDYIVLFGCGTKLTVLGGSGGSGGSGGSGGSGGSGGSEVQLVQSGAEVKKPGASVKVSCKASGYTFTNYYMQWVRQAPGQCLEWMGIINPSGGVTSYAQKFQGRVTMTRDTSTSTVYMELSSLRSEDTAVYYCARGSAYYYDFADYWGQGTLVTVSS

SEQ ID NO: 163 - protein, 213 aa, synthetic construct; identical to SEQ ID NO: 157
  source 1..213: note = scFv-IgAb_239_LC